(19) United States
(12) Patent Application Publication
(10) Pub. No.: US 2009/0104218 A1
(43) Pub. Date: Apr. 23, 2009

(54) Group B Streptococcus

(75) Inventors: Herve Tettelin, Ellicott City, MD (US); Vega Masignani, Siena (IT)

Correspondence Address: NOVARTIS VACCINES AND DIAGNOSTICS INC., INTELLECTUAL PROPERTY R338, P.O. BOX 8097, Emeryville, CA, 94662-8097, US

(73) Assignees: J. CRAIG VENTER INSTITUTE, Rockville, MD (US); NOVARTIS VACCINES AND DIAGNOSTICS, S.R.L., Siena (IT)

(21) Appl. No.: 11/793,435

(22) PCT Filed: Dec. 21, 2005
(86) PCT No.: PCT/US2005/046491

§ 371 Date: Aug. 25, 2008

Related U.S. Application Data
(60) Provisional application No. 60/638,943, filed on Dec. 22, 2004.
(60) Provisional application No. 60/640,438, filed on Dec. 30, 2004.

(51) Int. Cl. A61K 39/00; C07K 14/00; C07K 16/18; A61P 31/00; C07H 21/04; A61K 38/00

(52) U.S. Cl. 424/190.1; 530/350; 530/387.9; 536/23.7; 514/12

(57) ABSTRACT

Polypeptides and nucleic acids from Streptococcus agalactiae which can be used in the development of vaccines, for diagnostic purposes, and as targets for antibiotics.

This application incorporates by reference the contents of each of two duplicate CD-ROMs which contain an identical 90.1 MB file labeled “PP28007 PCT sequence listing.txt,” which is the sequence listing for this application. The CD-ROMs were created on Dec. 21, 2005. This application also incorporates by reference the contents of each of two duplicate CD-ROMs which contain an identical 681 KB file labeled “Table I.txt” and containing Table I. The CD-ROMs were created on Dec. 20, 2005.

All documents cited herein are incorporated by reference in their entirety.

### TECHNICAL FIELD

This invention is in the field of Streptococcus biology, and in particular relates to S. agalactiae, also known as ‘group B streptococcus’ (GBS).

### BACKGROUND ART

Once thought to infect only cows, the Gram-positive bacterium Streptococcus agalactiae (or “group B streptococcus”, abbreviated to “GBS”) is now known to cause serious disease, bacteremia and meningitis, in immunocompromised individuals and in neonates. There are two types of neonatal infection. The first (early onset, usually within 5 days of birth) is manifested by bacteremia and pneumonia. It is contracted vertically as a baby passes through the birth canal. GBS colonises the vagina of about 25% of young women, and approximately 1% of infants born via a vaginal birth to colonised mothers will become infected. Mortality is between 50-70%. The second is a meningitis that occurs 10 to 60 days after birth. If pregnant women are vaccinated with type III capsule so that the infants are passively immunised, the incidence of the late onset meningitis is reduced but is not entirely eliminated.

The “B” in “GBS” refers to the Lancefield classification, which is based on the antigenicity of a carbohydrate which is soluble in dilute acid and called the C carbohydrate. Lancefield identified 13 types of C carbohydrate, designated A to 0, that could be serologically differentiated. The organisms that most commonly infect humans are found in groups A, B, D, and G. Within group B, strains can be divided into 8 serotypes (Ia, Ib, Ia/c, II, III, IV, V, and VI) based on the structure of their polysaccharide capsule. The genome sequence of a serotype V strain of GBS has been published and analysed [1,2], including a comparative genome hybridization analysis of 19 disease-causing isolates of the same type V strain 2603V/R. The genome sequence of a serotype III strain is also known [3].

Current GBS vaccines are based on polysaccharide antigens, although these suffer from poor immunogenicity. Anti-idiotypic approaches have also been used (e.g. ref. 4). There remains a need, however, for effective adult vaccines against S. agalactiae infection.

It is an object of the invention to provide proteins which can be used in the development of such vaccines. The proteins may also be useful for diagnostic purposes, and as targets for antibiotics.

### DISCLOSURE OF THE INVENTION

### Polypeptides

The invention provides polypeptides comprising the GBS amino acid sequences disclosed in the examples. These amino acid sequences are the even SEQ ID NOs between 2 and 22740. There are thus 11370 amino acid sequences. The polypeptides encoded by sequences listed in Table IV have not previously been seen in GBS strains.

The invention also provides polypeptides comprising amino acid sequences that have sequence identity to the GBS amino acid sequences disclosed in the examples. Depending on the particular sequence, the degree of sequence identity is preferably greater than 50% (e.g. 60%, 70%, 75%, 80%, 85%, 90%, 91%, 92%, 93%, 94%, 95%, 96%, 97%, 98%, 99% or more). These polypeptides include homologs, orthologs, allelic variants and functional mutants. Typically, 50% identity or more between two polypeptide sequences is considered to be an indication of functional equivalence. Identity between polypeptides is preferably determined by the Smith-Waterman homology search algorithm as implemented in the MPSRCH program (Oxford Molecular), using an affine gap search with parameters gap open penalty=12 and gap extension penalty=1.

These polypeptide may, compared to the GBS sequences of the examples, include one or more (e.g. 1, 2, 3, 4, 5, 6, 7, 8, 9, 10, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. The polypeptides may have one or more (e.g. 1, 2, 3, 4, 5, 6, 7, 8, 9, 10, etc.) single amino acid deletions relative to the GBS sequences of the examples. The polypeptides may also include one or more (e.g. 1, 2, 3, 4, 5, 6, 7, 8, 9, 10, etc.) insertions (e.g. each of 1, 2, 3, 4 or 5 amino acids) relative to the GBS sequences of the examples. Some of these deletions, insertions or substitutions may convert one sequence of the invention to another sequence of the invention e.g. amino acids 180-230 of SEQ ID NO: 8614 (identical to amino acids 173-223 of SEQ ID NO: 14060 and amino acids 4-54 of SEQ ID NO: 3916) become amino acids 180-230 of SEQ ID NO: 12908 by conservative substitution of Ile-185 for Val.

Preferred polypeptides of the invention are listed below, including polypeptides that are lipidated, that are located in the outer membrane, that are located in the inner membrane, or that are located in the periplasm. Particularly preferred polypeptides are those that fall into more than one of these categories e.g. lipidated polypeptides that are located in the outer membrane. Lipoproteins may have a N-terminal cysteine to which lipid is covalently attached, following post-translational processing of the signal peptide.

The invention further provides polypeptides comprising fragments of the GBS amino acid sequences disclosed in the examples. The fragments should comprise at least n consecutive amino acids from the sequences and, depending on the particular sequence, n is 7 or more (e.g. 8, 10, 12, 14, 16, 18, 20, 22, 24, 26, 28, 30, 35, 40, 45, 50, 60, 70, 80, 90, 100 or more).

The fragment may comprise at least one T-cell or, preferably, a B-cell epitope of the sequence. T- and B-cell epitopes can be identified empirically (e.g. using PEPSCAN [5,6] or similar methods), or they can be predicted (e.g. using the Jameson-Wolf antigenic index [7], matrix-based approaches [8], TEPITOPE [9], neural networks [10], OptiMer & EpiMer [11, 12], ADEPT [13], Tsites [14], hydrophilicity [15], antigenic index [16] or the methods disclosed in reference 17, etc.). Other preferred fragments are (a) the N-terminal signal peptides of the GBS polypeptides of the invention, (b) the GBS polypeptides, but without their N-terminal signal peptides, (c) the GBS polypeptides, but without their N-terminal amino acid residue.

Further preferred fragments are those common to at least two (e.g. 2, 3, 4 or 5) homologous coding sequences, and in particular those common to homologous coding sequences within the sequence listing. Table II shows homologous SEQ ID numbers for nucleic acids within the sequence listing e.g. SEQ ID NOs: 88, 4374, 8834, 13214 and 17994 are homologous within the sequence listing, and are also homologous with prior art GI sequences 22533036 and 23094457. Simple alignments show that amino acids 1-131 of these five SEQ ID NOs are common, as are amino acids 133-176, 178-182, 184-190, 192-217, 219-250, 252-278, 280-322, 324-366, 368-373 and 375-434. Similarly, 1-176 are common to SEQ ID NOs: 88, 4374, 8834 and 13214, but not to 17994. Thus fragments 1-131, 1-176 and 133-176 are all preferred fragments of the invention. In some cases, where homologous sequences are 100% identical between strains along their complete lengths (e.g. SEQ ID NOs: 2, 8616, 12910, 14062 and 22384), the common ‘fragment’ will in fact be the complete sequence.

Other preferred fragments are those that begin with an amino acid encoded by a potential start codon (ATG, GTG, TTG). Fragments starting at the methionine encoded by a start codon downstream of the indicated start codon are polypeptides of the invention.

Polypeptides of the invention can be prepared in many ways e.g. by chemical synthesis (in whole or in part), by digesting longer polypeptides using proteases, by translation from RNA, by purification from cell culture (e.g. from recombinant expression), from the organism itself (e.g. after bacterial culture, or direct from patients), etc. A preferred method for production of peptides <40 amino acids long involves in vitro chemical synthesis [18, 19]. Solid-phase peptide synthesis is particularly preferred, such as methods based on tBoc or Fmoc [20] chemistry. Enzymatic synthesis [21] may also be used in part or in full. As an alternative to chemical synthesis, biological synthesis may be used e.g. the polypeptides may be produced by translation. This may be carried out in vitro or in vivo. Biological methods are in general restricted to the production of polypeptides based on L-amino acids, but manipulation of translation machinery (e.g. of aminoacyl tRNA molecules) can be used to allow the introduction of D-amino acids (or of other non natural amino acids, such as iodotyrosine or methylphenylalanine, azidohomoalanine, etc.) [22]. Where D-amino acids are included, however, it is preferred to use chemical synthesis. Polypeptides of the invention may have covalent modifications at the C-terminus and/or N-terminus.

Polypeptides of the invention can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.).

Polypeptides of the invention are preferably provided in purified or substantially purified form i.e. substantially free from other polypeptides (e.g. free from naturally-occurring polypeptides), particularly from other streptococcal or host cell polypeptides, and are generally at least about 50% pure (by weight), and usually at least about 90% pure i.e. less than about 50%, and more preferably less than about 10% (e.g. 5%) of a composition is made up of other expressed polypeptides.

Polypeptides of the invention are preferably GBS polypeptides. Polypeptides of the invention preferably have the function indicated in Table I for the relevant sequence.

Polypeptides of the invention may be attached to a solid support. Polypeptides of the invention may comprise a detectable label (e.g. a radioactive or fluorescent label, or a biotin label). The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

Polypeptides can occur as single chains or associated chains. Polypeptides of the invention can be naturally or non-naturally glycosylated (i.e. the polypeptide has a glycosylation pattern that differs from the glycosylation pattern found in the corresponding naturally occurring polypeptide). Polypeptides of the invention may be at least 40 amino acids long (e.g. at least 40, 50, 60, 70, 80, 90, 100, 120, 140, 160, 180, 200, 220, 240, 260, 280, 300, 350, 400, 450, 500 or more). Polypeptides of the invention may be shorter than 500 amino acids (e.g. no longer than 40, 50, 60, 70, 80, 90, 100, 120, 140, 160, 180, 200, 220, 240, 260, 280, 300, 350, 400 or 450 amino acids).

The invention provides polypeptides comprising a sequence —X—Y— or —Y—X—, wherein: —X— is an amino acid sequence as defined above and —Y— is not a sequence as defined above i.e. the invention provides fusion proteins. Where the N-terminus codon of a polypeptide-coding sequence is not ATG then that codon will be translated as the standard amino acid for that codon rather than as a Met, which occurs when the codon is translated as a start codon.

The invention provides a process for producing polypeptides of the invention, comprising the step of culturing a host cell of to the invention under conditions which induce polypeptide expression.

The invention provides a process for producing a polypeptide of the invention, wherein the polypeptide is synthesised in part or in whole using chemical means.

The invention provides a composition comprising two or more polypeptides of the invention.

The invention also provides a hybrid polypeptide represented by the formula NH₂-A-[-X-L-]ₙ-B—COOH, wherein X is a polypeptide of the invention as defined above, L is an optional linker amino acid sequence, A is an optional N-terminal amino acid sequence, B is an optional C-terminal amino acid sequence, and n is an integer greater than 1. The value of n is between 2 and x, and the value of x is typically 3, 4, 5, 6, 7, 8, 9 or 10. Preferably n is 2, 3 or 4; it is more preferably 2 or 3; most preferably, n=2. For each n instances, —X— may be the same or different. For each n instances of [—X-L-], linker amino acid sequence -L- may be present or absent. For instance, when n=2 the hybrid may be NH₂—X₁-L₁-X₂-L₂-COOH, NH₂—X₁—X₂—COOH, NH₂—X₁-L₁-X₂—COOH, NH₂—X₁—X₂-L₂-COOH, etc. Linker amino acid sequence(s)-L- will typically be short (e.g. 20 or fewer amino acids i.e. 19, 18, 17, 16, 15, 14, 13, 12, 11, 10, 9, 8, 7, 6, 5, 4, 3, 2, 1). Examples include short peptide sequences which facilitate cloning, poly-glycine linkers (i.e. Gly, where n=2, 3, 4, 5, 6, 7, 8, 9, 10 or more), and histidine tags (i.e. HiSₙ where n=3, 4, 5, 6, 7, 8, 9, 10 or more). Other suitable linker amino acid sequences will be apparent to those skilled in the art. -A- and -B- are optional sequences which will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct polypeptide trafficking, or short peptide sequences which facilitate cloning or purification (e.g. histidine tags i.e. Hisₙ where n=3, 4, 5, 6, 7, 8, 9, 10 or more). Other suitable N-terminal and C-terminal amino acid sequences will be apparent to those skilled in the art.

Various tests can be used to assess the in vivo immunogenicity of polypeptides of the invention. For example, polypeptides can be expressed recombinantly and used to screen patient sera by immunoblot. A positive reaction between the polypeptide and patient serum indicates that the patient has previously mounted an immune response to the protein in question i.e. the protein is an immunogen. This method can also be used to identify immunodominant proteins.

### Antibodies

The invention provides antibodies that bind to polypeptides of the invention. These may be polyclonal or monoclonal and may be produced by any suitable means (e.g. by recombinant expression). To increase compatibility with the human immune system, the antibodies may be chimeric or humanised [e.g. refs. 23 & 24], or fully human antibodies may be used. The antibodies may include a detectable label (e.g. for diagnostic assays). Antibodies of the invention may be attached to a solid support. Antibodies of the invention are preferably neutralising antibodies.

Monoclonal antibodies are particularly useful in identification and purification of the individual polypeptides against which they are directed. Monoclonal antibodies of the invention may also be employed as reagents in immunoassays, radioimmunoassays (RIA) or enzyme-linked immunosorbent assays (ELISA), etc. In these applications, the antibodies can be labelled with an analytically-detectable reagent such as a radioisotope, a fluorescent molecule or an enzyme. The monoclonal antibodies produced by the above method may also be used for the molecular identification and characterization (epitope mapping) of polypeptides of the invention. Antibodies of the invention are preferably specific to Streptococci i.e. they bind preferentially to Streptococci bacteria relative to non-Streptococci bacteria. More preferably, the antibodies are specific to GBS i.e. they bind preferentially to GBS bacteria relative to non-type-b streptococci.

Antibodies of the invention are preferably provided in purified or substantially purified form. Typically, the antibody will be present in a composition that is substantially free of other polypeptides e.g. where less than 90% (by weight), usually less than 60% and more usually less than 50% of the composition is made up of other polypeptides.

Antibodies of the invention can be of any isotype (e.g. IgA, IgG, IgM i.e. an α, γ or μ heavy chain), but will generally be IgG. Within the IgG isotype, antibodies may be IgG1, IgG2, IgG3 or IgG4 subclass. Antibodies of the invention may have a κ or a λ light chain.

Antibodies of the invention can take various forms, including whole antibodies, antibody fragments such as F(ab′)₂ and F(ab) fragments, Fv fragments (non-covalent heterodimers), single-chain antibodies such as single chain Fv molecules (scFv), minibodies, oligobodies, etc. The term “antibody” does not imply any particular origin, and includes antibodies obtained through non-conventional processes, such as phage display.

The invention provides a process for detecting polypeptides of the invention, comprising the steps of: (a) contacting an antibody of the invention with a biological sample under conditions suitable for the formation of an antibody-antigen complexes; and (b) detecting said complexes.

The invention provides a process for detecting antibodies of the invention, comprising the steps of: (a) contacting a polypeptide of the invention with a biological sample (e.g. a blood or serum sample) under conditions suitable for the formation of an antibody-antigen complexes; and (b) detecting said complexes.

For good cross-reactivity, preferred antibodies of the invention bind to epitopes within fragments that are common to at least two (e.g. 2, 3, 4 or 5) homologous coding sequences, as described in more detail above. Conversely, for good specificity, other preferred antibodies of the invention bind to epitopes that include an amino acid that differs between homologous coding sequences e.g. binds to Phe-132 in SEQ ID NO: 17994 to distinguish from SEQ ID NOs: 88, 4374, 8834 and 13214, all of which have a Serine residue at position 132.

### Nucleic Acids

The invention provides nucleic acid comprising the GBS nucleotide sequences disclosed in the examples. These nucleic acid sequences are the odd SEQ ID NOs between 1 and 22739.

The invention also provides nucleic acid comprising nucleotide sequences having sequence identity to the GBS nucleotide sequences disclosed in the examples. Identity between sequences is preferably determined by the Smith-Waterman homology search algorithm as described above.

The invention also provides nucleic acid which can hybridize to the GBS nucleic acid disclosed in the examples. Hybridization reactions can be performed under conditions of different “stringency”. Conditions that increase stringency of a hybridization reaction of widely known and published in the art [e.g. page 7.52 of reference 25]. Examples of relevant conditions include (in order of increasing stringency): incubation temperatures of 25° C., 37° C., 50° C., 55° C. and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or de-ionized water. Hybridization techniques and their optimization are well known in the art [e.g. see refs 25-28, etc.].

In some embodiments, nucleic acid of the invention hybridizes to a target of the invention under low stringency conditions; in other embodiments it hybridizes under intermediate stringency conditions; in preferred embodiments, it hybridizes under high stringency conditions. An exemplary set of low stringency hybridization conditions is 50° C. and 10×SSC. An exemplary set of intermediate stringency hybridization conditions is 55° C. and 1×SSC. An exemplary set of high stringency hybridization conditions is 68° C. and 0.1×SSC.

Nucleic acid comprising fragments of these sequences are also provided. These should comprise at least n consecutive nucleotides from the GBS sequences and, depending on the particular sequence, n is 10 or more (e.g. 12, 14, 15, 18, 20, 25, 30, 35, 40, 50, 60, 70, 80, 90, 100, 150, 200 or more).

The invention provides nucleic acid of formula 5′-X—Y-Z-3′, wherein: —X— is a nucleotide sequence consisting of x nucleotides; -Z- is a nucleotide sequence consisting of z nucleotides; —Y— is a nucleotide sequence consisting of either (a) a fragment of one of the odd-numbered SEQ ID NOs: 1 to 22739, or (b) the complement of (a); and said nucleic acid 5′-X—Y-Z-3′ is neither (i) a fragment of one of the odd-numbered SEQ ID NOs: 1 to 22739 nor (ii) the complement of (i). The —X— and/or -Z-moieties may comprise a promoter sequence (or its complement).

The invention also provides nucleic acid encoding the polypeptides and polypeptide fragments of the invention.

The invention includes nucleic acid comprising sequences complementary to the sequences disclosed in the sequence listing (e.g. for antisense or probing, or for use as primers), as well as the sequences in the orientation actually shown.

Nucleic acids of the invention can be used in hybridisation reactions (e.g. Northern or Southern blots, or in nucleic acid microarrays or ‘gene chips’) and amplification reactions (e.g. PCR, SDA, SSSR, LCR, TMA, NASBA, etc.) and other nucleic acid techniques.

Nucleic acid according to the invention can take various forms (e.g. single-stranded, double-stranded, vectors, primers, probes, labelled etc.). Nucleic acids of the invention may be circular or branched, but will generally be linear. Unless otherwise specified or required, any embodiment of the invention that utilizes a nucleic acid may utilize both the double-stranded form and each of two complementary single-stranded forms which make up the double-stranded form. Primers and probes are generally single-stranded, as are antisense nucleic acids.

Nucleic acids of the invention are preferably provided in purified or substantially purified form i.e. substantially free from other nucleic acids (e.g. free from naturally-occurring nucleic acids), particularly from other streptococcal or host cell nucleic acids, generally being at least about 50% pure (by weight), and usually at least about 90% pure. Nucleic acids of the invention are preferably GBS nucleic acids.

Nucleic acids of the invention may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

Nucleic acid of the invention may be attached to a solid support (e.g. a bead, plate, filter, film, slide, microarray support, resin, etc.). Nucleic acid of the invention may be labelled e.g. with a radioactive or fluorescent label, or a biotin label. This is particularly useful where the nucleic acid is to be used in detection techniques e.g. where the nucleic acid is a primer or as a probe.

The term “nucleic acid” includes in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. Thus the invention includes mRNA, tRNA, rRNA, ribozymes, DNA, cDNA, recombinant nucleic acids, branched nucleic acids, plasmids, vectors, probes, primers, etc. Where nucleic acid of the invention takes the form of RNA, it may or may not have a 5′ cap.

Nucleic acids of the invention comprise GBS sequences, but they may also comprise non-GBS sequences (e.g. in nucleic acids of formula 5′-X—Y-Z-3′, as defined above). This is particularly useful for primers, which may thus comprise a first sequence complementary to a GBS nucleic acid target and a second sequence which is not complementary to the nucleic acid target. Any such non-complementary sequences in the primer are preferably 5′ to the complementary sequences. Typical non-complementary sequences comprise restriction sites or promoter sequences.

Nucleic acids of the invention can be prepared in many ways e.g. by chemical synthesis (at least in part), by digesting longer nucleic acids using nucleases (e.g. restriction enzymes), by joining shorter nucleic acids (e.g. using ligases or polymerases), from genomic or cDNA libraries, etc.

Nucleic acids of the invention may be part of a vector i.e. part of a nucleic acid construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, “viral vectors” which is designed to result in the production of a recombinant virus or virus-like particle, or “shuttle vectors”, which comprise the attributes of more than one type of vector. Preferred vectors are plasmids. A “host cell” includes an individual cell or cell culture which can be or has been a recipient of exogenous nucleic acid. Host cells include progeny of a single host cell, and the progeny may not necessarily be completely identical (in morphology or in total DNA complement) to the original parent cell due to natural, accidental, or deliberate mutation and/or change. Host cells include cells transfected or infected in vivo or in vitro with nucleic acid of the invention.

Where a nucleic acid is DNA, it will be appreciated that “U” in a RNA sequence will be replaced by “T” in the DNA. Similarly, where a nucleic acid is RNA, it will be appreciated that “T” in a DNA sequence will be replaced by “U” in the RNA.

The term “complement” or “complementary” when used in relation to nucleic acids refers to Watson-Crick base pairing. Thus the complement of C is G, the complement of G is C, the complement of A is T (or U), and the complement of T (or U) is A. It is also possible to use bases such as I (the purine inosine) e.g. to complement pyrimidines (C or T). The terms also imply a direction the complement of 5′-ACAGT-3′ is 5′-ACTGT-3′ rather than 5′-TGTCA-3′.

Nucleic acids of the invention can be used, for example: to produce polypeptides; as hybridization probes for the detection of nucleic acid in biological samples; to generate additional copies of the nucleic acids; to generate ribozymes or antisense oligonucleotides; as single-stranded DNA primers or probes; or as triple-strand forming oligonucleotides.

The invention provides a process for producing nucleic acid of the invention, wherein the nucleic acid is synthesised in part or in whole using chemical means.

The invention provides vectors comprising nucleotide sequences of the invention (e.g. cloning or expression vectors) and host cells transformed with such vectors.

The invention also provides a kit comprising primers (e.g. PCR primers) for amplifying a template sequence contained within a streptococcus bacterium (e.g. GBS) nucleic acid sequence, the kit comprising a first primer and a second primer, wherein the first primer is substantially complementary to said template sequence and the second primer is substantially complementary to a complement of said template sequence, wherein the parts of said primers which have substantial complementarity define the termini of the template sequence to be amplified. The first primer and/or the second primer may include a detectable label (e.g. a fluorescent label).

The invention also provides a kit comprising first and second single-stranded oligonucleotides which allow amplification of a streptococcal template nucleic acid sequence contained in a single- or double-stranded nucleic acid (or mixture thereof), wherein: (a) the first oligonucleotide comprises a primer sequence which is substantially complementary to said template nucleic acid sequence; (b) the second oligonucleotide comprises a primer sequence which is substantially complementary to the complement of said template nucleic acid sequence; (c) the first oligonucleotide and/or the second oligonucleotide comprise(s) sequence which is not complementary to said template nucleic acid; and (d) said primer sequences define the termini of the template sequence to be amplified. The non-complementary sequence(s) of feature (c) are preferably upstream of (i.e. 5′ to) the primer sequences. One or both of these (c) sequences may comprise a restriction site [e.g. ref. 29] or a promoter sequence [e.g. 30]. The first oligonucleotide and/or the second oligonucleotide may include a detectable label (e.g. a fluorescent label).

The invention provides a process for detecting nucleic acid of the invention, comprising the steps of: (a) contacting a nucleic probe according to the invention with a biological sample under hybridising conditions to form duplexes; and (b) detecting said duplexes.

The invention provides a process for detecting GBS in a biological sample (e.g. blood), comprising the step of contacting nucleic acid according to the invention with the biological sample under hybridising conditions. The process may involve nucleic acid amplification (e.g. PCR, SDA, SSSR, LCR, TMA, NASBA, etc.) or hybridisation (e.g. microarrays, blots, hybridisation with a probe in solution etc.). PCR detection of GBS in clinical samples has been reported [e.g. see refs. 31 to 34]. Clinical assays based on nucleic acid are described in general in ref. 35.

The invention provides a process for preparing a fragment of a target sequence, wherein the fragment is prepared by extension of a nucleic acid primer. The target sequence and/or the primer are nucleic acids of the invention. The primer extension reaction may involve nucleic acid amplification (e.g. PCR, SDA, SSSR, LCR, TMA, NASBA, etc.).

Nucleic acid amplification according to the invention may be quantitative and/or real-time.

For certain embodiments of the invention, nucleic acids are preferably at least 7 nucleotides in length (e.g. 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 90, 100, 110, 120, 130, 140, 150, 160, 170, 180, 190, 200, 225, 250, 275, 300 nucleotides or longer).

For certain embodiments of the invention, nucleic acids are preferably at most 500 nucleotides in length (e.g. 450, 400, 350, 300, 250, 200, 150, 140, 130, 120, 110, 100, 90, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15 nucleotides or shorter).

Primers and probes of the invention, and other nucleic acids used for hybridization, are preferably between 10 and 30 nucleotides in length (e.g. 10, 11, 12, 13, 14, 15, 16, 17, 18, 19, 20, 21, 22, 23, 24, 25, 26, 27, 28, 29, or 30 nucleotides).

### Pharmaceutical Compositions

The invention provides compositions comprising: (a) polypeptide, antibody, and/or nucleic acid of the invention; and (b) a pharmaceutically acceptable carrier. These compositions may be suitable as immunogenic compositions, for instance, or as diagnostic reagents, or as vaccines. Vaccines according to the invention may either be prophylactic (i.e. to prevent infection) or therapeutic (i.e. to treat infection), but will typically be prophylactic.

A ‘pharmaceutically acceptable carrier’ includes any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, sucrose, trehalose, lactose, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. Sterile pyrogen-free, phosphate-buffered physiologic saline is a typical carrier. A thorough discussion of pharmaceutically acceptable excipients is available in ref. 155. Compositions of the invention may include an antimicrobial, particularly if packaged in a multiple dose format.

Compositions of the invention may comprise detergent e.g. a Tween (polysorbate), such as Tween 80. Detergents are generally present at low levels e.g. <0.01%. Compositions of the invention may include sodium salts (e.g. sodium chloride) to give tonicity. A concentration of 10+2 mg/ml NaCl is typical.

Compositions of the invention will generally include a buffer. A phosphate buffer is typical. Compositions of the invention may comprise a sugar alcohol (e.g. mannitol) or a disaccharide (e.g. sucrose or trehalose) e.g. at around 15-30 mg/ml (e.g. 25 mg/ml), particularly if they are to be lyophilised or if they include material which has been reconstituted from lyophilised material. The pH of a composition for lyophilisation may be adjusted to around 6.1 prior to lyophilisation. Polypeptides of the invention may be administered in conjunction with other immunoregulatory agents. In particular, composition will usually include a vaccine adjuvant. Adjuvants which may be used in compositions of the invention include, but are not limited to:

### A. Mineral-Containing Compositions

Mineral containing compositions suitable for use as adjuvants in the invention include mineral salts, such as aluminium salts and calcium salts. The invention includes mineral salts such as hydroxides (e.g. oxyhydroxides), phosphates (e.g. hydroxyphosphates, orthophosphates), sulphates, etc. [e.g. see chapters 8 & 9 of ref. 36], or mixtures of different mineral compounds (e.g. a mixture of a phosphate and a hydroxide adjuvant, optionally with an excess of the phosphate), with the compounds taking any suitable form (e.g., gel, crystalline, amorphous, etc.), and with adsorption to the salt(s) being preferred. Mineral containing compositions may also be formulated as a particle of metal salt [37].

Aluminum salts may be included in vaccines of the invention such that the dose of Al³⁺ is between 0.2 and 1.0 mg per dose.

A typical aluminium phosphate adjuvant is amorphous aluminium hydroxyphosphate with PO₄/Al molar ratio between 0.84 and 0.92, included at 0.6 mg Al³⁺/ml. Adsorption with a low dose of aluminium phosphate may be used e.g. between 50 and 100 μg Al³⁺ per conjugate per dose. Where an aluminium phosphate it used and it is desired not to adsorb an antigen to the adjuvant, this is favoured by including free phosphate ions in solution (e.g. by the use of a phosphate buffer).

### B. Oil Emulsions

Oil emulsion compositions suitable for use as adjuvants in the invention include squalene-water emulsions, such as MF59 (5% Squalene, 0.5% Tween 80, and 0.5% Span 85, formulated into submicron particles using a microfluidizer) [Chapter 10 of ref. 36; see also refs. 38-40]. MF59 is used as the adjuvant in the FLUAD™ influenza virus trivalent subunit vaccine.

Particularly preferred adjuvants for use in the compositions are submicron oil-in-water emulsions. Preferred submicron oil-in-water emulsions for use herein are squalene/water emulsions optionally containing varying amounts of MTP-PE, such as a submicron oil-in-water emulsion containing 4-5% w/v squalene, 0.25-1.0% w/v Tween 80 (polyoxyethylenesorbitan monooleate), and/or 0.25-1.0% Span 85 (sorbitan trioleate), and, optionally, N-acetylmuramyl-L-alanyl-D-isogluatminyl-L-alanine-2-(1′-2′-dipalmitoyl-sn-glycero-3-hydroxyphosphosphoryloxy)-ethylamine (MTP-PE). Submicron oil-in-water emulsions, methods of making the same and immunostimulating agents, such as muramyl peptides, for use in the compositions, are described in detail in references 38 & 41-42.

Complete Freund's adjuvant (CFA) and incomplete Freund's adjuvant (IFA) may also be used as adjuvants in the invention.

### C. Saponin Formulations [Chapter 22 of Ref 36]

Saponin formulations may also be used as adjuvants in the invention. Saponins are a heterologous group of sterol glycosides and triterpenoid glycosides that are found in the bark, leaves, stems, roots and even flowers of a wide range of plant species. Saponins isolated from the bark of the Quillaja saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs.

Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in ref. 43. Saponin formulations may also comprise a sterol, such as cholesterol [44].

Combinations of saponins and cholesterols can be used to form unique particles called immunostimulating complexes (ISCOMs) [chapter 23 of ref. 36]. ISCOMs typically also include a phospholipid such as phosphatidylethanolamine or phosphatidylcholine. Any known saponin can be used in ISCOMs. Preferably, the ISCOM includes one or more of QuilA, QHA and QHC. ISCOMs are further described in refs. 44-46. Optionally, the ISCOMs may be devoid of additional detergent(s) [47].

A review of the development of saponin based adjuvants can be found in refs. 48 & 49.

### D. Virosomes and Virus-Like Particles

Virosomes and virus-like particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in refs. 50-55. Virosomes are discussed further in, for example, ref 56

### E. Bacterial or Microbial Derivatives

Adjuvants suitable for use in the invention include bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof.

Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A preferred “small particle” form of 3 De-O-acylated monophosphoryl lipid A is disclosed in ref. 57. Such “small particles” of 3dMPL are small enough to be sterile filtered through a 0.22 μm membrane [57]. Other non-toxic LPS derivatives include monophosphoryl lipid A mimics, such as aminoalkyl glucosaminide phosphate derivatives e.g. RC-529 [58, 59].

Lipid A derivatives include derivatives of lipid A from Escherichia coli such as OM-174. OM-174 is described for example in refs. 60 & 61.

Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory.

The CpG's can include nucleotide modifications/analogs such as phosphorothioate modifications and can be double-stranded or single-stranded. References 62, 63 and 64 disclose possible analog substitutions e.g. replacement of guanosine with 2′-deoxy-7-deazaguanosine. The adjuvant effect of CpG oligonucleotides is further discussed in refs. 65-70.

The CpG sequence may be directed to TLR9, such as the motif GTCGTT or TTCGTT [71]. The CpG sequence may be specific for inducing a Th1 immune response, such as a CpG-A ODN, or it may be more specific for inducing a B cell response, such a CpG-B ODN. CpG-A and CpG-B ODNs are discussed in refs. 72-74. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, refs. 71 & 75-77.

Bacterial ADP-ribosylating toxins and detoxified derivatives thereof may be used as adjuvants in the invention. Preferably, the protein is derived from E. coli (E. coli heat labile enterotoxin “LT”), cholera (“CT”), or pertussis (“PT”). The use of detoxified ADP-ribosylating toxins as mucosal adjuvants is described in ref. 78 and as parenteral adjuvants in ref 79. The toxin or toxoid is preferably in the form of a holotoxin, comprising both A and B subunits. Preferably, the A subunit contains a detoxifying mutation; preferably the B subunit is not mutated. Preferably, the adjuvant is a detoxified LT mutant such as LT-K63, LT-R72, and LT-G192. The use of ADP-ribosylating toxins and detoxified derivatives thereof, particularly LT-K63 and LT-R72, as adjuvants can be found in refs. 80-87. Numerical reference for amino acid substitutions is preferably based on the alignments of the A and B subunits of ADP-ribosylating toxins set forth in ref. 88, specifically incorporated herein by reference in its entirety.

### F. Human Immunomodulators

Human immunomodulators suitable for use as adjuvants in the invention include cytokines, such as interleukins (e.g. IL-1, IL-2, IL-4, IL-5, IL-6, IL-7, IL-12 [89], etc.) [90], interferons (e.g. interferon-γ), macrophage colony stimulating factor, and tumor necrosis factor.

### G. Bioadhesives and Mucoadhesives

Bioadhesives and mucoadhesives may also be used as adjuvants in the invention. Suitable bioadhesives include esterified hyaluronic acid microspheres [91] or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention [92].

### H. Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charges surface (e.g. with SDS) or a positively-charged surface (e.g. with a cationic detergent, such as CTAB).

### I. Liposomes (Chapters 13 & 14 of ref 36)

Examples of liposome formulations suitable for use as adjuvants are described in refs. 93-95.

### J. Polyoxyethylene Ether and Polyoxyethylene Ester Formulations

Adjuvants suitable for use in the invention include polyoxyethylene ethers and polyoxyethylene esters [96]. Such formulations further include polyoxyethylene sorbitan ester surfactants in combination with an octoxynol [97] as well as polyoxyethylene alkyl ethers or ester surfactants in combination with at least one additional non-ionic surfactant such as an octoxynol [98]. Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

### K. Polyphosphazene (PCPP)

PCPP formulations are described, for example, in refs. 99 and 100.

L. Muramyl Peptides

Examples of muramyl peptides suitable for use as adjuvants in the invention include N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine MTP-PE).

### M. Imidazoquinolone Compounds.

Examples of imidazoquinolone compounds suitable for use adjuvants in the invention include Imiquamod and its homologues (e.g. “Resiquimod 3M”), described further in refs. 101 and 102.

### N. Thiosemicarbazone Compounds.

Examples of thiosemicarbazone compounds, as well as methods of formulating, manufacturing, and screening for compounds all suitable for use as adjuvants in the invention include those described in ref. 103. The thiosemicarbazones are particularly effective in the stimulation of human peripheral blood mononuclear cells for the production of cytokines, such as TNF-α.

### O. Tryptanthrin Compounds.

Examples of tryptanthrin compounds, as well as methods of formulating, manufacturing, and screening for compounds all suitable for use as adjuvants in the invention include those described in ref. 104. The tryptanthrin compounds are particularly effective in the stimulation of human peripheral blood mononuclear cells for the production of cytokines, such as TNF-α.

The invention may also comprise combinations of aspects of one or more of the adjuvants identified above. For example, the following combinations may be used as adjuvant compositions in the invention: (1) a saponin and an oil-in-water emulsion [105]; (2) a saponin (e.g. QS21)+a non-toxic LPS derivative (e.g. 3dMPL) [106]; (3) a saponin (e.g. QS21)+a non-toxic LPS derivative (e.g. 3dMPL)+a cholesterol; (4) a saponin (e.g. QS21)+3dMPL+IL-12 (optionally+a sterol) [107]; (5) combinations of 3dMPL with, for example, QS21 and/or oil-in-water emulsions [108]; (6) SAF, containing 10% squalane, 0.4% Tween 80™, 5% pluronic-block polymer L121, and thr-MDP, either microfluidized into a submicron emulsion or vortexed to generate a larger particle size emulsion. (7) Ribi™ adjuvant system (RAS), (Ribi Immunochem) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); (8) one or more mineral salts (such as an aluminum salt)+a non-toxic derivative of LPS (such as 3dMPL); and (9) one or more mineral salts (such as an aluminum salt)+an immunostimulatory oligonucleotide (such as a nucleotide sequence including a CpG motif).

Other substances that act as immunostimulating agents are disclosed in chapter 7 of ref. 36.

The use of an aluminium hydroxide or aluminium phosphate adjuvant is particularly preferred, and antigens are generally adsorbed to these salts. Calcium phosphate is another preferred adjuvant.

The pH of compositions of the invention is preferably between 6 and 8, preferably about 7. Stable pH may be maintained by the use of a buffer. Where a composition comprises an aluminium hydroxide salt, it is preferred to use a histidine buffer [109]. The composition may be sterile and/or pyrogen-free. Compositions of the invention may be isotonic with respect to humans.

Compositions may be presented in vials, or they may be presented in ready-filled syringes. The syringes may be supplied with or without needles. A syringe will include a single dose of the composition, whereas a vial may include a single dose or multiple doses. Injectable compositions will usually be liquid solutions or suspensions. Alternatively, they may be presented in solid form (e.g. freeze-dried) for solution or suspension in liquid vehicles prior to injection.

Compositions of the invention may be packaged in unit dose form or in multiple dose form. For multiple dose forms, vials are preferred to pre-filled syringes. Effective dosage volumes can be routinely established, but a typical human dose of the composition for injection has a volume of 0.5 ml.

Where a composition of the invention is to be prepared extemporaneously prior to use (e.g. where a component is presented in lyophilised form) and is presented as a kit, the kit may comprise two vials, or it may comprise one ready-filled syringe and one vial, with the contents of the syringe being used to reactivate the contents of the vial prior to injection.

Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials, and a typical quantity of each meningococcal saccharide antigen per dose is between 1 μg and 10 mg per antigen.

### Pharmaceutical Uses

The invention also provides a method of treating a patient, comprising administering to the patient a therapeutically effective amount of a composition of the invention. The patient may either be at risk from the disease themselves or may be a pregnant woman (‘maternal immunisation’ [110]).

The invention provides nucleic acid, polypeptide, or antibody of the invention for use as medicaments (e.g. as immunogenic compositions or as vaccines) or as diagnostic reagents. It also provides the use of nucleic acid, polypeptide, or antibody of the invention in the manufacture of: (i) a medicament for treating or preventing disease and/or infection caused by GBS; (ii) a diagnostic reagent for detecting the presence of GBS or of antibodies raised against GBS; and/or (iii) a reagent which can raise antibodies against GBS. Said GBS can be of any serotype or strain. Said disease may be, for instance, bacteremia, meningitis, puerperal fever, scarlet fever, erysipelas, pharyngitis, impetigo, necrotising fasciitis, myositis or toxic shock syndrome.

The patient is preferably a human. Where the vaccine is for prophylactic use, the human is preferably an adolescent (e.g. aged between 10 and 20 years); where the vaccine is for therapeutic use, the human is preferably an adult. A vaccine intended for children or adolescents may also be administered to adults e.g. to assess safety, dosage, immunogenicity, etc.

One way of checking efficacy of therapeutic treatment involves monitoring GBS infection after administration of the composition of the invention. One way of checking efficacy of prophylactic treatment involves monitoring immune responses against an administered polypeptide after administration. Immunogenicity of compositions of the invention can be determined by administering them to test subjects (e.g. children 12-16 months age, or animal models e.g. a mouse model) and then determining standard parameters including ELISA titres (GMT) of IgG. These immune responses will generally be determined around 4 weeks after administration of the composition, and compared to values determined before administration of the composition. Where more than one dose of the composition is administered, more than one post-administration determination may be made. A mouse neonatal sepsis model for protective efficacy against GBS infection is known e.g. see ref. 111.

Administration of polypeptide antigens is a preferred method of treatment for inducing immunity. Administration of antibodies of the invention is another preferred method of treatment. This method of passive immunisation is particularly useful for newborn children or for pregnant women. This method will typically use monoclonal antibodies, which will be humanised or fully human. Preferred compositions for use in immunisation include more than one GBS polypeptide. Multiple antigens can be included as separate admixed polypeptides in a single composition, and/or can be part of a hybrid polypeptide as described above. Preferred combinations of antigens include at least one (e.g. 1, 2, 3, 4, 5, 6 or more) ‘core’ polypeptide (as described below; Table V) and at least one (e.g. 1, 2, 3, 4, 5, 6 or more) ‘variable’ polypeptide (as described below; Table VI). Mixtures of one core polypeptide with more than one variable polypeptides are preferred. Examples of these combinations, using the nomenclature of reference 2, include (a) GBS322 (a core antigen) plus GBS80, GBS104 & GBS67 (all variable antigens); and (b) GBS322 plus GBS80 & GBS104. In some embodiments, this specific 3-valent combination [112] and this specific 4-valent combination [113] are excluded from the invention, although they illustrate the principle of combining core and variable antigens.

Compositions of the invention will generally be administered directly to a patient. Direct delivery may be accomplished by parenteral injection (e.g. subcutaneously, intraperitoneally, intravenously, intramuscularly, or to the interstitial space of a tissue), or by rectal, oral, vaginal, topical, transdermal, intranasal, sublingual, ocular, aural, pulmonary or other mucosal administration. Intramuscular administration to the thigh or the upper arm is preferred injection may be via a needle (e.g. a hypodermic needle), but needle-free injection may alternatively be used. A typical intramuscular dose is 0.5 ml.

The invention may be used to elicit systemic and/or mucosal immunity.

Dosage treatment can be a single dose schedule or a multiple dose schedule. Multiple doses may be used in a primary immunisation schedule and/or in a booster immunisation schedule. A primary dose schedule may be followed by a booster dose schedule. Suitable timing between priming doses (e.g. between 4-16 weeks), and between priming and boosting, can be routinely determined.

Bacterial infections affect various areas of the body and so compositions may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition). The composition may be prepared for topical administration e.g. as an ointment, cream or powder. The composition be prepared for oral administration e.g. as a tablet or capsule, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as spray, drops, gel or powder [e.g. refs 114 & 115].

### Further Antigenic Components of Compositions of the Invention

The invention also provides a composition comprising a polypeptide or the invention and one or more of the following further antigens:
a saccharide antigen form N. meningitudis serogroup A, C, W135 and/or Y (preferably all four), such as the oligosaccharide disclosed in ref. 116 from serogroup C [see also ref. 117] or the oligosaccharides of ref. 118.
a saccharide antigen from Streptococcus pneumoniae [e.g. 119, 120, 121].
an antigen from hepatitis A virus, such as inactivated virus [e.g. 122, 123].
an antigen from hepatitis B virus, such as the surface and/or core antigens [e.g. 123, 124].
a diphtheria antigen, such as a diphtheria toxoid [e.g. chapter 3 of ref. 125] e.g. the CRM₁₉₇ mutant [e.g. 126].
a tetanus antigen, such as a tetanus toxoid [e.g. chapter 4 of ref 125].
an antigen from Bordetella pertussis, such as pertussis holotoxin (PT) and filamentous haemagglutinin (FHA) from B. pertussis, optionally also in combination with pertactin and/or agglutinogens 2 and 3 [e.g. refs. 127 & 128].
a saccharide antigen from Haemophilus influenzae B [e.g. 117].
polio antigen(s) [e.g. 129, 130] such as IPV.
measles, mumps and/or rubella antigens [e.g. chapters 9, 10 & 11 of ref. 125].
influenza antigen(s) [e.g. chapter 19 of ref. 125], such as the haemagglutinin and/or neuraminidase surface proteins.
an antigen from Moraxella catarrhalis [e.g. 131].
a saccharide antigen from Streptococcus agalactiae (group B streptococcus).
an antigen from Streptococcus pyogenes (group A streptococcus) [e.g. 132, 133, 134].
an antigen from Staphylococcus aureus [e.g. 135].

The composition may comprise one or more of these further antigens.

In another embodiment, the GBS antigens of the invention are combined with one or more additional, non-GBS antigens suitable for use in a vaccine designed to protect elderly or immunocompromised individuals. For example, the GBS antigens may be combined with an antigen derived from the group consisting of Enterococcus faecalis, Staphylococcus aureus, Staphylococcus epidermis, Pseudomonas aeruginosa, Legionella pneumophila, Listeria monocytogenes, Neisseria meningitides, influenza, and Parainfluenza virus (‘PIV’).

Toxic protein antigens may be detoxified where necessary (e.g. detoxification of pertussis toxin by chemical and/or genetic means [128]).

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens. DTP combinations are thus preferred.

Saccharide antigens are preferably in the form of conjugates. Carrier proteins for the conjugates include bacterial toxins (such as diphtheria toxoid or tetanus toxoid), the N. meningitidis outer membrane protein [136], synthetic peptides [137, 138], heat shock proteins [139, 140], pertussis proteins [141, 142], protein D from H. influenzae [143, 144], cytokines [145], lymphokines [145], H. influenzae proteins, hormones [145], growth factors [145], toxin A or B from C. difficile [146], iron-uptake proteins [147], artificial proteins comprising multiple human CD4+ T cell epitopes from various pathogen-derived antigens [148] such as the N19 protein [149], pneumococcal surface protein PspA [150], pneumolysin [151], etc. A preferred carrier protein is the CRM197 protein [152].

Antigens in the composition will typically be present at a concentration of at least 1 μg/ml each. In general, the concentration of any given antigen will be sufficient to elicit an immune response against that antigen.

As an alternative to using proteins antigens in the immunogenic compositions of the invention, nucleic acid (preferably DNA e.g. in the form of a plasmid) encoding the antigen may be used.

Antigens are preferably adsorbed to an aluminium salt.

### Screening Methods

The invention provides a process for determining whether a test compound binds to a polypeptide of the invention. If a test compound binds to a polypeptide of the invention and this binding inhibits the life cycle of the GBS bacterium, then the test compound can be used as an antibiotic or as a lead compound for the design of antibiotics. The process will typically comprise the steps of contacting a test compound with a polypeptide of the invention, and determining whether the test compound binds to said polypeptide. Preferred polypeptides of the invention for use in these processes are enzymes (e.g. tRNA synthetases), membrane transporters and ribosomal polypeptides. Suitable test compounds include polypeptides, polypeptides, carbohydrates, lipids, nucleic acids (e.g. DNA, RNA, and modified forms thereof), as well as small organic compounds (e.g. MW between 200 and 2000 Da). The test compounds may be provided individually, but will typically be part of a library (e.g. a combinatorial library). Methods for detecting a binding interaction include NMR, filter-binding assays, gel-retardation assays, displacement assays, surface plasmon resonance, reverse two-hybrid etc. A compound which binds to a polypeptide of the invention can be tested for antibiotic activity by contacting the compound with GBS bacteria and then monitoring for inhibition of growth. The invention also provides a compound identified using these methods.

Preferably, the process comprises the steps of: (a) contacting a polypeptide of the invention with one or more candidate compounds to give a mixture; (b) incubating the mixture to allow polypeptide and the candidate compound(s) to interact; and (c) assessing whether the candidate compound binds to the polypeptide or modulates its activity.

Once a candidate compound has been identified in vitro as a compound that binds to a polypeptide of the invention then it may be desirable to perform further experiments to confirm the in vivo function of the compound in inhibiting bacterial growth and/or survival. Thus the method comprise the further step of contacting the compound with a GBS bacterium and assessing its effect.

The polypeptide used in the screening process may be free in solution, affixed to a solid support, located on a cell surface or located intracellularly. Preferably, the binding of a candidate compound to the polypeptide is detected by means of a label directly or indirectly associated with the candidate compound. The label may be a fluorophore, radioisotope, or other detectable label.

Preferred polypeptides for use in these screening methods are the ‘core’ sequences identified below.

### General

The invention provides a computer-readable medium (e.g. a floppy disk, a hard disk, a CD-ROM, a DVD etc.) and/or a computer memory and/or a computer database containing one or more of the sequences in the sequence listing.

The term “comprising” encompasses “including” as well as “consisting” e.g. a composition “comprising” X may consist exclusively of X or may include something additional e.g. X+Y.

The term “about” in relation to a numerical value x means, for example, x±10%.

The word “substantially” does not exclude “completely” e.g. a composition which is “substantially free” from Y may be completely free from Y. Where necessary, the word “substantially” may be omitted from the definition of the invention.

The N-terminus residues in the amino acid sequences in the sequence listing are given as the amino acid encoded by the first codon in the corresponding nucleotide sequence. Where the first codon is not ATG, it will be understood that it will be translated as methionine when the codon is a start codon, but will be translated as the indicated non-Met amino acid when the sequence is at the C-terminus of a fusion partner. The invention specifically discloses and encompasses each of the amino acid sequences of the sequence listing having a N-terminus methionine residue (e.g. a formyl-methionine residue) in place of any indicated non-Met residue. It also specifically discloses and encompasses each of the amino acid sequences of the sequence listing starting at any internal methionine residues in the sequences.

As indicated in the above text, nucleic acids and polypeptides of the invention may include sequences that:
(a) are identical (i.e. 100% identical) to the sequences disclosed in the sequence listing;
(b) share sequence identity with the sequences disclosed in the sequence listing;
(c) have 1, 2, 3, 4, 5, 6, 7, 8, 9 or 10 single nucleotide or amino acid alterations (deletions, insertions, substitutions), which may be at separate locations or may be contiguous, as compared to the sequences of (a) or (b); and
(d) when aligned with a particular sequence from the sequence listing using a pairwise alignment algorithm, a moving window of x monomers (amino acids or nucleotides) moving from start (N-terminus or 5′) to end (C-terminus of 3′), such that for an alignment that extends to p monomers (where p>x) there are p−x+1 such windows, each window has at least x·y identical aligned monomers, where: x is selected from 20, 25, 30, 35, 40, 45, 50, 60, 70, 80, 90, 100, 150, 200; y is selected from 0.50, 0.60, 0.70, 0.75, 0.80, 0.85, 0.90, 0.91, 0.92, 0.93, 0.94, 0.95, 0.96, 0.97, 0.98, 0.99; and if x·y is not an integer then it is rounded up to the nearest integer. The preferred pairwise alignment algorithm is the Needleman-Wunsch global alignment algorithm [153], using default parameters (e.g. with Gap opening penalty=10.0, and with Gap extension penalty=0.5, using the EBLOSUM62 scoring matrix). This algorithm is conveniently implemented in the needle tool in the EMBOSS package [154].

The nucleic acids and polypeptides of the invention may additionally have further sequences to the N-terminus/5′ and/or C-terminus/3′ of these sequences (a) to (d).

The practice of the present invention will employ, unless otherwise indicated, conventional methods of chemistry, biochemistry, molecular biology, immunology and pharmacology, within the skill of the art. Such techniques are explained fully in the literature. See, e.g., references 155-162, etc.

### BRIEF DESCRIPTION OF DRAWINGS

There are no drawings.

### MODES FOR CARRYING OUT THE INVENTION

Genome sequencing has been carried out on five strains of GBS from different serotypes: ‘18RS21’ (type II; MLST type ST19), ‘515’ (type Ia; MLST type ST23), ‘CJB111’ (type V; MLST type ST1), ‘COH1’ (type III; MLST type ST17) and ‘H36B’ (type Ib; MLST type ST6). Different numbers of coding sequences were identified in the five genomes:

| | Strain | | | | |
|---|---|---|---|---|---|
| | 18RS21 | 515 | CJB111 | COH1 | H36B |
| Coding | 2151 | 2249 | 2167 | 2410 | 2393 |
| seqs | | | | | |

These 11370 coding sequences are given in the sequence listing together with their inferred translation products. Annotation of these polypeptide sequences is given in Table I.

The sequence listing gives sequences in pairs, such that an odd-numbered sequence ‘n’ is a DNA coding sequence and the even-numbered sequence ‘n+1’ is the corresponding amino acid sequence:

| | Strain | | | | |
|---|---|---|---|---|---|
| | 18RS21 | 515 | CJB111 | COH1 | H36B |
| SEQ ID | 1-4302 | 4303-8800 | 8801-13134 | 13135-17954 | 17955-22740 |
| NO^{S} | | | | | |

The polypeptides and their epitopes can be used as antigens e.g. in vaccines or diagnostic tests.

Homologous coding sequences between strains are shown in Table II (listing SEQ ID numbers). For comparison, Table II also includes the ‘gi’ (GenInfo Identifier) accession numbers for strains 2603V/R (serotype V; MLST type ST106) [1] and NEM316 (serotype III; MLST type ST23) [3]. A single row in Table II includes all homologs and, where applicable, paralogs within a single strain.

In contrast to Table II, coding sequences without homologs in any of the other six sequenced genomes (i.e. unique to one strain within the six strains) are listed in Table III. These are preferred coding sequences of the invention e.g. when strain-specificity is desired. Each of the seven sequenced genomes contains between 13 and 61 sequences not present in any of the other strains. This variability exceeds that seen in the comparative genome hybridization analysis of reference 1.

Table IV lists coding sequences in the five new sequenced genomes that do not have any homologs in strains 2603V/R [1] or NEM316 [3]. These are preferred coding sequences of the invention e.g. when sequences not known in the prior art are desired.

Table V lists ‘core’ GBS genes, namely those that are found in all seven sequenced genomes. These ‘universal’ GBS coding sequences are preferred for use with the invention e.g. when strain-specificity is not desired, such as when designing a diagnostic test with high inter-strain cross-reactivity, or when preparing a composition which will elicit antibodies with high inter-strain cross-reactivity, or when screening for broad-range anti-GBS antibiotics. Table VI lists variable GBS genes, namely those that are found in at least two sequenced genomes, but not in all seven. The format of Tables V and VI follows that of Table II.

The GBS “pan-genome” can thus be divided in three parts: a core-genome, strain-specific sequences, and “dispensable genes” shared only by some of the strains. The core genes describe the basic aspects of GBS biology and major phenotypic traits, whereas dispensable and strain-specific genes contribute to the observed genetic diversity of the species and might confer selective advantages, such as adaptation to different niches, antibiotic resistance, and increased invasive capabilities.

The vast majority of genes making up the core genome belong to the groups of housekeeping functions, cell envelope, regulatory functions, and transport and binding proteins. However, about one third of the shared genes fall into the annotation class of hypothetical proteins and proteins of unknown function, thus suggesting that many aspects of basic GBS biology still need to be explored. Because of their ‘core’ nature, however, these sequences still have utility as they can be used in situations where inter-strain cross-reactivity is needed, without needing to know their true underlying biological function. Hypothetical genes and genes of unknown function are much more represented among the dispensable genes, probably due to the fact that more functions have been ascribed to better known (i.e. more frequently found) genes. This view is also supported by the strain-specific genes being predominantly of unknown function. Furthermore, genes associated with mobile and extrachromosomal elements are particularly abundant in this group, supporting the hypothesis that the majority of specific traits depend upon phenomena of lateral gene transfer. On the other hand, this class of genes is very poorly represented within the core genome, indicating that only a few of these rearrangements have remained stable during evolution of GBS.

The core shared by all isolates (Table V) accounts for only about 80% of any single genome, with the remaining 20% being absent in at least one other strain (Table VI). Approximately 1800 coding sequences are shared by the sequenced GBS strains. The criteria for gene identity between genomes was set low so that coding sequences were considered shared even if they were quite divergent in sequence. The size of the core is thus likely to be an overestimated, but it substantially defines the basic characteristics of the GBS species. As further GBS genome sequences become available then this “core” may decrease (by analogy a coding sequence would move from Table V to Table VI), but for the purposes of the present invention the “core” is the group given in Table V. Even using the sequences herein, the core decreases with the addition of each new genome, but extrapolation of the curve indicates that the core stabilizes at around 1800 coding sequences and will remain constant even as many more genomes are added.

One mechanism by which bacteria can modulate their lifestyle and virulence in response to variable stimuli, stress conditions and adaptation to different niches is phase variation [163, 164]. Such variation occurs by altering the length of short repeated DNA tracts within or immediately upstream of coding regions (contingency genes), thus causing frame-shifts and affecting protein synthesis. At least one important virulence-associated gene in GBS is regulated in this way [165], and so identification of further phase variable genes can identify new virulence factors. Virulence factors are particularly useful for vaccination, antibiotic targets, etc. Table VII shows such phase variable genes, and these are preferred polypeptides for use with the invention.

It will be understood that the invention has been described by way of example only and modifications may be made whilst remaining within the scope and spirit of the invention.

TABLE II
Homologs and paralogs

| 2603vr | 18rs21 | 515 | cjb11 | coh1 | h36b | NEM316 |
|---|---|---|---|---|---|---|
| 22535220 | 1, 3919 | 8615 | 12909 | 14061 | 22383 | 24413708 |
| 22535221 | 3 | 8617 | 12911 | 14063 | 22385, 22427 | 24413709 |
| 22535222 | 5 | 8619, 8703 | 12913 | 14065 | 22387 | 24413710 |
| 22535223 | 7 | 8621 | 12915 | 14067 | 22389 | 24413711 |
| 22535224 | 9 | 8623 | 12917 | 14069 | 22391 | 24413712 |
| 22535225 | 11 | 8625 | 12919 | 14071 | 22393 | 24413713 |
| 22533003 | 13 | 8627 | 12921 | 14073 | 22395 | 23094427 |
| 22533004 | 15 | 4303, 8629 | 12923 | 14075 | 22397 | 23094428 |
| 22533005 | 17 | 4305, 8631 | 12925, 12927 | 14077 | 22399 | 23094429 |
| 22533006, 22535061 | 19, 3631 | 4307, 8305, | — | 14079, 17271 | 22401 | 23094430 |
| | | 8323, 8325, | | | | |
| | | 8327, 8329, | | | | |
| | | 8787 | | | | |
| 22533007 | 21 | 4309 | 9607 | 14081 | 22403 | 23094431 |
| 22533008 | 23 | 4311 | 9609 | 14083 | 22405 | 23094432 |
| 22533009 | 25 | 4313 | 9611 | 14085 | 22407 | 23094433 |
| 22533010 | 27, 29 | 4315 | 9613 | 14087 | 22409, 22663 | 23094434 |
| 22533012 | 31 | 4317 | 9615 | 14089 | 22413 | 23094435 |
| 22533013 | 33 | 4319 | 9617 | 14091 | 22415 | 23094436 |
| 22533014 | 35 | 4321 | 9619 | 14093 | 22417 | 23094437 |
| 22533015 | 37 | 4323 | 9621 | 13135, 14095 | 22419 | 23094438 |
| 22533016, 22534732 | 39 | 4325 | 9623, 12021 | 13137, 14097, | 21493, 22421 | 23094439 |
| | | | | 16681 | | |
| 22533017 | 41 | 4327 | 9625 | 13139, 13143, | 22423 | 23094440 |
| | | | | 14099 | | |
| 22533018 | 43 | 4329 | 9627 | 13145, 13147, | 22425, 22623 | 23094441 |
| | | | | 13149, 14101 | | |
| 22533020 | 45, 47, 49, 51 | 4331 | 8801 | 13181 | 17963 | 23094442 |
| 22533021 | 53 | 4333, 4335 | 8803 | 13183 | 17965 | 23094443 |
| 22533022 | 55 | 4337 | 8805 | 13185 | 17967 | 23094444 |
| 22533023 | 57 | 4339, 4341 | 8807 | 13187, 13189 | 17969 | 23094445 |
| 22533024 | 59 | 4343 | 8809 | 13191 | 17971 | 23094446 |
| 22533025 | 61 | 4345 | 8811 | 13193 | 17973 | 23094447 |
| 22533026 | 63 | 4347 | 8813, 8815 | 13195 | 17975 | 23094448 |
| 22533027 | 65 | 4349 | 8817 | 13197 | 17977, 18033, | 23094449 |
| | | | | | 18035 | |
| 22533028 | 67 | 4351 | 8819 | 13199, 13245, | 17979, 18037, | 23094450 |
| | | | | 13247, 17811, | 22555 | |
| | | | | 17821, 17859 | | |
| 22533029 | 69, 71, 3965, | 4353 | 8821 | 13201, 17803, | 17981 | 23094451 |
| | 4017, 4061 | | | 17891 | | |
| 22533030 | 73, 75, 3963 | 4355 | 8823 | 13203 | 17983 | 23094452 |
| 22533032 | 77 | 4357, 4365 | 8825 | 13205 | 17985 | 23094453 |
| 22533033 | 79 | — | 8827 | 13207 | 17987 | 23094454 |
| 22533034 | 81 | 4361, 4369, | 8829 | 13209 | 17989, 22505 | 23094455 |
| | | 8707 | | | | |
| 22533035 | 83, 85 | 4371 | 8831 | 13211, 13249, | 17991 | 23094456 |
| | | | | 13251, 13253 | | |
| 22533036 | 87 | 4373 | 8833 | 13213 | 17993 | 23094457 |
| 22533037 | 89 | 4375 | 8835 | 13215, 17895 | 17995 | 23094458 |
| 22533038 | 91 | 4377, 4413, | 8837 | 13217, 13255, | 17997 | 23094459 |
| | | 4415, 4417 | | 17893 | | |
| 22533039 | 93 | 4379 | 8839 | 13219, 13257 | 17999 | 23094460 |
| 22533040 | 95 | 4381, 4419, | 8841 | 13221, 13259 | 18001 | 23094461 |
| | | 4421 | | | | |
| 22533041 | 97 | 4383 | 8843 | 13223, 13261 | 18003 | 23094462 |
| 22533042 | 99 | 4385 | 8845 | 13225 | 18005 | 23094463 |
| 22533043 | 101 | 4387 | 8847 | 13227 | 18007 | 23094464 |
| 22533044 | 103 | 4389 | 8849 | 13229 | 18009 | 23094465 |
| 22533045 | 105 | 4391 | 8851 | 13231 | 18011 | 23094466 |
| 22533046 | 107 | 4393 | 8853 | 13233 | 18013 | 23094467 |
| 22533047 | 109 | 4395 | 8855 | 13235, 17875, | 18015, 18039, | 23094468 |
| | | | | 17941 | 18041, 22631 | |
| 22533048 | 111 | 4397 | 8857, 13023 | 13237 | 18017 | 23094469 |
| 22533049 | 113, 115, 117, | 4399 | 8859 | 13239 | 18019 | 23094470 |
| | 3949 | | | | | |
| 22533050 | 119 | — | 8861 | — | 18021 | — |
| 22533051 | 121 | 4405 | 8863 | 13265 | 18023 | 23094473 |
| 22533053 | 123 | 4407 | 8865 | 13267 | — | 23094474 |
| 22533054 | 125 | 4409 | 8867 | 13269 | 18027 | 23094475 |
| 22533055 | 127 | 4411 | 8869 | 13271 | 18029 | 23094476 |
| 22533056 | 129 | 4423 | 8871 | 13273 | 18031 | 23094477 |
| 22533057 | 131 | 4425, 4427 | 8873 | 13275 | 18043 | 23094478 |
| 22533058 | 133 | 4429, 4431, | 8875 | 13277, 13279 | 18045, 18047, | 23094479 |
| | | 4433 | | | 18049, 18051 | |
| 22533059 | 135 | 4435, 4437 | 8877 | 13281 | 18053 | 23094480 |
| 22533060 | 137 | 4439 | 8879 | 17759 | 18055 | 23094481 |
| 22533061 | 139 | 4441 | 8881 | 13283 | 18057 | 23094482 |
| 22533062 | 141 | — | 8883 | — | 18059 | 23094483 |
| 22533063 | 143 | 4443 | 8885 | 13285 | 18061 | 23094484 |
| 22533064 | 145 | 4445 | 8887 | 13287, 13289 | 18063, 18071 | 23094485 |
| 22533065 | 147 | 4447 | 8889 | 13291 | 18065, 18073 | 23094486 |
| 22533066 | 149 | 4449 | 8891 | 13293 | 18067, 18069, | 23094487 |
| | | | | | 18075 | |
| 22533067 | 151 | 4451 | 8893 | 13295 | 18077 | 23094488 |
| 22533068 | 153 | 4453 | 8895 | 13297 | 18079 | 23094489 |
| 22533069 | 155 | 4455 | 8897 | 13299 | 18081 | 23094490 |
| 22533070 | 157 | 4457 | 8899 | 13301 | 18083 | 23094491 |
| 22533071 | 159 | 4461 | 8903 | 13155, 13305, | 18087 | 23094492 |
| | | | | 17925 | | |
| 22533072 | 161 | 4463 | 8905 | 13307 | 18089 | 23094493 |
| 22533073 | 163 | 4465 | 8907 | 13309 | 18091 | 23094494 |
| 22533074 | 165 | 4467 | 8909 | 13311 | 18093 | 23094495 |
| 22533075 | 167 | 4469 | 8911 | 13313 | 18095 | 23094496 |
| 22533076, 22534790 | 169, 3205 | 6299 | 12167 | 16801 | 21615 | 23094497, 24413378 |
| 22533077 | 171 | 4471 | 8913 | 13315 | 18097 | 23094498 |
| 22533078 | 173 | 4473 | 8915 | 13317 | 18099 | 23094499 |
| 22533080 | 175 | 4475 | 8917 | 13319 | 18101 | 23094500 |
| 22533081 | 177 | 4477 | 8919 | 13321, 17737 | 18103 | 23094501 |
| 22533082 | 179 | 4481 | 8923 | 13325 | 18107 | 23094502 |
| 22533083 | 181 | 4483 | 8925 | 13327 | 18109 | 23094503 |
| 22533084 | 183 | 4485 | 8927 | 13329 | 18111 | 23094504 |
| 22533085 | 185 | 4487 | 8929, 8931 | 13331 | 18113 | 23094505 |
| 22533086 | 187 | 4489 | 8933 | — | 18115 | 23094506 |
| 22533088 | 189 | 4491 | 8935 | — | 18117 | 23094508 |
| 22533089 | 191 | 4493 | 8937 | — | 18119 | 23094509 |
| 22533090 | 193 | 4495 | 8939 | 13333, 13335 | 18121, 18123 | 23094510 |
| 22533091 | 195 | 4497 | 8941 | 13337 | 18125 | 23094511 |
| 22533092, 22533093, | 199, 201, 503 | 4499, 4875 | 8943, 8945, | 13341, 13345, | 18127, 18135, | 23094512 |
| 22533257, | | | 9475, 9497 | 13663, 13681, | 18437, 18451 | |
| 22533270 | | | | 13685 | | |
| 22533094, 22533258, | 203, 505 | 4501 | 8947, 9477 | 13343, 13347, | 18129, 18137 | 23094513 |
| 22533260 | | | | 13665, 13687, | | |
| | | | | 17743 | | |
| 22533095 | 205 | 4503 | 8949 | 13349 | 18139 | 23094514 |
| 22533096 | 207 | 4505 | 8951 | 13339, 13351 | 18141 | 23094515 |
| 22533097 | 209 | 4507 | 8953 | 13353 | 18143 | 23094516 |
| 22533098 | 211 | 4509 | 8955 | 13355, 13357 | 18145 | 23094517 |
| 22533099 | 213 | 4511 | 8957 | 13359 | 18147 | 23094518 |
| 22533100 | 215 | 4513 | 8959 | 13361 | 18149 | 23094519 |
| 22533101 | 217 | 4515 | 8961 | 13363 | 18151, 18171 | 23094520 |
| 22533102 | 219 | 4517 | 8963 | 13365 | 18153 | 23094521 |
| 22533104 | 221 | 4519 | 8965 | 13367 | 18155 | 23094522 |
| 22533105 | 223 | 4521 | 8967 | 13369 | 18157 | 23094523 |
| 22533106 | 225 | 4523 | 8969 | 13371 | 18159 | 23094524 |
| 22533107 | 227 | 4525 | 8971 | 13373 | 18161 | 23094525 |
| 22533108 | 229 | 4527 | 8973 | 13375, 13385 | 18163 | 23094526 |
| 22533116 | 231 | 4549 | 8991 | 13399 | 18183 | 23094534 |
| 22533117 | 233 | 4551 | 8993 | 13401, 13403 | 18185 | 23094535 |
| 22533118 | 235 | 4553 | 8995 | 13405 | 18187 | 23094536 |
| 22533119 | 237 | 4555 | 8997 | 13407 | 18189 | 23094537 |
| 22533120 | 239 | 4557 | 8999 | 13413 | 18193 | 23094538 |
| 22533121 | 241 | 4559, 4561, | 9001 | 13415 | 18195 | 23094539 |
| | | 4563 | | | | |
| 22533122 | 243 | 4565 | 9003 | 13417, 13443 | 18197 | 23094540 |
| 22533123 | 245 | 4567 | 9005 | 13419, 13445 | 18199 | 23094541 |
| 22533124 | 247 | 4569 | 9007 | 13421 | 18201 | 23094542 |
| 22533125 | 249, 251, 253, | 4571 | 9009 | 13423 | 18203 | 23094543 |
| | 4055 | | | | | |
| 22533126 | 255 | 4573 | 9011 | 13425 | 18205 | 23094544 |
| 22533128 | 257 | 4577 | 9015 | 13429 | 18209 | 23094545 |
| 22533133 | 259, 261 | 4585 | 9023 | 13437 | 18217 | 23094549 |
| 22533134 | 263, 265, 267 | 4587 | 9025 | 13439 | 18219 | 23094550 |
| 22533139 | 269, 271 | 4591 | 9035 | 13451, 13487 | 18227 | 23094555 |
| 22533140 | 273 | 4593 | 9037 | 13453 | 18229 | 23094556 |
| 22533141, 22533904, | 275, 1701, | 4595, 6149, | 9039, 10563, | 13455, 15055, | 18231, 19849, | 23095333, 24413101 |
| 22534480 | 2693, 4111 | 7287, 8771 | 11555 | 16017 | 20993, 20995 | |
| 22533142 | 277 | 4597, 4599 | 9041 | 13457 | 22175 | — |
| 22533143 | 281, 285, 4051 | 4603 | 9045 | 13461 | 22173 | 23094558 |
| — | 283 | 4601 | 9043 | 13459 | — | — |
| 22533144 | 287, 4053 | 4605 | 9047 | 13463 | 22169, 22171 | 23094559 |
| 22533145 | 289, 4163 | 4607, 4609 | 9049 | 13465 | 22165, 22167 | 23094560 |
| 22533146 | 291 | 4611 | 9051 | 13467 | 22163 | 23094561 |
| 22533147 | 293 | 4613 | 9053 | 13469 | 22161 | 23094562 |
| 22533148 | 295, 297 | 4615 | 9055 | 13471 | 22159 | 23094563 |
| 22533149 | 299 | 4617 | 9057 | 13473, 17949 | 22157, 22647 | 23094564 |
| 22533150, 22534271 | 301 | 4619 | 9059 | 13475 | 22155 | 23094565 |
| 22533151, 22534154 | 303 | 4621, 6727, | 9061, 11015 | 13477, 13489, | 20309, 20311, | 23094566, 23095668 |
| | | 6729, 6731 | | 15575 | 20313, 22153 | |
| 22533152 | 305 | 4623 | 9063 | 13479 | 22151 | 23094567 |
| 22533154 | 307 | 4625 | 9065 | 13481 | 22149 | 23094568 |
| 22533155 | 309, 311 | 4627 | 9067 | 13483 | 22147 | 23094569 |
| 22533156 | 313 | 4629 | 9069 | 13485 | 22145 | 23094570 |
| | | 4631 | 9071 | 13491 | | |
| 22533158 | 315 | 4633 | 9073 | 13493 | 17955, 18239 | 23094572 |
| 22533159 | 317 | 4635 | 9075 | 13495 | 17957, 18241 | 23094573 |
| 22533160 | 319 | 4637 | 9077 | 13497 | 17959 | 23094574 |
| 22533161 | 321, 4077, | 4639, 8785 | 9079 | 13499 | 17961, 22541 | 23094575 |
| | 4101, 4113 | | | | | |
| 22533162 | 323 | 4641 | 9081 | 13501, 13503 | 18243, 18253 | 23094576 |
| 22533163 | 325 | 4643 | 9087 | 13505 | 18245 | 23094577 |
| 22533164 | 327 | 4645 | 9089 | 13507 | 18247 | 23094578 |
| 22533165 | 329 | 4647 | 9091 | 13509 | 18249 | 23094579 |
| — | 331 | 4649 | 9093 | 13511 | 18251 | 23094580 |
| 22533167 | 333 | 4655, 4657 | 9095 | 13513 | 18255 | 23094581 |
| 22533168 | 335 | 4659 | 9097 | 13515, 13517 | 18257 | 23094582 |
| 22533169 | 337 | 4661 | 9099, 13081 | 13519 | 18259 | 23094583 |
| 22533170 | 339, 343 | 4663, 4729 | 9083, 9085, | 13521, 17817 | 18261, 18281, | 23094584 |
| | | | 9101 | | 18283, 18285 | |
| 22533171 | 341, 345 | 4665 | 9103 | 13523 | 18263, 18287 | 23094585 |
| 22533172 | 347 | 4667 | 9105 | 13525, 13527 | 18265 | 23094586 |
| 22533173 | 349 | 4669 | 9107 | 13529 | 18267 | 23094587 |
| 22533174 | 351 | 4675 | 9115 | 13531 | 18269 | 23094591 |
| 22533175 | 353 | 4677 | 9117 | — | 18271 | 23094592 |
| 22533176 | 355 | 4679 | 9119 | 13533 | 18273 | 23094593 |
| 22533177 | 357 | 4681 | 9121 | 13535 | 18275 | 23094594 |
| 22533178 | 359 | 4683 | 9123 | 13537 | 18277 | 23094595 |
| 22533179 | 361 | 4685 | 9125 | 13539 | 18279 | — |
| 22533180 | 363 | 4687 | 9127 | 13541 | — | 23094596 |
| 22533181 | 365 | 4689 | 9129 | 13543 | 18289 | 23094597 |
| 22533182 | 367 | 4691 | 9131 | 13545 | 18291 | 23094598 |
| 22533183 | 369 | 4693 | 9133 | 13547, 13611, | 18293 | 23094599 |
| | | | | 13613 | | |
| 22533186 | 371 | 4697 | 9137 | 13551 | 18297 | 23094601 |
| 22533187 | 373 | 4699 | 9139 | 13553 | 18299 | 23094602 |
| 22533188 | 375, 377 | 4701 | 9141 | 13555, 13615, | 18301 | 23094603 |
| | | | | 13617 | | |
| 22533189 | 379 | 4703 | 9143 | 13557, 13619 | 18303 | 23094604 |
| 22533190, 22533587, | 381, 3121 | 4705, 5537, | 9145, 12049 | 13559 | 18305, 19283 | 23094605, 24413338 |
| 22534747, | | 7833 | | | | |
| 22534904 | | | | | | |
| 22533191 | 383 | 4707 | 9147 | 13561 | 18307 | 23094606 |
| 22533192 | 385 | 4709 | 9149 | 13563 | 18309 | 23094607 |
| 22533193 | 387 | 4711, 4731 | 9151 | 13565 | 18311 | 23094608 |
| 22533194 | 389 | 4713, 4733, | 9153 | 13567 | 18313 | 23094609 |
| | | 8679 | | | | |
| 22533195 | 391, 393 | 4715 | 9155 | 13569 | 18315, 18319 | 23094610 |
| 22533196 | 395 | 4717 | 9157 | 13571 | 18317, 18321 | — |
| 22533197 | 397 | 4719 | 9159 | 13573, 13621, | 18323 | 23094611 |
| | | | | 13623, 13625 | | |
| 22533198 | 399 | 4721 | 9161 | 13575, 13627, | 18325 | 23094612 |
| | | | | 13629 | | |
| 22533199 | 401 | 4723 | 9163 | 13577, 13631 | 18327 | 23094613 |
| 22533200 | 403 | 4725 | 9165 | 13579, 13633, | 18329 | 23094614 |
| | | | | 13635 | | |
| 22533201 | 405 | 4727, 4735 | 9167 | 13581 | 18331 | 23094615 |
| 22533202 | 407 | 4737, 4739 | 9169 | 13583 | 18333, 18693 | 23094616 |
| 22533203 | 409, 411 | 4741 | 9171 | 13585 | 18335, 18431 | 23094617 |
| 22533204, 22533207 | 413 | 4745, 4749 | 9173 | 13587, 13637 | 18337 | 23094618 |
| 22533208 | 415 | 4747, 4751 | 9175 | 13589, 13639 | 18339 | 23094619 |
| 22533209 | 417 | 4753, 8715 | 9177 | 13591, 13641 | 18341 | 23094620 |
| 22533210 | 419 | 4755 | 9179 | 13593 | 18343 | 23094621 |
| 22533211 | 421 | 4757 | 9181 | 13595 | 18345 | 23094622 |
| 22533212 | 423, 425 | 4759 | 9183 | 13597 | 18347 | 23094623 |
| 22533213 | 427 | 4761 | 9185 | 13599 | 18349 | 23094624 |
| 22533214 | 429 | 4763, 8793 | 9187, 13129 | 13601, 13643 | 18351, 18433 | 23094625 |
| 22533215 | 431 | 4765 | 9189 | 13603 | 18353 | 23094626 |
| 22533216 | 433 | 4767 | 9191 | 13605 | 18355, 22597 | 23094627 |
| 22533217 | 435 | 4769 | 9193 | 13607 | 18357, 22595 | 23094628 |
| 22533218 | 437, 439 | 4771 | 9195 | 13609, 13645 | 18359 | 23094629 |
| 22533219 | 441 | 4773 | 9197 | 13647 | 18361 | 23094630 |
| 22533220 | 443 | 4775 | 9199 | 13649 | 18363, 18435, | 23094631 |
| | | | | | 22635 | |
| 22533221 | 445 | 4777 | 9201 | 13651 | 18365 | 23094632 |
| 22533222 | 447 | 4779 | 9203 | 13653, 13655 | 18367 | 23094633 |
| 22533225 | 449 | 4787 | 9207 | 13659 | 18371 | 23094636 |
| 22533226 | 451 | 4789 | 9209 | 13661 | 18373 | 23094637 |
| 22533228 | 453 | 4791 | 9211 | 17729 | 18375 | 23094638 |
| — | 455 | — | — | — | 18377 | 23094639 |
| 22533230 | 457 | — | 9215 | — | 18379 | 23094640 |
| 22533231 | 459 | 4795 | 9217 | 17725 | 18381 | 23094641 |
| 22533234 | 461 | — | 9223 | — | 18385 | 23094643 |
| 22533235 | 463 | — | 9225 | — | 18387 | 23094644 |
| 22533236 | 465 | — | 9227 | — | 18389 | 23094645 |
| 22533238 | 467 | — | 9229 | — | 18391 | 23094646 |
| 22533239 | 469 | — | 9231 | — | 18393 | 23094647 |
| 22533240 | 471 | — | 9233 | — | 18395 | 23094648 |
| 22533241, 22534994 | 473, 3519 | 8203 | 9235, 12471, | 17133 | 18397, 21891 | 23094649, 24413519 |
| | | | 12939, 12941 | | | |
| 22533242 | 475 | 4815 | 9241, 12547 | — | 18399, 21965, | 23094650, 24413560 |
| | | | | | 22459 | |
| 22533243 | 477 | 4817 | 9243 | — | 18401 | 23094651, 23094652 |
| 22533244 | 479 | 4819 | 9245, 9247 | — | 18403 | 23094653 |
| 22533245 | 481 | 4821, 4823 | 9249, 12537, | 17327, 17331 | 18405, 18407, | 24413548, 24413559 |
| | | | 12539, 12543 | | 21953, 21955, | |
| | | | | | 21959, 21963 | |
| 22533246 | 483 | 4825 | 9251 | — | 18409 | 23094654 |
| 22533247 | 485 | 4827 | 9253 | — | 18411 | 23094655 |
| 22533248 | 487 | 4829 | 9255 | — | 18413 | — |
| 22533249 | 489 | 4831 | 9257 | 13163 | 18415 | — |
| 22533251 | 491 | 4835 | 9261 | 13167, 13169 | 18419 | 23094656 |
| 22533252 | 493 | 4837, 4839, | 9263 | 13171 | 18421 | 23094657 |
| | | 4841, 6645 | | | | |
| 22533253 | 495 | 4843 | 9265 | 13173 | 18423 | 23094658 |
| 22533254 | 497 | 4845 | 9267 | 13175 | 18425 | 23094659 |
| 22533255 | 499 | 4847 | 9269, 9271 | 13177 | 18427 | 23094660 |
| 22533256 | 501, 3975, | 4849 | 9273 | 13179, 17931 | 18429 | 23094661 |
| | 4215 | | | | | |
| 22533261 | 507 | — | 9479 | 13667 | 18131 | — |
| 22533262 | 509 | — | 9481 | 13669, 13689 | 18133 | — |
| 22533263 | 513 | — | 9483 | — | — | — |
| 22533264 | 515 | — | 9485 | — | 18439 | — |
| 22533265 | 517 | 4865 | 9487 | 13671 | 18441 | 23094671 |
| 22533266 | 519 | 4867 | 9489 | 13673 | 18443 | 23094672 |
| 22533267 | 521 | 4869 | 9491 | 13675 | 18445 | 23094673 |
| 22533268 | 523 | 4871 | 9493 | 13677, 17877 | 18447 | 23094674 |
| 22533269 | 525 | 4873 | 9495 | 13679 | 18449 | 23094675 |
| 22533271 | 527 | 4877 | 9499 | 13683 | 18453 | 23094676 |
| 22533272 | 529 | 4879, 4881 | 9317, 9501 | 13691 | 18455, 18459 | 23094677 |
| 22533276 | 531 | 4885 | 9321 | 13695 | 18463 | 23094679 |
| 22533277 | 533 | 4887 | 9323 | 13697 | 18465, 18471 | 23094680 |
| 22533278 | 535 | 4889 | 9325 | 13699 | 18467 | 23094681 |
| 22533279 | 537 | 4891 | 9327 | 13701 | 18469 | 23094682 |
| 22533280 | 539 | 4893 | 9329, 9331 | 13703 | 18473, 22133 | 23094683 |
| 22533281 | 541 | 4895 | 9333 | 13705 | 18475 | 23094684 |
| 22533282 | 543 | 4897 | 9335 | 13707 | 18477 | 23094685 |
| 22533283 | 545 | 4899 | 9337 | 13709 | 18479, 18609, | 23094686 |
| | | | | | 18613 | |
| 22533284 | 547 | 4901 | 9339 | 13711 | 18481, 18611, | 23094687 |
| | | | | | 18615 | |
| 22533285 | 549 | 4903 | 9341 | 13713 | 18483, 18617 | 23094688 |
| 22533286 | 551 | 4905 | 9343 | 13715 | 18485 | 23094689 |
| 22533287 | 553 | 4907 | 9345 | 13717 | 18487 | 23094690 |
| 22533288 | 555 | 4909 | 9347 | 13719, 13781 | 18489 | 23094691 |
| 22533290 | 557 | 4911 | 9349 | 13721, 13783 | 18491 | 23094692 |
| 22533291 | 559 | 4913 | 9351 | 13723, 13785 | 18493 | 23094693 |
| 22533292 | 561, 563, 565, | 4915 | 9353, 12121 | 13725 | 18495, 21593, | 23094694 |
| | 4153, 4155 | | | | 22429 | |
| 22533293 | 567 | 4917 | 9355 | 13727 | 18497 | 23094695 |
| 22533294 | 569, 571 | 4919 | 9357 | 13729 | 18499 | 23094696 |
| 22533295 | 573 | 4921 | 9359 | 13731 | 18501 | 23094697 |
| 22533296 | 575 | 4923, 4929 | 9361 | 13733, 13735, | 18503 | 23094698 |
| | | | | 13787, 13789 | | |
| 22533297 | 577 | 4925, 4931 | 9363 | 13737 | 18505 | 23094699 |
| 22533298 | 579 | 4927, 4933 | 9365 | 13739 | 18507 | 23094700 |
| 22533299 | 581 | 4935 | 9367 | 13741 | 18509 | 23094701 |
| 22533300 | 583 | 4937 | 9369 | 13743 | 18511 | 23094702 |
| 22533301 | 585 | 4939 | 9371 | 13745, 13791 | 18513 | 23094703 |
| 22533302 | 587 | 4941 | 9373 | 13747 | 18515 | 23094704 |
| 22533303 | 589, 591 | 4943, 4945, | 9375 | 13749 | 18517 | 23094705 |
| | | 8721 | | | | |
| 22533304 | 593 | 4947 | 9377 | 13751 | 18519 | 23094706 |
| 22533305 | 595 | 4949 | 9379 | 13753 | 18521 | 23094707 |
| 22533306 | 597 | 4951 | 9381 | 13755 | 18523 | 23094708 |
| 22533308 | 599 | 4953 | 9383 | 13757 | 18525 | 23094709 |
| 22533309 | 601 | 4955 | 9385 | 13759 | 18527 | 23094710 |
| 22533310 | 603 | 4957 | 9387 | 13761 | 18529 | 23094711 |
| 22533311 | 605 | 4959 | 9389 | 13763 | 18531 | 23094712 |
| 22533312 | 607 | 4961 | 9391 | 13765, 13793 | 18533 | 23094713 |
| 22533313 | 609 | 4963 | 9393 | 13767 | 18535, 18537 | 23094714 |
| 22533314 | 611 | 4965 | 9395 | 13769 | 18539 | 23094715 |
| 22533315 | 613 | 4967 | 9397 | 13771 | 18541 | 23094716 |
| 22533317 | 615 | 4971 | 9401 | 13775 | 18545 | 23094717 |
| 22533318 | 617 | 4973 | 9403 | 13777, 13779 | 18547 | 23094718 |
| 22533319 | 619, 621 | 4975 | 9405 | 13795 | 18549 | 23094719 |
| 22533320 | 623 | 4977 | 9407 | 13797 | 18551 | 23094720 |
| 22533321 | 625 | 4979 | 9409 | 13799 | 18553, 18619 | 23094721 |
| 22533323 | 627 | 4983 | 9413 | 13803 | 18557 | 23094722 |
| 22533324 | 629 | — | 9415 | 13805 | 18559 | 23094723 |
| 22533325 | 631 | 4985 | 9417 | 13807 | 18561 | 23094724 |
| — | 633 | 4987 | 9419 | 13809 | 18563 | 23094725 |
| 22533326 | 635 | 4989 | 9421 | 13811 | 18565 | 23094726 |
| 22533327 | 637 | 4991 | 9423 | 13813 | 18567 | 23094727 |
| 22533328 | 639 | 4993 | 9425 | 13815 | 18569 | 23094728 |
| 22533330 | 641 | 4995 | 9427 | 13817 | 18571 | 23094729 |
| 22533331 | 643 | 4997 | 9429 | 13819 | 18573 | 23094730 |
| 22533332 | 645 | 4999 | 9431 | 13821 | 18575, 18621 | 23094731 |
| 22533333 | 647 | 5001 | 9433 | 13823, 13871 | 18577, 22673 | 23094732 |
| 22533334 | 649 | 5003 | 9435 | 13825, 13873 | 18579 | 23094733 |
| 22533335 | 651 | 5005 | 9437 | 13827, 13875 | 18581 | 23094734 |
| 22533336 | 653 | 5007 | 9439 | 13829, 13877, | 18583 | 23094735 |
| | | | | 13879, 13881 | | |
| 22533337 | 655 | 5009, 5043 | 9441 | 13831 | 18585 | 23094736 |
| 22533338 | 657 | 5011 | 9443 | 13833 | 18587 | 23094737 |
| 22533339 | 659 | 5013 | 9445 | 13835 | 18589, 18623 | 23094738 |
| 22533340 | 661 | 5015 | 9447 | 13837 | 18591 | 23094739 |
| 22533341 | 663 | 5017 | 9449 | 13839 | 18593 | 23094740 |
| 22533342 | 665 | 5019 | 9451 | 13841 | 18595, 18625 | 23094741 |
| 22533343 | 667 | 5021 | 9453 | 13843 | 18597, 18627 | 23094742 |
| 22533344 | 669 | 5023 | 9455 | 13845 | 18599 | 23094743 |
| 22533345 | 671 | 5025 | 9457 | 13847 | 18601 | 23094744 |
| 22533346 | 673 | 5027 | 9459 | 13849 | 18603 | 23094745 |
| 22533347 | 675 | 5029 | 9461 | 13851 | 18605, 18629 | 23094746 |
| 22533348 | 677 | 5031 | 9463 | 13853, 17831 | 18607, 18631, | 23094747 |
| | | | | | 18633 | |
| 22533350 | 679 | 5033 | 9465, 9503 | 13855 | 18635, 18645 | 23094748 |
| 22533351 | 681 | 5035 | 9467 | 13857 | 18637 | 23094749 |
| 22533352 | 683 | 5037 | 9469 | 13859, 17911 | 18639 | 23094750 |
| 22533353 | 685 | 5039 | 9471 | 13861 | 18641 | 23094751 |
| 22533354 | 687 | 5041 | 9473 | 13863 | 18643 | 23094752 |
| 22533355 | 689, 3921 | 5045 | 9275 | 13865 | 18647 | 23094753 |
| 22533356 | 691 | 5047 | 9277 | 13867 | 18649 | 23094754 |
| 22533358 | 693 | 5049, 5079 | 9279 | 13869, 13883 | 18651, 18653 | 23094755 |
| 22533359 | 695 | 5051 | 9281 | 13885, 13887 | 18655 | 23094756 |
| 22533360 | 697 | 5053 | 9283 | 13889 | 18657 | 23094757 |
| 22533361 | 699 | 5055 | 9285 | 13891 | 18659 | 23094758 |
| 22533362 | 701, 4133, | 5057 | 9287 | 13893 | 18661 | 23094759 |
| | 4135 | | | | | |
| 22533363 | 703 | 5059, 5081 | 9289 | 13895, 17153 | 18663 | 23094760 |
| 22533364 | 705 | 5061, 5083 | 9291 | 13897 | 18665 | 23094761 |
| 22533365 | 707 | 5063 | 9293 | 13899 | 18667 | 23094762 |
| 22533366 | 709 | 5065 | 9295 | 13901 | 18669 | 23094763 |
| 22533367 | 711, 723, 4301 | 5067 | 9297 | 13903 | 18671, 18695 | 23094764 |
| 22533368 | 713, 725, 4299 | 5069 | 9299 | 13905 | 18673, 18697 | 23094765 |
| 22533369 | 715, 727, 4297 | 5071, 5087 | 9301 | 13907 | 18675, 18699, | 23094766 |
| | | | | | 18701 | |
| 22533370, 22534818 | 717, 729, | 5073, 5089, | 9303, 12225 | 13909, 16863, | 18677, 18703, | 23094767, 24413404 |
| | 3261, 3263, | 5091, 7953 | | 16893, 17849 | 21667 | |
| | 4165, 4167, | | | | | |
| | 4295 | | | | | |
| 22533372 | 719, 731, 4293 | 5075, 5093 | 9305 | 13911 | 18679 | 23094768 |
| 22533373 | 721, 733, 4291 | 5095 | 9307 | 13913 | 18681 | 23094769 |
| 22533374 | 735 | 5097 | 9309 | 13915 | 18683 | 23094770 |
| 22533375 | 737 | 5099, 5105 | 9311 | 13917 | 18685 | 23094771 |
| 22533376 | 739 | 5101, 5107, | 9313 | 13919 | 18687 | 23094772 |
| | | 5109 | | | | |
| 22533377 | 741 | 5111 | 9315 | 13921 | 18705 | 23094773 |
| 22533379 | 743 | 5115 | 9507 | 13925 | 18711 | 23094775 |
| 22533380 | 745 | 5117, 5149 | 9509 | 13927 | 18713 | 23094776 |
| 22533381 | 747 | 5119, 5151 | 9511 | 13929 | 18715 | 23094777 |
| 22533382 | 749 | 5121 | 9513 | 13931 | 18717 | 23094778 |
| 22533383 | 751 | 5123 | 9515 | 13933 | 18719 | 23094779 |
| 22533384 | 753 | 5125 | 9517 | 13935 | 18721 | 23094780 |
| 22533385 | 755 | 5127 | 9519 | 13937 | 18723 | 23094781 |
| 22533386 | 757 | 5129 | 9521 | 13939 | 18725 | 23094782 |
| 22533387 | 759 | 5131 | 9523 | 13941 | 18727 | 23094783 |
| 22533388 | 761 | 5133 | 9525 | 13943 | 18729 | 23094785 |
| 22533390 | 763 | 5137 | 9529 | 13947 | 18733 | 23094786 |
| 22533391 | 765 | 5139 | 9531 | 13949 | 18735 | 23094787 |
| 22533392 | 767 | 5141 | 9533 | 13951 | 18737 | 23094837 |
| 22533393 | 769 | 5143 | 9535 | 13953 | 18739 | 23094838 |
| 22533394 | 771 | 5145 | 9537 | 13955 | 18741 | 23094839 |
| 22533395 | 773 | 5147 | 9539 | 13957 | 18743 | 23094840 |
| 22533396 | 775 | 5153 | 9541 | 13959 | 18745 | 23094841 |
| 22533397 | 777 | 5155 | 9543 | 13961 | 18747 | 23094842 |
| 22533398 | 779, 781, 783 | 5157, 5159, | 9545 | 13963 | 18749, 18751 | 23094843 |
| | | 5161 | | | | |
| 22533400 | 785 | 5163 | 9547 | 13965 | 18753 | 23094844 |
| 22533401 | 787 | 5165 | 9549 | 13967 | 18755 | 23094845 |
| 22533402 | 789 | 5167, 5201 | 9551 | 13969 | 18757 | 23094846 |
| 22533403 | 791, 2369, | 5169 | 9553 | 13971 | 18759, 18783, | 23094847 |
| | 4147, 4149, | | | | 20597 | |
| | 4179 | | | | | |
| 22533404 | 793 | 5171 | 9555 | 13973 | 18761 | 23094848 |
| 22533405 | 795 | 5173 | 9557 | 13975 | 18763 | 23094849 |
| 22533406 | 797 | 5175 | 9559 | 13977 | 18765 | 23094850 |
| 22533407 | 799 | 5177 | 9561 | 13979 | 18767, 22527 | 23094851 |
| 22533408 | 801 | 5179 | 9563 | 13981 | 18769, 22525 | 23094852 |
| 22533409 | 803 | 5181 | 9565 | 13983 | 18771 | 23094853 |
| 22533410 | 805 | 5183, 5203, | 9567 | 13985, 17809 | 18773 | 23094854 |
| | | 5205 | | | | |
| 22533411 | 807 | 5185 | 9569 | 13987 | 18775 | 23094855 |
| 22533412 | 809 | 5187 | 9571 | 13989, 13991, | 18777 | 23094856 |
| | | | | 14011 | | |
| 22533413, 22534328 | 811 | 5189 | 9573 | 13993 | 18779 | 23094857 |
| 22533414 | 813 | 5191 | 9575, 9577 | 13995 | 18781 | 23094858 |
| 22533415 | 815 | 5193 | 9579 | 13997 | 18785 | 23094859 |
| 22533416 | 817 | 5195 | 9581 | 13999 | 18787 | 23094860 |
| 22533417 | 819 | 5197 | 9583 | 14001, 14003 | 18789, 18791 | 23094861 |
| 22533418 | 821 | 5199 | 9585 | 14005 | 18793 | 23094862 |
| 22533420 | 823 | 4651 | 9589 | 14007 | 18797 | 23094863 |
| 22533421 | 825 | 4653, 5209 | 9591 | 14009 | 18799 | 23094864 |
| 22533426 | 827 | 5217 | 9599 | 14019 | 18809 | 23094868 |
| 22533422 | 829 | 5211 | 9593 | 14013 | 18801 | 23094865 |
| 22533427 | 831 | 5219 | 9601 | 14021 | 18811 | 23094869 |
| 22533428 | 833 | 5221 | 9603 | 14023, 14025 | 18813 | 23094870 |
| 22533429 | 835 | 5223 | 9605 | 14027 | 18815 | 23094871 |
| 22533430 | 837, 4049 | 4851 | 9629 | 14103 | 18817 | 23094872 |
| 22533431 | 839 | 4853 | 9631 | 14105 | 18819 | 23094873 |
| 22533432 | 841, 843 | 4855, 8777 | 9633 | 14107 | 18821 | 23094874 |
| 22533433 | 845 | 4857 | 9635 | 14109 | 18823 | 23094875 |
| 22533434 | 847 | 4859 | 9637 | 14111 | 18825 | 23094876 |
| 22533435 | 849 | 4861, 4863, | 9639 | 14113, 14115, | 18827, 18867, | 23094877 |
| | | 5225 | | 14117, 14159, | 18869 | |
| | | | | 14161 | | |
| 22533436 | 851 | 5227 | 9641 | 14119, 14163 | 18829, 18871 | 23094878 |
| 22533438 | 853 | 5229 | 9643 | 14121 | 18831 | 23094879 |
| 22533439 | 855 | 5231 | 9645 | 14123 | 18833 | 23094880 |
| 22533440 | 857 | 5233 | 9647 | 14125 | 18835 | 23094881 |
| 22533441 | 859 | 5235 | 9649 | 14127 | 18837 | 23094882 |
| 22533442 | 861 | 5237 | 9651 | 14129 | 18839 | 23094883 |
| 22533443 | 863 | 5239 | 9653 | 14135 | 18841 | 23094884 |
| 22533444 | 865 | 5241 | 9655, 9657 | 14137 | 18843, 18845 | 23094885 |
| 22533445 | 867 | 5243 | 9659 | 14139 | 18847 | 23094886 |
| 22533446 | 869 | 5245 | 9661, 9663 | 14141 | 18849 | 23094887 |
| 22533447 | 871 | 5247 | 9665 | 14143 | 18851 | 23094888 |
| 22533448 | 873 | 5249, 5251 | 9667, 9669 | 14145, 14147 | 18853, 18855 | 23094889 |
| 22533449 | 875 | 5253, 5255 | 9671 | 14149 | 18857 | 23094890 |
| 22533450 | 877 | 5257, 5641 | 9673 | 14151 | 18859 | 23094891 |
| 22533275, 22533562, | 879, 1623, | 6713, 7067 | 12307, 12559, | — | 19961, 19985, | — |
| 22533988, | 1807, 2331 | | 12755, 13113 | | 20295, 20835, | |
| 22534486, 22534579, | | | | | 21979, 22125, | |
| 22535047 | | | | | 22177, 22379, | |
| | | | | | 22649 | |
| 22533274, 22533563, | 881, 883, | 6327, 6711, | 12309, 12561, | — | 19963, 19987, | — |
| 22533989, | 1621, 1809, | 7069 | 12753, 13041, | | 20293, 20833, | |
| 22534485, 22534580, | 2333 | | 13115 | | 21977, 22127, | |
| 22535046 | | | | | 22179, 22377 | |
| 22533451 | 885 | 5259 | 9675 | 14153 | 18861 | 23094892 |
| 22533452 | 887, 3709 | 5261, 5265, | 9677, 12617 | 14155, 17403, | 18863, 22043 | 23094893 |
| | | 8417 | | 17419 | | |
| 22533453 | 889, 1187 | 5263, 5267, | ########## | 14157, 14165, | 18865, 18875, | 23094894, 23095037 |
| | | 8691, 8693, | | 14475, 17699, | 22479, 22483, | |
| | | 8701, 8711, | | 17701, 17751, | 22511, 22513, | |
| | | 8723, 8731, | | 17777, 17795, | 22517, 22539, | |
| | | 8735, 8745, | | 17799, 17837, | 22645, 22655, | |
| | | 8753, 8755, | | 17861, 17863, | 22657, 22669, | |
| | | 8761 | | 17865, 17867, | 22681, 22735 | |
| | | | | 17869, 17901, | | |
| | | | | 17917, 17937 | | |
| 22533454, 22535028 | 891 | 8345 | 9683, 12557 | 14167, 17301 | 22473 | 23094895, 24413563 |
| 22533455 | 893 | 8343 | 9685 | 14169, 17299 | — | 23094896 |
| 22533457 | 895 | — | 9687 | 14171 | — | — |
| 22535029, 22535036 | 897, 3583, | 5269, 5271, | 9691, 9895 | 14173, 17215 | 18877, 18879, | 23094663, 23095697, |
| | 4039 | 5273, 8257 | | | 20369 | 24413550 |
| — | 899, 901 | 5275, 5277 | 9693 | 14175, 14177 | 18881, 18885, | 23094907, 23094909 |
| | | | | | 22563 | |
| 22533458 | 903 | 5279 | 9695 | 14179 | 18925 | 23094910 |
| 22533459 | 905 | 5281 | 9697 | — | 18927 | 23094911 |
| 22533460, 22533461 | 907, 909 | 5283 | 9699, 9701 | 14181, 14183 | 18929 | 23094912, 23094913 |
| 22533462 | 911 | — | 9703 | 14185 | — | 23094914 |
| 22533463 | 913 | 5287 | 9705 | 14187, 17695 | 18933 | 23094915 |
| 22533464 | 915 | 5297 | 9709 | 17689 | 18935 | 23094917 |
| 22533465 | 917 | 5299 | 9711 | 17687 | 18937, 18939 | 23094918 |
| 22533466 | 919 | — | — | — | 18941 | — |
| 22533467 | 921 | 5301 | 9713 | 17807 | 18943 | 23094919 |
| 22533468 | 923 | 5303 | 9715, 9717 | 14189 | 18945, 18947 | 23094920 |
| 22533469 | 925 | 5305 | 9719 | 14191 | 18949 | 23094921 |
| 22533470 | 927 | 5307 | 9721 | 14193 | 18951 | 23094922 |
| 22533471 | 929 | 5309 | 9723 | 14195 | 18953 | 23094923, 23095104, |
| | | | | | | 23095466 |
| 22533472 | 931 | 5311 | 9725 | 14197 | 18955 | 23094924 |
| 22533473 | 933 | 5313 | 9727 | 14199, 14267 | 18957 | 23094925 |
| — | 935, 937 | 5315 | 9729 | 14201 | 18959, 18961 | 23094926 |
| 22533474 | 939 | 5317 | 9731 | 14203 | 18963 | 23094927 |
| 22533475 | 941 | 5319, 5367 | 9733 | 14205 | 18965 | 23094928 |
| 22533476 | 943 | 5321 | 9735 | 14207 | 18967 | 23094929 |
| 22533478 | 945 | 5323 | 9737 | 14209, 14269, | 18969 | 23094930 |
| | | | | 14271 | | |
| 22533479 | 947 | 5325 | 9739 | 14211 | 18971 | 23094931 |
| 22533480 | 949 | 5327 | 9741 | 14213, 14273 | 18973 | 23094932 |
| 22533481 | 951 | 5329 | 9743 | 14215 | 18975 | 23094933 |
| 22533482 | 953 | 5331 | 9745 | 14217 | 18977 | 23094934 |
| 22533483 | 955 | 5333 | 9747 | 14219 | 18979 | 23094935 |
| 22533484 | 957 | 5335 | 9749 | 14221 | 18981 | — |
| 22533485 | 959 | 5337 | 9751 | 14223 | 18983, 19025 | 23094936 |
| 22533486 | 961 | 5339 | 9753 | 14225 | 18985 | 23094937 |
| 22533487 | 963 | 5341 | 9755, 10049 | 14227 | 18987 | 23094938 |
| 22533488 | 965 | 5343 | 9757 | 14229 | 18989 | 23094939 |
| 22533489 | 967 | 5345 | 9759 | 14231 | 18991, 19027 | 23094940 |
| 22533490 | 969, 971 | 5347 | 9761 | 14233 | 18993 | 23094941 |
| 22533491 | 973 | 5349 | 9763 | 14235 | 18995 | 23094942 |
| 22533492 | 975 | 5351 | 9765 | 14237 | 18997 | 23094943 |
| 22533493 | 977 | 5353 | 9767 | 14239, 14275, | 18999 | 28094944 |
| | | | | 14277 | | |
| 22533494 | 979 | 5355 | 9769 | 14241 | 19001 | 23094945 |
| 22533495 | 981 | 5357 | 9771 | 14243 | 19003 | 23094946 |
| 22533496 | 983 | 5359 | 9773 | 14245 | 19005 | 23094947 |
| 22533497 | 985 | 5361 | 9775 | 14247 | 19007 | 23094948 |
| 22533498 | 987 | 5363 | 9777 | 14249 | 19009 | 23094949 |
| 22533499 | 989 | 5365, 5371 | 9779 | 14251 | 19011 | 23094950 |
| 22533500 | 991 | — | — | — | — | 23094951 |
| 22533501 | 993, 995 | 5373 | 9781 | 14253 | 19013, 19029 | 23094952 |
| 22533502 | 997 | 5375, 8763 | 9783 | 14255 | 19015 | 23094953 |
| 22533504 | 999, 1001 | 5377 | 9785 | 14257, 14279, | 19017 | 23094954 |
| | | | | 14281, 14283, | | |
| | | | | 14285 | | |
| 22533505 | 1003 | 5379 | 9787 | 14259 | 19019 | 23094955 |
| 22533506 | 1005 | 5381 | 9789 | 14261 | 19021 | 23094956 |
| 22533507 | 1007 | 5383 | 9791 | 14263 | 19023 | 23094957 |
| 22533509 | 1009 | 8635 | 9793 | 14265 | 19031 | 23094958 |
| 22533511, 22534495 | 1011, 2721 | 5385, 7323 | 9797, 11587 | 14289, 16195, | 19035, 21023 | 23094960, 24413115 |
| | | | | 17739 | | |
| 22533512 | 1013 | 5387 | 9799, 9801 | 14291 | 19037 | 23094962 |
| 22533513 | 1015 | 5389 | 9803 | 14293 | 19039 | 23094963 |
| 22533514 | 1017, 4075 | 5391 | 9805 | 14295 | 19041 | 23094964 |
| 22533515 | 1019, 3925, | 5393 | 9807 | 14297 | 19043 | 23094965 |
| | 4073 | | | | | |
| 22533516 | 1021, 3923 | 5395 | 9809 | 14299 | 19045 | 23094966 |
| 22533517 | 1023 | 5397 | 9811 | 14301 | 19047 | 23094967 |
| 22533518 | 1025 | 5399 | 9813 | 14303 | 19049 | 23094968 |
| 22533519 | 1027 | 5401 | 9815 | 14305 | 19051 | 23094969 |
| 22533520 | 1029 | 5403 | 9817 | 14307 | 19053 | 23094970 |
| 22533521 | 1031 | 5405 | 9819, 9821 | 14309, 17779 | 19055 | 23094971 |
| 22533522 | 1033 | 5407 | 9823 | 14311 | 19057 | 23094972 |
| 22533523 | 1035 | 5409 | 9825 | 14313 | 19059 | 23094973 |
| 22533525 | 1037 | 5411 | — | — | 19061 | 23094974 |
| 22533526 | 1039 | 5413 | 9827 | 14315 | 19173 | 23094975 |
| 22533527 | 1041 | 5415 | 9829 | 14317 | 19175 | 23094976 |
| 22533528, 22534375 | 1043 | 5417, 7081, | 9831, 11341 | 14319, 15951 | 19177, 20781 | 23094977, 24412999 |
| | | 7097 | | | | |
| 22533529 | 1045 | 5419 | 9833 | 14321 | 19179 | 23094978 |
| 22533530 | 1047 | 5421 | 9835 | 14323 | 19181 | 23094979 |
| 22533531 | 1049 | 5423 | 9837 | 14325 | 19183 | 23094980 |
| 22533532 | 1051 | 5425, 8717 | 9839 | 14327 | 19185 | 23094981 |
| 22533533 | 1053 | 5427 | 9841 | 14329 | 19187 | 23094982 |
| 22533534 | 1055 | 5429, 5431 | 9843 | 14331 | 19189 | 23094983 |
| 22533535 | 1057 | 5433 | 9845 | 14333, 14335 | 19191 | 23094984 |
| 22533536 | 1059 | 5435 | 9847 | 14337 | 19193 | 23094985 |
| 22533537 | 1061 | 5437 | 9849 | 14339 | 19195 | 23094986 |
| — | 1063 | 5439 | 9851 | 14341 | 19197 | 23094987 |
| 22533538 | 1065 | 5441 | 9853 | 14343 | 19199, 19241 | 23094988 |
| 22533539 | 1067 | 5443 | 9855 | 14345 | 19201 | 23094989 |
| 22533540 | 1069 | 5445 | 9857 | 14351 | 19203 | 23094990 |
| 22533541 | 1071 | 5447 | 9859 | 14353 | 19205 | 23094991 |
| 22533542 | 1073 | 5449 | 9861 | 14355, 14357, | 19207 | 23094992 |
| | | | | 14359 | | |
| 22533544 | 1075, 1077 | 5451, 5481 | 9863 | 14361 | 19209, 19243 | 23094993 |
| 22533545 | 1079 | 5453 | 9865 | 14363 | 19211 | 23094994 |
| 22533546 | 1081 | 5455 | 9867 | 14365 | 19213 | 23094995 |
| 22533547 | 1083, 4257 | 5457 | 9869 | 14367, 14443 | 19215 | 23094996 |
| 22533548 | 1085, 1087, | 5459 | 9871 | 14369, 14445 | 19217 | 23094997 |
| | 4259 | | | | | |
| 22533550 | 1089, 1093 | 5461 | 9873 | 14371 | 19219 | 23094998 |
| 22533551 | 1091, 1095 | 5463 | 9875 | 14373 | 19221 | 23094999 |
| 22533552 | 1097 | 5465 | 9877 | 14375 | 19223 | 23095000 |
| 22533553 | 1099 | 5467 | 9879 | 14377 | 19225 | 23095001 |
| 22533554 | 1101 | 5469 | 9881 | 14379 | 19227, 19229 | 23095002 |
| 22533555 | 1103, 1105 | 5471 | 9883, 12785 | 14381 | 19231 | 23095003 |
| 22533556 | 1107 | 5473 | 9885 | 14383 | 19233 | 23095004 |
| 22533557 | 1109, 1111 | 5475 | 9887 | 14385 | 19235 | 23095005 |
| 22533558 | 1113 | 5477 | 9889 | 14387 | 19237 | 23095006 |
| 22533559 | 1115 | 5479 | 9891 | 14389 | 19239, 19245 | 23095007 |
| 22533560 | 1117 | 5483 | 9905 | 14391 | 19247 | 23095008 |
| 22533561 | 1119 | 5485, 8669 | 9907 | 14393 | 19249, 19251 | 23095009 |
| 22533564 | 1121 | 5487 | 9909 | 14395 | 19253 | 23095010 |
| — | 1123 | 5621 | 9911 | 14397 | 19255 | — |
| 22533629 | 1125 | 5623 | 9913 | 14399 | 19257 | 23095011 |
| — | 1127 | 5625 | 9915 | 14401 | 19259 | — |
| — | 1129 | 5627 | 9917 | 14403 | 19261 | — |
| 22533631 | 1131 | 5631 | 9921 | 14407 | 19265 | 23095012 |
| 22533632 | 1133 | 5633 | 9923 | 14409 | 19267 | 23095013 |
| 22533633 | 1135, 1141 | 5635 | 9925 | 14411, 14447 | 19269 | 23095014 |
| 22533626, 22533637, | 1137, 4079 | 5615, 5651, | 9935, 11035 | 14423 | 19159, 19299, | 23095018, 23095676 |
| 22534163 | | 6749 | | | 20331 | |
| 22533627 | 1139 | 5617 | — | — | — | — |
| 22533634 | 1143 | 5637, 5643 | 9927 | 14413, 14449, | 19271, 19273 | 23095015 |
| | | | | 14451 | | |
| 22533635 | 1145 | 5639, 5645 | 9929 | 14415 | 19275 | 23095016 |
| 22533636 | 1147 | 5647 | 9931 | 14417, 14419 | 19277, 19295 | 23095017 |
| 22533628, 22533638 | 1149, 3951 | 5619, 5653 | 9937 | 14425 | 19171, 19301 | — |
| 22533639 | 1151 | 5655 | 9939 | 14427 | 19303 | 23095019 |
| 22533640 | 1153 | 5657 | 9941 | 14429, 14453 | 19305 | 23095020 |
| 22533641 | 1155, 1157 | 5659, 5661 | 9943 | 14431 | 19307 | 23095021 |
| 22533642 | 1159 | 5663 | 9945 | 14433, 14435 | 19309 | 23095022 |
| 22533643 | 1161 | 5665 | 9947 | 14437 | 19311 | 23095023 |
| 22533644 | 1163 | 5667 | 9949 | 14439 | 19313 | 23095024 |
| 22533645 | 1165 | 5669 | 9951 | 14441 | 19315 | 23095025 |
| 22533646 | 1167 | 5671 | 9953 | 14455 | 19317 | 23095026 |
| 22533647 | 1169 | 5673 | 9955 | 14457 | 19319, 19321 | 23095027 |
| 22533648 | 1171 | 5675 | 9957, 10007 | 14459 | 19323 | 23095028 |
| 22533649 | 1173 | 5677 | 9959 | 14461 | 19325 | 23095029 |
| 22533650 | 1175 | 5679, 5681 | 9961 | 14463 | 19327 | 23095030 |
| 22533652 | 1177 | 5683 | 9963 | 14465 | 19329 | 23095031 |
| 22533653 | 1179 | — | 10059 | 14467 | — | 23095032 |
| 22533654 | 1181 | — | 10061 | 14469 | — | 23095033 |
| 22533655, 22535158 | 1183 | — | 9965, 10063 | 14471 | — | 23095034 |
| — | 1185 | — | 10065 | — | — | 23095035 |
| 22533656 | 1189 | 5649, 6959 | 9933, 10069, | 14421, 14477, | 19297, 20539 | — |
| | | | 11229 | 15787 | | |
| 22533657, 22534267 | 1191, 1193, | — | 10071 | 14479, 15805 | — | 23095038 |
| | 2323, 2325 | | | | | |
| — | 1195 | — | 10073 | 14481 | — | 23095039 |
| 22534863 | 1197, 3357 | 8041 | 10075, 10077, | 14485, 14487, | 21759 | 23095040, 24413446 |
| | | | 12319 | 16967 | | |
| 22533659 | 1199 | — | 10079 | 14489 | — | 23095041 |
| 22535098 | 1201, 3701 | 8407, 8409, | 12609 | 17393 | 22035 | 24413589 |
| | | 8495 | | | | |
| 22533660 | 1203, 1205 | — | 10081 | 14491 | — | 23095042 |
| 22533661 | 1207, 1211 | — | 10083 | 14493 | — | 23095043 |
| 22533662, 22533663, | 1209, 1213, | 7203, 7205 | 10085, 10087, | 14495, 14497, | 20899, 20901 | 23095044, 23095045, |
| 22533665, | 1215, 1221, | | 10091, 11457, | 14501, 17847 | | 24413056, |
| 22534434, 22534435 | 2623, 2625 | | 11459 | | | 24413057 |
| 22533664, 22534437, | 1217, 1219, | 7209 | 10089, 11463, | 14499, 14585 | 20905, 21501, | 23095046, 24413059 |
| 22534737 | 2629, 3099 | | 12027 | | 22537 | |
| 22533666 | 1223 | — | 10093 | 14503 | 19345 | 23095047 |
| 22534312 | 1225, 2403 | 5685 | 10095 | 14505 | 19331, 20649 | 24412939 |
| — | 1227 | 5689 | 10099 | 14509 | 19335 | 23095048 |
| 22533667 | 1229 | 5691 | 10101 | 14511 | 19337 | — |
| 22533671 | 1231 | 5693 | 10103 | 14513 | 19339 | 23095049 |
| 22533672 | 1233 | 5695 | 10105 | 14515 | 19341 | 23095050 |
| 22533673 | 1235 | 5697 | 10107 | 14517, 14519 | 19343 | 23095051 |
| 22533674 | 1237 | 5699 | 10109 | 14521 | 19347 | 23095052 |
| 22533675 | 1239 | 5701 | 10111 | 14523 | 19349, 19409 | 23095053 |
| 22533677 | 1241 | 5703 | 10113 | 14525 | 19351 | 23095055 |
| 22533678 | 1243 | 5705 | 10115 | 14527 | 19353 | 23095056 |
| 22533679 | 1245 | 5707 | 10117 | 14529 | 19355 | 23095057 |
| 22533680 | 1247 | 5709 | 10119 | 14531 | 19357 | 23095058 |
| 22533681 | 1249 | 5711 | 10121 | 14533 | 19359 | 23095059 |
| 22533682 | 1251 | 5713, 5737 | 10123 | 14535, 17815 | 19361 | 23095060 |
| 22533683 | 1253 | 5715 | 10125 | 14537, 14587 | 19363 | 23095061 |
| 22533684 | 1255 | 5717 | 10127 | 14539 | 19365 | 23095062 |
| 22533685 | 1257 | 5719 | 10129 | 14541, 14589, | 19367 | 23095063 |
| | | | | 17841 | | |
| 22533592, 22533686 | 1259, 1261 | 5721 | 10131 | 14543, 14591, | 19369 | 23095064 |
| | | | | 17839 | | |
| 22533687 | 1263 | 5723 | 10133 | 14545, 14593 | 19371 | 23095065 |
| 22533688 | 1265 | — | 10135 | 14547 | 19373 | — |
| 22533689 | 1267 | — | 10137 | 14549 | 19375 | — |
| 22533690 | 1269 | — | 10139 | 14551 | 19377, 19411 | — |
| 22533692 | 1271 | — | 10141 | 14553, 14555, | 19379 | — |
| | | | | 14595 | | |
| — | 1273 | — | 10143 | 14557 | 19381, 19383 | — |
| — | 1275 | — | 10145 | 14559 | 19385 | — |
| 22534930 | 1277, 3389 | 8071 | 10147, 12347 | 14561, 16997 | 19387, 21787, | 24413460 |
| | | | | | 22625 | |
| 22533693, 22533694 | 1279, 1281 | — | 10149, 10151 | 14563, 14565, | 19389, 19391, | — |
| | | | | 14567, 14569, | 19393 | |
| | | | | 14597, 14599 | | |
| 22533695 | 1283 | — | 10153, 10155 | 14571, 14601 | 19395 | — |
| 22533696 | 1285 | 5725 | 10157 | 14573 | 19397 | — |
| — | 1287, 1289, | 5727 | 10159, 10161 | 14575, 14577 | 19399, 19401 | 23095066 |
| | 1291 | | | | | |
| — | 1293 | 5729 | 10163 | 14579 | 19403 | 23095067 |
| 22533697 | 1295 | 5731 | 10165 | 14581 | 19405 | 23095068 |
| 22533698 | 1297 | 5733, 5735 | 10167, 10169, | 14583, 14603 | 19407 | 23095069 |
| | | | 10171 | | | |
| 22533699 | 1299 | 5739 | 10173 | 14605 | 19413 | 23095070 |
| 22533700 | 1301 | 5741, 5743 | 10175 | 14607 | 19415 | 23095071 |
| 22533701 | 1303 | 5745 | 10177 | 14609 | 19417, 19419 | 23095072 |
| 22533702 | 1305 | 5747, 5803 | 10179 | 14611, 14613 | 19421 | 23095073 |
| 22533703 | 1307 | 5749 | 10181 | 14615 | 19423 | 23095074 |
| 22533704 | 1309 | 5751 | 10183 | 14617 | 19425, 19427 | 23095075 |
| 22533705 | 1311 | 5755 | 10187 | 14621 | 19431 | 23095076 |
| 22533708 | 1313 | 5757 | 10189 | 14623 | 19433 | 23095077 |
| 22533709 | 1315 | 5759, 5761 | 10191 | 14625, 14683, | 19435 | 23095078 |
| | | | | 17733 | | |
| 22533710 | 1317, 1319 | 5763 | 10193 | 14627, 14685, | 19437 | 23095079 |
| | | | | 14689, 14695 | | |
| 22533711 | 1321 | 5765 | 10195 | 14629, 14687, | 19439 | 23095080 |
| | | | | 14691, 14693, | | |
| | | | | 14697, 14699 | | |
| 22533712 | 1323 | 5767 | 10197 | 14631 | 19441 | 23095081 |
| 22533713 | 1325 | 5769 | 10199 | 14633 | 19443 | 23095082 |
| 22533714 | 1327 | 5771 | 10201 | 14635 | 19445 | 23095083 |
| 22533715 | 1329 | 5773 | 10203 | 14637 | 19447 | 23095084 |
| 22533716 | 1331 | 5775 | 10205 | 14639, 17879 | 19449 | 23095085 |
| 22533717 | 1333 | 5777 | 10207 | 14641 | 19451 | 23095086 |
| 22533718 | 1335 | 5779 | 10209 | 14643, 17909 | 19453, 19477 | 23095087 |
| 22533719 | 1337 | 5781 | 10211 | 14645 | 19455 | 23095088 |
| 22533720 | 1339 | 5783 | 10213 | 14647 | 19457 | 23095089 |
| 22533721 | 1341 | 5785 | 10215 | 14649 | 19459 | 23095090 |
| 22533722 | 1343 | 5787 | 10217 | 14651 | 19461 | 23095091 |
| 22533723 | 1345, 1347 | 5789 | 10219 | 14653 | 19463, 19479 | 23095092 |
| 22533725 | 1349 | 5791 | 10221 | 14655 | 19465 | 23095093 |
| 22533726 | 1351 | 5793 | 10223 | 14657 | 19467 | 23095094 |
| 22533728 | 1353 | 5797, 8773 | 10227 | 14661 | 19471 | 23095096 |
| 22533729 | 1355 | 5799 | 10229 | 14663 | 19473 | 23095097 |
| 22533730 | 1357 | 5801 | 10231 | 14665 | 19475, 19481 | 23095098 |
| 22533731 | 1359 | 5805 | 10233 | 14667 | 19483, 19485 | 23095099 |
| 22533732 | 1361 | 5807 | 10235 | 14669 | 19487 | 23095100 |
| 22533733 | 1363 | 5809 | 10237 | 14671 | 19489 | 23095150 |
| 22533734 | 1365 | 5811 | 10239 | 14673 | 19491, 19493 | 23095151 |
| 22533735 | 1367 | 5813 | 10241 | 14675 | 19495 | 23095152 |
| 22533736 | 1369 | 5815 | 10243 | 14677 | 19497 | 23095153 |
| 22533737 | 1371 | 5817 | 10245 | 14679 | 19499 | 23095154 |
| 22533738 | 1373, 1375 | 5819 | 10247 | 14681, 14707 | 19501, 22659 | 23095155 |
| 22533739 | 1377 | 5821 | 10249 | 14709 | 19503 | 23095156 |
| 22533740 | 1379 | 5823 | 10251 | 14701, 14711 | 19505 | 23095157 |
| 22533741 | 1381 | 5825 | 10253 | 14703, 14713 | 19507 | 23095158 |
| 22533742 | 1383 | 5827 | 10255 | 14705, 14717 | 19509 | 23095159 |
| 22533743 | 1385 | 5829, 8779 | 10257 | 14719 | 19511 | 23095160 |
| 22533744 | 1387 | 5831 | 10259 | 14721 | 19513 | 23095161 |
| 22533746 | 1389 | 5833 | 10261 | 14723 | 19515, 19521 | 23095162 |
| 22533747 | 1391 | 5835 | 10263 | 14725 | 19517, 19519, | 23095163 |
| | | | | | 19523, 19525 | |
| 22533748 | 1393 | 5837 | 10265 | 14727 | 19527 | 23095164 |
| 22533751 | 1395 | 5843 | 10271 | 14733 | 19533 | 23095166 |
| 22533752 | 1397 | 5845 | 10273 | 14735 | 19535 | 23095167 |
| 22533753 | 1399 | 5847 | 10275 | 14737 | 19537 | 23095168 |
| 22533754 | 1401 | 5849 | 10277 | 14739 | 19539 | 23095169 |
| 22533756 | 1403 | 5853 | 10281 | 14743 | 19543 | 23095171 |
| 22533757 | 1405, 1407 | 5855 | 10283 | 14745, 14749 | 19545 | 23095172 |
| 22533758 | 1409 | 5857 | 10285 | 14747, 14751 | 19547 | 23095173 |
| 22533759 | 1411 | 5859 | 10287 | 14753 | 19549 | 23095174 |
| 22533760, 22535069 | 1413, 3647, | 5861, 8355, | 10289 | 14755, 17311 | 19551 | 23095175 |
| | 3649 | 8391 | | | | |
| 22533761 | 1415 | 5863 | 10291 | 14757 | 19553 | 23095201 |
| 22533762 | 1417 | 5865 | 10293 | 14759 | 19555 | 23095202 |
| 22533763 | 1419 | 5867 | 10295 | 14761 | 19557 | 23095203 |
| 22533764 | 1421 | 5869 | 10297 | 14763 | 19559 | 23095204 |
| 22533765 | 1423 | 5871 | 10299 | 14765 | 19561 | 23095205 |
| 22533766 | 1425 | 5873 | 10301 | 14767 | 19563 | 23095206 |
| 22533767 | 1427 | 5875 | 10303 | 14769 | 19565 | 23095207 |
| 22533768 | 1429 | 5877 | 10305 | 14771 | 19567 | 23095208 |
| 22533770 | 1431 | 5879 | 10307 | 14773 | 19569, 22591 | 23095209 |
| 22533772 | 1433 | 5883 | 10311 | 14781 | 19573 | 23095211 |
| 22533773 | 1435 | 5885 | 10313 | 14783 | 19575 | 23095212 |
| 22533774 | 1437 | 5887 | 10315 | 14785 | 19577 | 23095213 |
| 22533775 | 1439 | 5889 | 10317 | 14787 | 19579 | 23095214 |
| 22533777 | 1441 | 5891 | 10319, 10321 | 14789, 14809 | 19581 | 23095215 |
| 22533778 | 1443 | 5893 | 10323 | 14791 | 19583 | 23095216 |
| 22533779 | 1445 | 5895 | 10325 | 14793 | 19585 | 23095217 |
| 22533780 | 1447 | 5897 | 10327 | 14795 | 19587 | 23095218 |
| 22533781 | 1449 | 5899 | 10329 | 14797 | 19589 | 23095219 |
| 22533782 | 1451 | 5901 | 10331 | 14799 | 19591 | 23095220 |
| 22533783 | 1453 | 5903 | 10333 | 14801 | 19593 | 23095221 |
| 22533784 | 1455 | 5905 | 10335 | 14803 | 19595 | 23095222 |
| 22533787 | 1457 | 5909 | 10339 | 14807 | 19599 | 23095224 |
| 22533789, 22534806 | 1459, 1461, | 5913, 7927 | 10345, 12199, | 14817, 16835, | 19605, 21643 | 23095226, 24413392 |
| | 3235 | | 12201 | 16837 | | |
| 22533791 | 1463 | 5917 | 10349 | 14821 | 19609 | 23095228 |
| 22533792 | 1465 | 5919 | 10351 | 14823, 14825 | 19611 | 23095229 |
| 22533793 | 1467 | 5921 | 10353 | 14827 | 19613 | 23095230 |
| 22533794 | 1469 | 5923 | 10355 | 14829 | 19615, 19625 | 23095231 |
| 22533795 | 1471 | 5925 | 10357 | 14831 | 19617 | 23095232 |
| 22533796 | 1473 | 5927 | 10359 | 14833 | 19619 | 23095233 |
| 22533797 | 1475 | 5929 | 10361 | 14835 | 19621 | 23095234 |
| 22533798 | 1477, 1479 | 5931 | 10363 | 14837 | 19623, 19627 | 23095235 |
| 22533799 | 1481 | 5933 | 10365 | 14839 | 19629 | 23095236 |
| 22533803 | 1483 | 5941 | 10373 | 14847 | 19637 | 23095240 |
| 22533804 | 1485 | 5943 | 10375 | 14849 | 19639 | 23095241 |
| 22533805 | 1487 | 5945 | 10377 | 14851 | 19641 | 23095242 |
| 22533806 | 1489 | 5947 | 10379 | 14853 | 19643 | 23095243 |
| 22533807 | 1491, 1493 | 5949 | 10381 | 14855, 14881 | 19645 | 23095244 |
| 22533808 | 1495 | 5951 | 10383 | 14857, 14885 | 19647 | 23095245 |
| 22533809 | 1497 | 5953 | 10385 | 14859 | 19649, 19651 | 23095246 |
| 22533811 | 1499 | 5955 | 10387 | 14861 | 19653 | 23095247 |
| 22533812 | 1501 | 5957 | 10389 | 14863, 14887 | 19655, 19657 | 23095248 |
| 22533813 | 1503 | 5959 | 10391 | 14865, 14889 | 19659 | 23095249 |
| 22533814 | 1505 | 5961 | 10393 | 14867 | 19661 | 23095250 |
| 22533815 | 1507 | 5963 | 10395 | 14869 | 19663 | 23095251 |
| 22533816 | 1509 | 5965 | 10397 | 14871 | 19665 | 23095252 |
| 22533817 | 1511 | 5967 | 10399 | 14873 | 19667 | 23095253 |
| 22533818 | 1513 | 5969 | 10401 | 14875 | 19669 | 23095254 |
| 22533819 | 1515 | 8653 | 10403 | 14877 | 19671 | 23095255 |
| 22533826 | 1517 | 5987 | 10415, 10417 | 14901 | 19683 | 23095261 |
| 22533827 | 1519 | 5989 | 10419 | 14903 | 19685 | 23095262 |
| 22533828 | 1521 | 5991 | 10421 | 14905, 14951 | 19687, 19689 | 23095263 |
| 22533829 | 1523 | 5993 | 10423 | 14907 | 19691 | 23095264 |
| 22533830 | 1525 | 5995 | 10425 | 14909 | 19693 | 23095265 |
| 22533833 | 1527 | 5997, 5999 | 10427 | 14911 | 19695 | 23095266 |
| 22533834 | 1529 | 4783, 6001 | 10429 | 13657, 14913 | 19697 | 23094634, 23095267 |
| 22533835 | 1531 | 6003 | 10431 | 14915 | 19699 | 23095268 |
| 22533837 | 1533 | 6009 | 10435 | 14919 | 19703 | 23095270 |
| 22533838 | 1535 | 6011 | 10437 | 14921 | 19705 | 23095271 |
| 22533839 | 1537 | 6013 | 10439 | 14923 | 19707 | 23095272 |
| 22533840 | 1539 | 6015, 6017 | 10441 | 14925 | 19709 | 23095273 |
| 22533841 | 1541, 1543 | 6019 | 10443 | 14927 | 19711, 22693 | 23095274 |
| 22533842 | 1545 | 6021 | 10445 | 14929 | 19713 | 23095275 |
| 22533843 | 1547, 1549, | 6023 | 10447 | 14931 | 19715 | 23095276 |
| | 3971 | | | | | |
| 22533844 | 1551 | 6025 | 10449, 10795 | 14933, 15361 | 19717, 20093 | 23095277 |
| 22533845 | 1553, 1555 | 6027 | 10451 | 14935 | 19719 | 23095278 |
| 22533846 | 1557 | 6029 | 10453 | 14937 | 19721 | 23095279 |
| 22533847 | 1559, 4217 | 6031 | 10455 | 14939 | 19723 | 23095280 |
| 22533849 | 1561, 4011 | 6035 | 10459 | 14943 | 19727 | 23095282 |
| 22533848 | 1563 | 6033 | 10457 | 14941 | 19725 | 23095281 |
| 22533850 | 1565 | 6037 | 10461 | 14945 | 19729 | 23095283 |
| 22533851 | 1567 | 6039 | 10463 | 14947 | 19731, 19773 | 23095284 |
| 22533852 | 1569 | 6041 | 10465 | 14949, 14953 | 19733 | 23095285 |
| 22533855 | 1571 | 6045, 8699 | 10467 | 14955 | 19735 | 23095287 |
| 22533856 | 1573 | 6047, 8697 | 10469 | 14957 | 19737, 19775 | 23095288 |
| 22533857 | 1575, 1577 | 6049 | 10471 | 14959 | 19739 | 23095289 |
| 22533858 | 1581 | 6051 | 10473 | 14961 | 19741 | 23095290 |
| 22533859 | 1583 | 6053 | 10475 | 14963 | 19743 | 23095291 |
| 22533860 | 1585, 1593 | 6055 | 10477 | 14965 | 19745 | 23095292 |
| 22533861 | 1587, 1595, | 6057 | 10479 | 14967 | 19747 | 23095293 |
| | 4285 | | | | | |
| 22533862 | 1589, 1597, | 6059 | 10481 | 14969 | 19749 | 23095294 |
| | 4283 | | | | | |
| 22533863 | 1591, 1599, | 6061, 7307 | 10483 | 14971 | 19751, 21121, | 23095295 |
| | 4281 | | | | 22615, 22689 | |
| 22533864 | 1601 | 6063 | 10485 | 14973 | 19753, 19755 | 23095296 |
| 22533865 | 1603 | 6065 | 10487 | 14975 | 19757 | 23095297 |
| 22533866 | 1605 | 6067 | 10489 | 14977 | 19759, 19777 | 23095298 |
| 22533867 | 1607, 4025 | 6069, 6071 | 10491 | 14979 | 19761 | 23095299 |
| 22533868 | 1609, 4027 | 6073 | 10493 | 14981 | 19763 | 23095300 |
| 22533869 | 1611 | 6075 | 10495 | 14983 | 19765 | 23095301 |
| 22533870 | 1613 | 6077 | 10497 | 14985 | 19767, 19779 | 23095302 |
| 22533871 | 1615 | 6079 | 10499 | 14987 | 19769 | 23095303 |
| 22533872 | 1617 | 6081 | 10501 | 14989, 15001, | 19771 | 23095304 |
| | | | | 17905 | | |
| 22533873 | 1619 | 6083 | 10503 | 14991 | 19781 | 23095305 |
| 22533874 | 1625 | 6085 | 10505 | 14993 | 19783, 19785 | 23095306 |
| — | 1627 | 6087 | 10507 | 14995, 14997 | 19787 | 23095307 |
| 22533875 | 1629 | 6089 | 10509 | 14999 | 19789 | 23095308 |
| 22533877 | 1631 | 6091 | 10511 | — | 19791 | 23095309 |
| 22533878 | 1633, 1637, | 6093 | 10513 | 15003 | 19793 | 23095310 |
| | 4105 | | | | | |
| 22533879 | 1635, 1639, | 6095 | 10515 | 15005 | 19795 | 23095311 |
| | 4107 | | | | | |
| 22533880 | 1641 | 6097 | 10517 | 15007 | 19797 | 23095312 |
| 22533881 | 1643 | 6099 | 10519 | 15009 | 19799 | 23095313 |
| 22533882 | 1645 | 6101 | 10521 | 15011 | 19801, 19813, | 23095314 |
| | | | | | 19815 | |
| 22533883 | 1647 | 6103 | 10523 | 15013, 15083 | 19803 | 23095315 |
| 22533884 | 1649 | 6105 | 10525 | 15015 | 19805 | 23095316 |
| 22533886 | 1651, 1653, | 6107, 6109 | 10527 | 15017 | 19807, 19817 | 23095317 |
| | 1655 | | | | | |
| 22533887 | 1657 | 6111 | 10529 | 15019 | 19809 | 23095318 |
| 22533888 | 1659 | 6113 | 10531 | 15021 | 19811 | 23095319 |
| 22533889 | 1661 | 6115, 6119 | 10533 | 15023 | 19821 | 23095320 |
| 22533890 | 1663 | 6117, 6121 | 10535 | 15025, 15027 | 19823 | 23095321 |
| 22533891 | 1665, 1667, | 6123, 6125 | 10537 | 15029, 15081 | 19825 | 23095322 |
| | 4161 | | | | | |
| 22533892 | 1669 | 6127 | 10539 | 15031 | 19827 | — |
| 22533893 | 1671, 1673, | 6129 | 10541, 10543 | 15033 | 19829 | 23095323 |
| | 1675, 1677, | | | | | |
| | 4129 | | | | | |
| 22533894 | 1679, 1681 | 6131 | 10545 | 15035 | 19831 | 23095324 |
| 22533895 | 1683 | 6133 | 10547 | 15037 | 19833 | 23095325 |
| 22533896 | 1685 | 6135 | 10549 | 15039 | 19835 | 23095326 |
| 22533898 | 1687 | 6137 | 10551 | 15041, 15085 | 19837 | 23095327 |
| 22533899 | 1689 | 6139 | 10553 | 15043 | 19839 | 23095328 |
| 22533900 | 1691 | 6141 | 10555 | 15045 | 19841 | 23095329 |
| 22533901 | 1693, 1695 | 6143 | 10557 | 15047 | 19843, 19899 | 23095330 |
| 22533902 | 1697 | 6145 | 10559 | 15049 | 19845 | 23095331 |
| 22533903 | 1699 | 6147 | 10561 | 15051 | 19847 | 23095332 |
| 22533906 | 1703 | 6153 | 10567 | 15059 | 19853 | 23095335 |
| 22533907 | 1705 | 6155 | 10569, 10573 | 15061 | 19855 | 23095336 |
| 22533908 | 1707, 1709 | 6157 | 10575 | 15063 | 19857 | 23095337 |
| 22533909 | 1711 | 6159 | 10577 | 15065 | 19859 | 23095338 |
| 22533911 | 1713 | 6163 | 10581 | 15069 | 19863 | 23095340 |
| 22533912 | 1715 | 6165, 8677 | 10583 | 15071, 15087 | 19865 | 23095341 |
| 22533913 | 1717 | 6167 | 10585 | 15073 | 19867 | 23095342 |
| 22533914 | 1719 | 6169 | 10587 | 15075 | 19869 | 23095343 |
| 22533915 | 1721, 1723 | 6171 | 10589 | 15077 | 19871 | 23095344 |
| 22533916 | 1725 | 6173 | 10591 | 15079, 15089 | 19873 | 23095345 |
| 22533917 | 1727 | 6175 | 10593 | 15091 | 19875 | 23095346 |
| 22533918 | 1729 | 6177 | 10595 | 15093 | 19877 | 23095347 |
| — | 1731 | 6179 | — | — | 19879 | — |
| 22533927 | 1733 | 6185 | 10599 | 15097 | 19881 | 23095370 |
| 22533928 | 1735 | 6187 | 10601 | 15099 | 19883 | 23095371 |
| 22533929 | 1737 | 6189, 6225 | 10603, 10605 | 15101, 15103 | 19885 | 23095372 |
| 22533931 | 1739 | 6229 | 10609 | 15107 | 19889 | 23095374 |
| 22533932 | 1741 | 6231 | 10611 | 15109 | 19891 | 23095375 |
| 22533933 | 1743 | 6233 | 10613, 10615 | 15111, 15139 | 19893 | 23095376 |
| 22533934 | 1745 | 6235 | 10617 | 15113 | 19895 | 23095377 |
| 22533935 | 1747 | 6237 | 10619 | 15115 | 19897 | 23095378 |
| — | 1751 | 6241 | 10623 | 15119 | 19903 | 23095380 |
| — | 1753 | 6243 | 10625 | 15121 | 19905 | 23095381 |
| 22533959 | 1755 | 6245 | 10627 | 15123 | 19907 | 23095382 |
| 22533960 | 1757 | 6247 | 10629 | 15125 | 19909 | 23095383 |
| 22533961 | 1759 | 6249, 6325 | 10631 | 15127 | 19911 | 23095384 |
| 22533962 | 1761 | 6251 | 10633 | 15129 | 19913 | 23095385 |
| 22533963 | 1763 | 6253 | 10635 | 15131 | 19915 | 23095386 |
| 22533964 | 1765 | — | 10637 | 15133 | 19917 | — |
| 22533966 | 1767 | 6257 | 10639 | 15135 | 19919 | 23095387 |
| 22533968 | 1769 | 6259 | 10641 | 15137 | 19921 | 23095388 |
| 22533969 | 1771 | 6261 | 10643 | 15141 | 19923 | 23095389 |
| 22533970 | 1773 | 6263 | 10645 | 15143 | 19925 | 23095390 |
| 22533971 | 1775 | 6265 | 10647 | 15145 | 19927 | 23095391 |
| 22533972 | 1777 | 6267 | 10649 | 15147 | 19929 | 23095392 |
| 22533973 | 1779 | 6269 | 10651 | 15149 | 19931 | 23095393 |
| 22533974 | 1781 | 6271 | 10653 | 15151 | 19933 | 23095394 |
| 22533424, 22533976 | 1783 | 5213, 6275 | 9595, 10657 | 14015, 15155 | 18803, 19937 | 23094866, 23095396 |
| 22533977 | 1785, 3955 | 6277 | 10659 | 15157 | 19939 | 23095397 |
| 22533978 | 1787, 3957 | 6279 | 10661 | 15159 | 19941 | 23095398 |
| 22533979 | 1789 | 6281 | 10663 | 15161 | 19943 | 23095399 |
| 22533980 | 1791 | 6283 | 10665 | 15163, 15167 | 19945 | 23095400 |
| 22533981 | 1793 | 6285 | 10667 | 15165 | 19947 | 23095401 |
| 22533982 | 1795, 4007, | 6287 | 10669 | 15169 | 19949, 19951 | 23095402 |
| | 4009 | | | | | |
| 22533983 | 1797 | 6289 | 10671 | 15171 | 19953 | 23095403 |
| 22533984 | 1799 | 6291 | 10673 | 15173 | 19955 | 23095404 |
| 22533986 | 1801 | 6293 | 10675, 10677 | 15175, 15177 | 19957 | 23095405 |
| 22533987 | 1803, 1805 | 6295 | 10679 | 15179, 15181 | 19959 | 23095406 |
| 22533990 | 1811 | 6329 | 10683 | 15231 | 19965 | 23095407 |
| 22533991 | 1813 | 6331 | 10685 | 15233 | 19967 | 23095408 |
| 22533992 | 1815 | 6333 | 10687 | 15235 | 19969, 20041 | 23095409 |
| 22533993 | 1817 | 6335 | 10689 | 15237 | 19971 | 23095410 |
| 22533994 | 1819 | 6337 | 10691 | 15239 | 19973 | 23095411 |
| — | 1821 | 6339 | 10693 | 15241, 15243 | 19975 | 23095412 |
| 22533995 | 1823 | 6341 | 10695, 10697 | 15245 | 19977 | 23095413 |
| 22533996 | 1825 | 6343 | 10699 | 15247, 15259 | 19979 | 23095414 |
| 22533997 | 1827 | 6345 | 10701 | 15249, 15261, | 19981 | 23095415 |
| | | | | 15263 | | |
| 22533998 | 1829 | 6347 | 10703 | 15251 | 19983 | 23095416 |
| 22533999 | 1831 | 6349 | 10705 | 15253 | 19989 | 23095417 |
| 22534000 | 1833 | 6351 | 10707 | 15255 | 19991 | 23095418 |
| 22533157, 22534001 | 1835 | 4631, 6353, | 9071, 10709 | 13491, 15257, | 19993 | 23094571, 23095419 |
| | | 6355 | | 15265, 15293, | | |
| | | | | 15295, 17951 | | |
| 22534002 | 1837 | 6357 | 10711 | 15267, 15297, | 19995, 22611 | 23095420 |
| | | | | 15299 | | |
| 22534003 | 1839 | 6359 | 10713 | 15269 | 19997 | 23095421 |
| 22534004 | 1841 | 6361, 6383 | 10715 | 15271, 15301 | 19999 | 23095470 |
| 22534005 | 1843 | 6363 | 10717 | 15273 | 20001 | 23095471 |
| 22534007 | 1845 | 6365 | 10719 | 15275 | 20003 | 23095472 |
| 22534008 | 1847 | 6367 | 10721 | 15277 | 20005 | 23095473 |
| 22534009 | 1849 | 6369 | 10723 | 15279, 15303 | 20007 | 23095474 |
| 22534010 | 1851 | 6371 | 10725 | 15281 | 20009, 20043 | 23095475 |
| 22534011 | 1853 | 6373 | 10727 | 15283 | 20011 | 23095476 |
| 22534012 | 1855 | 6375 | 10729 | 15285 | 20013 | 23095477 |
| 22534013 | 1857 | 6377 | 10731 | 15287 | 20015 | 23095478 |
| 22534014 | 1859 | 6379 | 10733 | 15289 | 20017, 20045 | 23095479 |
| 22534015, 22535008 | 1861, 3547 | 6381, 6385, | 10735, 12503 | 15291, 17187, | 20019, 20021, | 23095480, 24413534 |
| | | 8233 | | 17229 | 20023, 21927 | |
| 22534016 | 1863 | 6387, 6389, | 10737 | 15305, 17697 | 20025 | 23095481 |
| | | 6391, 6393 | | | | |
| 22534017, 22534018 | 1865, 1867 | 6395 | 10739, 10741 | 15307, 15309 | 20027, 20029 | 23095482, 23095483 |
| 22534022 | 1869 | 6403 | 10749 | 15317 | 20037 | 23095487 |
| 22534023 | 1871, 1873 | 6405 | 10751 | 15319 | 20039 | 23095488 |
| 22534025 | 1875 | 6409 | 10755 | 15323 | 20049 | 23095490 |
| 22534026 | 1877 | 6411 | 10757, 10759, | 15325, 15327, | 20051, 20053 | 23095491 |
| | | | 10763 | 15329, 17801 | | |
| 22534027 | 1879 | 6413 | 10765 | 15331 | 20055 | 23095492 |
| 22534029 | 1881 | 6415, 6451, | 10767 | 15333, 15407 | 20057, 20059 | 23095493 |
| | | 6453 | | | | |
| 22534030 | 1883 | 6455 | 10769 | 15335 | 20061 | 23095494 |
| 22534031 | 1885 | 4743, 6457 | 10771 | 15337 | 20063 | 23095495 |
| 22534032 | 1887 | 6459 | 10773 | 15339 | 20065 | 23095496 |
| 22534033 | 1889 | 6461, 6465 | 10775 | 15341 | 20067 | 23095497 |
| 22534034 | 1891 | 6463, 6467, | 10777 | 15343 | 20069 | 23095498 |
| | | 6471 | | | | |
| 22534035 | 1893 | 6469, 6473 | 10779 | 15345 | 20071, 20079 | 23095499 |
| 22534036 | 1895 | 6475 | 10781 | 15347 | 20073 | 23095500 |
| 22534037 | 1897 | 6477 | 10783 | 15349 | 20075 | 23095501 |
| 22534038 | 1899 | 6479 | 10785 | 15351 | 20077, 20081 | 23095502 |
| 22534039 | 1901 | 6481 | 10787 | 15353 | 20083 | 23095503 |
| 22534040 | 1903 | 6483 | 10789 | 15355 | 20085 | 23095504 |
| 22534041 | 1905 | 6485, 8719 | 10761, 10791 | 15357 | 20087, 20089 | 23095505 |
| 22534042 | 1907 | 6487 | 10793 | 15359 | 20091 | — |
| 22534044 | 1909 | 6491 | — | 15363 | — | 23095506, 23095507 |
| 22534045 | 1911 | 6493 | — | 15365 | — | 23095508 |
| 22534046 | 1913 | 6495 | 10797 | 15373 | — | 23095509 |
| 22534048 | 1915 | 6499 | — | — | — | 23095510 |
| 22534049, 22534057, | 1917, 1941, | 6501, 6523, | 10801, 10835 | 15377 | 20113, 20115, | 23095518, 23095519 |
| 22534058 | 1945, 1947 | 6525 | | | 22549 | |
| 22534054, 22534717, | 1919, 1921, | 6503, 6505, | 10803, 10813, | 15369, 16655, | 20095, 20107, | 23095515, 24413307 |
| 22534736 | 1931, 1935 | 6515, 7761 | 10815, 10821, | 17945 | 21461 | |
| | | | 10823, 10829, | | | |
| | | | 11993, 11995 | | | |
| — | 1923 | 6507 | — | — | — | 23095511 |
| 22534051 | 1925 | 6509 | 10825 | — | — | 23095512 |
| 22534052 | 1927 | 6511 | — | — | — | 23095513 |
| 22534053, 22534738 | 1929, 1933, | 6513 | 12029 | 16689 | 21503 | 23095514 |
| | 3101 | | | | | |
| 22534055 | 1937 | 6517 | — | — | — | 23095516 |
| 22534056 | 1939, 1943 | 6519, 6521 | 10833 | — | 20111 | 23095517 |
| 22534059 | 1949 | 6527, 6539 | 10837 | 15379 | 20117 | 23095520 |
| 22534060 | 1951 | 6529 | 10839 | 15381 | 20119 | 23095521 |
| 22534062 | 1953 | 6533, 6535 | 10843 | 14883, 15385 | 20123 | 23095523 |
| 22534063 | 1955 | 6537, 6543 | 10845, 10847 | — | 20125, 20127 | 23095524 |
| 22534064 | 1957 | 6545 | 10849 | 15387 | 20129 | 23095525 |
| 22534065 | 1959 | 6547 | 10851 | 15389 | 20131 | 23095526 |
| 22534066 | 1961 | 6549 | 10853 | 15391, 15409 | 20133, 20149 | 23095527 |
| 22534067 | 1963 | 6551 | 10855 | 15393 | 20135 | 23095528 |
| 22534068 | 1965 | 6553 | 10857 | 15395 | 20137 | 23095529 |
| 22534069 | 1967 | 6555 | 10859 | 15397 | 20139 | 23095530 |
| 22534070 | 1969 | 6557 | 10861 | 15399 | 20141 | 23095531 |
| 22534071 | 1971 | 6559 | 10863 | 15401 | 20143 | 23095532 |
| 22534073 | 1973 | 6561 | 10865 | 15403 | 20145 | 23095533 |
| 22534074 | 1975 | 6563 | 10867 | 15405, 17745 | 20147 | 23095534 |
| — | 1977 | 6565 | 10869 | 15411 | 22495 | 23095535 |
| 22534076 | 1979 | 6569 | 10873 | 15417 | 20151, 22491 | 23095537 |
| 22534078 | 1981 | 6571 | 10875 | 15419 | 20153 | 23095538 |
| 22534079 | 1983 | 6573 | 10877 | 15421 | 20155 | 23095539 |
| 22534080 | 1985 | 6575 | 10879 | 15423 | 20157 | 23095540 |
| 22534081 | 1987 | 6577 | 10881 | 15425 | 20159, 20243 | 23095541 |
| 22534082 | 1989 | 6579 | 10883 | 15427 | 20161 | 23095542 |
| 22534083 | 1991 | 6581 | 10885 | 15429 | 20163 | 23095543 |
| 22534084 | 1993 | 6583 | 10887 | 15431 | 20165 | 23095544 |
| 22534085 | 1995 | 6585 | 10889 | 15433, 17835 | 20167 | 23095545 |
| 22534086 | 1997 | 6587 | 10891 | 15435 | 20169 | 23095546 |
| 22534087 | 1999 | 6589 | 10893 | 15437 | 20171 | 23095547 |
| 22534088 | 2001 | 6591 | 10895 | 15439 | 20173 | 23095548 |
| 22534089 | 2003 | 6593 | 10897 | 15441 | 20175 | 23095549 |
| 22534090 | 2005, 4063 | 6595 | 10899 | 15443 | 20177 | 23095550 |
| 22534095 | 2007 | 6597 | 10901 | 15445 | 20179, 22633 | 23095552 |
| 22534096 | 2009, 2011 | 6599 | 10903 | 15447 | 20181 | 23095553 |
| 22534097 | 2013 | 6601 | 10905 | 15449 | 20183, 20245 | 23095554 |
| 22534098 | 2015 | 6603 | 10907 | 15451 | 20185, 20247 | 23095555 |
| 22534099 | 2017 | 6605 | 10909 | 15453 | 20187 | 23095556 |
| 22534100 | 2019 | 6607 | 10911 | 15455 | 20189 | 23095557 |
| 22534101 | 2021 | 6609 | 10913 | 15457 | 20191 | 23095558 |
| 22534102 | 2023 | 6611, 8775 | 10915 | 15459 | 20193 | 23095559 |
| 22534103 | 2025 | 6613 | 10917 | 15461 | 20195 | 23095560 |
| 22534104 | 2027 | 6615 | 10919 | 15463 | 20197 | 23095561 |
| 22534106 | 2029 | 6617 | 10921 | 15465 | 20199 | 23095562 |
| 22534107 | 2031 | 6619 | 10923 | 15467 | 20201 | 23095563 |
| 22534108 | 2033 | 6621 | 10925 | 15469 | 20203 | 23095564 |
| 22534109 | 2035 | 6623, 6641 | 10927 | 15471 | 20205 | 23095565 |
| 22534110 | 2037 | 6625, 6643, | 10929 | 15473, 15481 | 20207 | 23095566 |
| | | 8729 | | | | |
| 22534111 | 2039 | 6627 | 10931 | 15475 | 20209 | 23095567 |
| 22534112 | 2041 | 6629 | 10933 | 15477 | 20211 | 23095629 |
| 22534113 | 2043, 2045 | 6631 | 10935 | 15479, 15483 | 20213 | 23095630 |
| 22534114 | 2047, 4137, | 6633 | 10937, 12801 | 15485, 17613 | 20215, 22273 | 23095631 |
| | 4261 | | | | | |
| 22534115 | 2049, 2051, | 6635 | 10939, 10941 | 15487, 17639 | 20217, 20219, | 23095632 |
| | 3837 | | | | 22297 | |
| 22534117 | 2053, 2055 | 6637 | 10943 | 15489, 17765, | 20221 | 23095633 |
| | | | | 17767 | | |
| 22534118 | 2057 | 6647 | 10945 | 15491 | 20223 | 23095634 |
| 22534119 | 2059 | 6649 | 10947 | 15493 | 20225 | 23095635 |
| 22534120 | 2061 | 6651 | 10949, 10951 | 15495 | 20227 | 23095636 |
| 22534121 | 2063 | 6653 | 10953 | 15497 | 20229 | 23095637 |
| 22534122 | 2065 | 6655 | 10955 | 15499 | 20231 | 23095638 |
| 22534123 | 2067 | 6657 | 10957 | 15501 | 20233 | 23095639 |
| 22534124 | 2069 | 6659 | 10959 | 15503 | 20235 | 23095640 |
| 22534125 | 2071 | 6661 | 10961 | 15505 | 20237 | 23095641 |
| 22534126 | 2073 | 6663 | 10963 | 15507 | 20239 | 23095642 |
| 22534127 | 2075 | 6665 | 10965 | 15509 | 20241 | 23095643 |
| 22534128 | 2077 | 6667 | 10967 | 15511 | 20249 | 23095644 |
| 22534129 | 2079 | 6669 | 10969, 11025 | 15513, 15563 | 20251 | 23095645 |
| 22534130 | 2081 | 6671 | 10971 | 15515 | 20253 | 23095646 |
| 22534131 | 2083 | 6673 | 10973 | 15517 | 20255 | 23095647 |
| 22534132 | 2085 | 6675, 6677 | 10975 | 15519 | 20257 | 23095648 |
| 22534133 | 2087 | 6679 | 10977, 13075 | 15521 | 20259 | 23095649 |
| 22534330 | 2089, 2437, | 6997 | 11253 | 15843 | 20687 | 24412958 |
| | 2439 | | | | | |
| 22534134 | 2091 | 6639, 6681 | 10979 | 15523, 15565, | 20261 | 23095650 |
| | | | | 15571 | | |
| 22534135 | 2093 | 6683 | 10981 | 15525, 15567 | 20263 | 23095651 |
| 22534136 | 2095 | 6685 | 10983 | 15527 | 20265 | 23095652 |
| 22534137 | 2097 | 6687 | 10985 | 15529, 15573 | 20267 | 23095653 |
| 22534138 | 2099 | 6689, 6767 | 10987 | 15531 | 20269 | 23095654 |
| 22534140 | 2101 | 6691 | 10989 | 15533 | 20271 | 23095655 |
| 22534141 | 2103 | 6693, 6769 | 10991 | 15535 | 20273 | 23095656 |
| 22534142 | 2105 | 6695 | 10993 | 15537 | 20275 | 23095657 |
| 22534143 | 2107 | 6697 | 10995 | 15539 | 20277 | 23095658 |
| 22534144 | 2109 | 6699 | 10997 | 15541 | 20279 | 23095659 |
| 22534145 | 2111 | 6701 | 10999 | 15543 | 20281 | 23095660 |
| 22534146 | 2113 | 6703 | 11001 | 15545 | 20283 | 23095661 |
| 22534147 | 2115 | 6705 | 11003 | 15547 | 20285 | 23095662 |
| 22534148 | 2117 | 6707 | 11005 | 15549, 15551 | 20287 | 23095663 |
| 22534149 | 2119, 2121 | 6709, 6715, | 11007 | 15553, 15555 | 20289, 20291, | 23095664 |
| | | 6717, 6719 | | | 20297, 20299, | |
| | | | | | 20301 | |
| 22534151 | 2123 | 6721 | 11009 | 15557, 15569 | 20303 | 23095665 |
| 22534152 | 2125 | 6723 | 11011 | 15559 | 20305 | 23095666 |
| 22534170 | 2127 | 6761 | 11047 | 15591 | 20343 | 23095682 |
| 22534171 | 2129 | 6763 | 11049 | 15593 | 20345, 20347 | 23095683 |
| 22534172 | 2131 | 6765, 6771, | 11051 | 15595 | 20349 | 23095684 |
| | | 8705 | | | | |
| 22534173 | 2133 | 6773 | 11053 | 15597, 15599, | 20351 | 23095685 |
| | | | | 15601 | | |
| 22534174 | 2135 | 6775, 6777 | 11055 | 15603 | 20353 | 23095686 |
| 22534175 | 2137 | 6779 | 11057 | 15605 | 20355 | 23095687 |
| 22534176 | 2139 | 6781 | 11059 | 15607 | 20357 | 23095688 |
| 22534177 | 2141, 4145 | 6783 | 11061, 11063 | 15609 | 20359 | 23095689 |
| — | 2143 | — | — | — | — | 23095690 |
| — | 2145 | — | — | — | — | 23095691 |
| — | 2147 | — | — | — | — | 23095692 |
| — | 2151 | — | — | — | — | 23095693 |
| 22534181 | 2153 | 6795 | 11071 | 15617 | 20379 | 23095701 |
| 22534182 | 2155 | 6797 | 11073 | 15619 | 20381 | 23095702 |
| 22534183 | 2157 | 6799 | 11075 | 15621 | 20383 | 23095703 |
| 22534184 | 2159 | 6787, 6801 | 11077 | 15623 | 20385 | 23095704 |
| 22534185 | 2161 | 6803 | 11079 | 15625 | 20387 | 23095705 |
| 22534186 | 2163, 2165 | 6805 | 11081 | 15627 | 20389 | 23095706 |
| 22534187 | 2167 | 6807 | 11083 | 15629 | 20391 | 23095707 |
| 22534188 | 2169 | 6809 | 11085 | 15631, 15633 | 20393 | 23095708 |
| 22534190 | 2171 | 6811 | 11087 | 15635 | 20395 | 23095709 |
| 22534200 | 2173, 4191 | 6827, 6943 | 11103 | 15651 | 20411 | 23095717 |
| 22534201 | 2175 | 6829 | 11105 | 15653, 15655 | 20413 | 23095718 |
| 22534202 | 2177 | 6831, 6945 | 11107 | 15657 | 20415 | 23095719 |
| 22534203 | 2179 | 6833 | 11109 | 15659 | 20417 | 23095720 |
| 22534204 | 2181 | 6835 | 11111 | 15661 | 20419 | 23095721 |
| 22534205 | 2183 | 6837 | 11113 | 15663 | 20421 | 23095722 |
| 22534206 | 2185 | 6839 | 11115 | 15671 | 20423 | 23095723 |
| 22534207 | 2187 | 6841, 6947 | 11117 | 15673 | 20425 | 23095724 |
| 22534208 | 2189 | 6843 | 11119 | 15675 | 20427 | 23095725 |
| 22534209 | 2191 | 6845 | 11121 | 15677 | 20429, 20441 | 23095726 |
| 22534210 | 2193 | 6847 | 11123 | 15679 | 20431, 20443 | 23095727 |
| 22534211, 22535115 | 2195, 2197, | 5103, 6849, | 11125, 12643 | 15681, 17443 | 20433, 22067 | 23095728, 24413604 |
| | 3735, 3737, | 8443 | | | | |
| | 3739 | | | | | |
| 22534212 | 2199 | 6851 | 11127 | 15683 | 20435 | 23095729 |
| 22534214 | 2201 | 6855 | 11129 | 15685 | 20437 | 23095730 |
| 22534215 | 2203 | 6857 | 11131, 11133 | 15687 | 20445 | 23095731 |
| 22534216 | 2205, 2207 | 6859 | 11135 | 15689 | 20447, 20449 | 23095732 |
| 22534217 | 2209 | 6861, 6863 | 11137 | 15691 | 20451 | 23095733 |
| 22534218 | 2211 | 6865 | 11139 | 15693 | 20453, 20525, | 23095734 |
| | | | | | 20527 | |
| 22534219 | 2213 | 6867 | 11141 | 15695, 17919 | 20455, 20529 | 23095735 |
| 22534220 | 2215 | 6869 | 11143 | 15697 | 20457 | 23095736 |
| 22534221 | 2217 | 6871 | 11145 | 15699, 15773 | 20459 | 23095737 |
| 22534222 | 2219 | 6873 | 11147 | 15701, 17755 | 20461 | 23095738 |
| 22534223 | 2221 | 6875 | 11149 | 15703 | 20463 | 23095739 |
| 22534224 | 2223 | 6877 | 11151 | 15705, 15775 | 20465 | 23095740 |
| 22534225 | 2225 | 6879 | 11153 | 15707 | 20467 | 23095741 |
| 22534226 | 2227 | 6881 | 11155 | 15709 | 20469 | 23095742 |
| 22534227 | 2229 | 6883 | 11157 | 15711 | 20471 | 23095743 |
| 22534228 | 2231 | 6885 | 11159 | 15713 | 20473 | 23095744 |
| 22534229 | 2233 | 6889 | 11161 | 15715 | 20475 | 24412859 |
| 22534230 | 2235 | 6891 | 11163 | 15717 | 20477 | 24412860 |
| 22534231 | 2237 | 6893 | 11165 | 15719 | 20479 | 24412861 |
| 22534232 | 2239 | 6895 | 11167 | 15721 | 20481 | 24412862 |
| 22534233 | 2241 | 6897 | 11169 | 15723 | 20483 | 24412863 |
| 22534235 | 2243 | 6899 | 11171 | 15725 | 20485 | 24412864 |
| 22534236 | 2245 | 6901 | 11173 | 15727 | 20487 | 24412865 |
| 22534237 | 2247, 2249 | 6903 | 11175 | 15729 | 20489 | 24412866 |
| 22534239 | 2253, 2255 | 6905 | 11179 | 15733 | 20491 | 24412867 |
| 22534240 | 2257, 2259 | 6907 | 11181 | 15735 | 20493 | 24412868 |
| 22534241 | 2261 | 6909 | 11183 | 15737 | 20495 | 24412869 |
| 22534242 | 2263 | 6911 | 11185 | 15739 | 20497 | 24412870 |
| 22534243 | 2265 | 6913 | 11187 | 15741 | 20499 | 24412871 |
| 22534244 | 2267 | 6915 | 11189 | 15743 | 20501, 22713 | 24412872 |
| 22534245 | 2269 | 6917 | 11191 | 15745 | 20503 | 24412873 |
| 22534246 | 2271 | 6919, 6921 | 11193, 11195 | 15747, 15749 | 20505 | 24412874 |
| 22534247 | 2273, 2275, | 6923 | 11197, 11199 | 15751, 17741 | 20507 | 24412875 |
| | 2277, 2279, | | | | | |
| | 2281 | | | | | |
| — | 2283 | 6925 | 11201 | 15753 | 20509 | 24412876 |
| — | 2285 | — | — | — | — | 24413553 |
| 22534248 | 2287 | 6927 | 11203 | 15755 | 20511 | 24412877 |
| 22534249 | 2289 | 6929 | 11205 | 15757 | 20513 | 24412878 |
| 22534250 | 2291 | 6931 | 11207 | 15759 | 20515 | 24412879 |
| — | 2293 | 6933 | 11209 | 15761 | 20517 | 24412880 |
| 22534257 | 2295 | 6949 | 11219 | 15777 | 20531 | 24412886 |
| 22534258, 22534268 | 2297, 2327 | 6951, 6973 | 11221, 11241 | 15779 | 20551 | 24412887, 24412893 |
| 22534259, 22534269 | 2299, 2301, | 6953, 6965, | 11223, 11243 | 15781, 15807 | 20533 | 24412888 |
| | 2329 | 6975 | | | | |
| 22534260 | 2303 | 6955 | 11225 | 15783 | 20535 | 24412889 |
| 22534261 | 2305 | 6961 | 11231 | 15789 | 20541 | 24412890 |
| 22534262 | 2307 | 6963 | 11233 | 15791 | 20543 | 24412891 |
| 22534263 | 2311 | — | — | 14715, 15053, | — | — |
| | | | | 15793 | | |
| — | 2313 | 6967 | 11235, 11237 | 15795 | 20545, 20547 | 24412892 |
| 22534264 | 2315, 3961 | 6969, 6971 | 11239 | 15797 | 20549 | 23094836, 23095149, |
| | | | | | | 23095422, |
| | | | | | | 23095568 |
| 22534265 | 2317 | — | — | 15799 | — | — |
| 22534266 | 2319 | — | — | 15801 | — | — |
| — | 2321 | — | — | 15803 | — | — |
| 22534272 | 2335 | 6979 | 11247 | 15809 | 22489, 22497 | 24412895 |
| 22534273 | 2337 | — | 13025, 13053 | 15811 | 20565, 22499 | 24412896, 24412905 |
| 22534274 | 2339 | — | 13027, 13055 | 15813 | 20567, 22501 | 24412897, 24412906 |
| 22534275 | 2341 | — | — | — | 20569 | 24412907 |
| 22534276 | 2343 | — | — | — | 20571 | 24412918 |
| 22533658, 22534277 | 2345 | — | — | 14483 | 20573 | 24412919 |
| 22534278 | 2347 | — | — | — | 20575 | — |
| 22534282 | 2353, 2355 | — | — | — | 20585, 20603 | — |
| 22534283 | 2357 | — | — | — | 20587 | — |
| 22534285 | 2359 | — | — | — | 20589, 20605 | — |
| 22534286 | 2361 | — | — | — | 20591 | — |
| 22534287 | 2363, 2365 | — | — | — | 20593 | — |
| — | 2367 | — | — | — | 20595 | — |
| 22534288 | 2371, 4151, | — | — | — | 20599 | — |
| | 4177 | | | | | |
| 22534290 | 2373 | — | — | 15189 | 20601 | — |
| 22534291 | 2375 | — | — | — | 20607 | — |
| 22534293 | 2377, 4103 | — | 9899, 9901, | — | 20361, 20363, | 23095694 |
| | | | 9903 | | 20365, 20611 | |
| 22534294 | 2379 | — | — | — | 20613 | — |
| 22534295 | 2381 | — | — | — | 20615 | — |
| 22534296 | 2383 | — | — | — | 20617 | — |
| 22534299 | 2385 | — | — | — | 20623 | 24412926 |
| 22534300 | 2387 | — | — | — | 20625 | 24412927 |
| 22534301 | 2389 | — | — | — | 20627 | 24412928 |
| 22534305 | 2391 | — | — | — | 20633, 20635, | 24412932, 24412933 |
| | | | | | 20637, 20675, | |
| | | | | | 22543 | |
| 22534306 | 2393 | — | — | — | 20639 | 24412934 |
| 22534307 | 2395 | — | — | — | 20641 | 24412935 |
| 22534308 | 2397 | — | — | — | 20643 | 24412936 |
| 22534310 | 2399 | — | — | — | 20645 | 24412937 |
| 22534311 | 2401 | — | — | — | 20647 | 24412938 |
| 22534313 | 2405 | — | — | — | 20651 | 24412940 |
| 22534314 | 2407 | — | — | — | 20655 | 24412942 |
| 22534315 | 2409 | — | — | — | 20657 | 24412943 |
| — | 2411 | — | — | — | 20659 | 24412944 |
| 22534316 | 2413 | — | — | — | 20661 | 24412945 |
| 22534317 | 2415 | — | — | — | 20663 | — |
| 22534318 | 2417 | — | — | — | 20665 | 24412947 |
| 22534321, 22534910 | 2419 | — | — | — | 20669 | 24412950 |
| 22534322 | 2421 | — | — | — | 20671 | — |
| 22534323 | 2423 | — | — | — | 20673 | 24412952 |
| 22534324 | 2425 | — | — | — | 20677 | 24412953 |
| 22534325 | 2427, 4031 | 6981 | 13003 | 15831 | 20679 | 24412954 |
| 22534326 | 2429, 2431, | 6983 | 13001 | 15835 | 20681 | 24412955 |
| | 4029 | | | | | |
| 22534327 | 2433 | 6985, 6991, | 11249 | 15837, 15839, | 20683 | 24412956 |
| | | 6993 | | 15845 | | |
| 22534329 | 2435 | 6995 | 11251 | 15841 | 20685 | 24412957 |
| 22534332 | 2441 | 6999 | 11255 | 15847 | 20689 | 24412959 |
| 22534333 | 2443 | 7001, 7003 | 11257, 11259 | 15849, 15851, | 20691, 20693 | 24412960 |
| | | | | 15857 | | |
| 22534335 | 2445 | 7005 | 11263 | 15855, 15859 | 20697 | 24412961 |
| 22534336 | 2447 | 7007 | 11265 | 15861 | 20699 | 24412962 |
| 22534337 | 2449 | 7009 | 11267 | 15863 | 20701 | 24412963 |
| 22534339 | 2451 | 7013 | 11271 | 15867 | 20705 | 24412964 |
| 22533322, 22534340 | 2453 | 4981, 7015 | 9411, 11273 | 13801, 15869 | 18555, 20707 | 24412965 |
| 22534341 | 2455 | 7017 | 11275 | 15871, 15899 | 20709 | 24412966 |
| 22534342 | 2457 | 7019 | 11277 | 15873 | 20711, 20713 | 24412967 |
| 22534343 | 2459 | 7021 | 11279 | 15875 | 20715 | 24412968 |
| 22534344 | 2461 | 7023 | 11281 | 15877 | 20717 | 24412969 |
| 22534345 | 2463 | 7025 | 11283 | 15879 | 20719 | 24412970 |
| 22534346 | 2465 | 7027 | 11285 | 15881 | 20721 | 24412971 |
| 22534347 | 2467 | 7029 | 11287 | 15883 | 20723 | 24412972 |
| 22534348 | 2469 | 7031 | 11289 | 15885 | 20725 | 24412973 |
| 22534349 | 2471 | 7033 | 11291 | 15887 | 20727 | 24412974 |
| 22534350 | 2473 | 7035 | 11293 | 15889 | 20729 | 24412975 |
| 22534351 | 2475 | 7037 | 11295 | 15891 | 20731 | 24412976 |
| 22534352 | 2477 | 7039 | 11297 | 15893 | 20733 | 24412977 |
| 22534353 | 2479 | 8663 | 11299 | 15895 | 20735 | 24412978 |
| 22534354 | 2481 | 8661 | 11301 | 15897, 15901 | 20737 | 24412979 |
| 22534359 | 2483, 2485 | 7041, 8739 | 11309 | 15909 | 20745 | 24412983 |
| 22534360 | 2487 | 7043 | 11311 | 15911, 15913 | 20747 | 24412984 |
| 22534361 | 2489 | 7045 | 11313 | 15915 | 20749 | 24412985 |
| 22534362 | 2491 | 7047 | 11315 | 15917 | 20751 | 24412986 |
| 22534363 | 2493, 2495 | 7049 | 11317 | 15919 | 20753, 20767 | 24412987 |
| 22534364 | 2497 | 7051 | 11319 | 15921 | 20755 | 24412988 |
| 22534365 | 2499 | 7053 | 11321 | 15923, 15925 | 20757 | 24412989 |
| 22534366 | 2501 | 7055, 7057 | 11323, 11351 | 15927 | 20759 | 24412990 |
| 22534367 | 2503 | 7059 | 11325 | 15929 | 20761 | 24412991 |
| 22534368 | 2505 | 7061 | 11327 | 15931, 15935 | 20763 | 24412992 |
| 22534369 | 2507 | 7063 | 11329 | 15933, 15937 | 20765 | 24412993 |
| 22534370 | 2509 | 7065 | 11331 | 15939 | 20769 | 24412994 |
| 22534371 | 2511 | 7071 | 11333 | 15941 | 20771 | 24412995 |
| 22534372 | 2513 | 7073, 7075 | 11335 | 15943, 15945 | 20773, 20775 | 24412996 |
| 22534373, 22534971 | 2515, 3467, | 4363, 7077, | 11337, 12427 | 15947, 17083 | 20777, 21847 | 24412914, 24412997, |
| | 3469, 3471, | 8149 | | | | 24413497 |
| | 3959 | | | | | |
| 22534377 | 2517 | 7083 | 11343 | 15953 | 20783 | 24413000 |
| 22534378 | 2519 | 7085 | 11345 | 15955 | 20785 | 24413001 |
| 22534379 | 2521 | 7087 | 11347 | 15957 | 20787 | 24413002 |
| 22534380 | 2523 | 7089 | 11349, 11353, | 15959, 15965 | 20789 | 24413003 |
| | | | 13065 | | | |
| 22534381 | 2525 | 7091 | 11355 | 15961 | 20791, 20793 | 24413004 |
| 22534382 | 2527 | 7093 | 11357, 11359 | 15963 | 20795 | 24413005 |
| 22534383 | 2529 | 7101, 7103 | 11361 | 15967 | 20797, 20799 | 24413006 |
| 22534384 | 2531 | 7105 | 11363 | — | — | 24413007 |
| 22534385 | 2533 | — | 11365 | — | 20801 | 24413008 |
| 22534386 | 2535 | 7107 | 11367 | 15969, 15971 | 20803 | 24413009 |
| 22534387 | 2537 | 7109 | 11369 | 15973 | 20805 | 24413010 |
| 22534388 | 2539 | 7111, 7113 | 11371, 11373 | 15975 | 20807 | 24413011 |
| 22534389 | 2541 | 7115 | 11375 | 15977 | 20809, 20811 | 24413012 |
| 22534390 | 2543 | 7117 | 11377 | 15979 | 20813 | 24413013 |
| 22534391 | 2545 | 7119 | 11379 | 15981 | 20815, 20817 | 24413014 |
| 22534392 | 2547 | 7121 | 11381 | 15983 | 20819 | 24413015 |
| 22534393 | 2549 | 7123 | 11383 | 15985 | 20821 | 24413016 |
| 22534394 | 2551 | 7125 | 11385 | 15987 | 20823 | 24413017 |
| 22534395 | 2553 | 7127 | 11387 | 15989 | 20825 | 24413018 |
| 22534396 | 2555 | 7129 | 11389 | 15991 | 20827 | 24413019 |
| 22534397 | 2557 | 7131 | 11391 | 15993 | 20829 | 24413020 |
| 22534398 | 2559 | 7133 | 11393 | 15995 | 20831 | 24413021 |
| 22534400 | 2561 | 7135 | 11395 | 15997 | 20837 | 24413022 |
| 22534401 | 2563 | 7137 | 11397 | 15999 | 20839 | 24413023 |
| 22534402 | 2567 | — | 11399 | 16001 | 20841 | 24413024 |
| 22534403 | 2569 | 7139 | 11401 | 16003 | 20843, 20845 | 24413025 |
| 22534404 | 2571 | 5973, 7141 | 11403 | 16027 | 20847 | 24413026 |
| 22534405 | 2573 | 5971, 7143, | 11405 | 16029 | 20849, 20851 | 24413027 |
| | | 7147 | | | | |
| 22534406 | 2575 | 7149 | 11407 | 16031 | 20853 | 24413028 |
| 22534407 | 2577 | 7151 | 11409 | 16033 | 20855 | 24413029 |
| 22534408 | 2579 | 7153 | 11411 | 16035 | 20857 | 24413030 |
| 22534409 | 2581 | 7155, 7163 | 11413 | 16037 | 20859, 20861 | 24413031 |
| 22534410 | 2583 | 7157 | 11415 | 16039 | 22523 | 24413032 |
| 22534411 | 2585 | 7159 | — | 16041 | 22521 | 24413033 |
| 22534412 | 2587 | 7161 | 13069 | 16043 | 22519 | 24413034 |
| 22534416 | 2589 | 7167 | 11421, 11423 | 16047 | 20865 | 24413038 |
| 22534417 | 2591 | 7169, 7171 | 11425 | 16049 | 20867 | 24413039 |
| 22534418 | 2593 | 7173 | 11427 | 16051 | 20869 | 24413040 |
| 22534419 | 2595 | 7175 | 11429 | 16053 | 20871 | 24413041 |
| 22534420 | 2597, 3977 | 7177 | 11431 | 16055 | 20873 | 24413043 |
| 22534421 | 2599, 3979 | 7179 | 11433 | 16057, 16059 | 20875 | 24413044 |
| 22534422 | 2601 | 7181 | 11435 | 16061 | 20877 | 24413045 |
| 22534423 | 2603 | 7183 | 11437 | 16063 | 20879 | 24413046 |
| 22534424 | 2605 | 7185 | 11439 | 16065 | 20881 | 24413047 |
| 22534425 | 2607 | 7187 | 11441, 11491 | 16067 | 20883 | 24413048 |
| 22534427 | 2609 | 7189 | 11443, 11493 | 16069, 16071 | 20885 | 24413049 |
| 22534428 | 2611 | 7191 | 11445 | 16073 | 20887 | 24413050 |
| 22534429 | 2613 | 7193 | 11447 | 16077 | 20889 | 24413051 |
| 22534156, 22534430 | 2615 | 6733, 7195 | 11017, 11449 | 16079 | 20315, 20891 | 24413052 |
| 22534431 | 2617 | 7197 | 11451 | 16081 | 20893 | 24413053 |
| 22534432 | 2619 | 7199 | 11453 | 16083 | 20895 | 24413054 |
| 22534433 | 2621 | 7201 | 11455 | — | 20897 | 24413055 |
| 22534436 | 2627 | 7207 | 11461 | — | 20903 | 24413058 |
| — | 2631, 2713 | 7211, 7315 | 11465, 11575, | — | 20907, 21015 | 24413060, 24413111 |
| | | | 11577, 11579 | | | |
| 22534438 | 2633 | 7213 | 11467 | 16099, 17731 | 20909 | 24413061 |
| 22534439 | 2635 | 7215 | 11469 | 16101 | 20911 | 24413062 |
| 22534440 | 2637 | 7217 | 11471 | 16103, 16111 | 20913 | 24413063 |
| 22534441 | 2639 | 7219 | 11473 | 16105 | 20915 | 24413064 |
| 22534442 | 2641 | 7221 | 11475 | 16107 | 20917 | 24413065 |
| 22534443 | 2643 | 7223 | 11477 | 16109, 16113 | 20919 | 24413066 |
| 22534444 | 2645 | 7225, 7237 | 11479 | 16115 | 20921 | 24413067 |
| 22534445 | 2647 | 7227 | 11481 | 16117 | 20923 | 24413068 |
| 22534446 | 2649 | 7229, 7235 | 11483 | 16119, 16127, | 20925 | 24413069 |
| | | | | 16129 | | |
| 22534447 | 2651, 2653 | 7231 | 11485 | 16121 | 20927, 20929 | 24413070 |
| 22534448 | 2655 | 7233, 8759 | 11487 | 16123 | 20931 | 24413071 |
| 22534449 | 2657 | 8757 | 11489 | 16125 | 20933 | 24413072 |
| 22534455 | 2659 | 7247 | 11503 | 16139 | 20943 | 24413077 |
| 22534456 | 2661, 2663 | 7249, 8751 | 11505 | 16141 | 20945 | 24413078 |
| 22534457 | 2665 | 8749 | 11507 | 16143, 17791 | 20947 | 24413079 |
| 22534458 | 2667 | — | 11509 | — | — | 24413080 |
| 22534461 | 2669 | 7251 | 11511, 11513 | 16145, 16147 | 20953, 20955, | 24413083 |
| | | | | | 22653 | |
| 22534462 | 2671, 2673 | 7253 | 11515 | 16149 | 20957 | 24413084 |
| 22534463 | 2675 | 7255 | 11517 | 16151 | 20959 | 24413085 |
| 22534465 | 2677 | 7259 | 11521 | 16157 | 20963 | 24413087 |
| 22534474 | 2679 | 7273 | 11539, 11541 | 16179 | 20979 | 24413094 |
| — | 2681 | 7275 | 11543 | 17709 | 20981 | 24413095 |
| 22534475 | 2683 | 7277 | 11545 | — | 20983 | 24413096 |
| 22534476 | 2685 | 7279 | 11547 | — | 20985 | 24413097 |
| 22534477 | 2687 | 7281 | 11549 | 16011 | 20987 | 24413098 |
| 22534478 | 2689 | 7283 | 11551 | 16013 | 20989 | 24413099 |
| 22534479 | 2691 | 7285 | 11553 | — | 20991 | 24413100 |
| 22534481 | 2695 | 7289 | 11557 | — | 20997 | 24413102 |
| 22534482 | 2697 | 7291 | 11559 | — | 20999 | 24413103 |
| 22534483 | 2699 | 7293 | 11561 | 16005 | 21001 | 24413104 |
| 22534484 | 2701 | 7295 | 11563 | — | 21003 | 24413105 |
| — | 2703 | 7297 | 11565 | — | 21005 | 24413106 |
| 22534487 | 2705 | 7299, 7309 | 11567 | — | 21007 | 24413107 |
| 22534488 | 2707 | 7301 | 11569 | — | 21009 | 24413108 |
| 22534489 | 2709 | 7303, 7311 | 11571 | — | 21011, 21119 | 24413109 |
| 22534491 | 2711 | 7305, 7313, | 11573 | 16025 | 21013, 22661 | 24413110 |
| | | 7347 | | | | |
| 22534492 | 2715 | 7317 | 11581 | 16185, 16191 | 21017, 21123 | 24413112 |
| 22534493 | 2717 | 7319 | 11583 | 16187 | 21019 | 24413113 |
| 22534494 | 2719 | 7321, 7349, | 11585 | 16189, 16193 | 21021 | 24413114 |
| | | 7351 | | | | |
| 22534496 | 2723 | 7325 | 11589 | 16197 | 21025 | 24413116 |
| 22534497 | 2725 | 7327 | 11591 | 16199 | 21027 | 24413117 |
| 22534498 | 2727 | 7329, 8747 | 11593 | 16201, 16217 | 21029 | 24413118 |
| 22534153, 22534499 | 2729 | 6725, 7331 | 11013, 11595 | 15561, 16203, | 20307, 21031 | 23095667 |
| | | | | 17845 | | |
| 22534500 | 2731 | 7333 | 11597 | 16205 | 21035 | 24413119 |
| 22534501 | 2733 | 7335 | 11599 | 16207 | 21037 | 24413120 |
| 22534502 | 2735 | 7337, 7355, | 11601 | 16209 | 21039, 22515 | 24413121 |
| | | 8737 | | | | |
| 22534503 | 2737 | 7339 | 11603 | 16213 | 21041 | 24413122 |
| 22534504 | 2739 | 7341, 8767 | 11605 | 16215, 16219 | 21043 | 24413123 |
| 22534505 | 2741 | 7343 | 11607 | 16221, 16299 | 21045 | 24413124 |
| 22534507 | 2743 | 7345 | 11609 | 16223 | 21047 | 24413125 |
| 22534508 | 2745 | 7357 | 11611 | 16225 | 21049 | 24413126 |
| 22534509 | 2747 | 7359 | 11613 | 16227 | 21051 | 24413127 |
| 22534510 | 2749 | 7361 | 11615 | 16229 | 21053 | 24413128 |
| 22534511 | 2751 | 7363 | 11617 | 16231 | 21055, 21125 | 24413129 |
| 22534512 | 2753 | 7365 | 11619 | 16233 | 21057, 21127 | 24413130 |
| 22534514 | 2755 | 7367 | 11621 | 16235 | 21059 | 24413131 |
| 22534523 | 2757 | 7387 | 11639 | 16251 | 21077 | — |
| 22534524 | 2759 | — | — | — | — | 24413139 |
| 22534528 | 2761 | 7395 | 11641 | 16257, 16301 | 21079 | 24413141 |
| 22534529 | 2763 | 7397 | 11643 | 16259, 16303 | 21081 | 24413142 |
| 22534530 | 2765, 2767 | 7399 | 11645 | 16261 | 21083 | 24413143 |
| 22534537 | 2769 | 7409 | 11653 | 16271 | 21091 | 24413147 |
| 22534538 | 2771 | 7411 | 11655 | 16273 | 21093 | 24413148 |
| 22534540 | 2773 | 7415 | 11659 | 16277, 16305, | 21097 | 24413150 |
| | | | | 17793 | | |
| 22534541 | 2775, 2777 | 7417 | 11661 | 16279 | 21099, 21133 | 24413151 |
| 22534542 | 2779 | 7419 | 11663 | 16281 | 21101 | 24413152 |
| 22534543 | 2781 | 7421 | 11665 | 16283 | 21103 | 24413153 |
| 22534544 | 2783 | 7423 | 11667 | 16285, 16287 | 21105 | 24413154 |
| 22534545 | 2785 | 7425 | 11669 | 16289 | 21107 | 24413155 |
| 22534546 | 2787 | 7427 | 11671 | 16291 | 21109 | 24413156 |
| 22534547 | 2789 | 7429 | 11673 | 16293 | 21111 | 24413157 |
| 22534548 | 2791 | 7431 | 11675 | 16295 | 21113 | 24413158 |
| 22534549 | 2793 | 7433 | 11677 | 16297 | 21115 | 24413159 |
| 22534550 | 2795 | 7435 | 11679 | — | 21117 | 24413160 |
| 22534551, 22534833 | 2797, 2799, | 4785 | — | 13409, 13411, | 18191 | 23094635 |
| | 2901, 2903, | | | 16075, 16211, | | |
| | 3293, 4289 | | | 16409, 16899, | | |
| | | | | 16901, 17407, | | |
| | | | | 17735, 17769, | | |
| | | | | 17771, 17855 | | |
| 22534552 | 2801 | 7437, 7439 | 11681 | 16307 | 21135 | 24413161 |
| 22534553 | 2803 | 7441, 8765 | 11683 | 16309 | 21139 | 24413162 |
| 22534554 | 2805 | 7443 | 11685 | 16311 | 21141 | 24413163 |
| 22534091, 22534556 | 2807 | — | — | 14133, 14347, | — | — |
| | | | | 15669, 15765, | | |
| | | | | 16181, 16313, | | |
| | | | | 17087, 17357 | | |
| 22534092, 22534557 | 2809 | — | — | 14131, 14349, | 20583 | — |
| | | | | 15665, 15667, | | |
| | | | | 15763, 16183, | | |
| | | | | 16315, 17085, | | |
| | | | | 17355, 17711, | | |
| | | | | 17789 | | |
| 22534558 | 2811 | 7445 | 11687 | 16317 | 21143 | 24413165 |
| 22534560 | 2813, 2815 | 7447 | 11689, 11691 | 16319 | 21145 | 24413166 |
| 22534561 | 2817 | 7449 | 11693 | 16321 | 21147 | 24413167 |
| 22534562 | 2819 | 7451 | 11695 | 16323 | 21149 | 24413168 |
| 22534563 | 2821, 2823 | 7453 | 11697 | 16325 | 21151, 21153 | 24413169 |
| 22534565 | 2825 | 7457 | 11701 | 16331 | 21157 | 24413171 |
| 22534566 | 2827 | 7459 | 11703 | 16333 | 21159 | 24413172 |
| 22534567 | 2829 | 7461 | 11705 | 16335 | 21161 | 24413173 |
| 22534568 | 2831 | 7463 | 11707 | 16337 | 21163 | 24413174 |
| 22534569 | 2833 | 7465 | 11709 | 16339 | 21165 | 24413175 |
| 22534571 | 2835 | 7467 | 11711 | 16341, 16421 | 21167 | 24413176 |
| 22534572 | 2837 | 7469 | 11713 | 16343, 16345, | 21169 | 24413177 |
| | | | | 16423 | | |
| 22534573 | 2839 | 7471 | 11715 | 16347 | 21171 | 24413178 |
| — | 2841, 2843 | 7473, 7475 | 11717 | 16349 | 21173 | 24413179, 24413180 |
| 22534574 | 2845 | 7477 | 11719 | 16351 | 21175 | 24413181 |
| 22534575 | 2847 | 7479 | 11721 | 16353 | 21177 | 24413182 |
| 22534576 | 2849 | 7481 | 11723 | 16355 | 21179 | 24413183 |
| 22534577 | 2851 | 7483 | 11725 | 16357 | 21181 | 24413184 |
| 22534578, 22534581 | 2853 | 7485, 7487 | 11727 | 16359, 16361 | 21183 | 24413185 |
| 22534582 | 2855, 2857 | 7489, 7533 | 11729 | 16363, 17775 | 21185 | 24413186 |
| 22534583 | 2859 | 7491 | 11731 | 16365 | 21187 | 24413187 |
| 22534584 | 2861 | 7493 | 11733 | 16367 | 21189 | 24413188 |
| 22534585 | 2863 | 7495 | 11735 | 16369 | 21191 | 24413189 |
| 22534587 | 2865 | 7497 | 11737 | 16371, 17787 | 21193 | 24413190 |
| 22534588 | 2867, 2869 | 7499, 8689 | 11739 | 16373 | 21195 | 24413191 |
| 22534589 | 2871 | 7501 | 11741 | 16375 | 21197 | 24413192 |
| 22534590 | 2873 | 7503 | 11743 | 16377 | 21199 | 24413193 |
| 22534592 | 2875 | 7505 | 11745 | 16379 | 21203 | 24413194 |
| 22534593 | 2877 | 7507 | 11747 | 16381 | 21205 | 24413195 |
| 22534594 | 2879 | 7509 | 11749 | 16383, 16425 | 21207 | 24413196 |
| 22534595 | 2881 | 7511 | 11751 | 16385, 16427 | 21209, 21211 | 24413197 |
| 22534597 | 2883 | 7515 | 11755 | 16389, 16391 | 21215 | 24413199 |
| 22534598 | 2885 | 7517 | 11757 | 16393 | 21217 | 24413200 |
| — | 2887 | 7519 | 11759 | 16395 | 21219 | 24413201 |
| 22534599 | 2889 | 7521 | 11761 | 16397 | 21221 | 24413202 |
| 22534602 | 2891 | 7525 | 11765 | 16401 | 21225 | 24413203 |
| 22534603 | 2893 | 7527 | 11767 | 16403 | 21227 | 24413204 |
| 22534604 | 2895, 2897 | 7529 | 11769 | 16405 | 21229 | 24413205 |
| 22534605 | 2899 | 7531, 7537 | 11771 | 16407 | 21231, 21243 | 24413206 |
| 22534606 | 2905 | 7539 | 11773 | 16411 | 21233 | 24413207 |
| 22534607, 22534608 | 2907, 2909 | 7541, 7543 | 11775, 11777 | 16413 | 21235, 21237 | 24413208, 24413209 |
| 22534609 | 2911 | 7545 | 11779 | 16415 | 21239 | 24413210 |
| 22534610 | 2913 | 7547 | 11781 | 16417 | 21241 | 24413211 |
| 22534612 | 2915 | 7549 | 11783 | 16419 | 21245, 21247 | 24413212 |
| 22534613 | 2917 | 7551 | 11785 | 16429 | 21249 | 24413213 |
| 22534615 | 2919 | 7553, 7555 | 11787 | 16431, 16433 | 21251 | 24413214 |
| 22534616 | 2921 | 7557 | 11789, 11791 | 16435 | 21253 | 24413215 |
| 22534617 | 2923 | 7559 | 11793 | 16437 | 21255 | 24413216 |
| 22534618 | 2925, 2927 | 7561 | 11795 | 16439 | 21257 | 24413217 |
| 22534619 | 2929 | 7563, 7565 | 11797 | 16441 | 21259, 21261 | 24413218 |
| 22534620 | 2931 | 7567 | 11799 | 16443 | 21263 | 24413219 |
| 22534621 | 2933 | 7569 | 11801, 13037 | 16445 | 21265 | 24413220 |
| 22534622 | 2935 | 7571 | 11803 | 16447 | 21267 | 24413221 |
| 22534623 | 2937 | 7573 | 11805 | 16449 | 21269 | 24413222 |
| 22534624 | 2939 | 7575 | 11807 | 16451, 16453 | 21271 | 24413223 |
| 22534625 | 2941 | 7577 | 11809 | 16455 | 21273 | 24413224 |
| 22534626 | 2943 | 7579 | 11811 | 16457 | 21275 | 24413225 |
| 22534627 | 2945 | 7581 | 11813 | 16459 | 21277 | 24413226 |
| 22534628 | 2947 | 7583 | 11815 | 16461 | 21279 | 24413227 |
| 22534629 | 2949, 4123, | 7585, 7587 | 11817 | 16463 | 21281, 21283 | 24413228 |
| | 4125 | | | | | |
| 22534630 | 2951, 4127 | 7589 | 11819, 11821 | 16465 | 21285 | 24413229 |
| 22534631 | 2953 | 7591 | 11823 | 16467 | — | 24413230 |
| 22534632 | 2955 | 7593 | 11825 | 16469 | 21287 | 24413231 |
| 22534633 | 2957 | 7595 | 11827 | 16471 | 21289 | 24413232 |
| 22534634 | 2959 | 7597 | 11829 | 16473 | 21291 | 24413233 |
| 22534635 | 2961 | 7599, 8713 | 11831 | 16475 | 21293 | 24413234 |
| 22534636 | 2963 | 7601 | 11833 | 16477 | 21295 | 24413235 |
| 22534637 | 2965 | 7603 | 11835 | 16479 | 21297 | 24413236 |
| 22534639 | 2967 | 7605 | 11837, 11841 | 16481 | 21299 | 24413237 |
| 22534640 | 2969 | 7607, 7609 | 11839, 11843 | 16483 | 21301 | 24413238 |
| 22534641 | 2971 | 7611 | 11845 | 15205, 16485 | 21303 | 24413239 |
| 22534642 | 2973 | 7613 | 11847 | 16487, 16535 | 21305 | 24413240 |
| 22534643 | 2975 | 7615 | 11849 | 16489 | 21307 | 24413241 |
| 22534644 | 2977 | 7617 | 11851 | 16491, 16537 | 21309 | 24413242 |
| 22534645 | 2979 | 7619 | 11853 | 16493 | 21311 | 24413243 |
| 22534646 | 2981 | 7621 | 11855 | 16495 | 21313 | 24413244 |
| 22534647 | 2983 | 7623 | 11857 | 16497 | 21315 | 24413245 |
| 22534648 | 2985 | 7625 | 11859 | 16499 | 21317 | — |
| 22534649 | 2987 | 7627 | 11861 | 16501 | 21321 | 24413246 |
| 22534651 | 2989 | 7629 | 11863 | 16503 | 21323, 21359 | 24413247 |
| 22534652 | 2991 | 7631 | 11865 | 16505 | 21325 | 24413248 |
| 22534653 | 2993 | 7633 | 11867 | 16507 | 21327 | 24413249 |
| 22534654 | 2995 | 7635 | 11869 | 16509 | 21329 | 24413250 |
| 22534655 | 2997 | 7637 | 11871 | 16511 | 21331 | 24413251 |
| 22534656 | 2999 | 7639 | 11873 | 16513 | 21333 | 24413252 |
| 22534658 | 3001 | 7641 | 11875 | 16515 | 21335 | 24413253 |
| 22534659 | 3003 | 7643, 7645 | 11877 | 16517 | 21337 | 24413254 |
| 22534660 | 3005 | 7647 | 11879, 13111 | 16519 | 21339 | 24413255 |
| 22534661 | 3007 | 7649 | 11881 | 16521 | 21341 | 24413256 |
| 22534662 | 3009 | 7651 | 11883 | 16523 | 21343 | 24413257 |
| 22534668 | 3011 | 7663 | 11895 | 16541 | 21361 | 24413263 |
| 22534669 | 3013 | 7665 | 11897 | 16543 | 21363 | 24413264 |
| 22534670 | 3015 | 7667 | 11899 | 16545 | 21365 | 24413265 |
| 22534671 | 3017 | 7669 | 11901 | 16547 | 21367 | 24413266 |
| 22534672 | 3019 | 7671 | 11903 | 16549, 17843 | 21369 | 24413267 |
| 22534673 | 3021 | 7673 | 11905 | 16551 | 21371 | 24413268 |
| 22534674 | 3023 | 7675 | 11907 | 16553 | 21373 | 24413269 |
| 22534675 | 3025 | 7677 | 11909 | 16555 | 21375 | 24413270 |
| 22534677 | 3027 | 7681 | 11913 | 16559 | 21377 | 24413271 |
| 22534679 | 3029 | 7683 | 11915 | 16573 | 21379 | 24413272 |
| 22534680 | 3031 | 7685 | 11917 | 16575 | 21381 | 24413273 |
| 22534681 | 3033 | 7687 | 11919 | 16577 | 21383 | — |
| 22534682 | 3035 | 7689 | 11921 | 16579, 16581 | 21385 | 24413274 |
| 22534683 | 3037 | 7691 | 11923 | 16583 | 21387 | 24413275 |
| 22534684 | 3039 | 7693 | 11925 | 16585 | 21389 | 24413276 |
| 22534685 | 3041 | 7695 | 11927 | 16587 | 21391 | 24413277 |
| 22534687 | 3043 | 7697 | 11929 | 16589 | 21393 | 24413278 |
| 22534688, 22535015 | 3045, 3563, | 7699, 8243 | 11931, 12513 | 16591, 16593, | 21395, 21937 | 24413279, 24413539 |
| | 3565, 4071 | | | 17199, 17231 | | |
| 22534689 | 3047 | 7701 | 11933 | 16595 | 21397 | 24413280 |
| 22534690 | 3049 | 7703 | 11935 | 16597 | 21399 | 24413281 |
| 22534691 | 3051 | 7705, 7707 | 11937 | 16599 | 21401 | 24413282 |
| 22534692 | 3053 | 7709 | 11939 | 16601 | 21403 | 24413283 |
| 22534699 | 3055 | 7725 | 11953 | 16615 | 21421 | 24413290 |
| 22534700 | 3057, 4169 | 7727 | 11955 | 16617 | 21423, 21439 | 24413291 |
| 22534701 | 3059, 4171 | 7729 | 11957 | 16619 | 21425 | 24413292 |
| 22534702 | 3061 | 7731 | 11959 | 16621 | 21427 | 24413293 |
| 22534703 | 3063 | 7733 | 11961, 11963 | 16623 | 21429, 21441 | 24413294 |
| 22534704 | 3065 | 7735 | 11965 | 16625 | 21431 | 24413295 |
| 22534705 | 3067 | 7737 | 11967 | 16627 | 21433 | 24413296 |
| 22534708 | 3069 | 7743 | 11973 | 16633, 16639, | 21445 | 24413299 |
| | | | | 16641 | | |
| 22534709 | 3071 | 7745 | 11975 | 16635 | 21447, 22613 | 24413300 |
| 22534711, 22534768 | 3073, 3163 | 7747, 7873 | 11977, 12089 | 16637, 16643, | 21449, 21557 | 24413301, 24413358 |
| | | | | 16753 | | |
| 22534712 | 3075 | 7749, 7751 | 11979 | 16645 | 21451 | 24413302 |
| 22534719 | 3077 | 7765 | 11999 | 16659 | 21465 | 24413309 |
| 22534720 | 3079 | 7767, 7771 | 12001 | 16661 | 21467 | 24413310 |
| 22534721 | 3081 | 7769, 7773, | 12003 | 16663 | 21469, 21477 | 24413311 |
| | | 7775 | | | | |
| 22534722 | 3083 | 7777 | 12005 | 16665 | 21471 | 24413312 |
| 22534723 | 3085 | 7779 | 12007 | 16667 | 21473 | 24413313 |
| 22534724 | 3087 | 7781 | 12009 | 16669 | 21475, 21481 | 24413314 |
| 22534725 | 3089 | 7785 | 12011 | 16671 | 21483 | 24413315 |
| 22534726 | 3091 | 7783, 7787 | 12013 | 16673 | 21485 | 24413316 |
| 22534728 | 3093 | 7789 | 12015 | 16675 | 21487 | 24413317 |
| 22534729 | 3095 | 7791 | 12017 | 16677 | 21489 | 24413318 |
| 22534730 | 3097 | 7793 | 12019 | 16679 | 21491 | 24413319 |
| 22534739 | 3103 | 7817 | 12031 | 16691 | 21505 | 24413330 |
| — | 3105, 3107 | 7819 | 12033 | 16693 | 21507, 21509 | 24413331 |
| 22534740 | 3109 | 7821 | 12035 | 16695, 16707 | 21511, 22627 | 24413332 |
| 22534741 | 3111 | 7823 | 12037, 12039 | 16697, 16699 | 21513, 21517 | 24413333 |
| 22534743 | 3113 | 7825 | 12041 | 16701, 16709 | 19293, 21519 | 24413334 |
| 22534744 | 3115 | 7827 | 12043 | 16703, 16711 | 19291 | 24413335 |
| 22534745 | 3117 | 7829 | 12045 | 16705, 16713 | 19289 | 24413336 |
| 22534746 | 3119 | 7831 | 12047 | 17903 | 19287 | 24413337 |
| 22534748 | 3123 | 7835 | 12051 | — | 19281 | 24413339 |
| 22534749 | 3125 | 7837 | 12053 | 16715 | 19279, 21521 | 24413340 |
| 22534750 | 3127 | 7839 | 12055 | 16717 | 21523 | 24413341 |
| 22534751 | 3129 | 7841 | 12057 | 16719 | 21525 | 24413342 |
| 22534752 | 3131 | 7843, 7887 | 12059 | 16721 | 21527 | 24413343 |
| 22534753 | 3133 | 7845, 7889 | 12061 | 16723, 16735 | 21529, 21573 | 24413344 |
| 22534755 | 3135 | 7847 | 12063 | 16725 | 21531 | 24413345 |
| 22534756 | 3137 | 7849 | 12065 | 16727 | 21533 | 24413346 |
| 22534757 | 3139 | 7851 | 12067 | 16729 | 21535 | 24413347 |
| 22534758 | 3141 | 7853, 7891 | 12069 | 16731 | 21537 | 24413348 |
| 22534759 | 3143 | 7855 | 12071 | 16733 | 21539 | 24413349 |
| 22534760 | 3145 | 7857 | 12073 | 16737 | 21541 | 24413350 |
| 22534761 | 3147 | 7859 | 12075 | 16739 | 21543 | 24413351 |
| 22534762 | 3149 | 7861 | 12077 | 16741 | 21545 | 24413352 |
| 22534763 | 3151 | 7863 | 12079 | 16743 | 21547 | 24413353 |
| 22534764 | 3153 | 7865 | 12081 | 16745 | 21549 | 24413354 |
| 22534765 | 3155 | 7867 | 12083 | 16747 | 21551 | 24413355 |
| 22534766 | 3157 | 7869 | 12085 | 16749 | 21553 | 24413356 |
| 22534767 | 3159, 3161 | 7871 | 12087 | 16751 | 21555 | 24413357 |
| 22534769 | 3165, 3167 | 7875 | 12091, 12093 | 16755 | 21559 | 24413359 |
| 22534770 | 3169 | 7877 | 12095 | 16757 | 21561 | 24413360 |
| 22534771 | 3171 | 7879 | 12097 | 16759 | 21563 | 24413361 |
| 22534772 | 3173 | 7881 | 12099 | 16761 | 21565 | 24413362 |
| 22534773 | 3175, 4065 | 7883 | 12101 | 16763 | 21567 | 24413363 |
| 22534774 | 3177, 3179, | 7885, 7893 | 12103 | 16765 | 21569 | 24413364 |
| | 3683, 4067 | | | | | |
| 22534775 | 3181 | 7895 | 12105, 12159 | 16767 | 21571, 21575 | 24413365 |
| 22534776 | 3183 | 7897 | 12107 | 16769, 16795 | 21577 | 24413366 |
| 22534777 | 3185, 3187 | 7899 | 12109 | 16771, 17829 | 21579, 21595, | 24413367 |
| | | | | | 21597 | |
| 22534779 | 3189 | 7901 | 12111 | 16773 | 21581 | 24413368 |
| 22534781 | 3191 | 7145, 7905 | 12115 | 16777 | 21585 | 24413370 |
| 22534782 | 3193 | — | — | 16779 | 22583 | — |
| 22534783 | 3195 | 7907 | 12151 | 16781 | 21601 | 24413371 |
| 22534784 | 3197 | 7909 | 12153 | 16783 | 21603 | 24413372 |
| 22534785 | 3199 | 7911 | 12155 | 16785, 16787 | 21605 | 24413373 |
| 22534786 | 3201 | 7913 | 12157 | 16789 | 21607 | 24413374 |
| 22534787 | 3203 | 7915 | 12161 | 16791 | 21609 | 24413375 |
| 22534792 | 3207 | 6301 | 12169 | 16803 | 21617 | 24413379 |
| 22534793 | 3209, 3211 | 6303, 8769 | 12171 | 16805, 16827 | 21619 | 24413380 |
| 22534794 | 3213 | 6305 | 12173 | 16807 | 21621 | 24413381 |
| 22534795 | 3215 | 6307 | 12175 | 16809, 16811 | 21623 | 24413382 |
| 22534796 | 3217 | 6309 | 12177 | 16813 | 21625 | 24413383 |
| 22534797 | 3219 | 6311 | 12179 | 16815 | 21627 | 24413384 |
| 22534798 | 3221 | 6313 | 12181 | 16817 | 21629 | 24413385 |
| 22534799 | 3223 | 6315 | 12183 | 16819 | 21631 | 24413386 |
| 22534800 | 3225 | 6317 | 12185 | 16821 | 21633 | 24413387 |
| 22534801 | 3227 | 6319 | 12187 | 16823 | 21635 | 24413388 |
| 22534802 | 3229 | 6321 | 12189 | 16825, 16829 | 21637 | 24413389 |
| 22534804 | 3231 | 6323, 7921 | 12191, 12193, | 16831 | 21639 | 24413390 |
| | | | 12195 | | | |
| 22534805 | 3233 | 7923, 7925 | 12197 | 16833 | 21641 | 24413391 |
| 22534807 | 3237 | 7929, 7931 | 12203 | 16839, 16841 | 21645 | 24413393 |
| 22534808 | 3239 | 7933 | 12205 | 16843 | 21647 | 24413394 |
| 22534809 | 3241 | 7935 | 12207 | 16845 | 21649 | 24413395 |
| 22534810 | 3243 | 7937 | 12209 | 16847 | 21651 | 24413396 |
| 22534811 | 3245 | 7939 | 12211 | 16849 | 21653 | 24413397 |
| 22534812 | 3247 | 7941 | 12213 | 16851 | 21655 | 24413398 |
| 22534813 | 3249 | 7943 | 12215 | 16853 | 21657 | 24413399 |
| 22534814 | 3251 | 7945 | 12217 | 16855 | 21659 | 24413400 |
| 22534815 | 3253 | 7947 | 12219 | 16857 | 21661 | 24413401 |
| 22534816 | 3255, 3257 | 7949 | 12221 | 16859 | 21663 | 24413402 |
| 22534817 | 3259 | — | — | 16861, 16891 | — | — |
| 22534819 | 3265 | 7955 | 12227 | 16865, 16895 | 21669 | 24413405 |
| 22534820 | 3267 | 5077, 7957 | 12229 | 16867 | 21671 | 24413406 |
| 22534821 | 3269 | 7959 | 12231 | 16869 | 21673 | 24413407 |
| 22534822 | 3271 | 7961 | 12233, 12249 | 16871, 16873 | 21675 | 24413408 |
| 22534823 | 3273 | 7963 | 12235, 12251 | 16875 | 21677 | 24413409 |
| 22534824 | 3275 | 7965 | 12237 | 16877 | 21679 | 24413410 |
| 22534825 | 3277 | 7967 | 12239 | 16879 | 21681 | 24413411 |
| 22534826 | 3279 | 7969 | 12241 | 16881 | 21683 | 24413412 |
| 22534827 | 3281 | 7971 | 12243 | 16883 | 21685 | 24413413 |
| 22534829 | 3283 | 7973 | 12245 | 16885 | 21687 | 24413414 |
| 22534830 | 3285 | 7975 | 12247 | 16887 | 21689 | 24413415 |
| 22534831 | 3287 | 7977 | 12253 | 16889 | — | 24413416 |
| 22534832 | 3289, 3291 | 7979 | 12255, 12257 | 16897, 17773 | 21691 | 24413417 |
| 22534834 | 3295 | 7981 | 12259 | 16903 | 21693 | 24413418 |
| 22534835 | 3297 | 7983, 7985 | 12261 | 16905 | 21695 | 24413419 |
| 22534836 | 3299 | — | — | — | — | — |
| 22534837 | 3301, 3969, | 7987 | 12263 | 16907, 16909, | 21697 | 24413420 |
| | 3973 | | | 17749 | | |
| 22534838 | 3303 | 7989 | 12265 | 16911 | 21699 | 24413421 |
| 22534839 | 3305 | 7991 | 12267, 12301 | 16913 | 21701 | 24413422 |
| 22534840 | 3307 | 7993 | 12269 | 16915 | 21703, 21705 | 24413423 |
| 22534841 | 3309, 3311 | 7995 | 12271 | 16917 | 21707 | 24413424 |
| 22534842 | 3313 | 7997 | 12273 | 16919, 16947 | 21709 | 24413425 |
| 22534843 | 3315, 3317 | 7999 | 12275 | 16921 | 21711 | 24413426 |
| 22534844 | 3319 | 8001 | 12277 | 16923 | 21713 | 24413427 |
| 22534845 | 3321 | 8003 | 12279 | 16925 | 21715, 21717 | 24413428 |
| 22534847 | 3323 | 8005 | 12281 | 16927 | 21719 | 24413429 |
| 22534848 | 3325 | 8007 | 12123, 12283 | 16929 | 21721, 22431 | 24413430 |
| 22534849 | 3327 | 8009 | 12285 | 16931 | 21723 | 24413431 |
| 22534850 | 3329 | 8011 | 12287 | 16933 | 21725 | 24413432 |
| 22534851 | 3331 | 8013 | 12289 | 16935 | 21727 | 24413433 |
| 22534852 | 3333 | 8015 | 12291 | 16937 | 21729 | 24413434 |
| 22534853 | 3335 | 8017, 8021 | 12293 | 16939 | 21731 | 24413435 |
| 22534854 | 3337 | 8019, 8023 | 12295 | 16941, 16945, | 21733 | 24413436 |
| | | | | 16949 | | |
| 22534855 | 3339 | 8025 | 12297 | 16951 | 21735 | 24413437 |
| 22534857 | 3341 | 8029 | 12303 | 16955 | 21739, 21741 | 24413440 |
| 22534858 | 3343 | 8031 | 12305 | 16957 | 21743 | 24413441 |
| 22534859 | 3345 | 8033 | 12311 | 16959 | 21745 | 24413442 |
| 22534860 | 3347, 3349, | 8035 | 12313 | 16961 | 21747, 21749 | 24413443 |
| | 3351 | | | | | |
| 22534861 | 3353 | 8037 | 12315 | 16963 | 21751, 21755 | 24413444 |
| 22534862 | 3355 | 8039, 8059 | 12317 | 16965 | 21753, 21757 | 24413445 |
| 22534864 | 3359 | 8043 | 12321 | 16969, 17757 | 21761 | 24413447 |
| 22534865 | 3361 | 8045 | 12323 | 16971 | 21763 | 24413448 |
| 22534866 | 3363 | 8047 | 12325 | 16973 | 21765 | 24413449 |
| 22534867 | 3365 | 8049 | 12327 | 16975 | 21767 | 24413450 |
| 22534868 | 3367 | 8051 | 12329 | 16977 | 21769 | 24413451 |
| 22534870 | 3369 | 8053 | 12331 | 16979 | 21771 | 24413452 |
| 22534871 | 3371 | 8055 | 12333 | 16981, 16983 | 21773 | 24413453 |
| 22534872 | 3373 | 8057 | 12335 | 16985 | 21775 | 24413454 |
| 22534873 | 3375 | 8061 | 12337 | 16987 | 21777 | 24413455 |
| 22534874 | 3377 | 8063 | 12339 | 16989 | 21779 | 24413456 |
| 22534927 | 3379, 3381, | 8065 | 12341 | 16991 | 21781, 22593 | 24413457 |
| | 3383 | | | | | |
| 22534928 | 3385 | 8067 | 12343 | 16993 | 21783 | 24413458 |
| 22534929 | 3387 | 8069 | 12345 | 16995 | 21785 | 24413459 |
| 22534932 | 3391 | 8073 | 12349 | 16999 | 21789 | 24413461 |
| 22534933 | 3393 | 8075 | 12351 | 17001 | 21791 | 24413462 |
| 22534935 | 3395 | 8079 | 12355 | 17005 | 21795 | 24413463 |
| 22534936 | 3397 | 8081 | 12357 | 17007 | 21797 | 24413464 |
| 22534937 | 3399 | 8083 | 12359 | 17009 | 21799 | 24413465 |
| 22534938 | 3401 | 8085 | 12361 | 17011 | 21801 | 24413466 |
| 22534939 | 3403 | 8087 | 12363 | 17013 | — | 24413467 |
| 22534940 | 3405 | 8089 | 12365 | 17015 | — | 24413468 |
| 22534941 | 3407 | 8091 | 12367 | 17017 | — | 24413469 |
| 22534942 | 3409 | 8093 | 12369 | 17019 | — | 24413470 |
| 22534943 | 3411 | 8095 | 12371 | 17021 | — | 24413471 |
| 22534945 | 3413 | 8097 | 12373 | 17023 | — | 24413472 |
| 22534946 | 3415 | 8099 | 12375 | 17025 | — | 24413473 |
| 22534947 | 3417 | 8101 | 12377 | 17027 | — | 24413474 |
| 22534948 | 3419 | 8103 | 12379 | 17029 | — | 24413475 |
| 22534949 | 3421, 3423 | 8105 | — | 17031, 17033 | 21803 | 24413476 |
| 22534950 | 3425 | 8107 | 12381 | 17035 | 21805 | 24413477 |
| 22534951 | 3427 | 8109 | 12383 | 17037 | 21807 | 24413478 |
| 22534952 | 3429 | 8111, 8113 | 12385, 12399 | 17039, 17043, | 21809, 21821 | 24413479 |
| | | | | 17935 | | |
| 22534954 | 3431 | 8115 | 12387 | 17041, 17045 | 21811 | 24413480 |
| 22534955 | 3433 | 8117 | 12389 | 17047, 17049 | 21813 | 24413481 |
| 22534956 | 3435 | 8119 | 12391, 12401 | 17051 | 21815 | 24413482 |
| 22534957 | 3437 | 8121 | 12393 | 17053 | 21817 | 24413483 |
| 22534958 | 3439 | 8123 | 12395 | 17055, 17057 | 21819 | 24413484 |
| 22534960 | 3441 | 8127 | 12405 | 17061 | 21825 | 24413486 |
| 22534961 | 3443 | 8129 | 12407 | 17063 | 21827 | 24413487 |
| 22534962 | 3445 | 8131 | 12409 | 17065 | 21829 | 24413488 |
| 22534963 | 3447 | 8133 | 12411 | 17067 | 21831 | 24413489 |
| 22534964 | 3449 | 8135 | 12413 | 17069 | 21833, 21969 | 24413490 |
| 22534965 | 3451, 3455 | 8137 | 12415 | 17071 | 18919, 21835 | 24413491 |
| 22534966 | 3453, 3457 | 8139 | 12417 | 17073 | 21837 | 24413492 |
| 22534967 | 3459 | 8141 | 12419 | 17075, 17157 | 21839, 21971 | 24413493 |
| 22534968 | 3461 | 8143 | 12421 | 17077 | 21841 | 24413494 |
| 22534969 | 3463 | 8145 | 12423 | 17079 | 21843 | 24412908, 24413495 |
| 22534970 | 3465 | 8147 | 12425 | 17081 | 21845 | 24412913, 24413496 |
| 22534972 | 3473 | 8151 | 12429 | 17089 | 21849 | 24412915, 24413498 |
| 22534715, 22534973 | 3475 | 7757, 8153 | 11989, 12431 | 16651, 17091, | 21457, 21851 | 24412916, 24413305, |
| | | | | 17833 | | 24413499 |
| 22534975 | 3477 | 8155 | 12433 | 17093 | 21853 | 24413500 |
| 22534976 | 3479 | 8157, 8159, | 12435 | 17097 | 21855 | 24413501 |
| | | 8161 | | | | |
| 22534977 | 3481 | 8163 | 12437 | 17099 | 21857 | 24413502 |
| 22534978 | 3483 | 8165 | 12439 | 17101 | 21859 | 24413503 |
| — | 3485, 3489 | 8169, 8171 | 12443 | 17105 | 21863 | 24413505 |
| 22534980 | 3491 | 8173 | 12445 | 17107 | 21865 | 24413506 |
| 22534981 | 3493 | 6191 | 12447 | 17109 | 21867 | 24413507 |
| 22534982 | 3495, 3497 | 6193 | 12449 | 17111 | 21869, 21973 | 24413508 |
| 22534983 | 3499 | 8183 | 12451 | 17113, 17159 | 21871 | 24413509 |
| 22534984 | 3501 | 8185 | 12453 | 17115, 17161 | 21873 | 24413510 |
| 22534985 | 3503 | 8187 | 12455 | 17117, 17163 | 21875 | 24413511 |
| 22534986 | 3505 | 8189 | 12457 | 17119 | 21877 | 24413512 |
| 22534987 | 3507 | 8191 | 12459 | 17121 | 21879 | 24413513 |
| 22534988 | 3509 | 8193 | 12461 | 17123 | 21881 | 24413514 |
| 22534989 | 3511 | 8195 | 12463 | 17125 | 21883 | 24413515 |
| 22534991 | 3513 | 8197 | 12465 | 17127, 17165 | 21885 | 24413516 |
| 22534992 | 3515 | 8199 | 12467 | 17129, 17167 | 21887 | 24413517 |
| 22534993 | 3517 | 8201 | 12469 | 17131 | 21889 | 24413518 |
| 22534995 | 3521 | 8205 | 12473 | 17135, 17169 | 21893 | 24413520 |
| 22534997 | 3523 | 8209 | 12477 | 17139 | 21897, 21899 | 24413521 |
| 22534998 | 3525 | 8211 | 12479 | 17141 | 21901 | 24413522 |
| — | 3527 | 8213 | 12483 | 17145 | 21905, 22551 | 24413523 |
| 22534999 | 3529 | 8215 | 12485 | 17147 | 21907, 21909 | 24413524 |
| 22535000 | 3531 | 8217 | 12487 | 17149, 17171 | 21911 | 24413526 |
| 22535001 | 3533 | 8219 | 12489 | 17151, 17173 | 21913 | 24413527 |
| 22535002 | 3535 | 8221 | 12491 | 17155, 17175 | 21915 | 24413528 |
| 22535003 | 3537 | 8223 | 12493 | 17177 | 21917 | 24413529 |
| 22535004 | 3539 | 8225 | 12495 | 17179 | 21919 | 24413530 |
| 22535005 | 3541 | 8227 | 12497 | 17181 | 21921 | 24413531 |
| 22535006 | 3543 | 8229 | 12499 | 17183, 17225 | 21923 | 24413532 |
| 22535007 | 3545 | 8231 | 12501 | 17185, 17227 | 21925 | 24413533 |
| 22535009 | 3549, 4013 | 8235 | 12505 | 17189, 17191 | 21929 | 24413535 |
| 22535010 | 3551, 3553, | 8237 | 12507 | 17193 | 21931 | 24413536 |
| | 4015 | | | | | |
| 22535011 | 3555, 3559 | 8239 | 12509 | 17195 | 21933 | 24413537 |
| 22535012 | 3557, 3561 | 8241 | 12511 | 17197 | 21935 | 24413538 |
| 22535016 | 3567, 4069 | 8245 | 12515 | 17201, 17233 | 21939 | 24413540 |
| 22535017 | 3569 | 8247 | 12517 | 17203 | 21941 | 24413541 |
| 22535018 | 3571 | 8249 | 12519 | 17205 | 21943 | 24413542 |
| 22535019 | 3573, 3575 | 8251 | 12521 | 17207 | 21945 | 24413543 |
| 22535020 | 3577, 3579 | 8253 | 12529 | 17209 | 21947 | 24413544 |
| 22535021 | 3581 | 8255 | 12531 | 17211 | 21949 | 24413545 |
| 22535037 | 3585 | 8259 | — | 17217 | — | 23094905 |
| 22535038 | 3587 | 8261 | — | 17219 | — | — |
| 22535039 | 3589 | 8265, 8271 | — | 17221 | — | 23094903 |
| 22535040 | 3591 | 8273 | — | 17223, 17235 | — | 23094902 |
| 22535041 | 3593, 3595 | 8275 | — | 17237 | — | 23094901 |
| 22535044 | 3597 | 8277 | — | 17239 | — | — |
| 22535045 | 3599, 3601 | 8279 | — | 17241, 17243 | — | — |
| 22535048 | 3603 | 8281 | — | 17245 | — | — |
| 22535049 | 3605 | 8283 | — | 17247 | — | — |
| 22535050 | 3607 | 8285 | — | 17249 | — | — |
| 22535056 | 3609 | — | — | — | — | — |
| 22535057 | 3611 | — | — | 17261, 17263 | — | — |
| 22535058 | 3613 | 8299 | — | 17265 | — | — |
| 22535059 | 3615 | 8301 | — | 17267 | — | — |
| 22535060 | 3617 | 8303 | — | 17269 | — | — |
| 22534280, 22535067 | 3629, 3643, | 8351 | — | 17307 | 20579, 22701 | — |
| | 4115 | | | | | |
| 22535062 | 3633, 3635 | 6989, 8307 | — | 17273 | — | — |
| 22535063 | 3637 | 6987, 8309 | — | 17275 | — | — |
| 22535064 | 3639 | 8311 | — | 17277 | — | 23094819, 23095132, |
| | | | | | | 23095439, |
| | | | | | | 23095618 |
| 22534279, 22535065 | 3641 | 8349 | — | 17305 | 20577 | — |
| 22534281, 22535068 | 3645, 4117 | 8353 | — | 17309, 17851 | 20581 | — |
| 22535070 | 3651 | 8357 | — | 17313 | 22267 | 24413566 |
| 22535071 | 3653 | 8359 | — | 17315 | 21983, 21987 | 24413567 |
| 22535073 | 3655 | 8363 | 12563 | 17337, 17371 | 21989 | 24413568 |
| 22535075 | 3657 | — | 12565 | 17341 | 21993 | 24413570 |
| 22535076 | 3659 | 8367, 8675 | 12567 | 17343 | 21995 | 24413571 |
| 22535077 | 3661 | 8369 | 12569, 12571 | 17345 | 21997 | 24413572 |
| 22535022, 22535078 | 3663 | 8371 | 12573 | 17347 | 21999, 22019, | 24413573 |
| | | | | | 22609 | |
| 22535023, 22535079 | 3665 | 8373 | 12575 | 17349 | 22001, 22607 | 24413574 |
| 22535025, 22535081 | 3667 | 8377 | 12579 | 17353 | 22005 | 24413575 |
| 22535082 | 3669 | 8379 | 12581 | 17359 | 22007 | 24413576 |
| 22535026, 22535083 | 3671 | 8381 | 12583 | 17361 | 22009 | 24413577 |
| 22535027, 22535084 | 3673 | 8383 | 12585 | 17363 | 22011, 22677 | 24413578 |
| 22535085 | 3675, 3677, | 8385 | 12587 | 17365 | 22013 | 24413579 |
| | 4173 | | | | | |
| 22535087 | 3681 | 8347, 8389 | 12591 | 17303, 17369 | 22017 | 24413581 |
| 22535089 | 3685 | 8393 | 12593 | 17377, 17409 | — | 24413582 |
| 22535090 | 3687 | 8395 | 12595 | 17379 | 22021 | 24413583 |
| 22535092 | 3689 | 8397 | 12597 | 17381 | 22023 | 24413584 |
| 22535093 | 3691, 3693 | 8399 | 12599, 12621 | 17383, 17411, | 22025 | 24413585 |
| | | | | 17413 | | |
| 22535094 | 3695 | 8401 | 12601 | 17385, 17415 | 22027, 22053 | 24413586 |
| 22535095 | 3697 | 8403 | 12603 | 17387 | 22029 | 24413587 |
| 22535096 | 3699 | 8405 | 12607 | 17391 | 22033 | 24413588 |
| 22535099 | 3703 | 8411 | 12611 | 17395, 17397 | 22037 | 24413590 |
| 22535100 | 3705 | 8413 | 12613 | 17399 | 22039, 22055 | 24413591 |
| 22535101 | 3707 | 8415 | 12615 | 17401, 17417 | 22041 | 24413592 |
| 22535102 | 3711 | 8419 | 12619 | 17405, 17421 | 22045 | 24413593 |
| 22535103 | 3713 | 8421 | 12623 | 17423 | 22047 | 24413594 |
| 22535104 | 3715 | 8423 | 12625, 12627 | 17425 | 22049 | 24413595 |
| 22535106 | 3717 | 8425 | — | 17427 | — | 24413596 |
| 22535107 | 3719 | 8427 | — | 17429 | — | 24413597 |
| 22535108 | 3721 | 8429 | 12629 | 17431 | 22051 | 24413598 |
| 22535109 | 3723 | 8431, 8433 | 12631, 12633 | 17433 | 22057 | 24413599 |
| 22535110 | 3725 | 8435 | 12635 | 17435 | 22059 | 24413600 |
| 22535111 | 3727 | — | — | — | — | — |
| 22535112 | 3729 | 8437, 8497 | 12637 | 17437 | 22061 | 24413601 |
| 22535113 | 3731 | 8439 | 12639 | 17439 | 22063 | 24413602 |
| 22535114 | 3733 | 8441 | 12641 | 17441, 17871 | 22065 | 24413603 |
| 22535116 | 3741 | 8445 | 12645 | 17445 | 22069 | 24413605 |
| 22535117 | 3743 | 8447, 8449 | 12647, 12649 | 17447, 17457 | 22071 | 24413606 |
| 22535118 | 3745 | 8451 | 12651 | 17449, 17459 | 22073 | 24413607 |
| 22535119 | 3747 | 8453 | 12653 | 17451 | 22075 | 24413608 |
| 22535120 | 3749 | 8455 | 12655 | 17453 | 22077, 22079 | 24413610 |
| 22535121 | 3751 | 8457 | 12657 | 17455 | 22081 | 24413611 |
| 22535122 | 3753 | 8459 | 12659 | 17461 | 22083 | 24413612 |
| 22535123 | 3755 | 8461 | 12661 | 17463 | 22085, 22087 | 24413613 |
| 22535124 | 3757 | 8463 | 12663 | 17465 | 22089 | 24413614 |
| 22535126 | 3759 | 8465, 8733 | 12665 | 17467 | 22091 | 24413616 |
| 22533224, 22535134 | 3761 | 4781, 8481 | 9205, 12681, | 17485 | 18369, 22107 | 24413624 |
| | | | 12683 | | | |
| 22535135 | 3763 | 8483 | 12685 | 17487 | 22109 | — |
| 22535136 | 3765 | 8485 | 12687 | 17489 | 22111 | 24413625 |
| 22535137 | 3767 | 8487 | 12689 | 17491 | 22113 | 24413626 |
| 22535138 | 3769 | 8489 | 12691 | 17493 | 22115 | 24413627 |
| 22535139 | 3771 | 8491 | 12693 | 17495 | 22117 | 24413628 |
| 22535140 | 3773 | 6449, 8493 | 12695 | 17497 | 22119 | 24413629 |
| — | 3775, 3777 | 6445, 6447 | 12697 | 17499 | 22121 | 24413630 |
| 22535141 | 3779 | 6443, 7353, | 12699 | 17501 | 22123 | 24413631 |
| | | 8781 | | | | |
| 22535142 | 3781 | 6441 | 12701 | 17503 | 21975, 22181 | 24413632 |
| 22535143 | 3783 | 6439 | 12703 | 17505 | 22183 | 24413633 |
| 22535144 | 3785 | 6437 | 12705 | 17507 | 22185 | 24413634 |
| 22535146 | 3787 | 6433 | 12709 | 17511, 17781 | 22189 | 24413635 |
| 22535147 | 3789 | 6431 | 12711 | 17513 | 22137, 22139, | 24413636 |
| | | | | | 22191 | |
| 22535148 | 3791 | 6429 | 12713 | 17515 | 22193 | 24413637 |
| 22535150 | 3793, 4109 | 6427 | 12715 | 17517 | 22195 | 24413638 |
| 22535151 | 3795 | 6425 | 12717 | 17519 | 22197 | 24413639 |
| 22535152 | 3797 | 6423 | 12719 | 17521 | 22199 | 24413640 |
| 22535153 | 3799 | 6421 | 12721 | 17523 | 22201 | 24413641 |
| 22535154 | 3801 | 6419 | 12723 | 17525 | 22203 | 24413642 |
| 22535155 | 3803 | 6417 | 12725 | 17527 | 22141, 22205 | 24413643 |
| 22535156 | 3805 | 8645 | 12727 | 17529 | 22143, 22207 | 24413644 |
| 22535157 | 3807 | 8643 | 12729 | 17531 | 22209 | 24413645 |
| 22535168 | 3809 | 8515 | 12757 | 17553 | 22231 | 24413659 |
| 22535169 | 3811 | 8517 | 12759 | 17555 | 22233 | 24413660 |
| 22535170 | 3813 | 8519 | 12761 | 17557, 17575, | 22235 | 24413661 |
| | | | | 17813 | | |
| 22535171 | 3815 | 8521 | 12763 | 17559 | 22237 | 24413662 |
| 22535173 | 3817 | 8523 | 12765 | 17561 | 22239 | 24413663 |
| 22535174 | 3819 | 8525 | 12767 | 17563, 17577, | 22241 | 24413664 |
| | | | | 17579, 17581, | | |
| | | | | 17883 | | |
| 22535175 | 3821 | 8527 | 12769 | 17565, 17583 | 22243 | 24413665 |
| 22535177 | 3823 | 8531 | 12773 | 17569, 17585 | 22247 | 24413667 |
| 22535178 | 3825 | 8533 | 12775, 12777 | 17571 | 22249 | 24413668 |
| 22535179 | 3827 | 8535 | 12779 | 17573, 17587 | 22251, 22289, | 24413669 |
| | | | | | 22291, 22665 | |
| 22535180 | 3829 | 8537 | 12781 | 17589, 17633 | 22253 | 24413670 |
| 22535181 | 3831 | 8539 | 12783 | 17591, 17635 | 22255 | 24413671 |
| 22535182 | 3833 | 8541 | 12787 | 17593, 17899 | 22257 | 24413672 |
| — | 3835 | — | — | 17637, 17823 | 22295 | 23095622 |
| — | 3839 | — | — | 17641 | — | — |
| — | 3841 | — | — | 17643 | — | — |
| — | 3843 | — | — | 17645 | 22299 | — |
| — | 3845 | — | 12831 | 17647 | 22301 | — |
| 22535185 | 3847 | 8547 | 12833 | 17649 | 22303 | 24413675 |
| 22535186 | 3849 | 8549 | 12835 | 17651 | 22305 | 24413676 |
| 22534913, 22535187 | 3851, 3853, | 8551 | 12837, 12839 | 17653 | 22307 | 24413677 |
| | 3855, 3857 | | | | | |
| 22535188 | 3859 | 8553 | 12841, 12843 | 17655 | 22309, 22313 | 24413678 |
| 22535189 | 3861, 3863 | 8555 | 12845 | 17657 | 22311, 22315 | 24413679 |
| 22535190 | 3865 | 6941, 8557 | 12847 | 17659 | 22317 | 24413680 |
| 22535192 | 3867 | 7535, 8559 | 12849 | 17661 | 22319 | 24413681 |
| 22535193 | 3869 | 8561 | 12851, 12873 | 17663 | 22321 | 24413682 |
| 22535194 | 3871 | 8563 | 12853 | 17665 | 22323 | 24413683 |
| 22535195 | 3873 | 8565 | 12855 | 17667 | 22325 | 24413684 |
| 22535196 | 3875 | 8567 | 12857 | 17669 | 22327 | 24413685 |
| 22535197 | 3877 | 8569 | 12859 | 17671, 17673 | 22329 | 24413686 |
| 22535198 | 3879 | 8571 | 12861 | 17675 | 22331 | 24413687 |
| 22535199 | 3881 | 8573 | 12863 | 17677 | 22333 | 24413688 |
| 22535200 | 3883 | 8575 | 12865, 12867 | 17679 | 22335 | 24413689 |
| 22535201 | 3885 | 8577 | 12869 | 17681 | 22337 | 24413690 |
| 22535202 | 3887 | 8579 | 12871, 12875 | 17683 | 22339 | 24413691 |
| 22535205 | 3889 | 8587 | 12881 | 14031 | 22345 | 24413694 |
| 22535206 | 3891 | 8589 | 12883 | 14033 | 22347 | 24413695 |
| 22535207 | 3893 | 8591 | 12885 | 14035 | 22349 | 24413696 |
| 22535208 | 3895 | 8593 | 12887 | 14037 | 22351, 22353, | 24413697 |
| | | | | | 22355 | |
| 22535209 | 3897 | 8595 | 12889 | 14039 | 22357 | 24413698 |
| 22535210 | 3899 | 8597 | 12891 | 14041 | 22359 | 24413699 |
| 22535211 | 3901 | 8599 | 12893 | 14043 | 22361, 22363 | 24413700 |
| 22535212 | 3903 | 8601 | 12895 | 14045 | 22365 | 24413701 |
| 22535213 | 3905 | 8603 | 12897 | 14047, 14049 | 22367 | 24413702 |
| 22535214 | 3907 | 8605 | 12899 | 14051 | 22369 | 24413703 |
| 22535215 | 3909 | 8607 | 12901 | 14053 | 22371 | 24413704 |
| 22535216 | 3911 | 8609 | 12903 | 14055 | 22373 | 24413705 |
| 22535218 | 3913 | 8611 | 12905 | 14057 | 22375 | 24413706 |
| 22535219 | 3915 | 8613 | 12907 | 14059 | 22381 | 24413707 |
| — | 3917, 4047, | 5595 | — | — | — | — |
| | 4099, 4131, | | | | | |
| | 4223 | | | | | |
| 22533593 | 3943, 4189 | — | — | — | — | — |
| — | 3947 | 8657 | 11305 | 15905 | 20741 | 24412981 |
| 22533936 | 3953 | 6239 | 10621 | 15117 | 19901 | 23095379 |
| 22533579 | 3983 | 5523 | — | — | — | — |
| 22533577 | 3987 | — | — | — | — | — |
| 22533575 | 3997 | — | — | — | — | — |
| 22533572 | 4001 | 5501 | — | — | — | — |
| 22535183 | 4019 | 8543 | 12789 | 17595 | 22259 | 24413673 |
| 22535184 | 4021 | 8545 | 12791 | 17597 | 22261 | 24413674 |
| 22534180 | 4033 | 6793 | 11069 | 15613, 15615 | 20375, 20377 | 23095700 |
| 22534179 | 4035 | 6791 | 11067 | 17761 | 20373 | 23095699 |
| 22534178 | 4037 | 6785, 6789 | 9893, 11065 | 15611, 17763, | 20371 | 23095698 |
| | | | | 17943 | | |
| — | 4041, 4043 | — | — | — | — | 23095696 |
| 22533619 | 4093 | 5601 | — | — | — | — |
| 22533618 | 4095 | 5599, 8795 | — | — | 22481, 22739 | — |
| 22533617 | 4097 | 5597 | — | — | — | — |
| 22534519 | 4119 | 7377, 7381 | 11633 | 16245 | 21069, 21071 | 24413136 |
| — | 4139 | — | 12799 | 17611 | — | — |
| — | 4141 | — | 12797 | 17607 | 22271 | — |
| — | 4159 | — | 12793 | — | — | — |
| — | 4181, 4255 | 5559, 5561 | — | — | — | — |
| 22533599 | 4183 | 5557 | — | — | — | — |
| 22533598 | 4185 | 5551 | — | — | — | — |
| 22534199 | 4193 | 6825 | 11101 | 15649 | 20409 | 23095716 |
| 22534198 | 4195 | 6823 | 11099 | 15647 | 20407 | 23095715 |
| 22534191 | 4209 | 6813 | 11089 | 15637 | 20397 | 23095710 |
| 22533569 | 4211 | 5495 | — | — | — | — |
| — | 4219, 4229 | 5589 | — | — | — | — |
| — | 4221, 4227 | 5591 | — | — | — | — |
| — | 4225 | 5593 | — | — | — | — |
| — | 4231 | 5587 | — | — | — | — |
| — | 4233 | 5585 | — | — | — | — |
| — | 4235 | 5581 | — | — | — | — |
| — | 4237 | 5579 | — | — | — | — |
| — | 4239 | 5577 | — | — | — | — |
| — | 4241 | 5575 | — | — | — | — |
| — | 4243 | 5573 | — | — | — | — |
| — | 4245 | 5569 | — | — | — | — |
| — | 4247 | 5567 | — | — | — | — |
| — | 4249 | 5565 | — | — | — | — |
| — | 4251 | 8665 | — | — | — | — |
| — | 4253 | 8667 | — | — | — | — |
| — | 4263 | — | 12803 | — | — | — |
| — | 4265 | — | 12805 | — | — | — |
| — | 4267 | — | 12807 | — | — | — |
| — | 4269 | — | 12809 | 17623 | 22283 | — |
| — | 4271 | — | 12813 | 17625 | 22285 | — |
| — | 4273 | — | 12815 | 17627 | 22287 | — |
| — | 4275 | — | 12819 | 17631 | 22507 | — |
| — | 4277 | — | 12821 | 17825 | 22293 | — |
| — | 4287 | — | — | — | 18893, 18895 | — |
| 22533011 | — | — | — | — | 22411 | — |
| 22533087 | — | — | — | — | — | 23094507 |
| 22533109 | — | 4529 | 8975 | 13377, 13387 | 18165 | 23094527 |
| 22533110 | — | 4531 | 8977 | 13379 | 18167 | 23094528 |
| 22533111 | — | 4533 | 8979 | 13381 | 18169 | 23094529 |
| 22533112 | — | 4535 | 8981 | 13383, 13389 | 18173, 18175 | 23094530 |
| 22533113 | — | 4537, 4539 | 8983, 8985 | 13391 | 18177 | 23094531 |
| 22533114 | — | 4541, 4543, | 8987 | 13393, 13397 | 18179 | 23094532 |
| | | 4545 | | | | |
| 22533115 | — | 4547 | 8989 | 13395 | 18181 | 23094533 |
| 22533127 | — | 4575 | 9013 | 13427 | 18207 | — |
| 22533129 | — | 4579 | 9017 | 13431 | 18211 | 23094546 |
| 22533130 | — | 4581 | 9019 | 13433 | 18213 | 23094547 |
| 22533131 | — | 4583 | 9021 | 13435, 17907 | 18215 | 23094548 |
| 22533135 | — | 4589 | 9027 | 13441 | 18221 | 23094551 |
| 22533136 | — | — | 9029 | — | — | 23094552 |
| 22533137 | — | — | 9031 | 13447 | 18223 | 23094553 |
| 22533138 | — | — | 9033 | 13449 | 18225 | 23094554 |
| 22533185 | — | 4695 | 9135 | 13549 | 18295, 19819 | 23094600 |
| 22533232 | — | — | 9219 | — | — | — |
| 22533233 | — | — | 9221 | — | 18383 | 23094642 |
| 22533250 | — | 4833 | 9259 | 13165 | 18417 | — |
| 22533273 | — | 4883 | 9319 | 13693 | 18461 | 23094678 |
| z22533316 | — | 4969 | 9399 | 13773 | 18543 | — |
| 22533378 | — | 5113 | 9505 | 13923 | 18707, 18709 | 23094774 |
| 22533389 | — | 5135 | 9527 | 13945 | 18731 | — |
| 22533419 | — | — | 9587 | — | 18795 | — |
| 22533425 | — | 5215 | 9597 | 14017 | 18805, 18807 | 23094867 |
| 22533510 | — | 8633 | 9795 | 14287 | 19033 | 23094959 |
| 22533566 | — | 5489 | — | — | — | — |
| 22533578 | — | 5521 | — | — | — | — |
| 22533580 | — | 5525 | — | — | — | — |
| 22533581 | — | 5527 | — | — | — | — |
| 22533582 | — | 5529 | — | — | — | — |
| 22533584 | — | 5531 | — | — | — | — |
| 22533585 | — | 5533, 8799 | — | — | — | — |
| 22533586 | — | 5535 | — | — | — | — |
| 22533588 | — | — | — | — | 19285 | — |
| 22533589 | — | 5539 | — | — | — | — |
| 22533590 | — | 5541 | — | — | — | — |
| 22533591 | — | 5543 | — | — | — | — |
| 22533594 | — | 5545 | — | — | — | — |
| 22533595 | — | 5547 | — | — | — | — |
| 22533596 | — | — | — | — | 19109 | — |
| 22533620 | — | 5603 | — | — | — | — |
| 22533621 | — | 5605 | — | — | — | — |
| 22533622 | — | 5607 | — | — | — | — |
| 22533623, 22534878 | — | 5609 | — | — | 19155 | — |
| 22533624 | — | 5611 | — | — | 19157 | — |
| 22533625 | — | 5613 | — | — | — | — |
| 22533676 | — | — | — | — | — | 23095054 |
| 22533727 | — | 5795 | 10225 | 14659 | 19469, 22651 | 23095095 |
| 22533749 | — | 5839 | 10267 | 14729 | 19529 | 23095165 |
| 22533750 | — | 5841 | 10269 | 14731 | 19531 | — |
| 22533755 | — | 5851 | 10279 | 14741 | 19541 | 23095170 |
| 22533771 | — | 5881 | 10309 | 14775, 14777, | 19571 | 23095210 |
| | | | | 14779 | | |
| 22533785 | — | 5907 | 10337 | 14805 | 19597 | 23095223 |
| 22533788 | — | 5911 | 10341, 10343 | 14811, 14813, | 19601, 19603 | 23095225 |
| | | | | 14815 | | |
| 22533790 | — | 5915 | 10347 | 14819 | 19607 | 23095227 |
| 22533800 | — | 5935, 5975 | 10367 | 14841 | 19631 | 23095237 |
| 22533801 | — | 5937 | 10369 | 14843 | 19633 | 23095238 |
| 22533802 | — | 5939 | 10371 | 14845 | 19635 | 23095239 |
| 22533821 | — | 5977, 8651 | 10405 | 14879, 14891 | 19673 | 23095256 |
| 22533822 | — | 5979 | 10407 | 14893 | 19675 | 23095257 |
| 22533823 | — | 5981 | 10409 | 14895 | 19677 | 23095258 |
| 22533824 | — | 5983 | 10411 | 14897 | 19679 | 23095259 |
| 22533825 | — | 5985 | 10413 | 14899 | 19681 | 23095260 |
| 22533836 | — | 6005, 6007 | 10433 | 14917 | 19701 | 23095269 |
| 22533854 | — | 6043 | — | — | — | 23095286 |
| 22533905 | — | 6151 | 10565 | 15057 | 19851 | 23095334 |
| 22533910 | — | 6161 | 10579 | 15067 | 19861 | 23095339 |
| 22533926 | — | 6183 | — | 15095 | — | — |
| 22533930 | — | 6227 | 10571, 10607 | 15105 | 19887 | 23095373 |
| 22533937 | — | 6195 | 9967 | — | — | — |
| 22533938 | — | 6197 | 9969 | — | — | — |
| 22533940 | — | — | — | 15225 | — | — |
| 22533941 | — | 6199 | 9971 | 15223 | — | — |
| 22533943 | — | 6201 | 9973 | 15221 | — | — |
| 22533945 | — | 6203 | 9975 | 15219 | — | — |
| 22533948 | — | 6205 | 9977 | 15217 | — | — |
| 22533949 | — | 5369, 6207 | 9979 | 15215 | — | — |
| 22533950 | — | — | 9983 | 15213 | — | — |
| 22533951 | — | 6213, 6217 | 9987 | 15209 | — | — |
| 22533952 | — | 6215, 6219 | 9989 | 15203 | — | — |
| 22533953 | — | 6221 | 9991 | 15199 | — | — |
| 22533954 | — | 6223, 8175 | 9993 | 15195, 17857 | — | — |
| 22533955 | — | 8177 | 9995 | 15193 | — | — |
| 22533956 | — | 8179 | 9997 | 15185 | — | — |
| 22533957 | — | 8181 | 9999 | 15183 | — | — |
| 22533975 | — | 6273 | 10655 | 15153 | 19935 | 23095395 |
| 22534019 | — | 6397 | 10743 | 15311 | 20031 | 23095484 |
| 22534020 | — | 6399 | 10745 | 15313 | 20033 | 23095485 |
| 22534021 | — | 6401 | 10747 | 15315 | 20035 | 23095486 |
| 22534024 | — | 6407 | 10753 | 15321 | 20047 | 23095489 |
| 22534043, 22534733 | — | 6489 | 10817 | 16683 | 20097, 21495 | — |
| 22534047 | — | 6497 | — | — | — | — |
| 22534061 | — | 6531, 6541 | 10841 | 15383 | 20121 | 23095522 |
| 22534075 | — | 6567 | 10871 | 15413, 15415 | 22493 | 23095536 |
| 22534094 | — | — | — | — | — | 23095551 |
| 22534155 | — | — | 12527 | — | — | 23095669 |
| 22534157 | — | 6735 | 11019 | 15577 | 20317 | 23095670 |
| 22534158 | — | 6737 | 11021 | 15579 | 20319 | 23095671 |
| 22534159 | — | 6739 | 11023 | 15581 | 20321 | 23095672 |
| 22534160 | — | 6741, 6743 | 11027, 11029 | 17715, 17717 | 20323, 20325 | 23095673 |
| 22534161 | — | 6745 | 11031 | 17713 | 20327 | 23095674 |
| 22534162, 22534164 | — | 6747, 6751 | 11033, 11037 | — | 20329, 20333 | 23095675, 23095677 |
| 22534165 | — | 6753 | 11039 | 15583 | 20335 | 23095678 |
| 22534166 | — | 6755 | 11041 | 15585 | 20337 | 23095679 |
| 22534167 | — | 6757 | 11043 | 15587 | 20339 | 23095680 |
| 22534168 | — | 6759 | 11045 | 15589 | 20341 | 23095681 |
| 22534192 | — | 6815 | 11091 | 15639 | 20399 | 23095711 |
| 22534193 | — | — | 11093 | — | — | — |
| 22534194 | — | — | 11095 | — | — | — |
| 22534195 | — | — | 11097 | — | — | — |
| 22534196 | — | — | 13039 | — | — | — |
| 22534238 | — | — | 11177 | 15731 | — | — |
| 22534251 | — | 6935 | 11211 | 15767 | 20519 | 24412881 |
| 22534253 | — | 6937 | 11213 | 15769 | 20521 | 24412882 |
| 22534254 | — | 6939 | 11215, 11217 | 15771 | 20523 | 24412883 |
| 22534255 | — | — | — | — | — | 24412884 |
| 22534256 | — | — | — | — | — | 24412885 |
| 22534284, 22534297 | — | — | — | — | 20619 | — |
| 22534292 | — | — | — | — | 20609 | — |
| 22534298 | — | — | — | — | 20621 | 24412925 |
| 22534302 | — | — | — | — | 20629 | 24412929 |
| 22534303 | — | — | — | — | 20631 | 24412930 |
| 22534304 | — | 4799 | — | — | — | 24412931 |
| 22534319 | — | — | — | — | — | 24412948 |
| 22534320 | — | — | — | — | 20667 | 24412949 |
| 22534334 | — | — | 11261 | 15853 | 20695 | — |
| 22534338 | — | 7011 | 11269 | 15865 | 20703 | — |
| 22534358 | — | 8655 | 11307 | 15907 | 20743 | 24412982 |
| 22534374 | — | 7079, 7095 | 11339 | 15949 | 20779 | 24412998 |
| 22534413 | — | — | — | — | — | 24413035 |
| 22534414 | — | — | 11417 | — | — | 24413036 |
| 22534415 | — | 7165 | 11419 | 16045 | 20863 | 24413037 |
| 22534450 | — | 7239 | 11495 | 16131 | 20935 | 24413073 |
| 22534451 | — | 7241 | 11497 | 16133 | 20937 | 24413074 |
| 22534453 | — | 7243 | 11499 | 16135 | 20939 | 24413075 |
| 22534454 | — | 7245 | 11501 | 16137 | 20941 | 24413076 |
| 22534459 | — | — | 13059 | — | 20949 | 24413081 |
| 22534460 | — | — | 13057 | — | 20951 | 24413082 |
| 22534464 | — | 7257 | 11519 | 16153, 16155 | 20961 | 24413086 |
| 22534467 | — | 7261 | 11523 | 16159 | 20965, 20967 | 24413088 |
| 22534468 | — | 7263 | 11525, 11527 | 16161 | 20969 | 24413089 |
| 22534469 | — | 7265 | 11529, 11531 | 16163 | 20971 | 24413090 |
| 22534470 | — | 7267 | 11533 | 16165, 16169, | 20973 | 24413091 |
| | | | | 16171 | | |
| 22534471 | — | 7099, 7269 | 11535 | 16167, 16173, | 20975 | 24413092 |
| | | | | 16175 | | |
| 22534472 | — | 7271 | 11537 | 16177 | 20977 | 24413093 |
| 22534515 | — | 7369 | 11623, 11625 | 16237 | 21061 | 24413132 |
| 22534516 | — | 7371 | 11627 | 16239 | 21063 | 24413133 |
| 22534517 | — | 7373 | 11629 | 16241 | 21065, 21129 | 24413134 |
| 22534518 | — | 7375 | 11631 | 16243 | 21067 | 24413135 |
| 22534520 | — | 7379, 7383 | 11635 | 16247 | 21073 | 24413137 |
| 22534521 | — | 7385 | 11637 | 16249 | 21075 | — |
| 22534525 | — | 7389 | — | 16253 | — | — |
| 22534526 | — | 7391 | — | — | — | — |
| 22534527 | — | 7393 | — | 16255 | — | 24413140 |
| 22534531 | — | 7401 | 11647 | 16263 | 21085 | 24413144 |
| 22534532 | — | 7403 | — | 16265 | — | — |
| 22534534 | — | 7405 | 11649 | 16267 | 21087 | 24413145 |
| 22534536 | — | 7407 | 11651 | 16269 | 21089 | 24413146 |
| 22534539 | — | 7413 | 11657 | 16275 | 21095, 21131 | 24413149 |
| 22534555 | — | — | — | — | — | 24413164 |
| 22534564 | — | 7455 | 11699 | 16327, 16329 | 21155 | 24413170 |
| 22534591 | — | — | — | — | 21201 | — |
| 22534596 | — | 7513 | 11753 | 16387 | 21213 | 24413198 |
| 22534600 | — | 7523 | 11763 | 16399 | 21223 | — |
| 22534663 | — | 7653 | 11885 | 16525 | 21345 | 24413258 |
| 22534664 | — | 7655 | 11887 | 16527 | 21347 | 24413259 |
| 22534665 | — | 7657 | 11889 | 16529 | 21349, 21351 | 24413260 |
| 22534666 | — | 7659 | 11891 | 16531 | 21353, 21355 | 24413261 |
| 22534667 | — | 7661 | 11893 | 16533 | 21357 | 24413262 |
| 22534676 | — | 7679 | 11911 | 16557 | — | — |
| 22534693 | — | 7711 | 11941 | 16603 | 21405 | 24413284 |
| 22534694 | — | 7713 | 11943 | 16605 | 21407, 21409 | 24413285 |
| 22534695 | — | 7715, 7717 | 11945 | 16607 | 21411, 21413 | 24413286 |
| 22534696 | — | 7719 | 11947 | 16609 | 21415 | 24413287 |
| 22534697 | — | 7721 | 11949 | 16611 | 21417, 22637, | 24413288 |
| | | | | | 22639 | |
| 22534698 | — | 7723 | 11951 | 16613 | 21419 | 24413289 |
| 22534706 | — | 7739 | 11969 | 16629 | 21435 | 24413297 |
| 22534707 | — | 7741 | 11971 | 16631 | 21437, 21443 | 24413298 |
| 22534713 | — | 7753 | 11981, 11983, | 16647 | 21453 | 24413303 |
| | | | 11985 | | | |
| 22534714 | — | 7755 | 11987 | 16649 | 21455 | 24413304 |
| 22534716 | — | 7759 | 11991 | 16653 | 21459 | 24413306 |
| 22534718 | — | 7763 | 11997 | 16657 | 21463 | 24413308 |
| 22534734 | — | — | 12023 | 16685 | 21497 | — |
| 22534735 | — | — | 12025 | 16687 | 21499 | — |
| 22534780 | — | 7903 | 12113 | 16775 | 21583 | 24413369 |
| 22534788 | — | 7917, 7919 | 12163 | 16793, 16797 | 21611 | 24413376 |
| 22534789 | — | 6297 | 12165 | 16799 | 21613 | 24413377 |
| 22534856 | — | 8027 | 12299 | 16953 | 21737 | 24413439 |
| 22534883 | — | — | 10039 | — | — | — |
| 22534884 | — | — | 10037, 13021 | — | — | — |
| 22534934 | — | 8077 | 12353 | 17003 | 21793 | — |
| 22534959 | — | 8125 | 12397, 12403 | 17059 | 21823 | 24413485 |
| 22534979 | — | 8167 | 12441 | 17103 | 21861 | 24413504 |
| 22534996 | — | 8207 | 12475 | 17137 | 21895 | — |
| 22535024, 22535080 | — | 8375 | 12577 | 17351 | 22003 | — |
| 22535031, 22535161 | — | — | — | — | — | — |
| 22535032, 22535162 | — | — | — | — | — | — |
| 22535033 | — | — | — | — | — | 24413551, 24413552 |
| 22535034 | — | 8297 | — | — | — | — |
| 22535051 | — | 8287 | — | 17251 | — | — |
| 22535052 | — | 8289 | — | 17253 | — | — |
| 22535053 | — | 8291 | — | 17255 | — | — |
| 22535054 | — | 8293 | — | 17257 | — | — |
| 22535055 | — | 8295 | — | 17259 | — | — |
| 22535072 | — | 8361 | — | 17317 | 21985 | — |
| 22535074 | — | 8365 | — | 17375 | 21991 | 24413569 |
| 22535086 | — | 8387 | 12589 | 17367 | 22015 | 24413580 |
| 22535127 | — | 8467 | 12667 | 17469 | 22093 | 24413617 |
| 22535128 | — | 8469 | 12669 | 17471 | 22095 | 24413618 |
| 22535129 | — | 8471 | 12671 | 17473 | 22097 | 24413619 |
| 22535130 | — | 8473 | 12673 | 17475 | 22099 | 24413620 |
| 22535131 | — | 8475 | 12675 | 17477 | 22101, 22675 | 24413621 |
| 22535132 | — | 8477 | 12677 | 17479 | 22103 | 24413622 |
| 22535133 | — | 8479 | 12679 | 17481, 17483 | 22105 | 24413623 |
| 22535145 | — | 6435 | 12707 | 17509, 17783 | 22187 | — |
| 22535176 | — | 8529 | 12771 | 17567 | 22245 | 24413666 |
| 22535203 | — | 8581, 8583 | 12877 | 17685 | 22341 | 24413692 |
| 22535204 | — | 8585 | 12879 | 14029 | 22343 | 24413693 |
| — | — | 4401 | — | — | — | 23094471 |
| — | — | 4403 | — | — | — | 23094472 |
| — | — | — | 9109 | — | — | 23094588 |
| — | — | 4671 | 9111 | — | — | 23094589 |
| — | — | 4673 | 9113 | — | — | 23094590 |
| — | — | — | — | 16569 | — | 23094789, 23095102, |
| | | | | | | 23095468 |
| — | — | 8341 | — | 17297 | — | 23094897 |
| — | — | 8339 | — | 17295 | — | 23094898 |
| — | — | 8337 | — | 17293 | — | 23094899 |
| — | — | 8263 | — | — | — | 23094904 |
| — | — | — | — | — | 18883 | 23094908 |
| — | — | — | 9707 | — | — | 23094916 |
| — | — | — | 10003 | — | — | 23095036 |
| — | — | 8313, 8687 | — | 17279, 17873 | — | 23095620 |
| — | — | 6817 | — | 15641 | — | 23095712 |
| — | — | 6819 | — | 15643 | 20403 | 23095713 |
| — | — | — | — | 15645 | — | 23095714 |
| — | — | 6977 | 11245 | — | 22485, 22487, | 24412894 |
| | | | | | 22553 | |
| — | — | 8509 | 12745, 13029 | 17547 | 22225, 22503 | 24412898, 24413653 |
| — | — | 8505 | 12741, 13013, | 17543 | 20553, 20555, | 24412900, 24413651 |
| | | | 13015 | | 22221 | |
| — | — | 8503 | 12739, 13011 | 17541 | 20557, 22219 | 24412901, 24413650 |
| — | — | — | 13009 | — | 20559 | 24412902 |
| — | — | — | 13007 | — | 20561 | 24412903 |
| — | — | — | 13005 | — | 20563, 22581 | 24412904 |
| — | — | — | — | — | 20653 | 24412941 |
| — | — | 8659 | 11303 | 15903 | 20739 | 24412980 |
| — | — | 7797 | — | — | — | 24413321 |
| — | — | 7799, 7801 | — | — | — | 24413322 |
| — | — | 7803 | — | — | — | 24413323 |
| — | — | 7805 | — | — | — | 24413324 |
| — | — | 7807 | — | — | — | 24413325 |
| — | — | 7809 | — | — | — | 24413327 |
| — | — | 7813 | — | — | — | 24413328 |
| — | — | 7815 | — | — | — | 24413329 |
| — | — | 7951 | 12223 | — | 21665 | 24413403 |
| — | — | — | 12533 | 17323 | — | 24413546 |
| — | — | — | 12535 | 17325 | — | 24413547 |
| — | — | — | 12541 | 17333 | 21961, 22475 | 24413549, 24413557, |
| | | | | | | 24413564 |
| — | — | — | 12549 | 17329 | 21957, 22461, | 24413561 |
| | | | | | 22465 | |
| — | — | — | 12553 | — | 22469, 22471 | 24413562 |
| — | — | — | — | 17335 | 22477 | 24413565 |
| — | — | 8641 | 12731 | 17533 | 22211 | 24413646 |
| — | — | 8639 | 12733 | 17535 | 22213 | 24413647 |
| — | — | 8499, 8637 | 12735 | 17537 | 22215 | 24413648 |
| — | — | 8501 | 12737 | 17539 | 22217 | 24413649 |
| — | — | 8507 | 12743, 13017 | 17545 | 22223 | 24413652 |
| — | — | 8511 | 12747 | 17549 | 22227 | 24413654 |
| — | — | 8513 | 12749, 12751 | 17551 | 22229 | 24413655 |
| — | — | 4459 | 8901 | 13303 | 18085 | — |
| — | — | 4479 | 8921 | 13323 | 18105 | — |
| — | — | 4793 | — | 17727 | — | — |
| — | — | 4797 | — | 17723 | — | — |
| — | — | 4801 | — | 17721 | — | — |
| — | — | 5285 | — | — | 18931 | — |
| — | — | 5291, 5293 | — | 17691 | — | — |
| — | — | 5629 | 9919 | 14405 | 19263 | — |
| — | — | 5687 | 10097 | 14507 | 19333 | — |
| — | — | 5753 | 10185 | 14619 | 19429 | — |
| — | — | 6181 | 10597 | — | — | — |
| — | — | 6209 | 9981 | — | — | — |
| — | — | 6211 | 9985 | 15211 | — | — |
| — | — | 6821 | — | — | 20405 | — |
| — | — | 6957 | 11227 | 15785 | 20537 | — |
| — | — | 8315 | — | 17281 | — | — |
| — | — | 8317 | — | 17283 | — | — |
| — | — | 8319 | — | 17285 | — | — |
| — | — | 8321, 8331 | — | 17287 | — | — |
| — | — | 8333 | — | 17289 | — | — |
| — | — | 8335 | — | 17291 | — | — |
| — | — | 8673, 8791 | — | 13161 | — | — |
| — | — | — | — | 17619 | 22279 | — |
| — | — | — | — | 17605 | 22269 | — |
| — | — | — | 12551 | — | 22467 | — |
| — | — | — | 12149 | — | 21599, 22457 | — |
| — | — | — | 12147 | — | 22455 | — |
| — | — | — | 12145 | — | 22453 | — |
| — | — | — | 12139 | — | 22447 | — |
| — | — | — | 12137 | — | 22445 | — |
| — | — | — | 12133 | — | 22441 | — |
| — | — | — | 12131 | — | 22439 | — |
| — | — | — | 12129 | — | 22437 | — |
| — | — | — | 12127 | — | 22435 | — |
| — | — | — | 12125 | — | 22433 | — |
| — | — | — | 12119 | — | 21589 | — |
| — | — | — | 12117 | — | 21587 | — |
| — | — | — | — | 16093, 17805, | — | — |
| | | | | 17929, 17939 | | |
| — | — | — | — | 16085, 17881 | — | — |
| — | — | — | 10831 | — | 20109 | — |
| — | — | — | 10827 | — | 20105 | — |
| — | — | — | 10017, 10019 | — | — | — |
| — | — | — | 10009, 12953 | — | — | — |
| — | — | — | — | — | 18237, 22135, | — |
| | | | | | 22643 | |
| — | — | — | 9897 | — | 20367 | — |
| — | — | — | 10799 | 15375 | — | — |
| — | — | — | 12135 | — | 22443 | — |
| — | — | — | 12141 | — | 22449 | — |
| — | — | — | 12143 | — | 22451 | — |
| — | — | — | 12481 | 17143, 17947 | 21903 | — |
| — | — | — | 12605 | 17389 | 22031 | — |
| — | — | — | 12817 | 17629 | 22509 | — |
| — | — | — | 12929 | — | 18873 | — |
| — | — | — | 13045 | 15817 | — | — |
| — | — | — | — | 17213 | 21951 | — |
| — | — | — | — | 17615 | 22275 | — |
| — | — | — | — | 17617 | 22277 | — |
| — | — | — | — | 17621 | 22281 | — |

TABLE III
Unique coding sequences

| | |
|---|---|
| 2603V/R | 22533205, 22533568, 22533571, 22533574, 22533576, 22533597, 22533706, |
| | 22533776, 22533967, 22534356, 22534614, 22534650, 22534875, 22534876, |
| | 22534877, 22534880, 22534882, 22534885, 22534886, 22534887, 22534889, |
| | 22534891, 22534892, 22534894, 22534895, 22534898, 22534900, 22534901, |
| | 22534903, 22534906, 22534907, 22534908, 22534911, 22534912, 22534915, |
| | 22534916, 22534917, 22534919, 22534924, 22535035, 22535159, 22535160, |
| | 22535163, 22535164, 22535165, 22535166, 22535167 |
| 18RS21 | 1749, 3623, 3981, 3985, 3989, 3991, 3999, 4057, 4059, 4081, 4203, 4205, 4207 |
| 515 | 4359, 4367, 4803, 4805, 4807, 4809, 4811, 4813, 5289, 5295, 5493, 5497, 5499, |
| | 5505, 5507, 5511, 5513, 5515, 5563, 6887, 8267, 8269, 8647, 8683, 8685, 8695, |
| | 8725, 8727, 8783, 8789, 8797 |
| CJB11 | 9213, 9237, 9239, 10005, 10681, 10809, 10811, 12795, 12823, 12827, 12949, |
| | 12951, 12967, 12997, 13043, 13047, 13087, 13091, 13093, 13105 |
| COH1 | 13153, 13157, 13159, 13241, 13243, 13263, 15187, 15201, 15227, 15819, 15821, |
| | 15823, 15825, 15827, 15829, 16015, 16019, 16021, 16023, 16539, 16561, 16565, |
| | 17319, 17321, 17693, 17705, 17707, 17719, 17753, 17785, 17797, 17819, 17897, |
| | 17921, 17933 |
| H36B | 18691, 19065, 19067, 19071, 19073, 19075, 19085, 19087, 19089, 19091, 19093, |
| | 19095, 19099, 19103, 19111, 19113, 19115, 19117, 19119, 19123, 19125, 19127, |
| | 19129, 19131, 19133, 19135, 19139, 19141, 19143, 19145, 19149, 19165, 20099, |
| | 20401, 22529, 22531, 22533, 22535, 22545, 22547, 22557, 22559, 22561, 22565, |
| | 22571, 22585, 22589, 22621, 22641, 22667, 22671, 22679, 22695, 22699, 22705, |
| | 22715, 22717, 22721, 22723, 22725, 22733 |
| NEM316 | 23094662, 23094664, 23094667, 23094668, 23094669, 23094670, 23094794, |
| | 23094796, 23094797, 23094798, 23094799, 23094802, 23094803, 23094806, |
| | 23094808, 23094809, 23094810, 23094811, 23094812, 23094813, 23094814, |
| | 23094815, 23094816, 23094818, 23094820, 23094821, 23094822, 23094823, |
| | 23094824, 23094825, 23094827, 23094828, 23094829, 23094830, 23094831, |
| | 23094832, 23094833, 23094835, 23095107, 23095109, 23095110, 23095111, |
| | 23095112, 23095115, 23095116, 23095119, 23095121, 23095122, 23095123, |
| | 23095124, 23095125, 23095126, 23095127, 23095128, 23095129, 23095131, |
| | 23095133, 23095134, 23095135, 23095136, 23095137, 23095138, 23095140, |
| | 23095141, 23095142, 23095143, 23095144, 23095145, 23095146, 23095148, |
| | 23095423, 23095425, 23095426, 23095427, 23095428, 23095429, 23095430, |
| | 23095431, 23095433, 23095434, 23095435, 23095436, 23095437, 23095438, |
| | 23095440, 23095442, 23095443, 23095444, 23095445, 23095446, 23095447, |
| | 23095448, 23095449, 23095450, 23095452, 23095455, 23095456, 23095459, |
| | 23095460, 23095461, 23095462, 23095463, 23095569, 23095570, 23095571, |
| | 23095572, 23095573, 23095574, 23095575, 23095576, 23095577, 23095578, |
| | 23095579, 23095580, 23095581, 23095582, 23095583, 23095584, 23095585, |
| | 23095586, 23095587, 23095614, 23095615, 23095617, 23095623, 23095624, |
| | 23095626, 23095627, 24412909, 24412910, 24412911, 24412912, 24412921, |
| | 24412922, 24412923, 24412924, 24413555 |

TABLE IV
Sequences without homologs in 2603V/R or NEM316

| | |
|---|---|
| 18RS21 | 283, 1123, 1127, 1129, 1273, 1275, 1731, 2321, 2367, 3839, 3841, 3843, 3845, |
| | 3917, 4047, 4099, 4131, 4139, 4141, 4159, 4181, 4219, 4221, 4223, 4225, 4227, |
| | 4229, 4231, 4233, 4235, 4237, 4239, 4241, 4243, 4245, 4247, 4249, 4251, 4253, |
| | 4255, 4263, 4265, 4267, 4269, 4271, 4273, 4275, 4277, 4287 |
| 515 | 4459, 4479, 4601, 4793, 4797, 4801, 5285, 5291, 5293, 5559, 5561, 5565, 5567, |
| | 5569, 5573, 5575, 5577, 5579, 5581, 5585, 5587, 5589, 5591, 5593, 5595, 5621, |
| | 5625, 5627, 5629, 5687, 5753, 6179, 6181, 6209, 6211, 6821, 6957, 8315, 8317, |
| | 8319, 8321, 8331, 8333, 8335, 8665, 8667, 8673, 8791 |
| CJB11 | 8901, 8921, 9043, 9897, 9911, 9915, 9917, 9919, 9981, 9985, 10009, 10017, 10019, |
| | 10097, 10143, 10145, 10185, 10597, 10799, 10827, 10831, 11227, 12117, 12119, |
| | 12125, 12127, 12129, 12131, 12133, 12135, 12137, 12139, 12141, 12143, 12145, |
| | 12147, 12149, 12481, 12551, 12605, 12793, 12797, 12799, 12803, 12805, 12807, |
| | 12809, 12813, 12815, 12817, 12819, 12821, 12831, 12929, 12953, 13045 |
| COH1 | 13161, 13303, 13323, 13459, 14397, 14401, 14403, 14405, 14507, 14557, 14559, |
| | 14619, 15211, 15375, 15785, 15803, 15817, 16085, 16093, 17143, 17213, 17281, |
| | 17283, 17285, 17287, 17289, 17291, 17389, 17605, 17607, 17611, 17615, 17617, |
| | 17619, 17621, 17623, 17625, 17627, 17629, 17631, 17641, 17643, 17645, 17647, |
| | 17691, 17721, 17723, 17727, 17805, 17825, 17881, 17929, 17939, 17947 |
| H36B | 18085, 18105, 18237, 18873, 18893, 18895, 18931, 19255, 19259, 19261, 19263, |
| | 19333, 19381, 19383, 19385, 19429, 19879, 20105, 20109, 20367, 20405, 20537, |
| | 20595, 21587, 21589, 21599, 21903, 21951, 22031, 22135, 22269, 22271, 22275, |
| | 22277, 22279, 22281, 22283, 22285, 22287, 22293, 22299, 22301, 22433, 22435, |
| | 22437, 22439, 22441, 22443, 22445, 22447, 22449, 22451, 22453, 22455, 22457, |
| | 22467, 22507, 22509, 22643 |

TABLE V
‘Core’ coding sequences

| 2603vr | 18rs21 | 515 | cjb111 | coh1 | h36b | NEM316 |
|---|---|---|---|---|---|---|
| 22535220 | 1, 3919 | 8615 | 12909 | 14061 | 22383 | 24413708 |
| 22535221 | 3 | 8617 | 12911 | 14063 | 22385, 22427 | 24413709 |
| 22535222 | 5 | 8619, 8703 | 12913 | 14065 | 22387 | 24413710 |
| 22535223 | 7 | 8621 | 12915 | 14067 | 22389 | 24413711 |
| 22535224 | 9 | 8623 | 12917 | 14069 | 22391 | 24413712 |
| 22535225 | 11 | 8625 | 12919 | 14071 | 22393 | 24413713 |
| 22533003 | 13 | 8627 | 12921 | 14073 | 22395 | 23094427 |
| 22533004 | 15 | 4303, 8629 | 12923 | 14075 | 22397 | 23094428 |
| 22533005 | 17 | 4305, 8631 | 12925, 12927 | 14077 | 22399 | 23094429 |
| 22533007 | 21 | 4309 | 9607 | 14081 | 22403 | 23094431 |
| 22533008 | 23 | 4311 | 9609 | 14083 | 22405 | 23094432 |
| 22533009 | 25 | 4313 | 9611 | 14085 | 22407 | 23094433 |
| 22533010 | 27, 29 | 4315 | 9613 | 14087 | 22409, 22663 | 23094434 |
| 22533012 | 31 | 4317 | 9615 | 14089 | 22413 | 23094435 |
| 22533013 | 33 | 4319 | 9617 | 14091 | 22415 | 23094436 |
| 22533014 | 35 | 4321 | 9619 | 14093 | 22417 | 23094437 |
| 22533015 | 37 | 4323 | 9621 | 13135, 14095 | 22419 | 23094438 |
| 22533016, 22534732 | 39 | 4325 | 9623, 12021 | 13137, 14097, | 21493, 22421 | 23094439 |
| | | | | 16681 | | |
| 22533017 | 41 | 4327 | 9625 | 13139, 13143, | 22423 | 23094440 |
| | | | | 14099 | | |
| 22533018 | 43 | 4329 | 9627 | 13145, 13147, | 22425, 22623 | 23094441 |
| | | | | 13149, 14101 | | |
| 22533020 | 45, 47, 49, 51 | 4331 | 8801 | 13181 | 17963 | 23094442 |
| 22533021 | 53 | 4333, 4335 | 8803 | 13183 | 17965 | 23094443 |
| 22533022 | 55 | 4337 | 8805 | 13185 | 17967 | 23094444 |
| 22533023 | 57 | 4339, 4341 | 8807 | 13187, 13189 | 17969 | 23094445 |
| 22533024 | 59 | 4343 | 8809 | 13191 | 17971 | 23094446 |
| 22533025 | 61 | 4345 | 8811 | 13193 | 17973 | 23094447 |
| 22533026 | 63 | 4347 | 8813, 8815 | 13195 | 17975 | 23094448 |
| 22533027 | 65 | 4349 | 8817 | 13197 | 17977, 18033, | 23094449 |
| | | | | | 18035 | |
| 22533028 | 67 | 4351 | 8819 | 13199, 13245, | 17979, 18037, | 23094450 |
| | | | | 13247, 17811, | 22555 | |
| | | | | 17821, 17859 | | |
| 22533029 | 69, 71, 3965, 4017, | 4353 | 8821 | 13201, 17803, | 17981 | 23094451 |
| | 4061 | | | 17891 | | |
| 22533030 | 73, 75, 3963 | 4355 | 8823 | 13203 | 17983 | 23094452 |
| 22533032 | 77 | 4357, 4365 | 8825 | 13205 | 17985 | 23094453 |
| 22533034 | 81 | 4361, 4369, 8707 | 8829 | 13209 | 17989, 22505 | 23094455 |
| 22533035 | 83, 85 | 4371 | 8831 | 13211, 13249, | 17991 | 23094456 |
| | | | | 13251, 13253 | | |
| 22533036 | 87 | 4373 | 8833 | 13213 | 17993 | 23094457 |
| 22533037 | 89 | 4375 | 8835 | 13215, 17895 | 17995 | 23094458 |
| 22533038 | 91 | 4377, 4413, 4415, 4417 | 8837 | 13217, 13255, | 17997 | 23094459 |
| | | | | 17893 | | |
| 22533039 | 93 | 4379 | 8839 | 13219, 13257 | 17999 | 23094460 |
| 22533040 | 95 | 4381, 4419, 4421 | 8841 | 13221, 13259 | 18001 | 23094461 |
| 22533041 | 97 | 4383 | 8843 | 13223, 13261 | 18003 | 23094462 |
| 22533042 | 99 | 4385 | 8845 | 13225 | 18005 | 23094463 |
| 22533043 | 101 | 4387 | 8847 | 13227 | 18007 | 23094464 |
| 22533044 | 103 | 4389 | 8849 | 13229 | 18009 | 23094465 |
| 22533045 | 105 | 4391 | 8851 | 13231 | 18011 | 23094466 |
| 22533046 | 107 | 4393 | 8853 | 13233 | 18013 | 23094467 |
| 22533047 | 109 | 4395 | 8855 | 13235, 17875, | 18015, 18039, | 23094468 |
| | | | | 17941 | 18041, 22631 | |
| 22533048 | 111 | 4397 | 8857, 13023 | 13237 | 18017 | 23094469 |
| 22533049 | 113, 115, 117, 3949 | 4399 | 8859 | 13239 | 18019 | 23094470 |
| 22533051 | 121 | 4405 | 8863 | 13265 | 18023 | 23094473 |
| 22533054 | 125 | 4409 | 8867 | 13269 | 18027 | 23094475 |
| 22533055 | 127 | 4411 | 8869 | 13271 | 18029 | 23094476 |
| 22533056 | 129 | 4423 | 8871 | 13273 | 18031 | 23094477 |
| 22533057 | 131 | 4425, 4427 | 8873 | 13275 | 18043 | 23094478 |
| 22533058 | 133 | 4429, 4431, 4433 | 8875 | 13277, 13279 | 18045, 18047, | 23094479 |
| | | | | | 18049, 18051 | |
| 22533059 | 135 | 4435, 4437 | 8877 | 13281 | 18053 | 23094480 |
| 22533060 | 137 | 4439 | 8879 | 17759 | 18055 | 23094481 |
| 22533061 | 139 | 4441 | 8881 | 13283 | 18057 | 23094482 |
| 22533063 | 143 | 4443 | 8885 | 13285 | 18061 | 23094484 |
| 22533064 | 145 | 4445 | 8887 | 13287, 13289 | 18063, 18071 | 23094485 |
| 22533065 | 147 | 4447 | 8889 | 13291 | 18065, 18073 | 23094486 |
| 22533066 | 149 | 4449 | 8891 | 13293 | 18067, 18069, | 23094487 |
| | | | | | 18075 | |
| 22533067 | 151 | 4451 | 8893 | 13295 | 18077 | 23094488 |
| 22533068 | 153 | 4453 | 8895 | 13297 | 18079 | 23094489 |
| 22533069 | 155 | 4455 | 8897 | 13299 | 18081 | 23094490 |
| 22533070 | 157 | 4457 | 8899 | 13301 | 18083 | 23094491 |
| 22533071 | 159 | 4461 | 8903 | 13155, 13305, | 18087 | 23094492 |
| | | | | 17925 | | |
| 22533072 | 161 | 4463 | 8905 | 13307 | 18089 | 23094493 |
| 22533073 | 163 | 4465 | 8907 | 13309 | 18091 | 23094494 |
| 22533074 | 165 | 4467 | 8909 | 13311 | 18093 | 23094495 |
| 22533075 | 167 | 4469 | 8911 | 13313 | 18095 | 23094496 |
| 22533076, 22534790 | 169, 3205 | 6299 | 12167 | 16801 | 21615 | 23094497, 24413378 |
| 22533077 | 171 | 4471 | 8913 | 13315 | 18097 | 23094498 |
| 22533078 | 173 | 4473 | 8915 | 13317 | 18099 | 23094499 |
| 22533080 | 175 | 4475 | 8917 | 13319 | 18101 | 23094500 |
| 22533081 | 177 | 4477 | 8919 | 13321, 17737 | 18103 | 23094501 |
| 22533082 | 179 | 4481 | 8923 | 13325 | 18107 | 23094502 |
| 22533083 | 181 | 4483 | 8925 | 13327 | 18109 | 23094503 |
| 22533084 | 183 | 4485 | 8927 | 13329 | 18111 | 23094504 |
| 22533085 | 185 | 4487 | 8929, 8931 | 13331 | 18113 | 23094505 |
| 22533090 | 193 | 4495 | 8939 | 13333, 13335 | 18121, 18123 | 23094510 |
| 22533091 | 195 | 4497 | 8941 | 13337 | 18125 | 23094511 |
| 22533092, 22533093, | 199, 201, 503 | 4499, 4875 | 8943, 8945, 9475, | 13341, 13345, | 18127, 18135, | 23094512 |
| 22533257, | | | 9497 | 13663, 13681, | 18437, 18451 | |
| 22533270 | | | | 13685 | | |
| 22533094, 22533258, | 203, 505 | 4501 | 8947, 9477 | 13343, 13347, | 18129, 18137 | 23094513 |
| 22533260 | | | | 13665, 13687, | | |
| | | | | 17743 | | |
| 22533095 | 205 | 4503 | 8949 | 13349 | 18139 | 23094514 |
| 22533096 | 207 | 4505 | 8951 | 13339, 13351 | 18141 | 23094515 |
| 22533097 | 209 | 4507 | 8953 | 13353 | 18143 | 23094516 |
| 22533098 | 211 | 4509 | 8955 | 13355, 13357 | 18145 | 23094517 |
| 22533099 | 213 | 4511 | 8957 | 13359 | 18147 | 23094518 |
| 22533100 | 215 | 4513 | 8959 | 13361 | 18149 | 23094519 |
| 22533101 | 217 | 4515 | 8961 | 13363 | 18151, 18171 | 23094520 |
| 22533102 | 219 | 4517 | 8963 | 13365 | 18153 | 23094521 |
| 22533104 | 221 | 4519 | 8965 | 13367 | 18155 | 23094522 |
| 22533105 | 223 | 4521 | 8967 | 13369 | 18157 | 23094523 |
| 22533106 | 225 | 4523 | 8969 | 13371 | 18159 | 23094524 |
| 22533107 | 227 | 4525 | 8971 | 13373 | 18161 | 23094525 |
| 22533108 | 229 | 4527 | 8973 | 13375, 13385 | 18163 | 23094526 |
| 22533116 | 231 | 4549 | 8991 | 13399 | 18183 | 23094534 |
| 22533117 | 233 | 4551 | 8993 | 13401, 13403 | 18185 | 23094535 |
| 22533118 | 235 | 4553 | 8995 | 13405 | 18187 | 23094536 |
| 22533119 | 237 | 4555 | 8997 | 13407 | 18189 | 23094537 |
| 22533120 | 239 | 4557 | 8999 | 13413 | 18193 | 23094538 |
| 22533121 | 241 | 4559, 4561, 4563 | 9001 | 13415 | 18195 | 23094539 |
| 22533122 | 243 | 4565 | 9003 | 13417, 13443 | 18197 | 23094540 |
| 22533123 | 245 | 4567 | 9005 | 13419, 13445 | 18199 | 23094541 |
| 22533124 | 247 | 4569 | 9007 | 13421 | 18201 | 23094542 |
| 22533125 | 249, 251, 253, 4055 | 4571 | 9009 | 13423 | 18203 | 23094543 |
| 22533126 | 255 | 4573 | 9011 | 13425 | 18205 | 23094544 |
| 22533128 | 257 | 4577 | 9015 | 13429 | 18209 | 23094545 |
| 22533133 | 259, 261 | 4585 | 9023 | 13437 | 18217 | 23094549 |
| 22533134 | 263, 265, 267 | 4587 | 9025 | 13439 | 18219 | 23094550 |
| 22533139 | 269, 271 | 4591 | 9035 | 13451, 13487 | 18227 | 23094555 |
| 22533140 | 273 | 4593 | 9037 | 13453 | 18229 | 23094556 |
| 22533141, 22533904, | 275, 1701, 2693, | 4595, 6149, 7287, | 9039, 10563, 11555 | 13455, 15055, | 18231, 19849, | 23095333, 24413101 |
| 22534480 | 4111 | 8771 | | 16017 | 20993, 20995 | |
| 22533143 | 281, 285, 4051 | 4603 | 9045 | 13461 | 22173 | 23094558 |
| 22533144 | 287, 4053 | 4605 | 9047 | 13463 | 22169, 22171 | 23094559 |
| 22533145 | 289, 4163 | 4607, 4609 | 9049 | 13465 | 22165, 22167 | 23094560 |
| 22533146 | 291 | 4611 | 9051 | 13467 | 22163 | 23094561 |
| 22533147 | 293 | 4613 | 9053 | 13469 | 22161 | 23094562 |
| 22533148 | 295, 297 | 4615 | 9055 | 13471 | 22159 | 23094563 |
| 22533149 | 299 | 4617 | 9057 | 13473, 17949 | 22157, 22647 | 23094564 |
| 22533150, 22534271 | 301 | 4619 | 9059 | 13475 | 22155 | 23094565 |
| 22533151, 22534154 | 303 | 4621, 6727, 6729, | 9061, 11015 | 13477, 13489, | 20309, 20311, | 23094566, 23095668 |
| | | 6731 | | 15575 | 20313, 22153 | |
| 22533152 | 305 | 4623 | 9063 | 13479 | 22151 | 23094567 |
| 22533154 | 307 | 4625 | 9065 | 13481 | 22149 | 23094568 |
| 22533155 | 309, 311 | 4627 | 9067 | 13483 | 22147 | 23094569 |
| 22533156 | 313 | 4629 | 9069 | 13485 | 22145 | 23094570 |
| 22533158 | 315 | 4633 | 9073 | 13493 | 17955, 18239 | 23094572 |
| 22533159 | 317 | 4635 | 9075 | 13495 | 17957, 18241 | 23094573 |
| 22533160 | 319 | 4637 | 9077 | 13497 | 17959 | 23094574 |
| 22533161 | 321, 4077, 4101, | 4639, 8785 | 9079 | 13499 | 17961, 22541 | 23094575 |
| | 4113 | | | | | |
| 22533162 | 323 | 4641 | 9081 | 13501, 13503 | 18243, 18253 | 23094576 |
| 22533163 | 325 | 4643 | 9087 | 13505 | 18245 | 23094577 |
| 22533164 | 327 | 4645 | 9089 | 13507 | 18247 | 23094578 |
| 22533165 | 329 | 4647 | 9091 | 13509 | 18249 | 23094579 |
| 22533167 | 333 | 4655, 4657 | 9095 | 13513 | 18255 | 23094581 |
| 22533168 | 335 | 4659 | 9097 | 13515, 13517 | 18257 | 23094582 |
| 22533169 | 337 | 4661 | 9099, 13081 | 13519 | 18259 | 23094583 |
| 22533170 | 339, 343 | 4663, 4729 | 9083, 9085, 9101 | 13521, 17817 | 18261, 18281, | 23094584 |
| | | | | | 18283, 18285 | |
| 22533171 | 341, 345 | 4665 | 9103 | 13523 | 18263, 18287 | 23094585 |
| 22533172 | 347 | 4667 | 9105 | 13525, 13527 | 18265 | 23094586 |
| 22533173 | 349 | 4669 | 9107 | 13529 | 18267 | 23094587 |
| 22533174 | 351 | 4675 | 9115 | 13531 | 18269 | 23094591 |
| 22533176 | 355 | 4679 | 9119 | 13533 | 18273 | 23094593 |
| 22533177 | 357 | 4681 | 9121 | 13535 | 18275 | 23094594 |
| 22533178 | 359 | 4683 | 9123 | 13537 | 18277 | 23094595 |
| 22533181 | 365 | 4689 | 9129 | 13543 | 18289 | 23094597 |
| 22533182 | 367 | 4691 | 9131 | 13545 | 18291 | 23094598 |
| 22533183 | 369 | 4693 | 9133 | 13547, 13611, | 18293 | 23094599 |
| | | | | 13613 | | |
| 22533186 | 371 | 4697 | 9137 | 13551 | 18297 | 23094601 |
| 22533187 | 373 | 4699 | 9139 | 13553 | 18299 | 23094602 |
| 22533188 | 375, 377 | 4701 | 9141 | 13555, 13615, | 18301 | 23094603 |
| | | | | 13617 | | |
| 22533189 | 379 | 4703 | 9143 | 13557, 13619 | 18303 | 23094604 |
| 22533190, 22533587, | 381, 3121 | 4705, 5537, 7833 | 9145, 12049 | 13559 | 18305, 19283 | 23094605, 24413338 |
| 22534747, | | | | | | |
| 22534904 | | | | | | |
| 22533191 | 383 | 4707 | 9147 | 13561 | 18307 | 23094606 |
| 22533192 | 385 | 4709 | 9149 | 13563 | 18309 | 23094607 |
| 22533193 | 387 | 4711, 4731 | 9151 | 13565 | 18311 | 23094608 |
| 22533194 | 389 | 4713, 4733, 8679 | 9153 | 13567 | 18313 | 23094609 |
| 22533195 | 391, 393 | 4715 | 9155 | 13569 | 18315, 18319 | 23094610 |
| 22533197 | 397 | 4719 | 9159 | 13573, 13621, | 18323 | 23094611 |
| | | | | 13623, 13625 | | |
| 22533198 | 399 | 4721 | 9161 | 13575, 13627, | 18325 | 23094612 |
| | | | | 13629 | | |
| 22533199 | 401 | 4723 | 9163 | 13577, 13631 | 18327 | 23094613 |
| 22533200 | 403 | 4725 | 9165 | 13579, 13633, | 18329 | 23094614 |
| | | | | 13635 | | |
| 22533201 | 405 | 4727, 4735 | 9167 | 13581 | 18331 | 23094615 |
| 22533202 | 407 | 4737, 4739 | 9169 | 13583 | 18333, 18693 | 23094616 |
| 22533203 | 409, 411 | 4741 | 9171 | 13585 | 18335, 18431 | 23094617 |
| 22533204, 22533207 | 413 | 4745, 4749 | 9173 | 13587, 13637 | 18337 | 23094618 |
| 22533208 | 415 | 4747, 4751 | 9175 | 13589, 13639 | 18339 | 23094619 |
| 22533209 | 417 | 4753, 8715 | 9177 | 13591, 13641 | 18341 | 23094620 |
| 22533210 | 419 | 4755 | 9179 | 13593 | 18343 | 23094621 |
| 22533211 | 421 | 4757 | 9181 | 13595 | 18345 | 23094622 |
| 22533212 | 423, 425 | 4759 | 9183 | 13597 | 18347 | 23094623 |
| 22533213 | 427 | 4761 | 9185 | 13599 | 18349 | 23094624 |
| 22533214 | 429 | 4763, 8793 | 9187, 13129 | 13601, 13643 | 18351, 18433 | 23094625 |
| 22533215 | 431 | 4765 | 9189 | 13603 | 18353 | 23094626 |
| 22533216 | 433 | 4767 | 9191 | 13605 | 18355, 22597 | 23094627 |
| 22533217 | 435 | 4769 | 9193 | 13607 | 18357, 22595 | 23094628 |
| 22533218 | 437, 439 | 4771 | 9195 | 13609, 13645 | 18359 | 23094629 |
| 22533219 | 441 | 4773 | 9197 | 13647 | 18361 | 23094630 |
| 22533220 | 443 | 4775 | 9199 | 13649 | 18363, 18435, | 23094631 |
| | | | | | 22635 | |
| 22533221 | 445 | 4777 | 9201 | 13651 | 18365 | 23094632 |
| 22533222 | 447 | 4779 | 9203 | 13653, 13655 | 18367 | 23094633 |
| 22533225 | 449 | 4787 | 9207 | 13659 | 18371 | 23094636 |
| 22533226 | 451 | 4789 | 9209 | 13661 | 18373 | 23094637 |
| 22533228 | 453 | 4791 | 9211 | 17729 | 18375 | 23094638 |
| 22533231 | 459 | 4795 | 9217 | 17725 | 18381 | 23094641 |
| 22533241, 22534994 | 473, 3519 | 8203 | 9235, 12471, 12939, | 17133 | 18397, 21891 | 23094649, 24413519 |
| | | | 12941 | | | |
| 22533245 | 481 | 4821, 4823 | 9249, 12537, 12539, | 17327, 17331 | 18405, 18407, | 24413548, 24413559 |
| | | | 12543 | | 21953, 21955, | |
| | | | | | 21959, 21963 | |
| 22533251 | 491 | 4835 | 9261 | 13167, 13169 | 18419 | 23094656 |
| 22533252 | 493 | 4837, 4839, 4841, | 9263 | 13171 | 18421 | 23094657 |
| | | 6645 | | | | |
| 22533253 | 495 | 4843 | 9265 | 13173 | 18423 | 23094658 |
| 22533254 | 497 | 4845 | 9267 | 13175 | 18425 | 23094659 |
| 22533255 | 499 | 4847 | 9269, 9271 | 13177 | 18427 | 23094660 |
| 22533256 | 501, 3975, 4215 | 4849 | 9273 | 13179, 17931 | 18429 | 23094661 |
| 22533265 | 517 | 4865 | 9487 | 13671 | 18441 | 23094671 |
| 22533266 | 519 | 4867 | 9489 | 13673 | 18443 | 23094672 |
| 22533267 | 521 | 4869 | 9491 | 13675 | 18445 | 23094673 |
| 22533268 | 523 | 4871 | 9493 | 13677, 17877 | 18447 | 23094674 |
| 22533269 | 525 | 4873 | 9495 | 13679 | 18449 | 23094675 |
| 22533271 | 527 | 4877 | 9499 | 13683 | 18453 | 23094676 |
| 22533272 | 529 | 4879, 4881 | 9317, 9501 | 13691 | 18455, 18459 | 23094677 |
| 22533276 | 531 | 4885 | 9321 | 13695 | 18463 | 23094679 |
| 22533277 | 533 | 4887 | 9323 | 13697 | 18465, 18471 | 23094680 |
| 22533278 | 535 | 4889 | 9325 | 13699 | 18467 | 23094681 |
| 22533279 | 537 | 4891 | 9327 | 13701 | 18469 | 23094682 |
| 22533280 | 539 | 4893 | 9329, 9331 | 13703 | 18473, 22133 | 23094683 |
| 22533281 | 541 | 4895 | 9333 | 13705 | 18475 | 23094684 |
| 22533282 | 543 | 4897 | 9335 | 13707 | 18477 | 23094685 |
| 22533283 | 545 | 4899 | 9337 | 13709 | 18479, 18609, | 23094686 |
| | | | | | 18613 | |
| 22533284 | 547 | 4901 | 9339 | 13711 | 18481, 18611, | 23094687 |
| | | | | | 18615 | |
| 22533285 | 549 | 4903 | 9341 | 13713 | 18483, 18617 | 23094688 |
| 22533286 | 551 | 4905 | 9343 | 13715 | 18485 | 23094689 |
| 22533287 | 553 | 4907 | 9345 | 13717 | 18487 | 23094690 |
| 22533288 | 555 | 4909 | 9347 | 13719, 13781 | 18489 | 23094691 |
| 22533290 | 557 | 4911 | 9349 | 13721, 13783 | 18491 | 23094692 |
| 22533291 | 559 | 4913 | 9351 | 13723, 13785 | 18493 | 23094693 |
| 22533292 | 561, 563, 565, | 4915 | 9353, 12121 | 13725 | 18495, 21593, | 23094694 |
| | 4153, 4155 | | | | 22429 | |
| 22533293 | 567 | 4917 | 9355 | 13727 | 18497 | 23094695 |
| 22533294 | 569, 571 | 4919 | 9357 | 13729 | 18499 | 23094696 |
| 22533295 | 573 | 4921 | 9359 | 13731 | 18501 | 23094697 |
| 22533296 | 575 | 4923, 4929 | 9361 | 13733, 13735, | 18503 | 23094698 |
| | | | | 13787, 13789 | | |
| 22533297 | 577 | 4925, 4931 | 9363 | 13737 | 18505 | 23094699 |
| 22533298 | 579 | 4927, 4933 | 9365 | 13739 | 18507 | 23094700 |
| 22533299 | 581 | 4935 | 9367 | 13741 | 18509 | 23094701 |
| 22533300 | 583 | 4937 | 9369 | 13743 | 18511 | 23094702 |
| 22533301 | 585 | 4939 | 9371 | 13745, 13791 | 18513 | 23094703 |
| 22533302 | 587 | 4941 | 9373 | 13747 | 18515 | 23094704 |
| 22533303 | 589, 591 | 4943, 4945, 8721 | 9375 | 13749 | 18517 | 23094705 |
| 22533304 | 593 | 4947 | 9377 | 13751 | 18519 | 23094706 |
| 22533305 | 595 | 4949 | 9379 | 13753 | 18521 | 23094707 |
| 22533306 | 597 | 4951 | 9381 | 13755 | 18523 | 23094708 |
| 22533308 | 599 | 4953 | 9383 | 13757 | 18525 | 23094709 |
| 22533309 | 601 | 4955 | 9385 | 13759 | 18527 | 23094710 |
| 22533310 | 603 | 4957 | 9387 | 13761 | 18529 | 23094711 |
| 22533311 | 605 | 4959 | 9389 | 13763 | 18531 | 23094712 |
| 22533312 | 607 | 4961 | 9391 | 13765, 13793 | 18533 | 23094713 |
| 22533313 | 609 | 4963 | 9393 | 13767 | 18535, 18537 | 23094714 |
| 22533314 | 611 | 4965 | 9395 | 13769 | 18539 | 23094715 |
| 22533315 | 613 | 4967 | 9397 | 13771 | 18541 | 23094716 |
| 22533317 | 615 | 4971 | 9401 | 13775 | 18545 | 23094717 |
| 22533318 | 617 | 4973 | 9403 | 13777, 13779 | 18547 | 23094718 |
| 22533319 | 619, 621 | 4975 | 9405 | 13795 | 18549 | 23094719 |
| 22533320 | 623 | 4977 | 9407 | 13797 | 18551 | 23094720 |
| 22533321 | 625 | 4979 | 9409 | 13799 | 18553, 18619 | 23094721 |
| 22533323 | 627 | 4983 | 9413 | 13803 | 18557 | 23094722 |
| 22533325 | 631 | 4985 | 9417 | 13807 | 18561 | 23094724 |
| 22533326 | 635 | 4989 | 9421 | 13811 | 18565 | 23094726 |
| 22533327 | 637 | 4991 | 9423 | 13813 | 18567 | 23094727 |
| 22533328 | 639 | 4993 | 9425 | 13815 | 18569 | 23094728 |
| 22533330 | 641 | 4995 | 9427 | 13817 | 18571 | 23094729 |
| 22533331 | 643 | 4997 | 9429 | 13819 | 18573 | 23094730 |
| 22533332 | 645 | 4999 | 9431 | 13821 | 18575, 18621 | 23094731 |
| 22533333 | 647 | 5001 | 9433 | 13823, 13871 | 18577, 22673 | 23094732 |
| 22533334 | 649 | 5003 | 9435 | 13825, 13873 | 18579 | 23094733 |
| 22533335 | 651 | 5005 | 9437 | 13827, 13875 | 18581 | 23094734 |
| 22533336 | 653 | 5007 | 9439 | 13829, 13877, | 18583 | 23094735 |
| | | | | 13879, 13881 | | |
| 22533337 | 655 | 5009, 5043 | 9441 | 13831 | 18585 | 23094736 |
| 22533338 | 657 | 5011 | 9443 | 13833 | 18587 | 23094737 |
| 22533339 | 659 | 5013 | 9445 | 13835 | 18589, 18623 | 23094738 |
| 22533340 | 661 | 5015 | 9447 | 13837 | 18591 | 23094739 |
| 22533341 | 663 | 5017 | 9449 | 13839 | 18593 | 23094740 |
| 22533342 | 665 | 5019 | 9451 | 13841 | 18595, 18625 | 23094741 |
| 22533343 | 667 | 5021 | 9453 | 13843 | 18597, 18627 | 23094742 |
| 22533344 | 669 | 5023 | 9455 | 13845 | 18599 | 23094743 |
| 22533345 | 671 | 5025 | 9457 | 13847 | 18601 | 23094744 |
| 22533346 | 673 | 5027 | 9459 | 13849 | 18603 | 23094745 |
| 22533347 | 675 | 5029 | 9461 | 13851 | 18605, 18629 | 23094746 |
| 22533348 | 677 | 5031 | 9463 | 13853, 17831 | 18607, 18631, | 23094747 |
| | | | | | 18633 | |
| 22533350 | 679 | 5033 | 9465, 9503 | 13855 | 18635, 18645 | 23094748 |
| 22533351 | 681 | 5035 | 9467 | 13857 | 18637 | 23094749 |
| 22533352 | 683 | 5037 | 9469 | 13859, 17911 | 18639 | 23094750 |
| 22533353 | 685 | 5039 | 9471 | 13861 | 18641 | 23094751 |
| 22533354 | 687 | 5041 | 9473 | 13863 | 18643 | 23094752 |
| 22533355 | 689, 3921 | 5045 | 9275 | 13865 | 18647 | 23094753 |
| 22533356 | 691 | 5047 | 9277 | 13867 | 18649 | 23094754 |
| 22533358 | 693 | 5049, 5079 | 9279 | 13869, 13883 | 18651, 18653 | 23094755 |
| 22533359 | 695 | 5051 | 9281 | 13885, 13887 | 18655 | 23094756 |
| 22533360 | 697 | 5053 | 9283 | 13889 | 18657 | 23094757 |
| 22533361 | 699 | 5055 | 9285 | 13891 | 18659 | 23094758 |
| 22533362 | 701, 4133, 4135 | 5057 | 9287 | 13893 | 18661 | 23094759 |
| 22533363 | 703 | 5059, 5081 | 9289 | 13895, 17153 | 18663 | 23094760 |
| 22533364 | 705 | 5061, 5083 | 9291 | 13897 | 18665 | 23094761 |
| 22533365 | 707 | 5063 | 9293 | 13899 | 18667 | 23094762 |
| 22533366 | 709 | 5065 | 9295 | 13901 | 18669 | 23094763 |
| 22533367 | 711, 723, 4301 | 5067 | 9297 | 13903 | 18671, 18695 | 23094764 |
| 22533368 | 713, 725, 4299 | 5069 | 9299 | 13905 | 18673, 18697 | 23094765 |
| 22533369 | 715, 727, 4297 | 5071, 5087 | 9301 | 13907 | 18675, 18699, | 23094766 |
| | | | | | 18701 | |
| 22533370, 22534818 | 717, 729, 3261, | 5073, 5089, 5091, | 9303, 12225 | 13909, 16863, | 18677, 18703, | 23094767, 24413404 |
| | 3263, 4165, 4167, | 7953 | | 16893, 17849 | 21667 | |
| | 4295 | | | | | |
| 22533372 | 719, 731, 4293 | 5075, 5093 | 9305 | 13911 | 18679 | 23094768 |
| 22533373 | 721, 733, 4291 | 5095 | 9307 | 13913 | 18681 | 23094769 |
| 22533374 | 735 | 5097 | 9309 | 13915 | 18683 | 23094770 |
| 22533375 | 737 | 5099, 5105 | 9311 | 13917 | 18685 | 23094771 |
| 22533376 | 739 | 5101, 5107, 5109 | 9313 | 13919 | 18687 | 23094772 |
| 22533377 | 741 | 5111 | 9315 | 13921 | 18705 | 23094773 |
| 22533379 | 743 | 5115 | 9507 | 13925 | 18711 | 23094775 |
| 22533380 | 745 | 5117, 5149 | 9509 | 13927 | 18713 | 23094776 |
| 22533381 | 747 | 5119, 5151 | 9511 | 13929 | 18715 | 23094777 |
| 22533382 | 749 | 5121 | 9513 | 13931 | 18717 | 23094778 |
| 22533383 | 751 | 5123 | 9515 | 13933 | 18719 | 23094779 |
| 22533384 | 753 | 5125 | 9517 | 13935 | 18721 | 23094780 |
| 22533385 | 755 | 5127 | 9519 | 13937 | 18723 | 23094781 |
| 22533386 | 757 | 5129 | 9521 | 13939 | 18725 | 23094782 |
| 22533387 | 759 | 5131 | 9523 | 13941 | 18727 | 23094783 |
| 22533388 | 761 | 5133 | 9525 | 13943 | 18729 | 23094785 |
| 22533390 | 763 | 5137 | 9529 | 13947 | 18733 | 23094786 |
| 22533391 | 765 | 5139 | 9531 | 13949 | 18735 | 23094787 |
| 22533392 | 767 | 5141 | 9533 | 13951 | 18737 | 23094837 |
| 22533393 | 769 | 5143 | 9535 | 13953 | 18739 | 23094838 |
| 22533394 | 771 | 5145 | 9537 | 13955 | 18741 | 23094839 |
| 22533395 | 773 | 5147 | 9539 | 13957 | 18743 | 23094840 |
| 22533396 | 775 | 5153 | 9541 | 13959 | 18745 | 23094841 |
| 22533397 | 777 | 5155 | 9543 | 13961 | 18747 | 23094842 |
| 22533398 | 779, 781, 783 | 5157, 5159, 5161 | 9545 | 13963 | 18749, 18751 | 23094843 |
| 22533400 | 785 | 5163 | 9547 | 13965 | 18753 | 23094844 |
| 22533401 | 787 | 5165 | 9549 | 13967 | 18755 | 23094845 |
| 22533402 | 789 | 5167, 5201 | 9551 | 13969 | 18757 | 23094846 |
| 22533403 | 791, 2369, 4147, | 5169 | 9553 | 13971 | 18759, 18783, | 23094847 |
| | 4149, 4179 | | | | 20597 | |
| 22533404 | 793 | 5171 | 9555 | 13973 | 18761 | 23094848 |
| 22533405 | 795 | 5173 | 9557 | 13975 | 18763 | 23094849 |
| 22533406 | 797 | 5175 | 9559 | 13977 | 18765 | 23094850 |
| 22533407 | 799 | 5177 | 9561 | 13979 | 18767, 22527 | 23094851 |
| 22533408 | 801 | 5179 | 9563 | 13981 | 18769, 22525 | 23094852 |
| 22533409 | 803 | 5181 | 9565 | 13983 | 18771 | 23094853 |
| 22533410 | 805 | 5183, 5203, 5205 | 9567 | 13985, 17809 | 18773 | 23094854 |
| 22533411 | 807 | 5185 | 9569 | 13987 | 18775 | 23094855 |
| 22533412 | 809 | 5187 | 9571 | 13989, 13991, | 18777 | 23094856 |
| | | | | 14011 | | |
| 22533413, 22534328 | 811 | 5189 | 9573 | 13993 | 18779 | 23094857 |
| 22533414 | 813 | 5191 | 9575, 9577 | 13995 | 18781 | 23094858 |
| 22533415 | 815 | 5193 | 9579 | 13997 | 18785 | 23094859 |
| 22533416 | 817 | 5195 | 9581 | 13999 | 18787 | 23094860 |
| 22533417 | 819 | 5197 | 9583 | 14001, 14003 | 18789, 18791 | 23094861 |
| 22533418 | 821 | 5199 | 9585 | 14005 | 18793 | 23094862 |
| 22533420 | 823 | 4651 | 9589 | 14007 | 18797 | 23094863 |
| 22533421 | 825 | 4653, 5209 | 9591 | 14009 | 18799 | 23094864 |
| 22533426 | 827 | 5217 | 9599 | 14019 | 18809 | 23094868 |
| 22533422 | 829 | 5211 | 9593 | 14013 | 18801 | 23094865 |
| 22533427 | 831 | 5219 | 9601 | 14021 | 18811 | 23094869 |
| 22533428 | 833 | 5221 | 9603 | 14023, 14025 | 18813 | 23094870 |
| 22533429 | 835 | 5223 | 9605 | 14027 | 18815 | 23094871 |
| 22533430 | 837, 4049 | 4851 | 9629 | 14103 | 18817 | 23094872 |
| 22533431 | 839 | 4853 | 9631 | 14105 | 18819 | 23094873 |
| 22533432 | 841, 843 | 4855, 8777 | 9633 | 14107 | 18821 | 23094874 |
| 22533433 | 845 | 4857 | 9635 | 14109 | 18823 | 23094875 |
| 22533434 | 847 | 4859 | 9637 | 14111 | 18825 | 23094876 |
| 22533435 | 849 | 4861, 4863, 5225 | 9639 | 14113, 14115, | 18827, 18867, | 23094877 |
| | | | | 14117, 14159, | 18869 | |
| | | | | 14161 | | |
| 22533436 | 851 | 5227 | 9641 | 14119, 14163, | 18829, 18871 | 23094878 |
| 22533438 | 853 | 5229 | 9643 | 14121 | 18831 | 23094879 |
| 22533439 | 855 | 5231 | 9645 | 14123 | 18833 | 23094880 |
| 22533440 | 857 | 5233 | 9647 | 14125 | 18835 | 23094881 |
| 22533441 | 859 | 5235 | 9649 | 14127 | 18837 | 23094882 |
| 22533442 | 861 | 5237 | 9651 | 14129 | 18839 | 23094883 |
| 22533443 | 863 | 5239 | 9653 | 14135 | 18841 | 23094884 |
| 22533444 | 865 | 5241 | 9655, 9657 | 14137 | 18843, 18845 | 23094885 |
| 22533445 | 867 | 5243 | 9659 | 14139 | 18847 | 23094886 |
| 22533446 | 869 | 5245 | 9661, 9663 | 14141 | 18849 | 23094887 |
| 22533447 | 871 | 5247 | 9665 | 14143 | 18851 | 23094888 |
| 22533448 | 873 | 5249, 5251 | 9667, 9669 | 14145, 14147 | 18853, 18855 | 23094889 |
| 22533449 | 875 | 5253, 5255 | 9671 | 14149 | 18857 | 23094890 |
| 22533450 | 877 | 5257, 5641 | 9673 | 14151 | 18859 | 23094891 |
| 22533451 | 885 | 5259 | 9675 | 14153 | 18861 | 23094892 |
| 22533452 | 887, 3709 | 5261, 5265, 8417 | 9677, 12617 | 14155, 17403, | 18863, 22043 | 23094893 |
| | | | | 17419 | | |
| 22533453 | 889, 1187 | 5263, 5267, 8691, | ######### | 14157, 14165, | 18865, 18875, | 23094894, 23095037 |
| | | 8693, 8701, | | 14475, 17699, | 22479, 22483, | |
| | | 8711, 8723, 8731, | | 17701, 17751, | 22511, 22513, | |
| | | 8735, 8745, | | 17777, 17795, | 22517, 22539, | |
| | | 8753, 8755, 8761 | | 17799, 17837, | 22645, 22655, | |
| | | | | 17861, 17863, | 22657, 22669, | |
| | | | | 17865, 17867, | 22681, 22735 | |
| | | | | 17869, 17901, | | |
| | | | | 17917, 17937 | | |
| 22533454, 22535028 | 891 | 8345 | 9683, 12557 | 14167, 17301 | 22473 | 23094895, 24413563 |
| 22535029, 22535036 | 897, 3583, 4039 | 5269, 5271, 5273, | 9691, 9895 | 14173, 17215 | 18877, 18879, | 23094663, 23095697, |
| | | 8257 | | | 20369 | 24413550 |
| 22533458 | 903 | 5279 | 9695 | 14179 | 18925 | 23094910 |
| 22533460, 22533461 | 907, 909 | 5283 | 9699, 9701 | 14181, 14183 | 18929 | 23094912, 23094913 |
| 22533463 | 913 | 5287 | 9705 | 14187, 17695 | 18933 | 23094915 |
| 22533464 | 915 | 5297 | 9709 | 17689 | 18935 | 23094917 |
| 22533465 | 917 | 5299 | 9711 | 17687 | 18937, 18939 | 23094918 |
| 22533467 | 921 | 5301 | 9713 | 17807 | 18943 | 23094919 |
| 22533468 | 923 | 5303 | 9715, 9717 | 14189 | 18945, 18947 | 23094920 |
| 22533469 | 925 | 5305 | 9719 | 14191 | 18949 | 23094921 |
| 22533470 | 927 | 5307 | 9721 | 14193 | 18951 | 23094922 |
| 22533471 | 929 | 5309 | 9723 | 14195 | 18953 | 23094923, 23095104, |
| | | | | | | 23095466 |
| 22533472 | 931 | 5311 | 9725 | 14197 | 18955 | 23094924 |
| 22533473 | 933 | 5313 | 9727 | 14199, 14267 | 18957 | 23094925 |
| 22533474 | 939 | 5317 | 9731 | 14203 | 18963 | 23094927 |
| 22533475 | 941 | 5319, 5367 | 9733 | 14205 | 18965 | 23094928 |
| 22533476 | 943 | 5321 | 9735 | 14207 | 18967 | 23094929 |
| 22533478 | 945 | 5323 | 9737 | 14209, 14269, | 18969 | 23094930 |
| | | | | 14271 | | |
| 22533479 | 947 | 5325 | 9739 | 14211 | 18971 | 23094931 |
| 22533480 | 949 | 5327 | 9741 | 14213, 14273 | 18973 | 23094932 |
| 22533481 | 951 | 5329 | 9743 | 14215 | 18975 | 23094933 |
| 22533482 | 953 | 5331 | 9745 | 14217 | 18977 | 23094934 |
| 22533483 | 955 | 5333 | 9747 | 14219 | 18979 | 23094935 |
| 22533484 | 958 | 5336 | 9750 | 14222 | 18982 | |
| 22533485 | 959 | 5337 | 9751 | 14223 | 18983, 19025 | 23094936 |
| 22533486 | 961 | 5339 | 9753 | 14225 | 18985 | 23094937 |
| 22533487 | 963 | 5341 | 9755, 10049 | 14227 | 18987 | 23094938 |
| 22533488 | 965 | 5343 | 9757 | 14229 | 18989 | 23094939 |
| 22533489 | 967 | 5345 | 9759 | 14231 | 18991, 19027 | 23094940 |
| 22533490 | 969, 971 | 5347 | 9761 | 14233 | 18993 | 23094941 |
| 22533491 | 973 | 5349 | 9763 | 14235 | 18995 | 23094942 |
| 22533492 | 975 | 5351 | 9765 | 14237 | 18997 | 23094943 |
| 22533493 | 977 | 5353 | 9767 | 14239, 14275, | 18999 | 23094944 |
| | | | | 14277 | | |
| 22533494 | 979 | 5355 | 9769 | 14241 | 19001 | 23094945 |
| 22533495 | 981 | 5357 | 9771 | 14243 | 19003 | 23094946 |
| 22533496 | 983 | 5359 | 9773 | 14245 | 19005 | 23094947 |
| 22533497 | 985 | 5361 | 9775 | 14247 | 19007 | 23094948 |
| 22533498 | 987 | 5363 | 9777 | 14249 | 19009 | 23094949 |
| 22533499 | 989 | 5365, 5371 | 9779 | 14251 | 19011 | 23094950 |
| 22533501 | 993, 995 | 5373 | 9781 | 14253 | 19013, 19029 | 23094952 |
| 22533502 | 997 | 5375, 8763 | 9783 | 14255 | 19015 | 23094953 |
| 22533504 | 999, 1001 | 5377 | 9785 | 14257, 14279, | 19017 | 23094954 |
| | | | | 14281, 14283, | | |
| | | | | 14285 | | |
| 22533505 | 1003 | 5379 | 9787 | 14259 | 19019 | 23094955 |
| 22533506 | 1005 | 5381 | 9789 | 14261 | 19021 | 23094956 |
| 22533507 | 1007 | 5383 | 9791 | 14263 | 19023 | 23094957 |
| 22533509 | 1009 | 8635 | 9793 | 14265 | 19031 | 23094958 |
| 22533511, 22534495 | 1011, 2721 | 5385, 7323 | 9797, 11587 | 14289, 16195, | 19035, 21023 | 23094960, 24413115 |
| | | | | 17739 | | |
| 22533512 | 1013 | 5387 | 9799, 9801 | 14291 | 19037 | 23094962 |
| 22533513 | 1015 | 5389 | 9803 | 14293 | 19039 | 23094963 |
| 22533514 | 1017, 4075 | 5391 | 9805 | 14295 | 19041 | 23094964 |
| 22533515 | 1019, 3925, 4073 | 5393 | 9807 | 14297 | 19043 | 23094965 |
| 22533516 | 1021, 3923 | 5395 | 9809 | 14299 | 19045 | 23094966 |
| 22533517 | 1023 | 5397 | 9811 | 14301 | 19047 | 23094967 |
| 22533518 | 1025 | 5399 | 9813 | 14303 | 19049 | 23094968 |
| 22533519 | 1027 | 5401 | 9815 | 14305 | 19051 | 23094969 |
| 22533520 | 1029 | 5403 | 9817 | 14307 | 19053 | 23094970 |
| 22533521 | 1031 | 5405 | 9819, 9821 | 14309, 17779 | 19055 | 23094971 |
| 22533522 | 1033 | 5407 | 9823 | 14311 | 19057 | 23094972 |
| 22533523 | 1035 | 5409 | 9825 | 14313 | 19059 | 23094973 |
| 22533526 | 1039 | 5413 | 9827 | 14315 | 19173 | 23094975 |
| 22533527 | 1041 | 5415 | 9829 | 14317 | 19175 | 23094976 |
| 22533528, 22534375 | 1043 | 5417, 7081, 7097 | 9831, 11341 | 14319, 15951 | 19177, 20781 | 23094977, 24412999 |
| 22533529 | 1045 | 5419 | 9833 | 14321 | 19179 | 23094978 |
| 22533530 | 1047 | 5421 | 9835 | 14323 | 19181 | 23094979 |
| 22533531 | 1049 | 5423 | 9837 | 14325 | 19183 | 23094980 |
| 22533532 | 1051 | 5425, 8717 | 9839 | 14327 | 19185 | 23094981 |
| 22533533 | 1053 | 5427 | 9841 | 14329 | 19187 | 23094982 |
| 22533534 | 1055 | 5429, 5431 | 9843 | 14331 | 19189 | 23094983 |
| 22533535 | 1057 | 5433 | 9845 | 14333, 14335 | 19191 | 23094984 |
| 22533536 | 1059 | 5435 | 9847 | 14337 | 19193 | 23094985 |
| 22533537 | 1061 | 5437 | 9849 | 14339 | 19195 | 23094986 |
| 22533538 | 1065 | 5441 | 9853 | 14343 | 19199, 19241 | 23094988 |
| 22533539 | 1067 | 5443 | 9855 | 14345 | 19201 | 23094989 |
| 22533540 | 1069 | 5445 | 9857 | 14351 | 19203 | 23094990 |
| 22533541 | 1071 | 5447 | 9859 | 14353 | 19205 | 23094991 |
| 22533542 | 1073 | 5449 | 9861 | 14355, 14357, | 19207 | 23094992 |
| | | | | 14359 | | |
| 22533544 | 1075, 1077 | 5451, 5481 | 9863 | 14361 | 19209, 19243 | 23094993 |
| 22533545 | 1079 | 5453 | 9865 | 14363 | 19211 | 23094994 |
| 22533546 | 1081 | 5455 | 9867 | 14365 | 19213 | 23094995 |
| 22533547 | 1083, 4257 | 5457 | 9869 | 14367, 14443 | 19215 | 23094996 |
| 22533548 | 1085, 1087, 4259 | 5459 | 9871 | 14369, 14445 | 19217 | 23094997 |
| 22533550 | 1089, 1093 | 5461 | 9873 | 14371 | 19219 | 23094998 |
| 22533551 | 1091, 1095 | 5463 | 9875 | 14373 | 19221 | 23094999 |
| 22533552 | 1097 | 5465 | 9877 | 14375 | 19223 | 23095000 |
| 22533553 | 1099 | 5467 | 9879 | 14377 | 19225 | 23095001 |
| 22533554 | 1101 | 5469 | 9881 | 14379 | 19227, 19229 | 23095002 |
| 22533555 | 1103, 1105 | 5471 | 9883, 12785 | 14381 | 19231 | 23095003 |
| 22533556 | 1107 | 5473 | 9885 | 14383 | 19233 | 23095004 |
| 22533557 | 1109, 1111 | 5475 | 9887 | 14385 | 19235 | 23095005 |
| 22533558 | 1113 | 5477 | 9889 | 14387 | 19237 | 23095006 |
| 22533559 | 1115 | 5479 | 9891 | 14389 | 19239, 19245 | 23095007 |
| 22533560 | 1117 | 5483 | 9905 | 14391 | 19247 | 23095008 |
| 22533561 | 1119 | 5485, 8669 | 9907 | 14393 | 19249, 19251 | 23095009 |
| 22533564 | 1121 | 5487 | 9909 | 14395 | 19253 | 23095010 |
| 22533629 | 1125 | 5623 | 9913 | 14399 | 19257 | 23095011 |
| 22533631 | 1131 | 5631 | 9921 | 14407 | 19265 | 23095012 |
| 22533632 | 1133 | 5633 | 9923 | 14409 | 19267 | 23095013 |
| 22533633 | 1135, 1141 | 5635 | 9925 | 14411, 14447 | 19269 | 23095014 |
| 22533626, 22533637, | 1137, 4079 | 5615, 5651, 6749 | 9935, 11035 | 14423 | 19159, 19299, | 23095018, 23095676 |
| 22534163 | | | | | 20331 | |
| 22533634 | 1143 | 5637, 5643 | 9927 | 14413, 14449, | 19271, 19273 | 23095015 |
| | | | | 14451 | | |
| 22533635 | 1145 | 5639, 5645 | 9929 | 14415 | 19275 | 23095016 |
| 22533636 | 1147 | 5647 | 9931 | 14417, 14419 | 19277, 19295 | 23095017 |
| 22533639 | 1151 | 5655 | 9939 | 14427 | 19303 | 23095019 |
| 22533640 | 1153 | 5657 | 9941 | 14429, 14453 | 19305 | 23095020 |
| 22533641 | 1155, 1157 | 5659, 5661 | 9943 | 14431 | 19307 | 23095021 |
| 22533642 | 1159 | 5663 | 9945 | 14433, 14435 | 19309 | 23095022 |
| 22533643 | 1161 | 5665 | 9947 | 14437 | 19311 | 23095023 |
| 22533644 | 1163 | 5667 | 9949 | 14439 | 19313 | 23095024 |
| 22533645 | 1165 | 5669 | 9951 | 14441 | 19315 | 23095025 |
| 22533646 | 1167 | 5671 | 9953 | 14455 | 19317 | 23095026 |
| 22533647 | 1169 | 5673 | 9955 | 14457 | 19319, 19321 | 23095027 |
| 22533648 | 1171 | 5675 | 9957, 10007 | 14459 | 19323 | 23095028 |
| 22533649 | 1173 | 5677 | 9959 | 14461 | 19325 | 23095029 |
| 22533650 | 1175 | 5679, 5681 | 9961 | 14463 | 19327 | 23095030 |
| 22533652 | 1177 | 5683 | 9963 | 14465 | 19329 | 23095031 |
| 22534863 | 1197, 3357 | 8041 | 10075, 10077, | 14485, 14487, | 21759 | 23095040, 24413446 |
| | | | 12319 | 16967 | | |
| 22535098 | 1201, 3701 | 8407, 8409, 8495 | 12609 | 17393 | 22035 | 24413589 |
| 22533662, 22533663, | 1209, 1213, 1215, | 7203, 7205 | 10085, 10087, | 14495, 14497, | 20899, 20901 | 23095044, 23095045, |
| 22533665, | 1221, 2623, | | 10091, 11457, | 14501, 17847 | | 24413056, |
| 22534434, | 2625 | | 11459 | | | 24413057 |
| 22534435 | | | | | | |
| 22533664, 22534437, | 1217, 1219, 2629, | 7209 | 10089, 11463, | 14499, 14585 | 20905, 21501, | 23095046, 24413059 |
| 22534737 | 3099 | | 12027 | | 22537 | |
| 22534312 | 1225, 2403 | 5685 | 10095 | 14505 | 19331, 20649 | 24412939 |
| 22533671 | 1231 | 5693 | 10103 | 14513 | 19339 | 23095049 |
| 22533672 | 1233 | 5695 | 10105 | 14515 | 19341 | 23095050 |
| 22533673 | 1235 | 5697 | 10107 | 14517, 14519 | 19343 | 23095051 |
| 22533674 | 1237 | 5699 | 10109 | 14521 | 19347 | 23095052 |
| 22533675 | 1239 | 5701 | 10111 | 14523 | 19349, 19409 | 23095053 |
| 22533677 | 1241 | 5703 | 10113 | 14525 | 19351 | 23095055 |
| 22533678 | 1243 | 5705 | 10115 | 14527 | 19353 | 23095056 |
| 22533679 | 1245 | 5707 | 10117 | 14529 | 19355 | 23095057 |
| 22533680 | 1247 | 5709 | 10119 | 14531 | 19357 | 23095058 |
| 22533681 | 1249 | 5711 | 10121 | 14533 | 19359 | 23095059 |
| 22533682 | 1251 | 5713, 5737 | 10123 | 14535, 17815 | 19361 | 23095060 |
| 22533683 | 1253 | 5715 | 10125 | 14537, 14587 | 19363 | 23095061 |
| 22533684 | 1255 | 5717 | 10127 | 14539 | 19365 | 23095062 |
| 22533685 | 1257 | 5719 | 10129 | 14541, 14589, | 19367 | 23095063 |
| | | | | 17841 | | |
| 22533592, 22533686 | 1259, 1261 | 5721 | 10131 | 14543, 14591, | 19369 | 23095064 |
| | | | | 17839 | | |
| 22533687 | 1263 | 5723 | 10133 | 14545, 14593 | 19371 | 23095065 |
| 22534930 | 1277, 3389 | 8071 | 10147, 12347 | 14561, 16997 | 19387, 21787, | 24413460 |
| | | | | | 22625 | |
| 22533697 | 1295 | 5731 | 10165 | 14581 | 19405 | 23095068 |
| 22533698 | 1297 | 5733, 5735 | 10167, 10169, | 14583, 14603 | 19407 | 23095069 |
| | | | 10171 | | | |
| 22533699 | 1299 | 5739 | 10173 | 14605 | 19413 | 23095070 |
| 22533700 | 1301 | 5741, 5743 | 10175 | 14607 | 19415 | 23095071 |
| 22533701 | 1303 | 5745 | 10177 | 14609 | 19417, 19419 | 23095072 |
| 22533702 | 1305 | 5747, 5803 | 10179 | 14611, 14613 | 19421 | 23095073 |
| 22533703 | 1307 | 5749 | 10181 | 14615 | 19423 | 23095074 |
| 22533704 | 1309 | 5751 | 10183 | 14617 | 19425, 19427 | 23095075 |
| 22533705 | 1311 | 5755 | 10187 | 14621 | 19431 | 23095076 |
| 22533708 | 1313 | 5757 | 10189 | 14623 | 19433 | 23095077 |
| 22533709 | 1315 | 5759, 5761 | 10191 | 14625, 14683, | 19435 | 23095078 |
| | | | | 17733 | | |
| 22533710 | 1317, 1319 | 5763 | 10193 | 14627, 14685, | 19437 | 23095079 |
| | | | | 14689, 14695 | | |
| 22533711 | 1321 | 5765 | 10195 | 14629, 14687, | 19439 | 23095080 |
| | | | | 14691, 14693, | | |
| | | | | 14697, 14699 | | |
| 22533712 | 1323 | 5767 | 10197 | 14631 | 19441 | 23095081 |
| 22533713 | 1325 | 5769 | 10199 | 14633 | 19443 | 23095082 |
| 22533714 | 1327 | 5771 | 10201 | 14635 | 19445 | 23095083 |
| 22533715 | 1329 | 5773 | 10203 | 14637 | 19447 | 23095084 |
| 22533716 | 1331 | 5775 | 10205 | 14639, 17879 | 19449 | 23095085 |
| 22533717 | 1333 | 5777 | 10207 | 14641 | 19451 | 23095086 |
| 22533718 | 1335 | 5779 | 10209 | 14643, 17909 | 19453, 19477 | 23095087 |
| 22533719 | 1337 | 5781 | 10211 | 14645 | 19455 | 23095088 |
| 22533720 | 1339 | 5783 | 10213 | 14647 | 19457 | 23095089 |
| 22533721 | 1341 | 5785 | 10215 | 14649 | 19459 | 23095090 |
| 22533722 | 1343 | 5787 | 10217 | 14651 | 19461 | 23095091 |
| 22533723 | 1345, 1347 | 5789 | 10219 | 14653 | 19463, 19479 | 23095092 |
| 22533725 | 1349 | 5791 | 10221 | 14655 | 19465 | 23095093 |
| 22533726 | 1351 | 5793 | 10223 | 14657 | 19467 | 23095094 |
| 22533728 | 1353 | 5797, 8773 | 10227 | 14661 | 19471 | 23095096 |
| 22533729 | 1355 | 5799 | 10229 | 14663 | 19473 | 23095097 |
| 22533730 | 1357 | 5801 | 10231 | 14665 | 19475, 19481 | 23095098 |
| 22533731 | 1359 | 5805 | 10233 | 14667 | 19483, 19485 | 23095099 |
| 22533732 | 1361 | 5807 | 10235 | 14669 | 19487 | 23095100 |
| 22533733 | 1363 | 5809 | 10237 | 14671 | 19489 | 23095150 |
| 22533734 | 1365 | 5811 | 10239 | 14673 | 19491, 19493 | 23095151 |
| 22533735 | 1367 | 5813 | 10241 | 14675 | 19495 | 23095152 |
| 22533736 | 1369 | 5815 | 10243 | 14677 | 19497 | 23095153 |
| 22533737 | 1371 | 5817 | 10245 | 14679 | 19499 | 23095154 |
| 22533738 | 1373, 1375 | 5819 | 10247 | 14681, 14707 | 19501, 22659 | 23095155 |
| 22533739 | 1377 | 5821 | 10249 | 14709 | 19503 | 23095156 |
| 22533740 | 1379 | 5823 | 10251 | 14701, 14711 | 19505 | 23095157 |
| 22533741 | 1381 | 5825 | 10253 | 14703, 14713 | 19507 | 23095158 |
| 22533742 | 1383 | 5827 | 10255 | 14705, 14717 | 19509 | 23095159 |
| 22533743 | 1385 | 5829, 8779 | 10257 | 14719 | 19511 | 23095160 |
| 22533744 | 1387 | 5831 | 10259 | 14721 | 19513 | 23095161 |
| 22533746 | 1389 | 5833 | 10261 | 14723 | 19515, 19521 | 23095162 |
| 22533747 | 1391 | 5835 | 10263 | 14725 | 19517, 19519, | 23095163 |
| | | | | | 19523, 19525 | |
| 22533748 | 1393 | 5837 | 10265 | 14727 | 19527 | 23095164 |
| 22533751 | 1395 | 5843 | 10271 | 14733 | 19533 | 23095166 |
| 22533752 | 1397 | 5845 | 10273 | 14735 | 19535 | 23095167 |
| 22533753 | 1399 | 5847 | 10275 | 14737 | 19537 | 23095168 |
| 22533754 | 1401 | 5849 | 10277 | 14739 | 19539 | 23095169 |
| 22533756 | 1403 | 5853 | 10281 | 14743 | 19543 | 23095171 |
| 22533757 | 1405, 1407 | 5855 | 10283 | 14745, 14749 | 19545 | 23095172 |
| 22533758 | 1409 | 5857 | 10285 | 14747, 14751 | 19547 | 23095173 |
| 22533759 | 1411 | 5859 | 10287 | 14753 | 19549 | 23095174 |
| 22533760, 22535069 | 1413, 3647, 3649 | 5861, 8355, 8391 | 10289 | 14755, 17311 | 19551 | 23095175 |
| 22533761 | 1415 | 5863 | 10291 | 14757 | 19553 | 23095201 |
| 22533762 | 1417 | 5865 | 10293 | 14759 | 19555 | 23095202 |
| 22533763 | 1419 | 5867 | 10295 | 14761 | 19557 | 23095203 |
| 22533764 | 1421 | 5869 | 10297 | 14763 | 19559 | 23095204 |
| 22533765 | 1423 | 5871 | 10299 | 14765 | 19561 | 23095205 |
| 22533766 | 1425 | 5873 | 10301 | 14767 | 19563 | 23095206 |
| 22533767 | 1427 | 5875 | 10303 | 14769 | 19565 | 23095207 |
| 22533768 | 1429 | 5877 | 10305 | 14771 | 19567 | 23095208 |
| 22533770 | 1431 | 5879 | 10307 | 14773 | 19569, 22591 | 23095209 |
| 22533772 | 1433 | 5883 | 10311 | 14781 | 19573 | 23095211 |
| 22533773 | 1435 | 5885 | 10313 | 14783 | 19575 | 23095212 |
| 22533774 | 1437 | 5887 | 10315 | 14785 | 19577 | 23095213 |
| 22533775 | 1439 | 5889 | 10317 | 14787 | 19579 | 23095214 |
| 22533777 | 1441 | 5891 | 10319, 10321 | 14789, 14809 | 19581 | 23095215 |
| 22533778 | 1443 | 5893 | 10323 | 14791 | 19583 | 23095216 |
| 22533779 | 1445 | 5895 | 10325 | 14793 | 19585 | 23095217 |
| 22533780 | 1447 | 5897 | 10327 | 14795 | 19587 | 23095218 |
| 22533781 | 1449 | 5899 | 10329 | 14797 | 19589 | 23095219 |
| 22533782 | 1451 | 5901 | 10331 | 14799 | 19591 | 23095220 |
| 22533783 | 1453 | 5903 | 10333 | 14801 | 19593 | 23095221 |
| 22533784 | 1455 | 5905 | 10335 | 14803 | 19595 | 23095222 |
| 22533787 | 1457 | 5909 | 10339 | 14807 | 19599 | 23095224 |
| 22533789, 22534806 | 1459, 1461, 3235 | 5913, 7927 | 10345, 12199, | 14817, 16835, | 19605, 21643 | 23095226, 24413392 |
| | | | 12201 | 16837 | | |
| 22533791 | 1463 | 5917 | 10349 | 14821 | 19609 | 23095228 |
| 22533792 | 1465 | 5919 | 10351 | 14823, 14825 | 19611 | 23095229 |
| 22533793 | 1467 | 5921 | 10353 | 14827 | 19613 | 23095230 |
| 22533794 | 1469 | 5923 | 10355 | 14829 | 19615, 19625 | 23095231 |
| 22533795 | 1471 | 5925 | 10357 | 14831 | 19617 | 23095232 |
| 22533796 | 1473 | 5927 | 10359 | 14833 | 19619 | 23095233 |
| 22533797 | 1475 | 5929 | 10361 | 14835 | 19621 | 23095234 |
| 22533798 | 1477, 1479 | 5931 | 10363 | 14837 | 19623, 19627 | 23095235 |
| 22533799 | 1481 | 5933 | 10365 | 14839 | 19629 | 23095236 |
| 22533803 | 1483 | 5941 | 10373 | 14847 | 19637 | 23095240 |
| 22533804 | 1485 | 5943 | 10375 | 14849 | 19639 | 23095241 |
| 22533805 | 1487 | 5945 | 10377 | 14851 | 19641 | 23095242 |
| 22533806 | 1489 | 5947 | 10379 | 14853 | 19643 | 23095243 |
| 22533807 | 1491, 1493 | 5949 | 10381 | 14855, 14881 | 19645 | 23095244 |
| 22533808 | 1495 | 5951 | 10383 | 14857, 14885 | 19647 | 23095245 |
| 22533809 | 1497 | 5953 | 10385 | 14859 | 19649, 19651 | 23095246 |
| 22533811 | 1499 | 5955 | 10387 | 14861 | 19653 | 23095247 |
| 22533812 | 1501 | 5957 | 10389 | 14863, 14887 | 19655, 19657 | 23095248 |
| 22533813 | 1503 | 5959 | 10391 | 14865, 14889 | 19659 | 23095249 |
| 22533814 | 1505 | 5961 | 10393 | 14867 | 19661 | 23095250 |
| 22533815 | 1507 | 5963 | 10395 | 14869 | 19663 | 23095251 |
| 22533816 | 1509 | 5965 | 10397 | 14871 | 19665 | 23095252 |
| 22533817 | 1511 | 5967 | 10399 | 14873 | 19667 | 23095253 |
| 22533818 | 1513 | 5969 | 10401 | 14875 | 19669 | 23095254 |
| 22533819 | 1515 | 8653 | 10403 | 14877 | 19671 | 23095255 |
| 22533826 | 1517 | 5987 | 10415, 10417 | 14901 | 19683 | 23095261 |
| 22533827 | 1519 | 5989 | 10419 | 14903 | 19685 | 23095262 |
| 22533828 | 1521 | 5991 | 10421 | 14905, 14951 | 19687, 19689 | 23095263 |
| 22533829 | 1523 | 5993 | 10423 | 14907 | 19691 | 23095264 |
| 22533830 | 1525 | 5995 | 10425 | 14909 | 19693 | 23095265 |
| 22533833 | 1527 | 5997, 5999 | 10427 | 14911 | 19695 | 23095266 |
| 22533834 | 1529 | 4783, 6001 | 10429 | 13657, 14913 | 19697 | 23094634, 23095267 |
| 22533835 | 1531 | 6003 | 10431 | 14915 | 19699 | 23095268 |
| 22533837 | 1533 | 6009 | 10435 | 14919 | 19703 | 23095270 |
| 22533838 | 1535 | 6011 | 10437 | 14921 | 19705 | 23095271 |
| 22533839 | 1537 | 6013 | 10439 | 14923 | 19707 | 23095272 |
| 22533840 | 1539 | 6015, 6017 | 10441 | 14925 | 19709 | 23095273 |
| 22533841 | 1541, 1543 | 6019 | 10443 | 14927 | 19711, 22693 | 23095274 |
| 22533842 | 1545 | 6021 | 10445 | 14929 | 19713 | 23095275 |
| 22533843 | 1547, 1549, 3971 | 6023 | 10447 | 14931 | 19715 | 23095276 |
| 22533844 | 1551 | 6025 | 10449, 10795 | 14933, 15361 | 19717, 20093 | 23095277 |
| 22533845 | 1553, 1555 | 6027 | 10451 | 14935 | 19719 | 23095278 |
| 22533846 | 1557 | 6029 | 10453 | 14937 | 19721 | 23095279 |
| 22533847 | 1559, 4217 | 6031 | 10455 | 14939 | 19723 | 23095280 |
| 22533849 | 1561, 4011 | 6035 | 10459 | 14943 | 19727 | 23095282 |
| 22533848 | 1563 | 6033 | 10457 | 14941 | 19725 | 23095281 |
| 22533850 | 1565 | 6037 | 10461 | 14945 | 19729 | 23095283 |
| 22533851 | 1567 | 6039 | 10463 | 14947 | 19731, 19773 | 23095284 |
| 22533852 | 1569 | 6041 | 10465 | 14949, 14953 | 19733 | 23095285 |
| 22533855 | 1571 | 6045, 8699 | 10467 | 14955 | 19735 | 23095287 |
| 22533856 | 1573 | 6047, 8697 | 10469 | 14957 | 19737, 19775 | 23095288 |
| 22533857 | 1575, 1577 | 6049 | 10471 | 14959 | 19739 | 23095289 |
| 22533858 | 1581 | 6051 | 10473 | 14961 | 19741 | 23095290 |
| 22533859 | 1583 | 6053 | 10475 | 14963 | 19743 | 23095291 |
| 22533860 | 1585, 1593 | 6055 | 10477 | 14965 | 19745 | 23095292 |
| 22533861 | 1587, 1595, 4285 | 6057 | 10479 | 14967 | 19747 | 23095293 |
| 22533862 | 1589, 1597, 4283 | 6059 | 10481 | 14969 | 19749 | 23095294 |
| 22533863 | 1591, 1599, 4281 | 6061, 7307 | 10483 | 14971 | 19751, 21121, | 23095295 |
| | | | | | 22615, 22689 | |
| 22533864 | 1601 | 6063 | 10485 | 14973 | 19753, 19755 | 23095296 |
| 22533865 | 1603 | 6065 | 10487 | 14975 | 19757 | 23095297 |
| 22533866 | 1605 | 6067 | 10489 | 14977 | 19759, 19777 | 23095298 |
| 22533867 | 1607, 4025 | 6069, 6071 | 10491 | 14979 | 19761 | 23095299 |
| 22533868 | 1609, 4027 | 6073 | 10493 | 14981 | 19763 | 23095300 |
| 22533869 | 1611 | 6075 | 10495 | 14983 | 19765 | 23095301 |
| 22533870 | 1613 | 6077 | 10497 | 14985 | 19767, 19779 | 23095302 |
| 22533871 | 1615 | 6079 | 10499 | 14987 | 19769 | 23095303 |
| 22533872 | 1617 | 6081 | 10501 | 14989, 15001, | 19771 | 23095304 |
| | | | | 17905 | | |
| 22533873 | 1619 | 6083 | 10503 | 14991 | 19781 | 23095305 |
| 22533874 | 1625 | 6085 | 10505 | 14993 | 19783, 19785 | 23095306 |
| 22533875 | 1629 | 6089 | 10509 | 14999 | 19789 | 23095308 |
| 22533878 | 1633, 1637, 4105 | 6093 | 10513 | 15003 | 19793 | 23095310 |
| 22533879 | 1635, 1639, 4107 | 6095 | 10515 | 15005 | 19795 | 23095311 |
| 22533880 | 1641 | 6097 | 10517 | 15007 | 19797 | 23095312 |
| 22533881 | 1643 | 6099 | 10519 | 15009 | 19799 | 23095313 |
| 22533882 | 1645 | 6101 | 10521 | 15011 | 19801, 19813, | 23095314 |
| | | | | | 19815 | |
| 22533883 | 1647 | 6103 | 10523 | 15013, 15083 | 19803 | 23095315 |
| 22533884 | 1649 | 6105 | 10525 | 15015 | 19805 | 23095316 |
| 22533886 | 1651, 1653, 1655 | 6107, 6109 | 10527 | 15017 | 19807, 19817 | 23095317 |
| 22533887 | 1657 | 6111 | 10529 | 15019 | 19809 | 23095318 |
| 22533888 | 1659 | 6113 | 10531 | 15021 | 19811 | 23095319 |
| 22533889 | 1661 | 6115, 6119 | 10533 | 15023 | 19821 | 23095320 |
| 22533890 | 1663 | 6117, 6121 | 10535 | 15025, 15027 | 19823 | 23095321 |
| 22533891 | 1665, 1667, 4161 | 6123, 6125 | 10537 | 15029, 15081 | 19825 | 23095322 |
| 22533893 | 1671, 1673, 1675, | 6129 | 10541, 10543 | 15033 | 19829 | 23095323 |
| | 1677, 4129 | | | | | |
| 22533894 | 1679, 1681 | 6131 | 10545 | 15035 | 19831 | 23095324 |
| 22533895 | 1683 | 6133 | 10547 | 15037 | 19833 | 23095325 |
| 22533896 | 1685 | 6135 | 10549 | 15039 | 19835 | 23095326 |
| 22533898 | 1687 | 6137 | 10551 | 15041, 15085 | 19837 | 23095327 |
| 22533899 | 1689 | 6139 | 10553 | 15043 | 19839 | 23095328 |
| 22533900 | 1691 | 6141 | 10555 | 15045 | 19841 | 23095329 |
| 22533901 | 1693, 1695 | 6143 | 10557 | 15047 | 19843, 19899 | 23095330 |
| 22533902 | 1697 | 6145 | 10559 | 15049 | 19845 | 23095331 |
| 22533903 | 1699 | 6147 | 10561 | 15051 | 19847 | 23095332 |
| 22533906 | 1703 | 6153 | 10567 | 15059 | 19853 | 23095335 |
| 22533907 | 1705 | 6155 | 10569, 10573 | 15061 | 19855 | 23095336 |
| 22533908 | 1707, 1709 | 6157 | 10575 | 15063 | 19857 | 23095337 |
| 22533909 | 1711 | 6159 | 10577 | 15065 | 19859 | 23095338 |
| 22533911 | 1713 | 6163 | 10581 | 15069 | 19863 | 23095340 |
| 22533912 | 1715 | 6165, 8677 | 10583 | 15071, 15087 | 19865 | 23095341 |
| 22533913 | 1717 | 6167 | 10585 | 15073 | 19867 | 23095342 |
| 22533914 | 1719 | 6169 | 10587 | 15075 | 19869 | 23095343 |
| 22533915 | 1721, 1723 | 6171 | 10589 | 15077 | 19871 | 23095344 |
| 22533916 | 1725 | 6173 | 10591 | 15079, 15089 | 19873 | 23095345 |
| 22533917 | 1727 | 6175 | 10593 | 15091 | 19875 | 23095346 |
| 22533918 | 1729 | 6177 | 10595 | 15093 | 19877 | 23095347 |
| 22533927 | 1733 | 6185 | 10599 | 15097 | 19881 | 23095370 |
| 22533928 | 1735 | 6187 | 10601 | 15099 | 19883 | 23095371 |
| 22533929 | 1737 | 6189, 6225 | 10603, 10605 | 15101, 15103 | 19885 | 23095372 |
| 22533931 | 1739 | 6229 | 10609 | 15107 | 19889 | 23095374 |
| 22533932 | 1741 | 6231 | 10611 | 15109 | 19891 | 23095375 |
| 22533933 | 1743 | 6233 | 10613, 10615 | 15111, 15139 | 19893 | 23095376 |
| 22533934 | 1745 | 6235 | 10617 | 15113 | 19895 | 23095377 |
| 22533935 | 1747 | 6237 | 10619 | 15115 | 19897 | 23095378 |
| 22533959 | 1755 | 6245 | 10627 | 15123 | 19907 | 23095382 |
| 22533960 | 1757 | 6247 | 10629 | 15125 | 19909 | 23095383 |
| 22533961 | 1759 | 6249, 6325 | 10631 | 15127 | 19911 | 23095384 |
| 22533962 | 1761 | 6251 | 10633 | 15129 | 19913 | 23095385 |
| 22533963 | 1763 | 6253 | 10635 | 15131 | 19915 | 23095386 |
| 22533966 | 1767 | 6257 | 10639 | 15135 | 19919 | 23095387 |
| 22533968 | 1769 | 6259 | 10641 | 15137 | 19921 | 23095388 |
| 22533969 | 1771 | 6261 | 10643 | 15141 | 19923 | 23095389 |
| 22533970 | 1773 | 6263 | 10645 | 15143 | 19925 | 23095390 |
| 22533971 | 1775 | 6265 | 10647 | 15145 | 19927 | 23095391 |
| 22533972 | 1777 | 6267 | 10649 | 15147 | 19929 | 23095392 |
| 22533973 | 1779 | 6269 | 10651 | 15149 | 19931 | 23095393 |
| 22533974 | 1781 | 6271 | 10653 | 15151 | 19933 | 23095394 |
| 22533424, 22533976 | 1783 | 5213, 6275 | 9595, 10657 | 14015, 15155 | 18803, 19937 | 23094866, 23095396 |
| 22533977 | 1785, 3955 | 6277 | 10659 | 15157 | 19939 | 23095397 |
| 22533978 | 1787, 3957 | 6279 | 10661 | 15159 | 19941 | 23095398 |
| 22533979 | 1789 | 6281 | 10663 | 15161 | 19943 | 23095399 |
| 22533980 | 1791 | 6283 | 10665 | 15163, 15167 | 19945 | 23095400 |
| 22533981 | 1793 | 6285 | 10667 | 15165 | 19947 | 23095401 |
| 22533982 | 1795, 4007, 4009 | 6287 | 10669 | 15169 | 19949, 19951 | 23095402 |
| 22533983 | 1797 | 6289 | 10671 | 15171 | 19953 | 23095403 |
| 22533984 | 1799 | 6291 | 10673 | 15173 | 19955 | 23095404 |
| 22533986 | 1801 | 6293 | 10675, 10677 | 15175, 15177 | 19957 | 23095405 |
| 22533987 | 1803, 1805 | 6295 | 10679 | 15179, 15181 | 19959 | 23095406 |
| 22533990 | 1811 | 6329 | 10683 | 15231 | 19965 | 23095407 |
| 22533991 | 1813 | 6331 | 10685 | 15233 | 19967 | 23095408 |
| 22533992 | 1815 | 6333 | 10687 | 15235 | 19969, 20041 | 23095409 |
| 22533993 | 1817 | 6335 | 10689 | 15237 | 19971 | 23095410 |
| 22533994 | 1819 | 6337 | 10691 | 15239 | 19973 | 23095411 |
| 22533995 | 1823 | 6341 | 10695, 10697 | 15245 | 19977 | 23095413 |
| 22533996 | 1825 | 6343 | 10699 | 15247, 15259 | 19979 | 23095414 |
| 22533997 | 1827 | 6345 | 10701 | 15249, 15261, | 19981 | 23095415 |
| | | | | 15263 | | |
| 22533998 | 1829 | 6347 | 10703 | 15251 | 19983 | 23095416 |
| 22533999 | 1831 | 6349 | 10705 | 15253 | 19989 | 23095417 |
| 22534000 | 1833 | 6351 | 10707 | 15255 | 19991 | 23095418 |
| 22533157, 22534001 | 1835 | 4631, 6353, 6355 | 9071, 10709 | 13491, 15257, | 19993 | 23094571, 23095419 |
| | | | | 15265, 15293, | | |
| | | | | 15295, 17951 | | |
| 22534002 | 1837 | 6357 | 10711 | 15267, 15297, | 19995, 22611 | 23095420 |
| | | | | 15299 | | |
| 22534003 | 1839 | 6359 | 10713 | 15269 | 19997 | 23095421 |
| 22534004 | 1841 | 6361, 6383 | 10715 | 15271, 15301 | 19999 | 23095470 |
| 22534005 | 1843 | 6363 | 10717 | 15273 | 20001 | 23095471 |
| 22534007 | 1845 | 6365 | 10719 | 15275 | 20003 | 23095472 |
| 22534008 | 1847 | 6367 | 10721 | 15277 | 20005 | 23095473 |
| 22534009 | 1849 | 6369 | 10723 | 15279, 15303 | 20007 | 23095474 |
| 22534010 | 1851 | 6371 | 10725 | 15281 | 20009, 20043 | 23095475 |
| 22534011 | 1853 | 6373 | 10727 | 15283 | 20011 | 23095476 |
| 22534012 | 1855 | 6375 | 10729 | 15285 | 20013 | 23095477 |
| 22534013 | 1857 | 6377 | 10731 | 15287 | 20015 | 23095478 |
| 22534014 | 1859 | 6379 | 10733 | 15289 | 20017, 20045 | 23095479 |
| 22534015, 22535008 | 1861, 3547 | 6381, 6385, 8233 | 10735, 12503 | 15291, 17187, | 20019, 20021 | 23095480, 24413534 |
| | | | | 17229 | 20023, 21927 | |
| — | 1862 | 6386 | 10736 | 15292 | 20024 | |
| 22534016 | 1863 | 6387, 6389, 6391, | 10737 | 15305, 17697 | 20025 | 23095481 |
| | | 6393 | | | | |
| 22534017, 22534018 | 1865, 1867 | 6395 | 10739, 10741 | 15307, 15309 | 20027, 20029 | 23095482, 23095483 |
| 22534022 | 1869 | 6403 | 10749 | 15317 | 20037 | 23095487 |
| 22534023 | 1871, 1873 | 6405 | 10751 | 15319 | 20039 | 23095488 |
| 22534025 | 1875 | 6409 | 10755 | 15323 | 20049 | 23095490 |
| 22534026 | 1877 | 6411 | 10757, 10759, | 15325, 15327, | 20051, 20053 | 23095491 |
| | | | 10763 | 15329, 17801 | | |
| 22534027 | 1879 | 6413 | 10765 | 15331 | 20055 | 23095492 |
| 22534029 | 1881 | 6415, 6451, 6453 | 10767 | 15333, 15407 | 20057, 20059 | 23095493 |
| 22534030 | 1883 | 6455 | 10769 | 15335 | 20061 | 23095494 |
| 22534031 | 1885 | 4743, 6457 | 10771 | 15337 | 20063 | 23095495 |
| 22534032 | 1887 | 6459 | 10773 | 15339 | 20065 | 23095496 |
| 22534033 | 1889 | 6461, 6465 | 10775 | 15341 | 20067 | 23095497 |
| 22534034 | 1891 | 6463, 6467, 6471 | 10777 | 15343 | 20069 | 23095498 |
| 22534035 | 1893 | 6469, 6473 | 10779 | 15345 | 20071, 20079 | 23095499 |
| 22534036 | 1895 | 6475 | 10781 | 15347 | 20073 | 23095500 |
| 22534037 | 1897 | 6477 | 10783 | 15349 | 20075 | 23095501 |
| 22534038 | 1899 | 6479 | 10785 | 15351 | 20077, 20081 | 23095502 |
| 22534039 | 1901 | 6481 | 10787 | 15353 | 20083 | 23095503 |
| 22534040 | 1903 | 6483 | 10789 | 15355 | 20085 | 23095504 |
| 22534041 | 1905 | 6485, 8719 | 10761, 10791 | 15357 | 20087, 20089 | 23095505 |
| 22534049, 22534057, | 1917, 1941, 1945, | 6501, 6523, 6525 | 10801, 10835 | 15377 | 20113, 20115, | 23095518, 23095519 |
| 22534058 | 1947 | | | | 22549 | |
| 22534054, 22534717, | 1919, 1921, 1931, | 6503, 6505, 6515, | 10803, 10813, | 15369, 16655, | 20095, 20107, | 23095515, 24413307 |
| 22534736 | 1935 | 7761 | 10815, 10821, | 17945 | 21461 | |
| | | | 10823, 10829, | | | |
| | | | 11993, 11995 | | | |
| 22534053, 22534738 | 1929, 1933, 3101 | 6513 | 12029 | 16689 | 21503 | 23095514 |
| | 1942 | 6524 | 10836 | 15378 | 20114 | |
| 22534059 | 1949 | 6527, 6539 | 10837 | 15379 | 20117 | 23095520 |
| 22534060 | 1951 | 6529 | 10839 | 15381 | 20119 | 23095521 |
| 22534062 | 1953 | 6533, 6535 | 10843 | 14883, 15385 | 20123 | 23095523 |
| 22534064 | 1957 | 6545 | 10849 | 15387 | 20129 | 23095525 |
| 22534065 | 1959 | 6547 | 10851 | 15389 | 20131 | 23095526 |
| 22534066 | 1961 | 6549 | 10853 | 15391, 15409 | 20133, 20149 | 23095527 |
| 22534067 | 1963 | 6551 | 10855 | 15393 | 20135 | 23095528 |
| 22534068 | 1965 | 6553 | 10857 | 15395 | 20137 | 23095529 |
| 22534069 | 1967 | 6555 | 10859 | 15397 | 20139 | 23095530 |
| 22534070 | 1969 | 6557 | 10861 | 15399 | 20141 | 23095531 |
| 22534071 | 1971 | 6559 | 10863 | 15401 | 20143 | 23095532 |
| 22534073 | 1973 | 6561 | 10865 | 15403 | 20145 | 23095533 |
| 22534074 | 1975 | 6563 | 10867 | 15405, 17745 | 20147 | 23095534 |
| 22534076 | 1979 | 6569 | 10873 | 15417 | 20151, 22491 | 23095537 |
| 22534078 | 1981 | 6571 | 10875 | 15419 | 20153 | 23095538 |
| 22534079 | 1983 | 6573 | 10877 | 15421 | 20155 | 23095539 |
| 22534080 | 1985 | 6575 | 10879 | 15423 | 20157 | 23095540 |
| 22534081 | 1987 | 6577 | 10881 | 15425 | 20159, 20243 | 23095541 |
| 22534082 | 1989 | 6579 | 10883 | 15427 | 20161 | 23095542 |
| 22534083 | 1991 | 6581 | 10885 | 15429 | 20163 | 23095543 |
| 22534084 | 1993 | 6583 | 10887 | 15431 | 20165 | 23095544 |
| 22534085 | 1995 | 6585 | 10889 | 15433, 17835 | 20167 | 23095545 |
| 22534086 | 1997 | 6587 | 10891 | 15435 | 20169 | 23095546 |
| 22534087 | 1999 | 6589 | 10893 | 15437 | 20171 | 23095547 |
| 22534088 | 2001 | 6591 | 10895 | 15439 | 20173 | 23095548 |
| 22534089 | 2003 | 6593 | 10897 | 15441 | 20175 | 23095549 |
| 22534090 | 2005, 4063 | 6595 | 10899 | 15443 | 20177 | 23095550 |
| 22534095 | 2007 | 6597 | 10901 | 15445 | 20179, 22633 | 23095552 |
| 22534096 | 2009, 2011 | 6599 | 10903 | 15447 | 20181 | 23095553 |
| 22534097 | 2013 | 6601 | 10905 | 15449 | 20183, 20245 | 23095554 |
| 22534098 | 2015 | 6603 | 10907 | 15451 | 20185, 20247 | 23095555 |
| 22534099 | 2017 | 6605 | 10909 | 15453 | 20187 | 23095556 |
| 22534100 | 2019 | 6607 | 10911 | 15455 | 20189 | 23095557 |
| 22534101 | 2021 | 6609 | 10913 | 15457 | 20191 | 23095558 |
| 22534102 | 2023 | 6611, 8775 | 10915 | 15459 | 20193 | 23095559 |
| 22534103 | 2025 | 6613 | 10917 | 15461 | 20195 | 23095560 |
| 22534104 | 2027 | 6615 | 10919 | 15463 | 20197 | 23095561 |
| 22534106 | 2029 | 6617 | 10921 | 15465 | 20199 | 23095562 |
| 22534107 | 2031 | 6619 | 10923 | 15467 | 20201 | 23095563 |
| 22534108 | 2033 | 6621 | 10925 | 15469 | 20203 | 23095564 |
| 22534109 | 2035 | 6623, 6641 | 10927 | 15471 | 20205 | 23095565 |
| 22534110 | 2037 | 6625, 6643, 8729 | 10929 | 15473, 15481 | 20207 | 23095566 |
| 22534111 | 2039 | 6627 | 10931 | 15475 | 20209 | 23095567 |
| 22534112 | 2041 | 6629 | 10933 | 15477 | 20211 | 23095629 |
| 22534113 | 2043, 2045 | 6631 | 10935 | 15479, 15483 | 20213 | 23095630 |
| 22534114 | 2047, 4137, 4261 | 6633 | 10937, 12801 | 15485, 17613 | 20215, 22273 | 23095631 |
| 22534115 | 2049, 2051, 3837 | 6635 | 10939, 10941 | 15487, 17639 | 20217, 20219, | 23095632 |
| | | | | | 22297 | |
| 22534117 | 2053, 2055 | 6637 | 10943 | 15489, 17765, | 20221 | 23095633 |
| | | | | 17767 | | |
| 22534118 | 2057 | 6647 | 10945 | 15491 | 20223 | 23095634 |
| 22534119 | 2059 | 6649 | 10947 | 15493 | 20225 | 23095635 |
| 22534120 | 2061 | 6651 | 10949, 10951 | 15495 | 20227 | 23095636 |
| 22534121 | 2063 | 6653 | 10953 | 15497 | 20229 | 23095637 |
| 22534122 | 2065 | 6655 | 10955 | 15499 | 20231 | 23095638 |
| 22534123 | 2067 | 6657 | 10957 | 15501 | 20233 | 23095639 |
| 22534124 | 2069 | 6659 | 10959 | 15503 | 20235 | 23095640 |
| 22534125 | 2071 | 6661 | 10961 | 15505 | 20237 | 23095641 |
| 22534126 | 2073 | 6663 | 10963 | 15507 | 20239 | 23095642 |
| 22534127 | 2075 | 6665 | 10965 | 15509 | 20241 | 23095643 |
| 22534128 | 2077 | 6667 | 10967 | 15511 | 20249 | 23095644 |
| 22534129 | 2079 | 6669 | 10969, 11025 | 15513, 15563 | 20251 | 23095645 |
| 22534130 | 2081 | 6671 | 10971 | 15515 | 20253 | 23095646 |
| 22534131 | 2083 | 6673 | 10973 | 15517 | 20255 | 23095647 |
| 22534132 | 2085 | 6675, 6677 | 10975 | 15519 | 20257 | 23095648 |
| 22534133 | 2087 | 6679 | 10977, 13075 | 15521 | 20259 | 23095649 |
| 22534330 | 2089, 2437, 2439 | 6997 | 11253 | 15843 | 20687 | 24412958 |
| 22534134 | 2091 | 6639, 6681 | 10979 | 15523, 15565, | 20261 | 23095650 |
| | | | | 15571 | | |
| 22534135 | 2093 | 6683 | 10981 | 15525, 15567 | 20263 | 23095651 |
| 22534136 | 2095 | 6685 | 10983 | 15527 | 20265 | 23095652 |
| 22534137 | 2097 | 6687 | 10985 | 15529, 15573 | 20267 | 23095653 |
| 22534138 | 2099 | 6689, 6767 | 10987 | 15531 | 20269 | 23095654 |
| 22534140 | 2101 | 6691 | 10989 | 15533 | 20271 | 23095655 |
| 22534141 | 2103 | 6693, 6769 | 10991 | 15535 | 20273 | 23095656 |
| 22534142 | 2105 | 6695 | 10993 | 15537 | 20275 | 23095657 |
| 22534143 | 2107 | 6697 | 10995 | 15539 | 20277 | 23095658 |
| 22534144 | 2109 | 6699 | 10997 | 15541 | 20279 | 23095659 |
| 22534145 | 2111 | 6701 | 10999 | 15543 | 20281 | 23095660 |
| 22534146 | 2113 | 6703 | 11001 | 15545 | 20283 | 23095661 |
| 22534147 | 2115 | 6705 | 11003 | 15547 | 20285 | 23095662 |
| 22534148 | 2117 | 6707 | 11005 | 15549, 15551 | 20287 | 23095663 |
| 22534149 | 2119, 2121 | 6709, 6715, 6717, | 11007 | 15553, 15555 | 20289, 20291, | 23095664 |
| | | 6719 | | | 20297, 20299, | |
| | | | | | 20301 | |
| 22534151 | 2123 | 6721 | 11009 | 15557, 15569 | 20303 | 23095665 |
| 22534152 | 2125 | 6723 | 11011 | 15559 | 20305 | 23095666 |
| | | 6774 | 11030 | 17716 | 20326 | |
| 22534170 | 2127 | 6761 | 11047 | 15591 | 20343 | 23095682 |
| 22534171 | 2129 | 6763 | 11049 | 15593 | 20345, 20347 | 23095683 |
| 22534172 | 2131 | 6765, 6771, 8705 | 11051 | 15595 | 20349 | 23095684 |
| 22534173 | 2133 | 6773 | 11053 | 15597, 15599, | 20351 | 23095685 |
| | | | | 15601 | | |
| 22534174 | 2135 | 6775, 6777 | 11055 | 15603 | 20353 | 23095686 |
| 22534175 | 2137 | 6779 | 11057 | 15605 | 20355 | 23095687 |
| 22534176 | 2139 | 6781 | 11059 | 15607 | 20357 | 23095688 |
| 22534177 | 2141, 4145 | 6783 | 11061, 11063 | 15609 | 20359 | 23095689 |
| | 2142 | 6784 | 11064 | 15610 | 20360 | |
| 22534181 | 2153 | 6795 | 11071 | 15617 | 20379 | 23095701 |
| 22534182 | 2155 | 6797 | 11073 | 15619 | 20381 | 23095702 |
| 22534183 | 2157 | 6799 | 11075 | 15621 | 20383 | 23095703 |
| 22534184 | 2159 | 6787, 6801 | 11077 | 15623 | 20385 | 23095704 |
| 22534185 | 2161 | 6803 | 11079 | 15625 | 20387 | 23095705 |
| 22534186 | 2163, 2165 | 6805 | 11081 | 15627 | 20389 | 23095706 |
| 22534187 | 2167 | 6807 | 11083 | 15629 | 20391 | 23095707 |
| 22534188 | 2169 | 6809 | 11085 | 15631, 15633 | 20393 | 23095708 |
| 22534190 | 2171 | 6811 | 11087 | 15635 | 20395 | 23095709 |
| 22534200 | 2173, 4191 | 6827, 6943 | 11103 | 15651 | 20411 | 23095717 |
| 22534201 | 2175 | 6829 | 11105 | 15653, 15655 | 20413 | 23095718 |
| 22534202 | 2177 | 6831, 6945 | 11107 | 15657 | 20415 | 23095719 |
| 22534203 | 2179 | 6833 | 11109 | 15659 | 20417 | 23095720 |
| 22534204 | 2181 | 6835 | 11111 | 15661 | 20419 | 23095721 |
| 22534205 | 2183 | 6837 | 11113 | 15663 | 20421 | 23095722 |
| 22534206 | 2185 | 6839 | 11115 | 15671 | 20423 | 23095723 |
| 22534207 | 2187 | 6841, 6947 | 11117 | 15673 | 20425 | 23095724 |
| 22534208 | 2189 | 6843 | 11119 | 15675 | 20427 | 23095725 |
| 22534209 | 2191 | 6845 | 11121 | 15677 | 20429, 20441 | 23095726 |
| 22534210 | 2193 | 6847 | 11123 | 15679 | 20431, 20443 | 23095727 |
| 22534211, 22535115 | 2195, 2197, 3735, | 5103, 6849, 8443 | 11125, 12643 | 15681, 17443 | 20433, 22067 | 23095728, 24413604 |
| | 3737, 3739 | | | | | |
| 22534212 | 2199 | 6851 | 11127 | 15683 | 20435 | 23095729 |
| 22534214 | 2201 | 6855 | 11129 | 15685 | 20437 | 23095730 |
| 22534215 | 2203 | 6857 | 11131, 11133 | 15687 | 20445 | 23095731 |
| 22534216 | 2205, 2207 | 6859 | 11135 | 15689 | 20447, 20449 | 23095732 |
| 22534217 | 2209 | 6861, 6863 | 11137 | 15691 | 20451 | 23095733 |
| 22534218 | 2211 | 6865 | 11139 | 15693 | 20453, 20525, | 23095734 |
| | | | | | 20527 | |
| 22534219 | 2213 | 6867 | 11141 | 15695, 17919 | 20455, 20529 | 23095735 |
| 22534220 | 2215 | 6869 | 11143 | 15697 | 20457 | 23095736 |
| 22534221 | 2217 | 6871 | 11145 | 15699, 15773 | 20459 | 23095737 |
| 22534222 | 2219 | 6873 | 11147 | 15701, 17755 | 20461 | 23095738 |
| 22534223 | 2221 | 6875 | 11149 | 15703 | 20463 | 23095739 |
| 22534224 | 2223 | 6877 | 11151 | 15705, 15775 | 20465 | 23095740 |
| 22534225 | 2225 | 6879 | 11153 | 15707 | 20467 | 23095741 |
| 22534226 | 2227 | 6881 | 11155 | 15709 | 20469 | 23095742 |
| 22534227 | 2229 | 6883 | 11157 | 15711 | 20471 | 23095743 |
| 22534228 | 2231 | 6885 | 11159 | 15713 | 20473 | 23095744 |
| 22534229 | 2233 | 6889 | 11161 | 15715 | 20475 | 24412859 |
| 22534230 | 2235 | 6891 | 11163 | 15717 | 20477 | 24412860 |
| 22534231 | 2237 | 6893 | 11165 | 15719 | 20479 | 24412861 |
| 22534232 | 2239 | 6895 | 11167 | 15721 | 20481 | 24412862 |
| 22534233 | 2241 | 6897 | 11169 | 15723 | 20483 | 24412863 |
| 22534235 | 2243 | 6899 | 11171 | 15725 | 20485 | 24412864 |
| 22534236 | 2245 | 6901 | 11173 | 15727 | 20487 | 24412865 |
| 22534237 | 2247, 2249 | 6903 | 11175 | 15729 | 20489 | 24412866 |
| 22534239 | 2253, 2255 | 6905 | 11179 | 15733 | 20491 | 24412867 |
| 22534240 | 2257, 2259 | 6907 | 11181 | 15735 | 20493 | 24412868 |
| 22534241 | 2261 | 6909 | 11183 | 15737 | 20495 | 24412869 |
| 22534242 | 2263 | 6911 | 11185 | 15739 | 20497 | 24412870 |
| 22534243 | 2265 | 6913 | 11187 | 15741 | 20499 | 24412871 |
| 22534244 | 2267 | 6915 | 11189 | 15743 | 20501, 22713 | 24412872 |
| 22534245 | 2269 | 6917 | 11191 | 15745 | 20503 | 24412873 |
| 22534246 | 2271 | 6919, 6921 | 11193, 11195 | 15747, 15749 | 20505 | 24412874 |
| 22534247 | 2273, 2275, 2277, | 6923 | 11197, 11199 | 15751, 17741 | 20507 | 24412875 |
| | 2279, 2281 | | | | | |
| 22534248 | 2287 | 6927 | 11203 | 15755 | 20511 | 24412877 |
| 22534249 | 2289 | 6929 | 11205 | 15757 | 20513 | 24412878 |
| 22534250 | 2291 | 6931 | 11207 | 15759 | 20515 | 24412879 |
| 22534257 | 2295 | 6949 | 11219 | 15777 | 20531 | 24412886 |
| 22534258, 22534268 | 2297, 2327 | 6951, 6973 | 11221, 11241 | 15779 | 20551 | 24412887, 24412893 |
| 22534259, 22534269 | 2299, 2301, 2329 | 6953, 6965, 6975 | 11223, 11243 | 15781, 15807 | 20533 | 24412888 |
| 22534260 | 2303 | 6955 | 11225 | 15783 | 20535 | 24412889 |
| 22534261 | 2305 | 6961 | 11231 | 15789 | 20541 | 24412890 |
| 22534262 | 2307 | 6963 | 11233 | 15791 | 20543 | 24412891 |
| | 2313 | 6967 | 11235 | 15795 | 20545 | |
| 22534264 | 2315, 3961 | 6969, 6971 | 11239 | 15797 | 20549 | 23094836, 23095149, |
| | | | | | | 23095422, |
| | | | | | | 23095568 |
| 22534272 | 2335 | 6979 | 11247 | 15809 | 22489, 22497 | 24412895 |
| 22534325 | 2427, 4031 | 6981 | 13003 | 15831 | 20679 | 24412954 |
| 22534326 | 2429, 2431, 4029 | 6983 | 13001 | 15835 | 20681 | 24412955 |
| 22534327 | 2433 | 6985, 6991, 6993 | 11249 | 15837, 15839, | 20683 | 24412956 |
| | | | | 15845 | | |
| 22534329 | 2435 | 6995 | 11251 | 15841 | 20685 | 24412957 |
| 22534332 | 2441 | 6999 | 11255 | 15847 | 20689 | 24412959 |
| 22534333 | 2443 | 7001, 7003 | 11257, 11259 | 15849, 15851, | 20691, 20693 | 24412960 |
| | | | | 15857 | | |
| 22534335 | 2445 | 7005 | 11263 | 15855, 15859 | 20697 | 24412961 |
| 22534336 | 2447 | 7007 | 11265 | 15861 | 20699 | 24412962 |
| 22534337 | 2449 | 7009 | 11267 | 15863 | 20701 | 24412963 |
| 22534339 | 2451 | 7013 | 11271 | 15867 | 20705 | 24412964 |
| 22533322, 22534340 | 2453 | 4981, 7015 | 9411, 11273 | 13801, 15869 | 18555, 20707 | 24412965 |
| 22534341 | 2455 | 7017 | 11275 | 15871, 15899 | 20709 | 24412966 |
| 22534342 | 2457 | 7019 | 11277 | 15873 | 20711, 20713 | 24412967 |
| 22534343 | 2459 | 7021 | 11279 | 15875 | 20715 | 24412968 |
| 22534344 | 2461 | 7023 | 11281 | 15877 | 20717 | 24412969 |
| 22534345 | 2463 | 7025 | 11283 | 15879 | 20719 | 24412970 |
| 22534346 | 2465 | 7027 | 11285 | 15881 | 20721 | 24412971 |
| 22534347 | 2467 | 7029 | 11287 | 15883 | 20723 | 24412972 |
| 22534348 | 2469 | 7031 | 11289 | 15885 | 20725 | 24412973 |
| 22534349 | 2471 | 7033 | 11291 | 15887 | 20727 | 24412974 |
| 22534350 | 2473 | 7035 | 11293 | 15889 | 20729 | 24412975 |
| 22534351 | 2475 | 7037 | 11295 | 15891 | 20731 | 24412976 |
| 22534352 | 2477 | 7039 | 11297 | 15893 | 20733 | 24412977 |
| 22534353 | 2479 | 8663 | 11299 | 15895 | 20735 | 24412978 |
| 22534354 | 2481 | 8661 | 11301 | 15897, 15901 | 20737 | 24412979 |
| 22534359 | 2483, 2485 | 7041, 8739 | 11309 | 15909 | 20745 | 24412983 |
| 22534360 | 2487 | 7043 | 11311 | 15911, 15913 | 20747 | 24412984 |
| 22534361 | 2489 | 7045 | 11313 | 15915 | 20749 | 24412985 |
| 22534362 | 2491 | 7047 | 11315 | 15917 | 20751 | 24412986 |
| 22534363 | 2493, 2495 | 7049 | 11317 | 15919 | 20753, 20767 | 24412987 |
| 22534364 | 2497 | 7051 | 11319 | 15921 | 20755 | 24412988 |
| 22534365 | 2499 | 7053 | 11321 | 15923, 15925 | 20757 | 24412989 |
| 22534366 | 2501 | 7055, 7057 | 11323, 11351 | 15927 | 20759 | 24412990 |
| 22534367 | 2503 | 7059 | 11325 | 15929 | 20761 | 24412991 |
| 22534368 | 2505 | 7061 | 11327 | 15931, 15935 | 20763 | 24412992 |
| 22534369 | 2507 | 7063 | 11329 | 15933, 15937 | 20765 | 24412993 |
| 22534370 | 2509 | 7065 | 11331 | 15939 | 20769 | 24412994 |
| 22534371 | 2511 | 7071 | 11333 | 15941 | 20771 | 24412995 |
| 22534372 | 2513 | 7073, 7075 | 11335 | 15943, 15945 | 20773, 20775 | 24412996 |
| 22534373, 22534971 | 2515, 3467, 3469, | 4363, 7077, 8149 | 11337, 12427 | 15947, 17083 | 20777, 21847 | 24412914, 24412997, |
| | 3471, 3959 | | | | | 24413497 |
| 22534377 | 2517 | 7083 | 11343 | 15953 | 20783 | 24413000 |
| 22534378 | 2519 | 7085 | 11345 | 15955 | 20785 | 24413001 |
| 22534379 | 2521 | 7087 | 11347 | 15957 | 20787 | 24413002 |
| 22534380 | 2523 | 7089 | 11349, 11353, | 15959, 15965 | 20789 | 24413003 |
| | | | 13065 | | | |
| 22534381 | 2525 | 7091 | 11355 | 15961 | 20791, 20793 | 24413004 |
| 22534382 | 2527 | 7093 | 11357, 11359 | 15963 | 20795 | 24413005 |
| 22534383 | 2529 | 7101, 7103 | 11361 | 15967 | 20797, 20799 | 24413006 |
| 22534386 | 2535 | 7107 | 11367 | 15969, 15971 | 20803 | 24413009 |
| 22534387 | 2537 | 7109 | 11369 | 15973 | 20805 | 24413010 |
| 22534388 | 2539 | 7111, 7113 | 11371, 11373 | 15975 | 20807 | 24413011 |
| 22534389 | 2541 | 7115 | 11375 | 15977 | 20809, 20811 | 24413012 |
| 22534390 | 2543 | 7117 | 11377 | 15979 | 20813 | 24413013 |
| 22534391 | 2545 | 7119 | 11379 | 15981 | 20815, 20817 | 24413014 |
| 22534392 | 2547 | 7121 | 11381 | 15983 | 20819 | 24413015 |
| 22534393 | 2549 | 7123 | 11383 | 15985 | 20821 | 24413016 |
| 22534394 | 2551 | 7125 | 11385 | 15987 | 20823 | 24413017 |
| 22534395 | 2553 | 7127 | 11387 | 15989 | 20825 | 24413018 |
| 22534396 | 2555 | 7129 | 11389 | 15991 | 20827 | 24413019 |
| 22534397 | 2557 | 7131 | 11391 | 15993 | 20829 | 24413020 |
| 22534398 | 2559 | 7133 | 11393 | 15995 | 20831 | 24413021 |
| 22534400 | 2561 | 7135 | 11395 | 15997 | 20837 | 24413022 |
| 22534401 | 2563 | 7137 | 11397 | 15999 | 20839 | 24413023 |
| 22534403 | 2569 | 7139 | 11401 | 16003 | 20843, 20845 | 24413025 |
| 22534404 | 2571 | 5973, 7141 | 11403 | 16027 | 20847 | 24413026 |
| 22534405 | 2573 | 5971, 7143, 7147 | 11405 | 16029 | 20849, 20851 | 24413027 |
| 22534406 | 2575 | 7149 | 11407 | 16031 | 20853 | 24413028 |
| 22534407 | 2577 | 7151 | 11409 | 16033 | 20855 | 24413029 |
| 22534408 | 2579 | 7153 | 11411 | 16035 | 20857 | 24413030 |
| 22534409 | 2581 | 7155, 7163 | 11413 | 16037 | 20859, 20861 | 24413031 |
| 22534410 | 2583 | 7157 | 11415 | 16039 | 22523 | 24413032 |
| 22534412 | 2587 | 7161 | 13069 | 16043 | 22519 | 24413034 |
| 22534416 | 2589 | 7167 | 11421, 11423 | 16047 | 20865 | 24413038 |
| 22534417 | 2591 | 7169, 7171 | 11425 | 16049 | 20867 | 24413039 |
| 22534418 | 2593 | 7173 | 11427 | 16051 | 20869 | 24413040 |
| 22534419 | 2595 | 7175 | 11429 | 16053 | 20871 | 24413041 |
| 22534420 | 2597, 3977 | 7177 | 11431 | 16055 | 20873 | 24413043 |
| 22534421 | 2599, 3979 | 7179 | 11433 | 16057, 16059 | 20875 | 24413044 |
| 22534422 | 2601 | 7181 | 11435 | 16061 | 20877 | 24413045 |
| 22534423 | 2603 | 7183 | 11437 | 16063 | 20879 | 24413046 |
| 22534424 | 2605 | 7185 | 11439 | 16065 | 20881 | 24413047 |
| 22534425 | 2607 | 7187 | 11441, 11491 | 16067 | 20883 | 24413048 |
| 22534427 | 2609 | 7189 | 11443, 11493 | 16069, 16071 | 20885 | 24413049 |
| 22534428 | 2611 | 7191 | 11445 | 16073 | 20887 | 24413050 |
| 22534429 | 2613 | 7193 | 11447 | 16077 | 20889 | 24413051 |
| 22534156, 22534430 | 2615 | 6733, 7195 | 11017, 11449 | 16079 | 20315, 20891 | 24413052 |
| 22534431 | 2617 | 7197 | 11451 | 16081 | 20893 | 24413053 |
| 22534432 | 2619 | 7199 | 11453 | 16083 | 20895 | 24413054 |
| 22534438 | 2633 | 7213 | 11467 | 16099, 17731 | 20909 | 24413061 |
| 22534439 | 2635 | 7215 | 11469 | 16101 | 20911 | 24413062 |
| 22534440 | 2637 | 7217 | 11471 | 16103, 16111 | 20913 | 24413063 |
| 22534441 | 2639 | 7219 | 11473 | 16105 | 20915 | 24413064 |
| 22534442 | 2641 | 7221 | 11475 | 16107 | 20917 | 24413065 |
| 22534443 | 2643 | 7223 | 11477 | 16109, 16113 | 20919 | 24413066 |
| 22534444 | 2645 | 7225, 7237 | 11479 | 16115 | 20921 | 24413067 |
| 22534445 | 2647 | 7227 | 11481 | 16117 | 20923 | 24413068 |
| 22534446 | 2649 | 7229, 7235 | 11483 | 16119, 16127, | 20925 | 24413069 |
| | | | | 16129 | | |
| 22534447 | 2651, 2653 | 7231 | 11485 | 16121 | 20927, 20929 | 24413070 |
| 22534448 | 2655 | 7233, 8759 | 11487 | 16123 | 20931 | 24413071 |
| 22534449 | 2657 | 8757 | 11489 | 16125 | 20933 | 24413072 |
| 22534455 | 2659 | 7247 | 11503 | 16139 | 20943 | 24413077 |
| 22534456 | 2661, 2663 | 7249, 8751 | 11505 | 16141 | 20945 | 24413078 |
| 22534457 | 2665 | 8749 | 11507 | 16143, 17791 | 20947 | 24413079 |
| 22534461 | 2669 | 7251 | 11511, 11513 | 16145, 16147 | 20953, 20955, | 24413083 |
| | | | | | 22653 | |
| 22534462 | 2671, 2673 | 7253 | 11515 | 16149 | 20957 | 24413084 |
| 22534463 | 2675 | 7255 | 11517 | 16151 | 20959 | 24413085 |
| 22534465 | 2677 | 7259 | 11521 | 16157 | 20963 | 24413087 |
| 22534474 | 2679 | 7273 | 11539, 11541 | 16179 | 20979 | 24413094 |
| 22534477 | 2687 | 7281 | 11549 | 16011 | 20987 | 24413098 |
| 22534478 | 2689 | 7283 | 11551 | 16013 | 20989 | 24413099 |
| 22534483 | 2699 | 7293 | 11561 | 16005 | 21001 | 24413104 |
| 22534491 | 2711 | 7305, 7313, 7347 | 11573 | 16025 | 21013, 22661 | 24413110 |
| 22534492 | 2715 | 7317 | 11581 | 16185, 16191 | 21017, 21123 | 24413112 |
| 22534493 | 2717 | 7319 | 11583 | 16187 | 21019 | 24413113 |
| 22534494 | 2719 | 7321, 7349, 7351 | 11585 | 16189, 16193 | 21021 | 24413114 |
| 22534496 | 2723 | 7325 | 11589 | 16197 | 21025 | 24413116 |
| 22534497 | 2725 | 7327 | 11591 | 16199 | 21027 | 24413117 |
| 22534498 | 2727 | 7329, 8747 | 11593 | 16201, 16217 | 21029 | 24413118 |
| 22534153, 22534499 | 2729 | 6725, 7331 | 11013, 11595 | 15561, 16203, | 20307, 21031 | 23095667 |
| | | | | 17845 | | |
| 22534500 | 2731 | 7333 | 11597 | 16205 | 21035 | 24413119 |
| 22534501 | 2733 | 7335 | 11599 | 16207 | 21037 | 24413120 |
| 22534502 | 2735 | 7337, 7355, 8737 | 11601 | 16209 | 21039, 22515 | 24413121 |
| 22534503 | 2737 | 7339 | 11603 | 16213 | 21041 | 24413122 |
| 22534504 | 2739 | 7341, 8767 | 11605 | 16215, 16219 | 21043 | 24413123 |
| 22534505 | 2741 | 7343 | 11607 | 16221, 16299 | 21045 | 24413124 |
| 22534507 | 2743 | 7345 | 11609 | 16223 | 21047 | 24413125 |
| 22534508 | 2745 | 7357 | 11611 | 16225 | 21049 | 24413126 |
| 22534509 | 2747 | 7359 | 11613 | 16227 | 21051 | 24413127 |
| 22534510 | 2749 | 7361 | 11615 | 16229 | 21053 | 24413128 |
| 22534511 | 2751 | 7363 | 11617 | 16231 | 21055, 21125 | 24413129 |
| 22534512 | 2753 | 7365 | 11619 | 16233 | 21057, 21127 | 24413130 |
| 22534514 | 2755 | 7367 | 11621 | 16235 | 21059 | 24413131 |
| 22534528 | 2761 | 7395 | 11641 | 16257, 16301 | 21079 | 24413141 |
| 22534529 | 2763 | 7397 | 11643 | 16259, 16303 | 21081 | 24413142 |
| 22534530 | 2765, 2767 | 7399 | 11645 | 16261 | 21083 | 24413143 |
| 22534537 | 2769 | 7409 | 11653 | 16271 | 21091 | 24413147 |
| 22534538 | 2771 | 7411 | 11655 | 16273 | 21093 | 24413148 |
| 22534540 | 2773 | 7415 | 11659 | 16277, 16305, | 21097 | 24413150 |
| | | | | 17793 | | |
| 22534541 | 2775, 2777 | 7417 | 11661 | 16279 | 21099, 21133 | 24413151 |
| 22534542 | 2779 | 7419 | 11663 | 16281 | 21101 | 24413152 |
| 22534543 | 2781 | 7421 | 11665 | 16283 | 21103 | 24413153 |
| 22534544 | 2783 | 7423 | 11667 | 16285, 16287 | 21105 | 24413154 |
| 22534545 | 2785 | 7425 | 11669 | 16289 | 21107 | 24413155 |
| 22534546 | 2787 | 7427 | 11671 | 16291 | 21109 | 24413156 |
| 22534547 | 2789 | 7429 | 11673 | 16293 | 21111 | 24413157 |
| 22534548 | 2791 | 7431 | 11675 | 16295 | 21113 | 24413158 |
| 22534549 | 2793 | 7433 | 11677 | 16297 | 21115 | 24413159 |
| 22534552 | 2801 | 7437, 7439 | 11681 | 16307 | 21135 | 24413161 |
| 22534553 | 2803 | 7441, 8765 | 11683 | 16309 | 21139 | 24413162 |
| 22534554 | 2805 | 7443 | 11685 | 16311 | 21141 | 24413163 |
| 22534558 | 2811 | 7445 | 11687 | 16317 | 21143 | 24413165 |
| 22534560 | 2813, 2815 | 7447 | 11689, 11691 | 16319 | 21145 | 24413166 |
| 22534561 | 2817 | 7449 | 11693 | 16321 | 21147 | 24413167 |
| 22534562 | 2819 | 7451 | 11695 | 16323 | 21149 | 24413168 |
| 22534563 | 2821, 2823 | 7453 | 11697 | 16325 | 21151, 21153 | 24413169 |
| 22534565 | 2825 | 7457 | 11701 | 16331 | 21157 | 24413171 |
| 22534566 | 2827 | 7459 | 11703 | 16333 | 21159 | 24413172 |
| 22534567 | 2829 | 7461 | 11705 | 16335 | 21161 | 24413173 |
| 22534568 | 2831 | 7463 | 11707 | 16337 | 21163 | 24413174 |
| 22534569 | 2833 | 7465 | 11709 | 16339 | 21165 | 24413175 |
| 22534571 | 2835 | 7467 | 11711 | 16341, 16421 | 21167 | 24413176 |
| 22534572 | 2837 | 7469 | 11713 | 16343, 16345, | 21169 | 24413177 |
| | | | | 16423 | | |
| 22534573 | 2839 | 7471 | 11715 | 16347 | 21171 | 24413178 |
| 22534574 | 2845 | 7477 | 11719 | 16351 | 21175 | 24413181 |
| 22534575 | 2847 | 7479 | 11721 | 16353 | 21177 | 24413182 |
| 22534576 | 2849 | 7481 | 11723 | 16355 | 21179 | 24413183 |
| 22534577 | 2851 | 7483 | 11725 | 16357 | 21181 | 24413184 |
| 22534578, 22534581 | 2853 | 7485, 7487 | 11727 | 16359, 16361 | 21183 | 24413185 |
| 22534582 | 2855, 2857 | 7489, 7533 | 11729 | 16363, 17775 | 21185 | 24413186 |
| 22534583 | 2859 | 7491 | 11731 | 16365 | 21187 | 24413187 |
| 22534584 | 2861 | 7493 | 11733 | 16367 | 21189 | 24413188 |
| 22534585 | 2863 | 7495 | 11735 | 16369 | 21191 | 24413189 |
| | 2864 | 7496 | 11736 | 16370 | 21192 | |
| 22534587 | 2865 | 7497 | 11737 | 16371, 17787 | 21193 | 24413190 |
| 22534588 | 2867, 2869 | 7499, 8689 | 11739 | 16373 | 21195 | 24413191 |
| 22534589 | 2871 | 7501 | 11741 | 16375 | 21197 | 24413192 |
| 22534590 | 2873 | 7503 | 11743 | 16377 | 21199 | 24413193 |
| 22534592 | 2875 | 7505 | 11745 | 16379 | 21203 | 24413194 |
| 22534593 | 2877 | 7507 | 11747 | 16381 | 21205 | 24413195 |
| 22534594 | 2879 | 7509 | 11749 | 16383, 16425 | 21207 | 24413196 |
| 22534595 | 2881 | 7511 | 11751 | 16385, 16427 | 21209, 21211 | 24413197 |
| 22534597 | 2883 | 7515 | 11755 | 16389, 16391 | 21215 | 24413199 |
| 22534598 | 2885 | 7517 | 11757 | 16393 | 21217 | 24413200 |
| | 2887 | 7520 | 11760 | 16395 | 21220 | |
| 22534599 | 2889 | 7521 | 11761 | 16397 | 21221 | 24413202 |
| 22534602 | 2891 | 7525 | 11765 | 16401 | 21225 | 24413203 |
| 22534603 | 2893 | 7527 | 11767 | 16403 | 21227 | 24413204 |
| 22534604 | 2895, 2897 | 7529 | 11769 | 16405 | 21229 | 24413205 |
| 22534605 | 2899 | 7531, 7537 | 11771 | 16407 | 21231, 21243 | 24413206 |
| 22534606 | 2905 | 7539 | 11773 | 16411 | 21233 | 24413207 |
| 22534607, 22534608 | 2907, 2909 | 7541, 7543 | 11775, 11777 | 16413 | 21235, 21237 | 24413208, 24413209 |
| 22534609 | 2911 | 7545 | 11779 | 16415 | 21239 | 24413210 |
| 22534610 | 2913 | 7547 | 11781 | 16417 | 21241 | 24413211 |
| 22534612 | 2915 | 7549 | 11783 | 16419 | 21245, 21247 | 24413212 |
| 22534613 | 2917 | 7551 | 11785 | 16429 | 21249 | 24413213 |
| 22534615 | 2919 | 7553, 7555 | 11787 | 16431, 16433 | 21251 | 24413214 |
| 22534616 | 2921 | 7557 | 11789, 11791 | 16435 | 21253 | 24413215 |
| 22534617 | 2923 | 7559 | 11793 | 16437 | 21255 | 24413216 |
| 22534618 | 2925, 2927 | 7561 | 11795 | 16439 | 21257 | 24413217 |
| 22534619 | 2929 | 7563, 7565 | 11797 | 16441 | 21259, 21261 | 24413218 |
| 22534620 | 2931 | 7567 | 11799 | 16443 | 21263 | 24413219 |
| 22534621 | 2933 | 7569 | 11801, 13037 | 16445 | 21265 | 24413220 |
| 22534622 | 2935 | 7571 | 11803 | 16447 | 21267 | 24413221 |
| 22534623 | 2937 | 7573 | 11805 | 16449 | 21269 | 24413222 |
| 22534624 | 2939 | 7575 | 11807 | 16451, 16453 | 21271 | 24413223 |
| 22534625 | 2941 | 7577 | 11809 | 16455 | 21273 | 24413224 |
| 22534626 | 2943 | 7579 | 11811 | 16457 | 21275 | 24413225 |
| 22534627 | 2945 | 7581 | 11813 | 16459 | 21277 | 24413226 |
| 22534628 | 2947 | 7583 | 11815 | 16461 | 21279 | 24413227 |
| 22534629 | 2949, 4123, 4125 | 7585, 7587 | 11817 | 16463 | 21281, 21283 | 24413228 |
| 22534630 | 2951, 4127 | 7589 | 11819, 11821 | 16465 | 21285 | 24413229 |
| 22534632 | 2955 | 7593 | 11825 | 16469 | 21287 | 24413231 |
| 22534633 | 2957 | 7595 | 11827 | 16471 | 21289 | 24413232 |
| 22534634 | 2959 | 7597 | 11829 | 16473 | 21291 | 24413233 |
| 22534635 | 2961 | 7599, 8713 | 11831 | 16475 | 21293 | 24413234 |
| 22534636 | 2963 | 7601 | 11833 | 16477 | 21295 | 24413235 |
| 22534637 | 2965 | 7603 | 11835 | 16479 | 21297 | 24413236 |
| 22534639 | 2967 | 7605 | 11837, 11841 | 16481 | 21299 | 24413237 |
| 22534640 | 2969 | 7607, 7609 | 11839, 11843 | 16483 | 21301 | 24413238 |
| 22534641 | 2971 | 7611 | 11845 | 15205, 16485 | 21303 | 24413239 |
| 22534642 | 2973 | 7613 | 11847 | 16487, 16535 | 21305 | 24413240 |
| 22534643 | 2975 | 7615 | 11849 | 16489 | 21307 | 24413241 |
| 22534644 | 2977 | 7617 | 11851 | 16491, 16537 | 21309 | 24413242 |
| 22534645 | 2979 | 7619 | 11853 | 16493 | 21311 | 24413243 |
| 22534646 | 2981 | 7621 | 11855 | 16495 | 21313 | 24413244 |
| 22534647 | 2983 | 7623 | 11857 | 16497 | 21315 | 24413245 |
| 22534649 | 2987 | 7627 | 11861 | 16501 | 21321 | 24413246 |
| 22534651 | 2989 | 7629 | 11863 | 16503 | 21323, 21359 | 24413247 |
| 22534652 | 2991 | 7631 | 11865 | 16505 | 21325 | 24413248 |
| 22534653 | 2993 | 7633 | 11867 | 16507 | 21327 | 24413249 |
| 22534654 | 2995 | 7635 | 11869 | 16509 | 21329 | 24413250 |
| 22534655 | 2997 | 7637 | 11871 | 16511 | 21331 | 24413251 |
| 22534656 | 2999 | 7639 | 11873 | 16513 | 21333 | 24413252 |
| 22534658 | 3001 | 7641 | 11875 | 16515 | 21335 | 24413253 |
| 22534659 | 3003 | 7643, 7645 | 11877 | 16517 | 21337 | 24413254 |
| 22534660 | 3005 | 7647 | 11879, 13111 | 16519 | 21339 | 24413255 |
| 22534661 | 3007 | 7649 | 11881 | 16521 | 21341 | 24413256 |
| 22534662 | 3009 | 7651 | 11883 | 16523 | 21343 | 24413257 |
| 22534668 | 3011 | 7663 | 11895 | 16541 | 21361 | 24413263 |
| 22534669 | 3013 | 7665 | 11897 | 16543 | 21363 | 24413264 |
| 22534670 | 3015 | 7667 | 11899 | 16545 | 21365 | 24413265 |
| 22534671 | 3017 | 7669 | 11901 | 16547 | 21367 | 24413266 |
| 22534672 | 3019 | 7671 | 11903 | 16549, 17843 | 21369 | 24413267 |
| 22534673 | 3021 | 7673 | 11905 | 16551 | 21371 | 24413268 |
| 22534674 | 3023 | 7675 | 11907 | 16553 | 21373 | 24413269 |
| 22534675 | 3025 | 7677 | 11909 | 16555 | 21375 | 24413270 |
| 22534677 | 3027 | 7681 | 11913 | 16559 | 21377 | 24413271 |
| 22534679 | 3029 | 7683 | 11915 | 16573 | 21379 | 24413272 |
| 22534680 | 3031 | 7685 | 11917 | 16575 | 21381 | 24413273 |
| 22534682 | 3035 | 7689 | 11921 | 16579, 16581 | 21385 | 24413274 |
| 22534683 | 3037 | 7691 | 11923 | 16583 | 21387 | 24413275 |
| 22534684 | 3039 | 7693 | 11925 | 16585 | 21389 | 24413276 |
| 22534685 | 3041 | 7695 | 11927 | 16587 | 21391 | 24413277 |
| 22534687 | 3043 | 7697 | 11929 | 16589 | 21393 | 24413278 |
| 22534688, 22535015 | 3045, 3563, 3565, | 7699, 8243 | 11931, 12513 | 16591, 16593, | 21395, 21937 | 24413279, 24413539 |
| | 4071 | | | 17199, 17231 | | |
| 22534689 | 3047 | 7701 | 11933 | 16595 | 21397 | 24413280 |
| 22534690 | 3049 | 7703 | 11935 | 16597 | 21399 | 24413281 |
| 22534691 | 3051 | 7705, 7707 | 11937 | 16599 | 21401 | 24413282 |
| 22534692 | 3053 | 7709 | 11939 | 16601 | 21403 | 24413283 |
| 22534699 | 3055 | 7725 | 11953 | 16615 | 21421 | 24413290 |
| 22534700 | 3057, 4169 | 7727 | 11955 | 16617 | 21423, 21439 | 24413291 |
| 22534701 | 3059, 4171 | 7729 | 11957 | 16619 | 21425 | 24413292 |
| 22534702 | 3061 | 7731 | 11959 | 16621 | 21427 | 24413293 |
| 22534703 | 3063 | 7733 | 11961, 11963 | 16623 | 21429, 21441 | 24413294 |
| 22534704 | 3065 | 7735 | 11965 | 16625 | 21431 | 24413295 |
| 22534705 | 3067 | 7737 | 11967 | 16627 | 21433 | 24413296 |
| 22534708 | 3069 | 7743 | 11973 | 16633, 16639, | 21445 | 24413299 |
| | | | | 16641 | | |
| 22534709 | 3071 | 7745 | 11975 | 16635 | 21447, 22613 | 24413300 |
| 22534711, 22534768 | 3073, 3163 | 7747, 7873 | 11977, 12089 | 16637, 16643, | 21449, 21557 | 24413301, 24413358 |
| | | | | 16753 | | |
| 22534712 | 3075 | 7749, 7751 | 11979 | 16645 | 21451 | 24413302 |
| 22534719 | 3077 | 7765 | 11999 | 16659 | 21465 | 24413309 |
| 22534720 | 3079 | 7767, 7771 | 12001 | 16661 | 21467 | 24413310 |
| 22534721 | 3081 | 7769, 7773, 7775 | 12003 | 16663 | 21469, 21477 | 24413311 |
| 22534722 | 3083 | 7777 | 12005 | 16665 | 21471 | 24413312 |
| 22534723 | 3085 | 7779 | 12007 | 16667 | 21473 | 24413313 |
| 22534724 | 3087 | 7781 | 12009 | 16669 | 21475, 21481 | 24413314 |
| 22534725 | 3089 | 7785 | 12011 | 16671 | 21483 | 24413315 |
| 22534726 | 3091 | 7783, 7787 | 12013 | 16673 | 21485 | 24413316 |
| 22534728 | 3093 | 7789 | 12015 | 16675 | 21487 | 24413317 |
| 22534729 | 3095 | 7791 | 12017 | 16677 | 21489 | 24413318 |
| 22534730 | 3097 | 7793 | 12019 | 16679 | 21491 | 24413319 |
| 22534739 | 3103 | 7817 | 12031 | 16691 | 21505 | 24413330 |
| 22534740 | 3109 | 7821 | 12035 | 16695, 16707 | 21511, 22627 | 24413332 |
| 22534741 | 3111 | 7823 | 12037, 12039 | 16697, 16699 | 21513, 21517 | 24413333 |
| 22534743 | 3113 | 7825 | 12041 | 16701, 16709 | 19293, 21519 | 24413334 |
| 22534744 | 3115 | 7827 | 12043 | 16703, 16711 | 19291 | 24413335 |
| 22534745 | 3117 | 7829 | 12045 | 16705, 16713 | 19289 | 24413336 |
| 22534746 | 3119 | 7831 | 12047 | 17903 | 19287 | 24413337 |
| 22534749 | 3125 | 7837 | 12053 | 16715 | 19279, 21521 | 24413340 |
| 22534750 | 3127 | 7839 | 12055 | 16717 | 21523 | 24413341 |
| 22534751 | 3129 | 7841 | 12057 | 16719 | 21525 | 24413342 |
| 22534752 | 3131 | 7843, 7887 | 12059 | 16721 | 21527 | 24413343 |
| 22534753 | 3133 | 7845, 7889 | 12061 | 16723, 16735 | 21529, 21573 | 24413344 |
| 22534755 | 3135 | 7847 | 12063 | 16725 | 21531 | 24413345 |
| 22534756 | 3137 | 7849 | 12065 | 16727 | 21533 | 24413346 |
| 22534757 | 3139 | 7851 | 12067 | 16729 | 21535 | 24413347 |
| 22534758 | 3141 | 7853, 7891 | 12069 | 16731 | 21537 | 24413348 |
| 22534759 | 3143 | 7855 | 12071 | 16733 | 21539 | 24413349 |
| 22534760 | 3145 | 7857 | 12073 | 16737 | 21541 | 24413350 |
| 22534761 | 3147 | 7859 | 12075 | 16739 | 21543 | 24413351 |
| 22534762 | 3149 | 7861 | 12077 | 16741 | 21545 | 24413352 |
| 22534763 | 3151 | 7863 | 12079 | 16743 | 21547 | 24413353 |
| 22534764 | 3153 | 7865 | 12081 | 16745 | 21549 | 24413354 |
| 22534765 | 3155 | 7867 | 12083 | 16747 | 21551 | 24413355 |
| 22534766 | 3157 | 7869 | 12085 | 16749 | 21553 | 24413356 |
| 22534767 | 3159, 3161 | 7871 | 12087 | 16751 | 21555 | 24413357 |
| 22534769 | 3165, 3167 | 7875 | 12091, 12093 | 16755 | 21559 | 24413359 |
| 22534770 | 3169 | 7877 | 12095 | 16757 | 21561 | 24413360 |
| 22534771 | 3171 | 7879 | 12097 | 16759 | 21563 | 24413361 |
| 22534772 | 3173 | 7881 | 12099 | 16761 | 21565 | 24413362 |
| 22534773 | 3175, 4065 | 7883 | 12101 | 16763 | 21567 | 24413363 |
| 22534774 | 3177, 3179, 3683, | 7885, 7893 | 12103 | 16765 | 21569 | 24413364 |
| | 4067 | | | | | |
| 22534775 | 3181 | 7895 | 12105, 12159 | 16767 | 21571, 21575 | 24413365 |
| 22534776 | 3183 | 7897 | 12107 | 16769, 16795 | 21577 | 24413366 |
| 22534777 | 3185, 3187 | 7899 | 12109 | 16771, 17829 | 21579, 21595, | 24413367 |
| | | | | | 21597 | |
| 22534779 | 3189 | 7901 | 12111 | 16773 | 21581 | 24413368 |
| 22534781 | 3191 | 7145, 7905 | 12115 | 16777 | 21585 | 24413370 |
| 22534783 | 3195 | 7907 | 12151 | 16781 | 21601 | 24413371 |
| 22534784 | 3197 | 7909 | 12153 | 16783 | 21603 | 24413372 |
| 22534785 | 3199 | 7911 | 12155 | 16785, 16787 | 21605 | 24413373 |
| 22534786 | 3201 | 7913 | 12157 | 16789 | 21607 | 24413374 |
| 22534787 | 3203 | 7915 | 12161 | 16791 | 21609 | 24413375 |
| 22534792 | 3207 | 6301 | 12169 | 16803 | 21617 | 24413379 |
| 22534793 | 3209, 3211 | 6303, 8769 | 12171 | 16805, 16827 | 21619 | 24413380 |
| 22534794 | 3213 | 6305 | 12173 | 16807 | 21621 | 24413381 |
| 22534795 | 3215 | 6307 | 12175 | 16809, 16811 | 21623 | 24413382 |
| 22534796 | 3217 | 6309 | 12177 | 16813 | 21625 | 24413383 |
| 22534797 | 3219 | 6311 | 12179 | 16815 | 21627 | 24413384 |
| 22534798 | 3221 | 6313 | 12181 | 16817 | 21629 | 24413385 |
| 22534799 | 3223 | 6315 | 12183 | 16819 | 21631 | 24413386 |
| 22534800 | 3225 | 6317 | 12185 | 16821 | 21633 | 24413387 |
| 22534801 | 3227 | 6319 | 12187 | 16823 | 21635 | 24413388 |
| 22534802 | 3229 | 6321 | 12189 | 16825, 16829 | 21637 | 24413389 |
| 22534804 | 3231 | 6323, 7921 | 12191, 12193, | 16831 | 21639 | 24413390 |
| | | | 12195 | | | |
| 22534805 | 3233 | 7923, 7925 | 12197 | 16833 | 21641 | 24413391 |
| 22534807 | 3237 | 7929, 7931 | 12203 | 16839, 16841 | 21645 | 24413393 |
| 22534808 | 3239 | 7933 | 12205 | 16843 | 21647 | 24413394 |
| 22534809 | 3241 | 7935 | 12207 | 16845 | 21649 | 24413395 |
| 22534810 | 3243 | 7937 | 12209 | 16847 | 21651 | 24413396 |
| 22534811 | 3245 | 7939 | 12211 | 16849 | 21653 | 24413397 |
| 22534812 | 3247 | 7941 | 12213 | 16851 | 21655 | 24413398 |
| 22534813 | 3249 | 7943 | 12215 | 16853 | 21657 | 24413399 |
| 22534814 | 3251 | 7945 | 12217 | 16855 | 21659 | 24413400 |
| 22534815 | 3253 | 7947 | 12219 | 16857 | 21661 | 24413401 |
| 22534816 | 3255, 3257 | 7949 | 12221 | 16859 | 21663 | 24413402 |
| 22534819 | 3265 | 7955 | 12227 | 16865, 16895 | 21669 | 24413405 |
| 22534820 | 3267 | 5077, 7957 | 12229 | 16867 | 21671 | 24413406 |
| 22534821 | 3269 | 7959 | 12231 | 16869 | 21673 | 24413407 |
| 22534822 | 3271 | 7961 | 12233, 12249 | 16871, 16873 | 21675 | 24413408 |
| 22534823 | 3273 | 7963 | 12235, 12251 | 16875 | 21677 | 24413409 |
| 22534824 | 3275 | 7965 | 12237 | 16877 | 21679 | 24413410 |
| 22534825 | 3277 | 7967 | 12239 | 16879 | 21681 | 24413411 |
| 22534826 | 3279 | 7969 | 12241 | 16881 | 21683 | 24413412 |
| 22534827 | 3281 | 7971 | 12243 | 16883 | 21685 | 24413413 |
| 22534829 | 3283 | 7973 | 12245 | 16885 | 21687 | 24413414 |
| 22534830 | 3285 | 7975 | 12247 | 16887 | 21689 | 24413415 |
| 22534832 | 3289, 3291 | 7979 | 12255, 12257 | 16897, 17773 | 21691 | 24413417 |
| 22534834 | 3295 | 7981 | 12259 | 16903 | 21693 | 24413418 |
| 22534835 | 3297 | 7983, 7985 | 12261 | 16905 | 21695 | 24413419 |
| 22534837 | 3301, 3969, 3973 | 7987 | 12263 | 16907, 16909, | 21697 | 24413420 |
| | | | | 17749 | | |
| 22534838 | 3303 | 7989 | 12265 | 16911 | 21699 | 24413421 |
| 22534839 | 3305 | 7991 | 12267, 12301 | 16913 | 21701 | 24413422 |
| 22534840 | 3307 | 7993 | 12269 | 16915 | 21703, 21705 | 24413423 |
| 22534841 | 3309, 3311 | 7995 | 12271 | 16917 | 21707 | 24413424 |
| 22534842 | 3313 | 7997 | 12273 | 16919, 16947 | 21709 | 24413425 |
| 22534843 | 3315, 3317 | 7999 | 12275 | 16921 | 21711 | 24413426 |
| 22534844 | 3319 | 8001 | 12277 | 16923 | 21713 | 24413427 |
| 22534845 | 3321 | 8003 | 12279 | 16925 | 21715, 21717 | 24413428 |
| 22534847 | 3323 | 8005 | 12281 | 16927 | 21719 | 24413429 |
| 22534848 | 3325 | 8007 | 12123, 12283 | 16929 | 21721, 22431 | 24413430 |
| 22534849 | 3327 | 8009 | 12285 | 16931 | 21723 | 24413431 |
| 22534850 | 3329 | 8011 | 12287 | 16933 | 21725 | 24413432 |
| 22534851 | 3331 | 8013 | 12289 | 16935 | 21727 | 24413433 |
| 22534852 | 3333 | 8015 | 12291 | 16937 | 21729 | 24413434 |
| 22534853 | 3335 | 8017, 8021 | 12293 | 16939 | 21731 | 24413435 |
| 22534854 | 3337 | 8019, 8023 | 12295 | 16941, 16945, | 21733 | 24413436 |
| | | | | 16949 | | |
| 22534855 | 3339 | 8025 | 12297 | 16951 | 21735 | 24413437 |
| 22534857 | 3341 | 8029 | 12303 | 16955 | 21739, 21741 | 24413440 |
| 22534858 | 3343 | 8031 | 12305 | 16957 | 21743 | 24413441 |
| 22534859 | 3345 | 8033 | 12311 | 16959 | 21745 | 24413442 |
| 22534860 | 3347, 3349, 3351 | 8035 | 12313 | 16961 | 21747, 21749 | 24413443 |
| 22534861 | 3353 | 8037 | 12315 | 16963 | 21751, 21755 | 24413444 |
| 22534862 | 3355 | 8039, 8059 | 12317 | 16965 | 21753, 21757 | 24413445 |
| 22534864 | 3359 | 8043 | 12321 | 16969, 17757 | 21761 | 24413447 |
| 22534865 | 3361 | 8045 | 12323 | 16971 | 21763 | 24413448 |
| 22534866 | 3363 | 8047 | 12325 | 16973 | 21765 | 24413449 |
| 22534867 | 3365 | 8049 | 12327 | 16975 | 21767 | 24413450 |
| 22534868 | 3367 | 8051 | 12329 | 16977 | 21769 | 24413451 |
| 22534870 | 3369 | 8053 | 12331 | 16979 | 21771 | 24413452 |
| 22534871 | 3371 | 8055 | 12333 | 16981, 16983 | 21773 | 24413453 |
| 22534872 | 3373 | 8057 | 12335 | 16985 | 21775 | 24413454 |
| 22534873 | 3375 | 8061 | 12337 | 16987 | 21777 | 24413455 |
| 22534874 | 3377 | 8063 | 12339 | 16989 | 21779 | 24413456 |
| 22534927 | 3379, 3381, 3383 | 8065 | 12341 | 16991 | 21781, 22593 | 24413457 |
| 22534928 | 3385 | 8067 | 12343 | 16993 | 21783 | 24413458 |
| 22534929 | 3387 | 8069 | 12345 | 16995 | 21785 | 24413459 |
| 22534932 | 3391 | 8073 | 12349 | 16999 | 21789 | 24413461 |
| 22534933 | 3393 | 8075 | 12351 | 17001 | 21791 | 24413462 |
| 22534935 | 3395 | 8079 | 12355 | 17005 | 21795 | 24413463 |
| 22534936 | 3397 | 8081 | 12357 | 17007 | 21797 | 24413464 |
| 22534937 | 3399 | 8083 | 12359 | 17009 | 21799 | 24413465 |
| 22534938 | 3401 | 8085 | 12361 | 17011 | 21801 | 24413466 |
| 22534950 | 3425 | 8107 | 12381 | 17035 | 21805 | 24413477 |
| 22534951 | 3427 | 8109 | 12383 | 17037 | 21807 | 24413478 |
| 22534952 | 3429 | 8111, 8113 | 12385, 12399 | 17039, 17043, | 21809, 21821 | 24413479 |
| | | | | 17935 | | |
| 22534954 | 3431 | 8115 | 12387 | 17041, 17045 | 21811 | 24413480 |
| 22534955 | 3433 | 8117 | 12389 | 17047, 17049 | 21813 | 24413481 |
| 22534956 | 3435 | 8119 | 12391, 12401 | 17051 | 21815 | 24413482 |
| 22534957 | 3437 | 8121 | 12393 | 17053 | 21817 | 24413483 |
| 22534958 | 3439 | 8123 | 12395 | 17055, 17057 | 21819 | 24413484 |
| 22534960 | 3441 | 8127 | 12405 | 17061 | 21825 | 24413486 |
| 22534961 | 3443 | 8129 | 12407 | 17063 | 21827 | 24413487 |
| 22534962 | 3445 | 8131 | 12409 | 17065 | 21829 | 24413488 |
| 22534963 | 3447 | 8133 | 12411 | 17067 | 21831 | 24413489 |
| 22534964 | 3449 | 8135 | 12413 | 17069 | 21833, 21969 | 24413490 |
| 22534965 | 3451, 3455 | 8137 | 12415 | 17071 | 18919, 21835 | 24413491 |
| 22534966 | 3453, 3457 | 8139 | 12417 | 17073 | 21837 | 24413492 |
| 22534967 | 3459 | 8141 | 12419 | 17075, 17157 | 21839, 21971 | 24413493 |
| 22534968 | 3461 | 8143 | 12421 | 17077 | 21841 | 24413494 |
| 22534969 | 3463 | 8145 | 12423 | 17079 | 21843 | 24412908, 24413495 |
| 22534970 | 3465 | 8147 | 12425 | 17081 | 21845 | 24412913, 24413496 |
| 22534972 | 3473 | 8151 | 12429 | 17089 | 21849 | 24412915, 24413498 |
| 22534715, 22534973 | 3475 | 7757, 8153 | 11989, 12431 | 16651, 17091, | 21457, 21851 | 24412916, 24413305, |
| | | | | 17833 | | 24413499 |
| 22534975 | 3477 | 8155 | 12433 | 17093 | 21853 | 24413500 |
| 22534976 | 3479 | 8157, 8159, 8161 | 12435 | 17097 | 21855 | 24413501 |
| 22534977 | 3481 | 8163 | 12437 | 17099 | 21857 | 24413502 |
| 22534978 | 3483 | 8165 | 12439 | 17101 | 21859 | 24413503 |
| 22534980 | 3491 | 8173 | 12445 | 17107 | 21865 | 24413506 |
| 22534981 | 3493 | 6191 | 12447 | 17109 | 21867 | 24413507 |
| 22534982 | 3495, 3497 | 6193 | 12449 | 17111 | 21869, 21973 | 24413508 |
| 22534983 | 3499 | 8183 | 12451 | 17113, 17159 | 21871 | 24413509 |
| 22534984 | 3501 | 8185 | 12453 | 17115, 17161 | 21873 | 24413510 |
| 22534985 | 3503 | 8187 | 12455 | 17117, 17163 | 21875 | 24413511 |
| 22534986 | 3505 | 8189 | 12457 | 17119 | 21877 | 24413512 |
| 22534987 | 3507 | 8191 | 12459 | 17121 | 21879 | 24413513 |
| 22534988 | 3509 | 8193 | 12461 | 17123 | 21881 | 24413514 |
| 22534989 | 3511 | 8195 | 12463 | 17125 | 21883 | 24413515 |
| 22534991 | 3513 | 8197 | 12465 | 17127, 17165 | 21885 | 24413516 |
| 22534992 | 3515 | 8199 | 12467 | 17129, 17167 | 21887 | 24413517 |
| 22534993 | 3517 | 8201 | 12469 | 17131 | 21889 | 24413518 |
| 22534995 | 3521 | 8205 | 12473 | 17135, 17169 | 21893 | 24413520 |
| 22534997 | 3523 | 8209 | 12477 | 17139 | 21897, 21899 | 24413521 |
| 22534998 | 3525 | 8211 | 12479 | 17141 | 21901 | 24413522 |
| 22534999 | 3529 | 8215 | 12485 | 17147 | 21907, 21909 | 24413524 |
| 22535000 | 3531 | 8217 | 12487 | 17149, 17171 | 21911 | 24413526 |
| 22535001 | 3533 | 8219 | 12489 | 17151, 17173 | 21913 | 24413527 |
| 22535002 | 3535 | 8221 | 12491 | 17155, 17175 | 21915 | 24413528 |
| 22535003 | 3537 | 8223 | 12493 | 17177 | 21917 | 24413529 |
| 22535004 | 3539 | 8225 | 12495 | 17179 | 21919 | 24413530 |
| 22535005 | 3541 | 8227 | 12497 | 17181 | 21921 | 24413531 |
| 22535006 | 3543 | 8229 | 12499 | 17183, 17225 | 21923 | 24413532 |
| 22535007 | 3545 | 8231 | 12501 | 17185, 17227 | 21925 | 24413533 |
| 22535009 | 3549, 4013 | 8235 | 12505 | 17189, 17191 | 21929 | 24413535 |
| 22535010 | 3551, 3553, 4015 | 8237 | 12507 | 17193 | 21931 | 24413536 |
| 22535011 | 3555, 3559 | 8239 | 12509 | 17195 | 21933 | 24413537 |
| 22535012 | 3557, 3561 | 8241 | 12511 | 17197 | 21935 | 24413538 |
| 22535016 | 3567, 4069 | 8245 | 12515 | 17201, 17233 | 21939 | 24413540 |
| 22535017 | 3569 | 8247 | 12517 | 17203 | 21941 | 24413541 |
| 22535018 | 3571 | 8249 | 12519 | 17205 | 21943 | 24413542 |
| 22535019 | 3573, 3575 | 8251 | 12521 | 17207 | 21945 | 24413543 |
| 22535020 | 3577, 3579 | 8253 | 12529 | 17209 | 21947 | 24413544 |
| 22535021 | 3581 | 8255 | 12531 | 17211 | 21949 | 24413545 |
| 22535073 | 3655 | 8363 | 12563 | 17337, 17371 | 21989 | 24413568 |
| 22535076 | 3659 | 8367, 8675 | 12567 | 17343 | 21995 | 24413571 |
| 22535077 | 3661 | 8369 | 12569, 12571 | 17345 | 21997 | 24413572 |
| 22535022, 22535078 | 3663 | 8371 | 12573 | 17347 | 21999, 22019, | 24413573 |
| | | | | | 22609 | |
| 22535023, 22535079 | 3665 | 8373 | 12575 | 17349 | 22001, 22607 | 24413574 |
| 22535025, 22535081 | 3667 | 8377 | 12579 | 17353 | 22005 | 24413575 |
| 22535082 | 3669 | 8379 | 12581 | 17359 | 22007 | 24413576 |
| 22535026, 22535083 | 3671 | 8381 | 12583 | 17361 | 22009 | 24413577 |
| 22535027, 22535084 | 3673 | 8383 | 12585 | 17363 | 22011, 22677 | 24413578 |
| 22535085 | 3675, 3677, 4173 | 8385 | 12587 | 17365 | 22013 | 24413579 |
| 22535087 | 3681 | 8347, 8389 | 12591 | 17303, 17369 | 22017 | 24413581 |
| 22535090 | 3687 | 8395 | 12595 | 17379 | 22021 | 24413583 |
| 22535092 | 3689 | 8397 | 12597 | 17381 | 22023 | 24413584 |
| 22535093 | 3691, 3693 | 8399 | 12599, 12621 | 17383, 17411, | 22025 | 24413585 |
| | | | | 17413 | | |
| 22535094 | 3695 | 8401 | 12601 | 17385, 17415 | 22027, 22053 | 24413586 |
| 22535095 | 3697 | 8403 | 12603 | 17387 | 22029 | 24413587 |
| 22535096 | 3699 | 8405 | 12607 | 17391 | 22033 | 24413588 |
| 22535099 | 3703 | 8411 | 12611 | 17395, 17397 | 22037 | 24413590 |
| 22535100 | 3705 | 8413 | 12613 | 17399 | 22039, 22055 | 24413591 |
| 22535101 | 3707 | 8415 | 12615 | 17401, 17417 | 22041 | 24413592 |
| 22535102 | 3711 | 8419 | 12619 | 17405, 17421 | 22045 | 24413593 |
| 22535103 | 3713 | 8421 | 12623 | 17423 | 22047 | 24413594 |
| 22535104 | 3715 | 8423 | 12625, 12627 | 17425 | 22049 | 24413595 |
| 22535108 | 3721 | 8429 | 12629 | 17431 | 22051 | 24413598 |
| 22535109 | 3723 | 8431, 8433 | 12631, 12633 | 17433 | 22057 | 24413599 |
| 22535110 | 3725 | 8435 | 12635 | 17435 | 22059 | 24413600 |
| 22535112 | 3729 | 8437, 8497 | 12637 | 17437 | 22061 | 24413601 |
| 22535113 | 3731 | 8439 | 12639 | 17439 | 22063 | 24413602 |
| 22535114 | 3733 | 8441 | 12641 | 17441, 17871 | 22065 | 24413603 |
| 22535116 | 3741 | 8445 | 12645 | 17445 | 22069 | 24413605 |
| 22535117 | 3743 | 8447, 8449 | 12647, 12649 | 17447, 17457 | 22071 | 24413606 |
| 22535118 | 3745 | 8451 | 12651 | 17449, 17459 | 22073 | 24413607 |
| 22535119 | 3747 | 8453 | 12653 | 17451 | 22075 | 24413608 |
| 22535120 | 3749 | 8455 | 12655 | 17453 | 22077, 22079 | 24413610 |
| 22535121 | 3751 | 8457 | 12657 | 17455 | 22081 | 24413611 |
| 22535122 | 3753 | 8459 | 12659 | 17461 | 22083 | 24413612 |
| 22535123 | 3755 | 8461 | 12661 | 17463 | 22085, 22087 | 24413613 |
| 22535124 | 3757 | 8463 | 12663 | 17465 | 22089 | 24413614 |
| 22535126 | 3759 | 8465, 8733 | 12665 | 17467 | 22091 | 24413616 |
| 22533224, 22535134 | 3761 | 4781, 8481 | 9205, 12681, 12683 | 17485 | 18369, 22107 | 24413624 |
| 22535136 | 3765 | 8485 | 12687 | 17489 | 22111 | 24413625 |
| 22535137 | 3767 | 8487 | 12689 | 17491 | 22113 | 24413626 |
| 22535138 | 3769 | 8489 | 12691 | 17493 | 22115 | 24413627 |
| 22535139 | 3771 | 8491 | 12693 | 17495 | 22117 | 24413628 |
| 22535140 | 3773 | 6449, 8493 | 12695 | 17497 | 22119 | 24413629 |
| | 3776 | 6448 | 12698 | 17500 | 22122 | |
| 22535141 | 3779 | 6443, 7353, 8781 | 12699 | 17501 | 22123 | 24413631 |
| 22535142 | 3781 | 6441 | 12701 | 17503 | 21975, 22181 | 24413632 |
| 22535143 | 3783 | 6439 | 12703 | 17505 | 22183 | 24413633 |
| 22535144 | 3785 | 6437 | 12705 | 17507 | 22185 | 24413634 |
| 22535146 | 3787 | 6433 | 12709 | 17511, 17781 | 22189 | 24413635 |
| 22535147 | 3789 | 6431 | 12711 | 17513 | 22137, 22139, | 24413636 |
| | | | | | 22191 | |
| 22535148 | 3791 | 6429 | 12713 | 17515 | 22193 | 24413637 |
| 22535150 | 3793, 4109 | 6427 | 12715 | 17517 | 22195 | 24413638 |
| 22535151 | 3795 | 6425 | 12717 | 17519 | 22197 | 24413639 |
| 22535152 | 3797 | 6423 | 12719 | 17521 | 22199 | 24413640 |
| 22535153 | 3799 | 6421 | 12721 | 17523 | 22201 | 24413641 |
| 22535154 | 3801 | 6419 | 12723 | 17525 | 22203 | 24413642 |
| 22535155 | 3803 | 6417 | 12725 | 17527 | 22141, 22205 | 24413643 |
| 22535156 | 3805 | 8645 | 12727 | 17529 | 22143, 22207 | 24413644 |
| 22535157 | 3807 | 8643 | 12729 | 17531 | 22209 | 24413645 |
| 22535168 | 3809 | 8515 | 12757 | 17553 | 22231 | 24413659 |
| 22535169 | 3811 | 8517 | 12759 | 17555 | 22233 | 24413660 |
| 22535170 | 3813 | 8519 | 12761 | 17557, 17575, | 22235 | 24413661 |
| | | | | 17813 | | |
| 22535171 | 3815 | 8521 | 12763 | 17559 | 22237 | 24413662 |
| 22535173 | 3817 | 8523 | 12765 | 17561 | 22239 | 24413663 |
| 22535174 | 3819 | 8525 | 12767 | 17563, 17577, | 22241 | 24413664 |
| | | | | 17579, 17581, | | |
| | | | | 17883 | | |
| 22535175 | 3821 | 8527 | 12769 | 17565, 17583 | 22243 | 24413665 |
| 22535177 | 3823 | 8531 | 12773 | 17569, 17585 | 22247 | 24413667 |
| 22535178 | 3825 | 8533 | 12775, 12777 | 17571 | 22249 | 24413668 |
| 22535179 | 3827 | 8535 | 12779 | 17573, 17587 | 22251, 22289, | 24413669 |
| | | | | | 22291, 22665 | |
| 22535180 | 3829 | 8537 | 12781 | 17589, 17633 | 22253 | 24413670 |
| 22535181 | 3831 | 8539 | 12783 | 17591, 17635 | 22255 | 24413671 |
| 22535182 | 3833 | 8541 | 12787 | 17593, 17899 | 22257 | 24413672 |
| 22535185 | 3847 | 8547 | 12833 | 17649 | 22303 | 24413675 |
| 22535186 | 3849 | 8549 | 12835 | 17651 | 22305 | 24413676 |
| 22534913, 22535187 | 3851, 3853, 3855, | 8551 | 12837, 12839 | 17653 | 22307 | 24413677 |
| | 3857 | | | | | |
| 22535188 | 3859 | 8553 | 12841, 12843 | 17655 | 22309, 22313 | 24413678 |
| 22535189 | 3861, 3863 | 8555 | 12845 | 17657 | 22311, 22315 | 24413679 |
| 22535190 | 3865 | 6941, 8557 | 12847 | 17659 | 22317 | 24413680 |
| 22535192 | 3867 | 7535, 8559 | 12849 | 17661 | 22319 | 24413681 |
| 22535193 | 3869 | 8561 | 12851, 12873 | 17663 | 22321 | 24413682 |
| 22535194 | 3871 | 8563 | 12853 | 17665 | 22323 | 24413683 |
| 22535195 | 3873 | 8565 | 12855 | 17667 | 22325 | 24413684 |
| 22535196 | 3875 | 8567 | 12857 | 17669 | 22327 | 24413685 |
| 22535197 | 3877 | 8569 | 12859 | 17671, 17673 | 22329 | 24413686 |
| 22535198 | 3879 | 8571 | 12861 | 17675 | 22331 | 24413687 |
| 22535199 | 3881 | 8573 | 12863 | 17677 | 22333 | 24413688 |
| 22535200 | 3883 | 8575 | 12865, 12867 | 17679 | 22335 | 24413689 |
| 22535201 | 3885 | 8577 | 12869 | 17681 | 22337 | 24413690 |
| 22535202 | 3887 | 8579 | 12871, 12875 | 17683 | 22339 | 24413691 |
| 22535205 | 3889 | 8587 | 12881 | 14031 | 22345 | 24413694 |
| 22535206 | 3891 | 8589 | 12883 | 14033 | 22347 | 24413695 |
| 22535207 | 3893 | 8591 | 12885 | 14035 | 22349 | 24413696 |
| 22535208 | 3895 | 8593 | 12887 | 14037 | 22351, 22353, | 24413697 |
| | | | | | 22355 | |
| 22535209 | 3897 | 8595 | 12889 | 14039 | 22357 | 24413698 |
| 22535210 | 3899 | 8597 | 12891 | 14041 | 22359 | 24413699 |
| 22535211 | 3901 | 8599 | 12893 | 14043 | 22361, 22363 | 24413700 |
| 22535212 | 3903 | 8601 | 12895 | 14045 | 22365 | 24413701 |
| 22535213 | 3905 | 8603 | 12897 | 14047, 14049 | 22367 | 24413702 |
| 22535214 | 3907 | 8605 | 12899 | 14051 | 22369 | 24413703 |
| 22535215 | 3909 | 8607 | 12901 | 14053 | 22371 | 24413704 |
| 22535216 | 3911 | 8609 | 12903 | 14055 | 22373 | 24413705 |
| 22535218 | 3913 | 8611 | 12905 | 14057 | 22375 | 24413706 |
| 22535219 | 3915 | 8613 | 12907 | 14059 | 22381 | 24413707 |
| 22533936 | 3953 | 6239 | 10621 | 15117 | 19901 | 23095379 |
| 22535183 | 4019 | 8543 | 12789 | 17595 | 22259 | 24413673 |
| 22535184 | 4021 | 8545 | 12791 | 17597 | 22261 | 24413674 |
| 22534180 | 4033 | 6793 | 11069 | 15613, 15615 | 20375, 20377 | 23095700 |
| 22534179 | 4035 | 6791 | 11067 | 17761 | 20373 | 23095699 |
| 22534178 | 4037 | 6785, 6789 | 9893, 11065 | 15611, 17763, | 20371 | 23095698 |
| | | | | 17943 | | |
| 22534519 | 4119 | 7377, 7381 | 11633 | 16245 | 21069, 21071 | 24413136 |
| 22534199 | 4193 | 6825 | 11101 | 15649 | 20409 | 23095716 |
| 22534198 | 4195 | 6823 | 11099 | 15647 | 20407 | 23095715 |
| 22534191 | 4209 | 6813 | 11089 | 15637 | 20397 | 23095710 |

TABLE VI
Variable coding sequences

| 2603vr | 18rs21 | 515 | cjb111 | coh1 | h36b | NEM316 |
|---|---|---|---|---|---|---|
| 22533006 | 19, 3631 | 4307, 8305, 8323, | — | 14079, 17271 | 22401 | 23094430 |
| | | 8325, 8327, | | | | |
| | | 8329, 8787 | | | | |
| 22533033 | 79 | — | 8827 | 13207 | 17987 | 23094454 |
| 22533050 | 119 | — | 8861 | — | 18021 | — |
| 22533053 | 123 | 4407 | 8865 | 13267 | — | 23094474 |
| 22533062 | 141 | — | 8883 | — | 18059 | 23094483 |
| 22533086 | 187 | 4489 | 8933 | — | 18115 | 23094506 |
| 22533088 | 189 | 4491 | 8935 | — | 18117 | 23094508 |
| 22533089 | 191 | 4493 | 8937 | — | 18119 | 23094509 |
| 22533142 | 277 | 4597, 4599 | 9041 | 13457 | 22175 | — |
| — | 283 | 4601 | 9043 | 13459 | — | — |
| — | 331 | 4649 | 9093 | 13511 | 18251 | 23094580 |
| 22533175 | 353 | 4677 | 9117 | — | 18271 | 23094592 |
| 22533179 | 361 | 4685 | 9125 | 13539 | 18279 | — |
| 22533180 | 363 | 4687 | 9127 | 13541 | — | 23094596 |
| 22533196 | 395 | 4717 | 9157 | 13571 | 18317, 18321 | — |
| — | 455 | — | — | — | 18377 | 23094639 |
| 22533230 | 457 | — | 9215 | — | 18379 | 23094640 |
| 22533234 | 461 | — | 9223 | — | 18385 | 23094643 |
| 22533235 | 463 | — | 9225 | — | 18387 | 23094644 |
| 22533236 | 465 | — | 9227 | — | 18389 | 23094645 |
| 22533238 | 467 | — | 9229 | — | 18391 | 23094646 |
| 22533239 | 469 | — | 9231 | — | 18393 | 23094647 |
| 22533240 | 471 | — | 9233 | — | 18395 | 23094648 |
| 22533242 | 475 | 4815 | 9241, 12547 | — | 18399, 21965, | 23094650 |
| | | | | | 22459 | |
| 22533243 | 477 | 4817 | 9243 | — | 18401 | 23094651 |
| 22533244 | 479 | 4819 | 9245, 9247 | — | 18403 | 23094653 |
| 22533246 | 483 | 4825 | 9251 | — | 18409 | 23094654 |
| 22533247 | 485 | 4827 | 9253 | — | 18411 | 23094655 |
| 22533248 | 487 | 4829 | 9255 | — | 18413 | — |
| 22533249 | 489 | 4831 | 9257 | 13163 | 18415 | — |
| 22533261 | 507 | — | 9479 | 13667 | 18131 | — |
| 22533262 | 509 | — | 9481 | 13669, 13689 | 18133 | — |
| 22533263 | 513 | — | 9483 | — | — | — |
| 22533264 | 515 | — | 9485 | — | 18439 | — |
| 22533324 | 629 | — | 9415 | 13805 | 18559 | 23094723 |
| — | 633 | 4987 | 9419 | 13809 | 18563 | 23094725 |
| 22533275, 22533562, | 879, 1623, 1807, | 6713, 7067 | 12307, 12559, | — | 19961, 19985, | — |
| 22533988, 22534486, | 2331 | | 12755, 13113 | | 20295, 20835, | |
| 22534579, 22535047 | | | | | 21979, 22125, | |
| | | | | | 22177, 22379, | |
| | | | | | 22649 | |
| 22533274, 22533563, | 881, 883, 1621, | 6327, 6711, 7069 | 12309, 12561, | — | 19963, 19987, | — |
| 22533989, 22534485, | 1809, 2333 | | 12753, 13041, | | 20293, 20833, | |
| 22534580, 22535046 | | | 13115 | | 21977, 22127, | |
| | | | | | 22179, 22377 | |
| 22533455 | 893 | 8343 | 9685 | 14169, 17299 | — | 23094896 |
| 22533457 | 895 | — | 9687 | 14171 | — | — |
| — | 899, 901 | 5275, 5277 | 9693 | 14175, 14177 | 18881, 18885, | 23094907 |
| | | | | | 22563 | |
| 22533459 | 905 | 5281 | 9697 | — | 18927 | 23094911 |
| 22533462 | 911 | — | 9703 | 14185 | — | 23094914 |
| 22533466 | 919 | — | — | — | 18941 | — |
| — | 935, 937 | 5315 | 9729 | 14201 | 18959, 18961 | 23094926 |
| 22533484 | 957 | 5335 | 9749 | 14221 | 18981 | — |
| 22533500 | 991 | — | — | — | — | 23094951 |
| 22533525 | 1037 | 5411 | — | — | 19061 | 23094974 |
| — | 1063 | 5439 | 9851 | 14341 | 19197 | 23094987 |
| — | 1123 | 5621 | 9911 | 14397 | 19255 | — |
| — | 1127 | 5625 | 9915 | 14401 | 19259 | — |
| — | 1129 | 5627 | 9917 | 14403 | 19261 | — |
| 22533627 | 1139 | 5617 | — | — | — | — |
| 22533628 | 1149, 3951 | 5619, 5653 | 9937 | 14425 | 19171, 19301 | — |
| 22533653 | 1179 | — | 10059 | 14467 | — | 23095032 |
| 22533654 | 1181 | — | 10061 | 14469 | — | 23095033 |
| 22533655 | 1183 | — | 9965, 10063 | 14471 | — | 23095034 |
| — | 1185 | — | 10065 | — | — | 23095035 |
| 22533656 | 1189 | 5649, 6959 | 9933, 10069, | 14421, 14477, | 19297, 20539 | — |
| | | | 11229 | 15787 | | |
| 22533657 | 1191, 1193, 2323, | — | 10071 | 14479, 15805 | — | 23095038 |
| | 2325 | | | | | |
| — | 1195 | — | 10073 | 14481 | — | 23095039 |
| 22533659 | 1199 | — | 10079 | 14489 | — | 23095041 |
| 22533660 | 1203, 1205 | — | 10081 | 14491 | — | 23095042 |
| 22533661 | 1207, 1211 | — | 10083 | 14493 | — | 23095043 |
| 22533666 | 1223 | — | 10093 | 14503 | 19345 | 23095047 |
| — | 1227 | 5689 | 10099 | 14509 | 19335 | 23095048 |
| 22533667 | 1229 | 5691 | 10101 | 14511 | 19337 | — |
| 22533688 | 1265 | — | 10135 | 14547 | 19373 | — |
| 22533689 | 1267 | — | 10137 | 14549 | 19375 | — |
| 22533690 | 1269 | — | 10139 | 14551 | 19377, 19411 | — |
| 22533692 | 1271 | — | 10141 | 14553, 14555, | 19379 | — |
| | | | | 14595 | | |
| — | 1273 | — | 10143 | 14557 | 19381, 19383 | — |
| — | 1275 | — | 10145 | 14559 | 19385 | — |
| 22533693 | 1279, 1281 | — | 10149, 10151 | 14563, 14565, | 19389, 19391, | — |
| | | | | 14567, 14569, | 19393 | |
| | | | | 14597, 14599 | | |
| 22533695 | 1283 | — | 10153, 10155 | 14571, 14601 | 19395 | — |
| 22533696 | 1285 | 5725 | 10157 | 14573 | 19397 | — |
| — | 1287, 1289, 1291 | 5727 | 10159, 10161 | 14575, 14577 | 19399, 19401 | 23095066 |
| — | 1293 | 5729 | 10163 | 14579 | 19403 | 23095067 |
| — | 1627 | 6087 | 10507 | 14995, 14997 | 19787 | 23095307 |
| 22533877 | 1631 | 6091 | 10511 | — | 19791 | 23095309 |
| 22533892 | 1669 | 6127 | 10539 | 15031 | 19827 | — |
| — | 1731 | 6179 | — | — | 19879 | — |
| — | 1751 | 6241 | 10623 | 15119 | 19903 | 23095380 |
| — | 1753 | 6243 | 10625 | 15121 | 19905 | 23095381 |
| 22533964 | 1765 | — | 10637 | 15133 | 19917 | — |
| — | 1821 | 6339 | 10693 | 15241, 15243 | 19975 | 23095412 |
| 22534042 | 1907 | 6487 | 10793 | 15359 | 20091 | — |
| 22534044 | 1909 | 6491 | — | 15363 | — | 23095506 |
| 22534045 | 1911 | 6493 | — | 15365 | — | 23095508 |
| 22534046 | 1913 | 6495 | 10797 | 15373 | — | 23095509 |
| 22534048 | 1915 | 6499 | — | — | — | 23095510 |
| — | 1923 | 6507 | — | — | — | 23095511 |
| 22534051 | 1925 | 6509 | 10825 | — | — | 23095512 |
| 22534052 | 1927 | 6511 | — | — | — | 23095513 |
| 22534055 | 1937 | 6517 | — | — | — | 23095516 |
| 22534056 | 1939, 1943 | 6519, 6521 | 10833 | — | 20111 | 23095517 |
| 22534063 | 1955 | 6537, 6543 | 10845, 10847 | — | 20125, 20127 | 23095524 |
| — | 1977 | 6565 | 10869 | 15411 | 22495 | 23095535 |
| — | 2143 | — | — | — | — | 23095690 |
| — | 2145 | — | — | — | — | 23095691 |
| — | 2147 | — | — | — | — | 23095692 |
| — | 2151 | — | — | — | — | 23095693 |
| — | 2283 | 6925 | 11201 | 15753 | 20509 | 24412876 |
| — | 2285 | — | — | — | — | 24413553 |
| — | 2293 | 6933 | 11209 | 15761 | 20517 | 24412880 |
| 22534263 | 2311 | — | — | 14715, 15053, | — | — |
| | | | | 15793 | | |
| — | 2313 | 6967 | 11235, 11237 | 15795 | 20545, 20547 | 24412892 |
| 22534265 | 2317 | — | — | 15799 | — | — |
| 22534266 | 2319 | — | — | 15801 | — | — |
| — | 2321 | — | — | 15803 | — | — |
| 22534273 | 2337 | — | 13025, 13053 | 15811 | 20565, 22499 | 24412896 |
| 22534274 | 2339 | — | 13027, 13055 | 15813 | 20567, 22501 | 24412897 |
| 22534275 | 2341 | — | — | — | 20569 | 24412907 |
| 22534276 | 2343 | — | — | — | 20571 | 24412918 |
| 22533658 | 2345 | — | — | 14483 | 20573 | 24412919 |
| 22534278 | 2347 | — | — | — | 20575 | — |
| 22534282 | 2353, 2355 | — | — | — | 20585, 20603 | — |
| 22534283 | 2357 | — | — | — | 20587 | — |
| 22534285 | 2359 | — | — | — | 20589, 20605 | — |
| 22534286 | 2361 | — | — | — | 20591 | — |
| 22534287 | 2363, 2365 | — | — | — | 20593 | — |
| — | 2367 | — | — | — | 20595 | — |
| 22534288 | 2371, 4151, 4177 | — | — | — | 20599 | — |
| 22534290 | 2373 | — | — | 15189 | 20601 | — |
| 22534291 | 2375 | — | — | — | 20607 | — |
| 22534293 | 2377, 4103 | — | 9899, 9901, | — | 20361, 20363, | 23095694 |
| | | | 9903 | | 20365, 20611 | |
| 22534294 | 2379 | — | — | — | 20613 | — |
| 22534295 | 2381 | — | — | — | 20615 | — |
| 22534296 | 2383 | — | — | — | 20617 | — |
| 22534299 | 2385 | — | — | — | 20623 | 24412926 |
| 22534300 | 2387 | — | — | — | 20625 | 24412927 |
| 22534301 | 2389 | — | — | — | 20627 | 24412928 |
| 22534305 | 2391 | — | — | — | 20633, 20635, | 24412932 |
| | | | | | 20637, 20675, | |
| | | | | | 22543 | |
| 22534306 | 2393 | — | — | — | 20639 | 24412934 |
| 22534307 | 2395 | — | — | — | 20641 | 24412935 |
| 22534308 | 2397 | — | — | — | 20643 | 24412936 |
| 22534310 | 2399 | — | — | — | 20645 | 24412937 |
| 22534311 | 2401 | — | — | — | 20647 | 24412938 |
| 22534313 | 2405 | — | — | — | 20651 | 24412940 |
| 22534314 | 2407 | — | — | — | 20655 | 24412942 |
| 22534315 | 2409 | — | — | — | 20657 | 24412943 |
| — | 2411 | — | — | — | 20659 | 24412944 |
| 22534316 | 2413 | — | — | — | 20661 | 24412945 |
| 22534317 | 2415 | — | — | — | 20663 | — |
| 22534318 | 2417 | — | — | — | 20665 | 24412947 |
| 22534321 | 2419 | — | — | — | 20669 | 24412950 |
| 22534322 | 2421 | — | — | — | 20671 | — |
| 22534323 | 2423 | — | — | — | 20673 | 24412952 |
| 22534324 | 2425 | — | — | — | 20677 | 24412953 |
| 22534384 | 2531 | 7105 | 11363 | — | — | 24413007 |
| 22534385 | 2533 | — | 11365 | — | 20801 | 24413008 |
| 22534402 | 2567 | — | 11399 | 16001 | 20841 | 24413024 |
| 22534411 | 2585 | 7159 | — | 16041 | 22521 | 24413033 |
| 22534433 | 2621 | 7201 | 11455 | — | 20897 | 24413055 |
| 22534436 | 2627 | 7207 | 11461 | — | 20903 | 24413058 |
| — | 2631, 2713 | 7211, 7315 | 11465, 11575, | — | 20907, 21015 | 24413060 |
| | | | 11577, 11579 | | | |
| 22534458 | 2667 | — | 11509 | — | — | 24413080 |
| — | 2681 | 7275 | 11543 | 17709 | 20981 | 24413095 |
| 22534475 | 2683 | 7277 | 11545 | — | 20983 | 24413096 |
| 22534476 | 2685 | 7279 | 11547 | — | 20985 | 24413097 |
| 22534479 | 2691 | 7285 | 11553 | — | 20991 | 24413100 |
| 22534481 | 2695 | 7289 | 11557 | — | 20997 | 24413102 |
| 22534482 | 2697 | 7291 | 11559 | — | 20999 | 24413103 |
| 22534484 | 2701 | 7295 | 11563 | — | 21003 | 24413105 |
| — | 2703 | 7297 | 11565 | — | 21005 | 24413106 |
| 22534487 | 2705 | 7299, 7309 | 11567 | — | 21007 | 24413107 |
| 22534488 | 2707 | 7301 | 11569 | — | 21009 | 24413108 |
| 22534489 | 2709 | 7303, 7311 | 11571 | — | 21011, 21119 | 24413109 |
| 22534523 | 2757 | 7387 | 11639 | 16251 | 21077 | — |
| 22534524 | 2759 | — | — | — | — | 24413139 |
| 22534550 | 2795 | 7435 | 11679 | — | 21117 | 24413160 |
| 22534551 | 2797, 2799, 2901, | 4785 | — | 13409, 13411, | 18191 | 23094635 |
| | 2903, 3293, | | | 16075, 16211, | | |
| | 4289 | | | 16409, 16899, | | |
| | | | | 16901, 17407, | | |
| | | | | 17735, 17769, | | |
| | | | | 17771, 17855 | | |
| 22534091 | 2807 | — | — | 14133, 14347, | — | — |
| | | | | 15669, 15765, | | |
| | | | | 16181, 16313, | | |
| | | | | 17087, 17357 | | |
| 22534092 | 2809 | — | — | 14131, 14349, | 20583 | — |
| | | | | 15665, 15667, | | |
| | | | | 15763, 16183, | | |
| | | | | 16315, 17085, | | |
| | | | | 17355, 17711, | | |
| | | | | 17789 | | |
| — | 2841, 2843 | 7473, 7475 | 11717 | 16349 | 21173 | 24413179 |
| — | 2887 | 7519 | 11759 | 16395 | 21219 | 24413201 |
| 22534631 | 2953 | 7591 | 11823 | 16467 | — | 24413230 |
| 22534648 | 2985 | 7625 | 11859 | 16499 | 21317 | — |
| 22534681 | 3033 | 7687 | 11919 | 16577 | 21383 | — |
| — | 3105, 3107 | 7819 | 12033 | 16693 | 21507, 21509 | 24413331 |
| 22534748 | 3123 | 7835 | 12051 | — | 19281 | 24413339 |
| 22534782 | 3193 | — | — | 16779 | 22583 | — |
| 22534817 | 3259 | — | — | 16861, 16891 | — | — |
| 22534831 | 3287 | 7977 | 12253 | 16889 | — | 24413416 |
| 22534836 | 3299 | — | — | — | — | — |
| 22534939 | 3403 | 8087 | 12363 | 17013 | — | 24413467 |
| 22534940 | 3405 | 8089 | 12365 | 17015 | — | 24413468 |
| 22534941 | 3407 | 8091 | 12367 | 17017 | — | 24413469 |
| 22534942 | 3409 | 8093 | 12369 | 17019 | — | 24413470 |
| 22534943 | 3411 | 8095 | 12371 | 17021 | — | 24413471 |
| 22534945 | 3413 | 8097 | 12373 | 17023 | — | 24413472 |
| 22534946 | 3415 | 8099 | 12375 | 17025 | — | 24413473 |
| 22534947 | 3417 | 8101 | 12377 | 17027 | — | 24413474 |
| 22534948 | 3419 | 8103 | 12379 | 17029 | — | 24413475 |
| 22534949 | 3421, 3423 | 8105 | — | 17031, 17033 | 21803 | 24413476 |
| — | 3485, 3489 | 8169, 8171 | 12443 | 17105 | 21863 | 24413505 |
| — | 3527 | 8213 | 12483 | 17145 | 21905, 22551 | 24413523 |
| 22535037 | 3585 | 8259 | — | 17217 | — | 23094905 |
| 22535038 | 3587 | 8261 | — | 17219 | — | — |
| 22535039 | 3589 | 8265, 8271 | — | 17221 | — | 23094903 |
| 22535040 | 3591 | 8273 | — | 17223, 17235 | — | 23094902 |
| 22535041 | 3593, 3595 | 8275 | — | 17237 | — | 23094901 |
| 22535044 | 3597 | 8277 | — | 17239 | — | — |
| 22535045 | 3599, 3601 | 8279 | — | 17241, 17243 | — | — |
| 22535048 | 3603 | 8281 | — | 17245 | — | — |
| 22535049 | 3605 | 8283 | — | 17247 | — | — |
| 22535050 | 3607 | 8285 | — | 17249 | — | — |
| 22535056 | 3609 | — | — | — | — | — |
| 22535057 | 3611 | — | — | 17261, 17263 | — | — |
| 22535058 | 3613 | 8299 | — | 17265 | — | — |
| 22535059 | 3615 | 8301 | — | 17267 | — | — |
| 22535060 | 3617 | 8303 | — | 17269 | — | — |
| 22534280 | 3629, 3643, 4115 | 8351 | — | 17307 | 20579, 22701 | — |
| 22535062 | 3633, 3635 | 6989, 8307 | — | 17273 | — | — |
| 22535063 | 3637 | 6987, 8309 | — | 17275 | — | — |
| 22535064 | 3639 | 8311 | — | 17277 | — | 23094819, 23095132, |
| | | | | | | 23095439, 23095618 |
| 22534279 | 3641 | 8349 | — | 17305 | 20577 | — |
| 22534281 | 3645, 4117 | 8353 | — | 17309, 17851 | 20581 | — |
| 22535070 | 3651 | 8357 | — | 17313 | 22267 | 24413566 |
| 22535071 | 3653 | 8359 | — | 17315 | 21983, 21987 | 24413567 |
| 22535075 | 3657 | — | 12565 | 17341 | 21993 | 24413570 |
| 22535089 | 3685 | 8393 | 12593 | 17377, 17409 | — | 24413582 |
| 22535106 | 3717 | 8425 | — | 17427 | — | 24413596 |
| 22535107 | 3719 | 8427 | — | 17429 | — | 24413597 |
| 22535111 | 3727 | — | — | — | — | — |
| 22535135 | 3763 | 8483 | 12685 | 17487 | 22109 | — |
| — | 3775, 3777 | 6445, 6447 | 12697 | 17499 | 22121 | 24413630 |
| — | 3835 | — | — | 17637, 17823 | 22295 | 23095622 |
| — | 3839 | — | — | 17641 | — | — |
| — | 3841 | — | — | 17643 | — | — |
| — | 3843 | — | — | 17645 | 22299 | — |
| — | 3845 | — | 12831 | 17647 | 22301 | — |
| — | 3917, 4047, 4099, | 5595 | — | — | — | — |
| | 4131, 4223 | | | | | |
| 22533593 | 3943, 4189 | — | — | — | — | — |
| — | 3947 | 8657 | 11305 | 15905 | 20741 | 24412981 |
| 22533579 | 3983 | 5523 | — | — | — | — |
| 22533577 | 3987 | — | — | — | — | — |
| 22533575 | 3997 | — | — | — | — | — |
| 22533572 | 4001 | 5501 | — | — | — | — |
| — | 4041, 4043 | — | — | — | — | 23095696 |
| 22533619 | 4093 | 5601 | — | — | — | — |
| 22533618 | 4095 | 5599, 8795 | — | — | 22481, 22739 | — |
| 22533617 | 4097 | 5597 | — | — | — | — |
| — | 4139 | — | 12799 | 17611 | — | — |
| — | 4141 | — | 12797 | 17607 | 22271 | — |
| — | 4159 | — | 12793 | — | — | — |
| — | 4181, 4255 | 5559, 5561 | — | — | — | — |
| 22533599 | 4183 | 5557 | — | — | — | — |
| 22533598 | 4185 | 5551 | — | — | — | — |
| 22533569 | 4211 | 5495 | — | — | — | — |
| — | 4219, 4229 | 5589 | — | — | — | — |
| — | 4221, 4227 | 5591 | — | — | — | — |
| — | 4225 | 5593 | — | — | — | — |
| — | 4231 | 5587 | — | — | — | — |
| — | 4233 | 5585 | — | — | — | — |
| — | 4235 | 5581 | — | — | — | — |
| — | 4237 | 5579 | — | — | — | — |
| — | 4239 | 5577 | — | — | — | — |
| — | 4241 | 5575 | — | — | — | — |
| — | 4243 | 5573 | — | — | — | — |
| — | 4245 | 5569 | — | — | — | — |
| — | 4247 | 5567 | — | — | — | — |
| — | 4249 | 5565 | — | — | — | — |
| — | 4251 | 8665 | — | — | — | — |
| — | 4253 | 8667 | — | — | — | — |
| — | 4263 | — | 12803 | — | — | — |
| — | 4265 | — | 12805 | — | — | — |
| — | 4267 | — | 12807 | — | — | — |
| — | 4269 | — | 12809 | 17623 | 22283 | — |
| — | 4271 | — | 12813 | 17625 | 22285 | — |
| — | 4273 | — | 12815 | 17627 | 22287 | — |
| — | 4275 | — | 12819 | 17631 | 22507 | — |
| — | 4277 | — | 12821 | 17825 | 22293 | — |
| — | 4287 | — | — | — | 18893, 18895 | — |
| 22533011 | — | — | — | — | 22411 | — |
| 22533087 | — | — | — | — | — | 23094507 |
| 22533109 | — | 4529 | 8975 | 13377, 13387 | 18165 | 23094527 |
| 22533110 | — | 4531 | 8977 | 13379 | 18167 | 23094528 |
| 22533111 | — | 4533 | 8979 | 13381 | 18169 | 23094529 |
| 22533112 | — | 4535 | 8981 | 13383, 13389 | 18173, 18175 | 23094530 |
| 22533113 | — | 4537, 4539 | 8983, 8985 | 13391 | 18177 | 23094531 |
| 22533114 | — | 4541, 4543, 4545 | 8987 | 13393, 13397 | 18179 | 23094532 |
| 22533115 | — | 4547 | 8989 | 13395 | 18181 | 23094533 |
| 22533127 | — | 4575 | 9013 | 13427 | 18207 | — |
| 22533129 | — | 4579 | 9017 | 13431 | 18211 | 23094546 |
| 22533130 | — | 4581 | 9019 | 13433 | 18213 | 23094547 |
| 22533131 | — | 4583 | 9021 | 13435, 17907 | 18215 | 23094548 |
| 22533135 | — | 4589 | 9027 | 13441 | 18221 | 23094551 |
| 22533136 | — | — | 9029 | — | — | 23094552 |
| 22533137 | — | — | 9031 | 13447 | 18223 | 23094553 |
| 22533138 | — | — | 9033 | 13449 | 18225 | 23094554 |
| 22533185 | — | 4695 | 9135 | 13549 | 18295, 19819 | 23094600 |
| 22533232 | — | — | 9219 | — | — | — |
| 22533233 | — | — | 9221 | — | 18383 | 23094642 |
| 22533250 | — | 4833 | 9259 | 13165 | 18417 | — |
| 22533273 | — | 4883 | 9319 | 13693 | 18461 | 23094678 |
| 22533316 | — | 4969 | 9399 | 13773 | 18543 | — |
| 22533378 | — | 5113 | 9505 | 13923 | 18707, 18709 | 23094774 |
| 22533389 | — | 5135 | 9527 | 13945 | 18731 | — |
| 22533419 | — | — | 9587 | — | 18795 | — |
| 22533425 | — | 5215 | 9597 | 14017 | 18805, 18807 | 23094867 |
| 22533510 | — | 8633 | 9795 | 14287 | 19033 | 23094959 |
| 22533566 | — | 5489 | — | — | — | — |
| 22533578 | — | 5521 | — | — | — | — |
| 22533580 | — | 5525 | — | — | — | — |
| 22533581 | — | 5527 | — | — | — | — |
| 22533582 | — | 5529 | — | — | — | — |
| 22533584 | — | 5531 | — | — | — | — |
| 22533585 | — | 5533, 8799 | — | — | — | — |
| 22533586 | — | 5535 | — | — | — | — |
| 22533588 | — | — | — | — | 19285 | — |
| 22533589 | — | 5539 | — | — | — | — |
| 22533590 | — | 5541 | — | — | — | — |
| 22533591 | — | 5543 | — | — | — | — |
| 22533594 | — | 5545 | — | — | — | — |
| 22533595 | — | 5547 | — | — | — | — |
| 22533596 | — | — | — | — | 19109 | — |
| 22533620 | — | 5603 | — | — | — | — |
| 22533621 | — | 5605 | — | — | — | — |
| 22533622 | — | 5607 | — | — | — | — |
| 22533623 | — | 5609 | — | — | 19155 | — |
| 22533624 | — | 5611 | — | — | 19157 | — |
| 22533625 | — | 5613 | — | — | — | — |
| 22533676 | — | — | — | — | — | 23095054 |
| 22533727 | — | 5795 | 10225 | 14659 | 19469, 22651 | 23095095 |
| 22533749 | — | 5839 | 10267 | 14729 | 19529 | 23095165 |
| 22533750 | — | 5841 | 10269 | 14731 | 19531 | — |
| 22533755 | — | 5851 | 10279 | 14741 | 19541 | 23095170 |
| 22533771 | — | 5881 | 10309 | 14775, 14777, | 19571 | 23095210 |
| | | | | 14779 | | |
| 22533785 | — | 5907 | 10337 | 14805 | 19597 | 23095223 |
| 22533788 | — | 5911 | 10341, 10343 | 14811, 14813, | 19601, 19603 | 23095225 |
| | | | | 14815 | | |
| 22533790 | — | 5915 | 10347 | 14819 | 19607 | 23095227 |
| 22533800 | — | 5935, 5975 | 10367 | 14841 | 19631 | 23095237 |
| 22533801 | — | 5937 | 10369 | 14843 | 19633 | 23095238 |
| 22533802 | — | 5939 | 10371 | 14845 | 19635 | 23095239 |
| 22533821 | — | 5977, 8651 | 10405 | 14879, 14891 | 19673 | 23095256 |
| 22533822 | — | 5979 | 10407 | 14893 | 19675 | 23095257 |
| 22533823 | — | 5981 | 10409 | 14895 | 19677 | 23095258 |
| 22533824 | — | 5983 | 10411 | 14897 | 19679 | 23095259 |
| 22533825 | — | 5985 | 10413 | 14899 | 19681 | 23095260 |
| 22533836 | — | 6005, 6007 | 10433 | 14917 | 19701 | 23095269 |
| 22533854 | — | 6043 | — | — | — | 23095286 |
| 22533905 | — | 6151 | 10565 | 15057 | 19851 | 23095334 |
| 22533910 | — | 6161 | 10579 | 15067 | 19861 | 23095339 |
| 22533926 | — | 6183 | — | 15095 | — | — |
| 22533930 | — | 6227 | 10571, 10607 | 15105 | 19887 | 23095373 |
| 22533937 | — | 6195 | 9967 | — | — | — |
| 22533938 | — | 6197 | 9969 | — | — | — |
| 22533940 | — | — | — | 15225 | — | — |
| 22533941 | — | 6199 | 9971 | 15223 | — | — |
| 22533943 | — | 6201 | 9973 | 15221 | — | — |
| 22533945 | — | 6203 | 9975 | 15219 | — | — |
| 22533948 | — | 6205 | 9977 | 15217 | — | — |
| 22533949 | — | 5369, 6207 | 9979 | 15215 | — | — |
| 22533950 | — | — | 9983 | 15213 | — | — |
| 22533951 | — | 6213, 6217 | 9987 | 15209 | — | — |
| 22533952 | — | 6215, 6219 | 9989 | 15203 | — | — |
| 22533953 | — | 6221 | 9991 | 15199 | — | — |
| 22533954 | — | 6223, 8175 | 9993 | 15195, 17857 | — | — |
| 22533955 | — | 8177 | 9995 | 15193 | — | — |
| 22533956 | — | 8179 | 9997 | 15185 | — | — |
| 22533957 | — | 8181 | 9999 | 15183 | — | — |
| 22533975 | — | 6273 | 10655 | 15153 | 19935 | 23095395 |
| 22534019 | — | 6397 | 10743 | 15311 | 20031 | 23095484 |
| 22534020 | — | 6399 | 10745 | 15313 | 20033 | 23095485 |
| 22534021 | — | 6401 | 10747 | 15315 | 20035 | 23095486 |
| 22534024 | — | 6407 | 10753 | 15321 | 20047 | 23095489 |
| 22534043 | — | 6489 | 10817 | 16683 | 20097, 21495 | — |
| 22534047 | — | 6497 | — | — | — | — |
| 22534061 | — | 6531, 6541 | 10841 | 15383 | 20121 | 23095522 |
| 22534075 | — | 6567 | 10871 | 15413, 15415 | 22493 | 23095536 |
| 22534094 | — | — | — | — | — | 23095551 |
| 22534155 | — | — | 12527 | — | — | 23095669 |
| 22534157 | — | 6735 | 11019 | 15577 | 20317 | 23095670 |
| 22534158 | — | 6737 | 11021 | 15579 | 20319 | 23095671 |
| 22534159 | — | 6739 | 11023 | 15581 | 20321 | 23095672 |
| 22534160 | — | 6741, 6743 | 11027, 11029 | 17715, 17717 | 20323, 20325 | 23095673 |
| 22534161 | — | 6745 | 11031 | 17713 | 20327 | 23095674 |
| 22534162 | — | 6747, 6751 | 11033, 11037 | — | 20329, 20333 | 23095675 |
| 22534165 | — | 6753 | 11039 | 15583 | 20335 | 23095678 |
| 22534166 | — | 6755 | 11041 | 15585 | 20337 | 23095679 |
| 22534167 | — | 6757 | 11043 | 15587 | 20339 | 23095680 |
| 22534168 | — | 6759 | 11045 | 15589 | 20341 | 23095681 |
| 22534192 | — | 6815 | 11091 | 15639 | 20399 | 23095711 |
| 22534193 | — | — | 11093 | — | — | — |
| 22534194 | — | — | 11095 | — | — | — |
| 22534195 | — | — | 11097 | — | — | — |
| 22534196 | — | — | 13039 | — | — | — |
| 22534238 | — | — | 11177 | 15731 | — | — |
| 22534251 | — | 6935 | 11211 | 15767 | 20519 | 24412881 |
| 22534253 | — | 6937 | 11213 | 15769 | 20521 | 24412882 |
| 22534254 | — | 6939 | 11215, 11217 | 15771 | 20523 | 24412883 |
| 22534255 | — | — | — | — | — | 24412884 |
| 22534256 | — | — | — | — | — | 24412885 |
| 22534284 | — | — | — | — | 20619 | — |
| 22534292 | — | — | — | — | 20609 | — |
| 22534298 | — | — | — | — | 20621 | 24412925 |
| 22534302 | — | — | — | — | 20629 | 24412929 |
| 22534303 | — | — | — | — | 20631 | 24412930 |
| 22534304 | — | 4799 | — | — | — | 24412931 |
| 22534319 | — | — | — | — | — | 24412948 |
| 22534320 | — | — | — | — | 20667 | 24412949 |
| 22534334 | — | — | 11261 | 15853 | 20695 | — |
| 22534338 | — | 7011 | 11269 | 15865 | 20703 | — |
| 22534358 | — | 8655 | 11307 | 15907 | 20743 | 24412982 |
| 22534374 | — | 7079, 7095 | 11339 | 15949 | 20779 | 24412998 |
| 22534413 | — | — | — | — | — | 24413035 |
| 22534414 | — | — | 11417 | — | — | 24413036 |
| 22534415 | — | 7165 | 11419 | 16045 | 20863 | 24413037 |
| 22534450 | — | 7239 | 11495 | 16131 | 20935 | 24413073 |
| 22534451 | — | 7241 | 11497 | 16133 | 20937 | 24413074 |
| 22534453 | — | 7243 | 11499 | 16135 | 20939 | 24413075 |
| 22534454 | — | 7245 | 11501 | 16137 | 20941 | 24413076 |
| 22534459 | — | — | 13059 | — | 20949 | 24413081 |
| 22534460 | — | — | 13057 | — | 20951 | 24413082 |
| 22534464 | — | 7257 | 11519 | 16153, 16155 | 20961 | 24413086 |
| 22534467 | — | 7261 | 11523 | 16159 | 20965, 20967 | 24413088 |
| 22534468 | — | 7263 | 11525, 11527 | 16161 | 20969 | 24413089 |
| 22534469 | — | 7265 | 11529, 11531 | 16163 | 20971 | 24413090 |
| 22534470 | — | 7267 | 11533 | 16165, 16169, | 20973 | 24413091 |
| | | | | 16171 | | |
| 22534471 | — | 7099, 7269 | 11535 | 16167, 16173, | 20975 | 24413092 |
| | | | | 16175 | | |
| 22534472 | — | 7271 | 11537 | 16177 | 20977 | 24413093 |
| 22534515 | — | 7369 | 11623, 11625 | 16237 | 21061 | 24413132 |
| 22534516 | — | 7371 | 11627 | 16239 | 21063 | 24413133 |
| 22534517 | — | 7373 | 11629 | 16241 | 21065, 21129 | 24413134 |
| 22534518 | — | 7375 | 11631 | 16243 | 21067 | 24413135 |
| 22534520 | — | 7379, 7383 | 11635 | 16247 | 21073 | 24413137 |
| 22534521 | — | 7385 | 11637 | 16249 | 21075 | — |
| 22534525 | — | 7389 | — | 16253 | — | — |
| 22534526 | — | 7391 | — | — | — | — |
| 22534527 | — | 7393 | — | 16255 | — | 24413140 |
| 22534531 | — | 7401 | 11647 | 16263 | 21085 | 24413144 |
| 22534532 | — | 7403 | — | 16265 | — | — |
| 22534534 | — | 7405 | 11649 | 16267 | 21087 | 24413145 |
| 22534536 | — | 7407 | 11651 | 16269 | 21089 | 24413146 |
| 22534539 | — | 7413 | 11657 | 16275 | 21095, 21131 | 24413149 |
| 22534555 | — | — | — | — | — | 24413164 |
| 22534564 | — | 7455 | 11699 | 16327, 16329 | 21155 | 24413170 |
| 22534591 | — | — | — | — | 21201 | — |
| 22534596 | — | 7513 | 11753 | 16387 | 21213 | 24413198 |
| 22534600 | — | 7523 | 11763 | 16399 | 21223 | — |
| 22534663 | — | 7653 | 11885 | 16525 | 21345 | 24413258 |
| 22534664 | — | 7655 | 11887 | 16527 | 21347 | 24413259 |
| 22534665 | — | 7657 | 11889 | 16529 | 21349, 21351 | 24413260 |
| 22534666 | — | 7659 | 11891 | 16531 | 21353, 21355 | 24413261 |
| 22534667 | — | 7661 | 11893 | 16533 | 21357 | 24413262 |
| 22534676 | — | 7679 | 11911 | 16557 | — | — |
| 22534693 | — | 7711 | 11941 | 16603 | 21405 | 24413284 |
| 22534694 | — | 7713 | 11943 | 16605 | 21407, 21409 | 24413285 |
| 22534695 | — | 7715, 7717 | 11945 | 16607 | 21411, 21413 | 24413286 |
| 22534696 | — | 7719 | 11947 | 16609 | 21415 | 24413287 |
| 22534697 | — | 7721 | 11949 | 16611 | 21417, 22637, | 24413288 |
| | | | | | 22639 | |
| 22534698 | — | 7723 | 11951 | 16613 | 21419 | 24413289 |
| 22534706 | — | 7739 | 11969 | 16629 | 21435 | 24413297 |
| 22534707 | — | 7741 | 11971 | 16631 | 21437, 21443 | 24413298 |
| 22534713 | — | 7753 | 11981, 11983, | 16647 | 21453 | 24413303 |
| | | | 11985 | | | |
| 22534714 | — | 7755 | 11987 | 16649 | 21455 | 24413304 |
| 22534716 | — | 7759 | 11991 | 16653 | 21459 | 24413306 |
| 22534718 | — | 7763 | 11997 | 16657 | 21463 | 24413308 |
| 22534734 | — | — | 12023 | 16685 | 21497 | — |
| 22534735 | — | — | 12025 | 16687 | 21499 | — |
| 22534780 | — | 7903 | 12113 | 16775 | 21583 | 24413369 |
| 22534788 | — | 7917, 7919 | 12163 | 16793, 16797 | 21611 | 24413376 |
| 22534789 | — | 6297 | 12165 | 16799 | 21613 | 24413377 |
| 22534856 | — | 8027 | 12299 | 16953 | 21737 | 24413439 |
| 22534883 | — | — | 10039 | — | — | — |
| 22534884 | — | — | 10037, 13021 | — | — | — |
| 22534934 | — | 8077 | 12353 | 17003 | 21793 | — |
| 22534959 | — | 8125 | 12397, 12403 | 17059 | 21823 | 24413485 |
| 22534979 | — | 8167 | 12441 | 17103 | 21861 | 24413504 |
| 22534996 | — | 8207 | 12475 | 17137 | 21895 | — |
| 22535024 | — | 8375 | 12577 | 17351 | 22003 | — |
| 22535033 | — | — | — | — | — | 24413551 |
| 22535034 | — | 8297 | — | — | — | — |
| 22535051 | — | 8287 | — | 17251 | — | — |
| 22535052 | — | 8289 | — | 17253 | — | — |
| 22535053 | — | 8291 | — | 17255 | — | — |
| 22535054 | — | 8293 | — | 17257 | — | — |
| 22535055 | — | 8295 | — | 17259 | — | — |
| 22535072 | — | 8361 | — | 17317 | 21985 | — |
| 22535074 | — | 8365 | — | 17375 | 21991 | 24413569 |
| 22535086 | — | 8387 | 12589 | 17367 | 22015 | 24413580 |
| 22535127 | — | 8467 | 12667 | 17469 | 22093 | 24413617 |
| 22535128 | — | 8469 | 12669 | 17471 | 22095 | 24413618 |
| 22535129 | — | 8471 | 12671 | 17473 | 22097 | 24413619 |
| 22535130 | — | 8473 | 12673 | 17475 | 22099 | 24413620 |
| 22535131 | — | 8475 | 12675 | 17477 | 22101, 22675 | 24413621 |
| 22535132 | — | 8477 | 12677 | 17479 | 22103 | 24413622 |
| 22535133 | — | 8479 | 12679 | 17481, 17483 | 22105 | 24413623 |
| 22535145 | — | 6435 | 12707 | 17509, 17783 | 22187 | — |
| 22535176 | — | 8529 | 12771 | 17567 | 22245 | 24413666 |
| 22535203 | — | 8581, 8583 | 12877 | 17685 | 22341 | 24413692 |
| 22535204 | — | 8585 | 12879 | 14029 | 22343 | 24413693 |
| — | — | 4401 | — | — | — | 23094471 |
| — | — | 4403 | — | — | — | 23094472 |
| — | — | — | 9109 | — | — | 23094588 |
| — | — | 4671 | 9111 | — | — | 23094589 |
| — | — | 4673 | 9113 | — | — | 23094590 |
| — | — | — | — | 16569 | — | 23094789, 23095102, |
| | | | | | | 23095468 |
| — | — | 8341 | — | 17297 | — | 23094897 |
| — | — | 8339 | — | 17295 | — | 23094898 |
| — | — | 8337 | — | 17293 | — | 23094899 |
| — | — | 8263 | — | — | — | 23094904 |
| — | — | — | — | — | 18883 | 23094908 |
| — | — | — | 9707 | — | — | 23094916 |
| — | — | — | 10003 | — | — | 23095036 |
| — | — | 8313, 8687 | — | 17279, 17873 | — | 23095620 |
| — | — | 6817 | — | 15641 | — | 23095712 |
| — | — | 6819 | — | 15643 | 20403 | 23095713 |
| — | — | — | — | 15645 | — | 23095714 |
| — | — | 6977 | 11245 | — | 22485, 22487, | 24412894 |
| | | | | | 22553 | |
| — | — | 8509 | 12745, 13029 | 17547 | 22225, 22503 | 24412898 |
| — | — | 8505 | 12741, 13013, | 17543 | 20553, 20555, | 24412900 |
| | | | 13015 | | 22221 | |
| — | — | 8503 | 12739, 13011 | 17541 | 20557, 22219 | 24412901 |
| — | — | — | 13009 | — | 20559 | 24412902 |
| — | — | — | 13007 | — | 20561 | 24412903 |
| — | — | — | 13005 | — | 20563, 22581 | 24412904 |
| — | — | — | — | — | 20653 | 24412941 |
| — | — | 8659 | 11303 | 15903 | 20739 | 24412980 |
| — | — | 7797 | — | — | — | 24413321 |
| — | — | 7799, 7801 | — | — | — | 24413322 |
| — | — | 7803 | — | — | — | 24413323 |
| — | — | 7805 | — | — | — | 24413324 |
| — | — | 7807 | — | — | — | 24413325 |
| — | — | 7809 | — | — | — | 24413327 |
| — | — | 7813 | — | — | — | 24413328 |
| — | — | 7815 | — | — | — | 24413329 |
| — | — | 7951 | 12223 | — | 21665 | 24413403 |
| — | — | — | 12533 | 17323 | — | 24413546 |
| — | — | — | 12535 | 17325 | — | 24413547 |
| — | — | — | 12541 | 17333 | 21961, 22475 | 24413549, 24413557, |
| | | | | | | 24413564 |
| — | — | — | 12549 | 17329 | 21957, 22461, | 24413561 |
| | | | | | 22465 | |
| — | — | — | 12553 | — | 22469, 22471 | 24413562 |
| — | — | — | — | 17335 | 22477 | 24413565 |
| — | — | 8641 | 12731 | 17533 | 22211 | 24413646 |
| — | — | 8639 | 12733 | 17535 | 22213 | 24413647 |
| — | — | 8499, 8637 | 12735 | 17537 | 22215 | 24413648 |
| — | — | 8501 | 12737 | 17539 | 22217 | 24413649 |
| — | — | 8507 | 12743, 13017 | 17545 | 22223 | 24413652 |
| — | — | 8511 | 12747 | 17549 | 22227 | 24413654 |
| — | — | 8513 | 12749, 12751 | 17551 | 22229 | 24413655 |
| — | — | 4459 | 8901 | 13303 | 18085 | — |
| — | — | 4479 | 8921 | 13323 | 18105 | — |
| — | — | 4793 | — | 17727 | — | — |
| — | — | 4797 | — | 17723 | — | — |
| — | — | 4801 | — | 17721 | — | — |
| — | — | 5285 | — | — | 18931 | — |
| — | — | 5291, 5293 | — | 17691 | — | — |
| — | — | 5629 | 9919 | 14405 | 19263 | — |
| — | — | 5687 | 10097 | 14507 | 19333 | — |
| — | — | 5753 | 10185 | 14619 | 19429 | — |
| — | — | 6181 | 10597 | — | — | — |
| — | — | 6209 | 9981 | — | — | — |
| — | — | 6211 | 9985 | 15211 | — | — |
| — | — | 6821 | — | — | 20405 | — |
| — | — | 6957 | 11227 | 15785 | 20537 | — |
| — | — | 8315 | — | 17281 | — | — |
| — | — | 8317 | — | 17283 | — | — |
| — | — | 8319 | — | 17285 | — | — |
| — | — | 8321, 8331 | — | 17287 | — | — |
| — | — | 8333 | — | 17289 | — | — |
| — | — | 8335 | — | 17291 | — | — |
| — | — | 8673, 8791 | — | 13161 | — | — |
| — | — | — | — | 17619 | 22279 | — |
| — | — | — | — | 17605 | 22269 | — |
| — | — | — | 12551 | — | 22467 | — |
| — | — | — | 12149 | — | 21599, 22457 | — |
| — | — | — | 12147 | — | 22455 | — |
| — | — | — | 12145 | — | 22453 | — |
| — | — | — | 12139 | — | 22447 | — |
| — | — | — | 12137 | — | 22445 | — |
| — | — | — | 12133 | — | 22441 | — |
| — | — | — | 12131 | — | 22439 | — |
| — | — | — | 12129 | — | 22437 | — |
| — | — | — | 12127 | — | 22435 | — |
| — | — | — | 12125 | — | 22433 | — |
| — | — | — | 12119 | — | 21589 | — |
| — | — | — | 12117 | — | 21587 | — |
| — | — | — | 10831 | — | 20109 | — |
| — | — | — | 10827 | — | 20105 | — |
| — | — | — | 9897 | — | 20367 | — |
| — | — | — | 10799 | 15375 | — | — |
| — | — | — | 12135 | — | 22443 | — |
| — | — | — | 12141 | — | 22449 | — |
| — | — | — | 12143 | — | 22451 | — |
| — | — | — | 12481 | 17143, 17947 | 21903 | — |
| — | — | — | 12605 | 17389 | 22031 | — |
| — | — | — | 12817 | 17629 | 22509 | — |
| — | — | — | 12929 | — | 18873 | — |
| — | — | — | 13045 | 15817 | — | — |
| — | — | — | — | 17213 | 21951 | — |
| — | — | — | — | 17615 | 22275 | — |
| — | — | — | — | 17617 | 22277 | — |
| — | — | — | — | 17621 | 22281 | — |

TABLE VII
Phase-variable coding sequences

| | Location | |
|---|---|---|
| Variable gene⁽¹⁾ | of repeat | Position of repeat⁽²⁾ |
| SEQ ID NO: 1821 | 5′ end | 145 |
| SEQ ID NO: 1861 | 5′ end | 2 |
| SEQ ID NO: 957 | 5′ end | 116 |
| SEQ ID NO: 1941 | 5′ end | 13 |
| SEQ ID NO: 2141 | 5′ end | 11 |
| SEQ ID NO: 2863 | 5′ end | 21 |
| SEQ ID NO: 2887 | 5′ end | 295 |
| SEQ ID NO: 3191 | 5′ end | 18 |
| SEQ ID NO: 2447 | 5′ end | 3 |
| SEQ ID NO: 3775 | 5′ end | 301 |
| SEQ ID NO: 6773 | 5′ end | 76 |
| SEQ ID NO: 3723 | Middle | 1120 |
| SEQ ID NO: 2313 | 3′ end | 3185 |
| SEQ ID NO: 719 | Promoter | 40 |
| SEQ ID NO: 4631 | Promoter | 103 |
| SEQ ID NO: 2373 | Promoter | 1 |

⁽¹⁾Given for one strain only; Table II can be used to find any homologs in other strains.
⁽²⁾relative to ATG

### REFERENCES

### The Contents of which are Hereby Incorporated by Reference

[1] Tettelin et al. (2002) Proc Natl Acad Sci USA 99:12391-6.
[2] WO02/34771.
[3] Glaser et al. (2002) Mol Microbiol 45:1499-1513.
[4] WO99/54457.
[5] Geysen et al. (1984) PNAS USA 81:3998-4002.
[6] Carter (1994) Methods Mol Biol 36:207-23.
[7] Jameson, B A et al. 1988, CABIOS 4(1):181-186.
[8] Raddrizzani & Hammer (2000) Brief Bioinform 1(2):179-89.
[9] De Lalla et al. (1999) J. Immunol. 163:1725-29.
[10] Brusic et al. (1998) Bioinformatics 14(2):121-30
[11] Meister et al. (1995) Vaccine 13(6):581-91.
[12] Roberts et al. (1996) AIDS Res Hum Retroviruses 12(7):593-610.
[13] Maksyutov & Zagrebelnaya (1993) Comput Appl Biosci 9(3):291-7.
[14] Feller & de la Cruz (1991) Nature 349(6311):720-1.
[15] Hopp (1993) Peptide Research 6:183-190.
[16] Welling et al. (1985) FEBS Lett. 188:215-218.
[17] Davenport et al. (1995) Immunogenetics 42:392-297.
[18] Bodanszky (1993) Principles of Peptide Synthesis (ISBN: 0387564314).
[19] Fields et al (1997) Meth Enzymol 289: Solid-Phase Peptide Synthesis. ISBN: 0121821900.
[20] Chan & White (2000) Fmoc Solid Phase Peptide Synthesis. ISBN: 0199637245.
[21] Kullmann (1987) Enzymatic Peptide Synthesis. ISBN: 0849368413.
[22] Ibba (1996) Biotechnol Genet Eng Rev 13:197-216.
[23] Breedveld (2000) Lancet 355(9205):735-740.
[24] Gorman & Clark (1990) Semin. Immunol. 2:457-466.
[25] Sambrook et al. (1989) Molecular Cloning: A Laboratory Manual.
[26] Shortprotocols in molecular biology (4th ed, 1999) Ausubel et al. eds. ISBN 0-471-32938-X.
[27] U.S. Pat. No. 5,707,829
[28] Current Protocols in Molecular Biology (F. M. Ausubel et al. eds., 1987) Supplement 30.
[29] EP-B-0509612.
[30] EP-B-0505012.
[31] Golden et al. (2004) Diagn Microbiol InfectDis 50:7-13.
[32] Picard & Bergeron (2004) Eur J Clin Microbiol Infect Dis 23:665-71.
[33] Creti et al (2004) J Clin Microbiol 42:1326-9.
[34] Bergeron & Ke (2004) Expert Rev Mol Med 2001:1-14
[35] Tang et al. (1997) Clin. Chem. 43:2021-2038.
[36] Vaccine Design . . . (1995) eds. Powell & Newman. ISBN: 030644867X. Plenum.
[37] WO00/23105.
[38] WO90/14837.
[39] Podda (2001) Vaccine 19:2673-80.
[40] Frey et al. (2003) Vaccine 21:4234-7.
[41] U.S. Pat. No. 6,299,884.
[42] U.S. Pat. No. 6,451,325
[43] U.S. Pat. No. 5,057,540.
[44] WO96/33739.
[45] EP-A-0109942.
[46] WO96/11711.
[47] WO00/07621.
[48] Barr et al. (1998) Advanced Drug Delivery Reviews 32:247-271.
[49] Sjolanderet et al. (1998) Advanced Drug Delivery Reviews 32:321-338.
[50] NiikLira et al. (2002) Virology 293:273-280.
[51] Lenz et al. (2001) J Immunol 166:5346-5355.
[52] Pinto et al. (2003) J Infect Dis 188:327-338.
[53] Gerber et al. (2001) Virol 75:4752-4760.
[54] WO03/024480
[55] WO03/024481
[56] Gluck et al. (2002) Vaccine 20:B10-B16.
[57] EP-A-0689454.
[58] Johnson et al. (1999) Bioorg Med Chem Lett 9:2273-2278.
[59] Evans et al. (2003) Expert Rev Vaccines 2:219-229.
[60] Meraldi et al. (2003) Vaccine 21:2485-2491.
[61] Pajak et al. (2003) Vaccine 21:836-842.
[62] Kandimalla et al. (2003) Nucleic Acids Research 31:2393-2400.
[63] WO02/26757.
[64] WO99/62923.
[65] Krieg (2003) Nature Medicine 9:831-835.
[66] McCluskie et al. (2002) FEMS Immunology and Medical Microbiology 32:179-185.
[67] WO98/40100.
[68] U.S. Pat. No. 6,207,646.
[69] U.S. Pat. No. 6,239,116.
[70] U.S. Pat. No. 6,429,199.
[71] Kandimalla et al. (2003) Biochemical Society Transactions 31 (part 3):654-658.
[72] Blackwell et al. (2003) J Immunol 170:4061-4068.
[73] Krieg (2002) Trends Immunol 23:64-65.
[74] WO01/95935.
[75] Kandimalla et al. (2003) BBRC 306:948-953.
[76] Bhagat et al. (2003) BBRC 300:853-861.
[77] WO03/035836.
[78] WO95/17211.
[79] WO98/42375.
[80] Beignon et al. (2002) Infect Immun 70:3012-3019.
[81] Pizza et al. (2001) Vaccine 19:2534-2541.
[82] Pizza et al. (2000) Int J Med Microbiol 290:455-461.
[83] Scharton-Kersten et al. (2000) Infect Immun 68:5306-5313.
[84] Ryan et al. (1999) Infect Immun 67:6270-6280.
[85] Partidos et al. (1999) Immunol Lett 67:209-216.
[86] Peppoloni et al. (2003) Expert Rev Vaccines 2:285-293.
[87] Pine et al. (2002) J Control Release 85:263-270.
[88] Domenighini et al. (1995) Mol Microbiol 15:1165-1167.
[89] WO99/40936.
[90] WO99/44636.
[91] Singh et al] (2001) J Cont Release 70:267-276.
[92] WO99/27960.
[93] U.S. Pat. No. 6,090,406
[94] U.S. Pat. No. 5,916,588
[95] EP-A-0626169.
[96] WO99/52549.
[97] WO01/21207.
[98] WO01/21152.
[99] Andrianov et al (1998) Biomaterials 19:109-115.
[100] Payne et al. (1998) Adv Drug Delivery Review 31:185-196.
[101] Stanley (2002) Clin Exp Dermatol 27:571-577.
[102] Jones (2003) Curr Opin Investig Drugs 4:214-218.
[103] WO04/60308
[104] WO04/64759.
[105] WO99/11241.
[106] WO94/00153.
[107] WO98/57659.
[108] European patent applications 0835318, 0735898 and 0761231.
[109] WO03/009869.
[110] Glezen & Alpers (1999) Clin. Infect. Dis. 28:219-224.
[111] Martin et al. (2002) Infect Immun 70:4897-901.
[112] WO2004/041157.
[113] US provisional application.
[114] Almeida & Alpar (1996) J. Drug Targeting 3:455-467.
[115] Agarwal & Mishra (1999) Indian J Exp Biol 37:6-16.
[116] Costantino et al. (1992) Vaccine 10:691-698.
[117] Costantino et al. (1999) Vaccine 17:1251-1263.
[118] International patent application WO03/007985.
[119] Watson (2000) Pediatr Infect Dis J 19:331-332.
[120] Rubin (2000) Pediatr Clin North Am 47:269-285, v.
[121] Jedrzejas (2001) Microbiol Mol Biol Rev 65:187-207.
[122] Bell (2000) Pediatr Infect Dis J 19:1187-1188.
[123] Iwarson (1995) APMIS 103:321-326.
[124] Gerlich et al. (1990) Vaccine 8 Suppl:S63-68 & 79-80.
[125] Vaccines (1988) eds. Plotkin & Mortimer. ISBN 0-7216-1946-0.
[126] Del Guidice et al. (1998) Molecular Aspects of Medicine 19:1-70.
[127] Gustafsson et al. (1996) N. Engl. J. Med. 334:349-355.
[128] Rappuoli et al. (1991) TIBTECH 9:232-238.
[129] Sutter et al. (2000) Pediatr Clin North Am 47:287-308.
Zimmerman & Spann (1999) Am Fam Physician 59:113-118, 125-126.
[131] McMichael (2000) Vaccine 19 Suppl 1:S101-107.
[132] International patent application WO02/34771.
[133] Dale (1999) Infect Dis Clin North Am 13:227-43, viii.
[134] Ferretti et al. (2001) PNAS USA 98: 4658-4663.
[135] Kuroda et al. (2001) Lancet 357(9264):1225-1240; see also pages 1218-1219.
[136] EP-A-0372501
[137] EP-A-0378881
[138] EP-A-0427347
[139] WO93/17712
[140] WO94/03208
[141] WO98/58668
[142] EP-A-0471177
[143] EP-A-0594610.
[144] WO00/56360
[145] WO91/01146
[146] WO00/61761
[147] WO01/72337
[148] Falugi et al. (2001) Eur J Immunol 31:3816-3824.
[149] Baraldo et al, (2004) Infect Immun. 72:4884-7
[150] WO02/091998.
[151] Kuo et al. (1995) Infect Immun 63:2706-13.
[152] Research Disclosure, 453077 (January 2002)
[153] Needleman & Wunsch (1970) J. Mol. Biol. 48, 443-453.
[154] Rice et al. (2000) Trends Genet 16:276-277.
[155] Gennaro (2000) Remington: The Science and Practice of Pharmacy. 20th edition, ISBN: 0683306472.
[156] Methods In Enzymology (S. Colowick and N. Kaplan, eds., Academic Press, Inc.)
[157] Handbook of Experimental Immunology, Vols. I-IV (D. M. Weir and C. C. Blackwell, eds., 1986, Blackwell Scientific Publications)
[158] Sambrook, et al., Molecular Cloning: A Laboratory Manual (2nd Edition, 1989).
[159] Handbook of Surface and Colloidal Chemistry (Birdi, K. S. ed., CRC Press, 1997)
[160] Short Protocols in Molecular Biolog, 4th ed. (Ausubel et al. eds., 1999, John Wiley & Sons)
[161] Molecular Biology Techniques: An Intensive Laboratory Course, (Ream et al., eds., 1998, Academic Press)
[162] PCR (Introduction to Biotechniques Series), 2nd ed. (Newton & Graham eds., 1997, Springer Verlag)
[163] Wren (2000)
[164] Henderson (1999)
[165] Puopolo & Madoff (2003) Mol Microbiol 50:977-91.

1. An isolated polypeptide comprising an amino acid sequence which has at least 75% sequence identity to one or more of the even-numbered amino acid sequences selected from the group consisting of SEQ ID NOS:2-22740.

2. The polypeptide of claim 1, comprising one or more of the even-numbered amino acid sequences selected from the group consisting of SEQ ID NOS:2-22740.

3. An isolated polypeptide comprising a fragment of at least 7 consecutive amino acids from one or more of the even-numbered amino acid sequences selected from the group consisting of SEQ ID NOS:2-22740.

4. The polypeptide of claim 3, wherein the fragment comprises a T-cell or a B-cell epitope from an even-numbered amino acid sequence selected from the group consisting of SEQ ID NOS:2-22740.

5. An antibody which binds to a polypeptide selected from:
(a) a polypeptide comprising an amino acid sequence which has at least 75% sequence identity to one or more of the even-numbered amino acid sequences selected from the group consisting of SEQ ID NOS:2-22740;
(b) a polypeptide comprising one or more of the even-numbered amino acid sequences selected from the group consisting of SEQ ID NOS:2-22740;
(c) a polypeptide comprising a fragment of at least 7 consecutive amino acids from one or more of the even-numbered amino acid sequences selected from the group consisting of SEQ ID NOS:2-22740; and
(d) a polypeptide comprising a fragment of at least 7 consecutive amino acids from one or more of the even-numbered amino acid sequences selected from the group consisting of SEQ ID NOS:2-22740, wherein the fragment comprises a T-cell or a B-cell epitope from an even-numbered amino acid sequence selected from the group consisting of SEQ ID NOS:2-22740.

6. The antibody of claim 5 which is monoclonal.

7. An isolated nucleic acid comprising a nucleotide sequence which has at least 75% sequence identity to one or more of the odd-numbered nucleotide sequences selected from the group consisting of SEQ ID NOS:1-22739.

8. The nucleic acid of claim 7, comprising a nucleotide sequence which is an odd-numbered nucleotide sequence selected from the group consisting of SEQ ID NOS:1-22739.

9. An isolated nucleic acid which can hybridize to the nucleic acid of claim 8 under high stringency conditions.

10. An isolated nucleic acid comprising a fragment of 10 or more consecutive nucleotides from one or more of the odd-numbered nucleotide sequences selected from the group consisting of SEQ ID NOS:1-22739.

11. An isolated nucleic acid encoding a polypeptide selected from:
(a) a polypeptide comprising an amino acid sequence which has at least 75% sequence identity to one or more of the even-numbered amino acid sequences selected from the group consisting of SEQ ID NOS:2-22740;
(b) a polypeptide comprising one or more of the even-numbered amino acid sequences selected from the group consisting of SEQ ID NOS:2-22740;
(c) a polypeptide comprising a fragment of at least 7 consecutive amino acids from one or more of the even-numbered amino acid sequences selected from the group consisting of SEQ ID NOS:2-22740; and
(d) a polypeptide comprising a fragment of at least 7 consecutive amino acids from one or more of the even-numbered amino acid sequences selected from the group consisting of SEQ ID NOS:2-22740, wherein the fragment comprises a T-cell or a B-cell epitope from an even-numbered amino acid sequence selected from the group consisting of SEQ ID NOS:2-22740.

12. A composition comprising:
(a) an active agent selected from:
(i) a polypeptide comprising an amino acid sequence which has at least 75% sequence identity to one or more of the even-numbered amino acid sequences selected from the group consisting of SEQ ID NOS:2-22740;
(ii) a polypeptide comprising one or more of the even-numbered amino acid sequences selected from the group consisting of SEQ ID NOS:2-22740;
(iii) a polypeptide comprising a fragment of at least 7 consecutive amino acids from one or more of the even-numbered amino acid sequences selected from the group consisting of SEQ ID NOS:2-22740; and
(iv) a polypeptide comprising a fragment of at least 7 consecutive amino acids from one or more of the even-numbered amino acid sequences selected from the group consisting of SEQ ID NOS:2-22740, wherein the fragment comprises a T-cell or a B-cell epitope from an even-numbered amino acid sequence selected from the group consisting of SEQ ID NOS:2-22740;
(v) an antibody which binds to (i), (ii), (iii), or (iv);
(vi) an isolated nucleic acid comprising a nucleotide sequence which has at least 75% sequence identity to one or more of the odd-numbered nucleotide sequences selected from the group consisting of SEQ ID NOS:1-22739;
(vii) an isolated nucleic acid comprising a nucleotide sequence which is an odd-numbered nucleotide sequence selected from the group consisting of SEQ ID NOS:1-22739;
(viii) an isolated nucleic acid which can hybridize to (vii) under high stringency conditions;
(ix) a nucleic acid comprising a fragment of 10 or more consecutive nucleotides from one or more of the odd-numbered nucleotide sequences selected from the group consisting of SEQ ID NOS:1-22739; and
(x) a nucleic acid encoding (i), (ii), (iii), or (ix); and
(b) a pharmaceutically acceptable carrier.

13. The composition of claim 12, further comprising a vaccine adjuvant.

14. (canceled)

15. A method of treating a patient, comprising administering to the patient a therapeutically effective amount of the composition of claim 12.

16. (canceled)
